(19) United States
(12) Patent Application Publication
(10) Pub. No.: US 2020/0113995 A1
(43) Pub. Date: Apr. 16, 2020

(54) ARENAVIRUS PARTICLES TO TREAT SOLID TUMORS

(71) Applicant: Hookipa Biotech GmbH, Wien (AT)

(72) Inventors: Klaus Orlinger, Wien (AT); Sarah Schmidt, Wien (AT); Ahmed El-Gazzar, Wien (AT); Lukas Roland Flatz, Schaan (LI); Sandra Stephanie Ring, Unterföhring (DE)

(21) Appl. No.: 16/500,648

(22) PCT Filed: Apr. 6, 2018
(86) PCT No.: PCT/EP2018/058900

§ 371 Date: Oct. 3, 2019

Related U.S. Application Data
(60) Provisional application No. 62/483,067, filed on Apr. 7, 2017.

(51) Int. Cl. A61K 39/12; A61P 35/00; A61K 9/00; A61K 31/664; A61K 39/395

(52) U.S. Cl. CPC A61K 39/12; A61P 35/00; A61K 9/0019; A61K 2039/5256; A61K 39/3955; C12N 2760/10023; C12N 2760/10034; A61K 31/664

(57) ABSTRACT

The present application relates generally to genetically modified arenaviruses that are suitable for treating solid tumors, for example, via intratumoral administration. The arenaviruses described herein may be suitable for vaccines and/or treatment of solid tumors and/or for the use in immunotherapies. In particular, provided herein are methods and compositions for treating a solid tumor by administering a first arenavirus alone or in combination with another agent, including a second arenavirus, wherein the first and/or second arenavirus has been engineered to include a nucleotide sequence encoding a tumor antigen, tumor associated antigen or antigenic fragment thereof.

This application claims benefit of priority from U.S. provisional application No. 62/483,067 filed on Apr. 7, 2017, which is herein incorporated by reference in its entirety.

### 1. INTRODUCTION

The present application relates generally to genetically modified arenaviruses that are suitable for treating solid tumors, for example, via intratumoral administration. The arenaviruses described herein may be suitable for vaccines and/or treatment of solid tumors and/or for the use in immunotherapies. In particular, provided herein are methods and compositions for treating a solid tumor by administering a first arenavirus alone or in combination with another agent, including a second arenavirus, wherein the first and/or second arenavirus has been engineered to include a nucleotide sequence encoding a tumor antigen, tumor associated antigen or antigenic fragment thereof.

### 2. BACKGROUND

The generation of recombinant negative-stranded RNA viruses expressing foreign genes of interest has been pursued for a long time. Recently, it has been shown that an infectious arenavirus particle can be engineered to contain a genome with the ability to amplify and express its genetic material in infected cells but unable to produce further progeny in normal, not genetically engineered cells (i.e., an infectious, replication-deficient arenavirus particle) (International Publication No.: WO 2009/083210 A1 and International Publication No.: WO 2014/140301 A1).

Recently published International Publication No.: WO 2016/075250 A1 shows that arenavirus genomic segments may be engineered to form tri-segmented arenavirus particles with rearrangements of their open reading frames (“ORF”), wherein the arenavirus genomic segment carries a viral ORF in a position other than the wild-type position of the ORF, comprising one L segment and two S segments or two L segments and one S segment that do not recombine into a replication-competent bi-segmented arenavirus particle.

Although treatment options for solid tumors continue to grow beyond the traditional options of surgery and chemotherapy, better treatment options are still needed to more effectively treat solid tumors while minimizing side effects. The potential of viruses as anti-cancer agents was realized several decades ago. Especially, oncolytic viruses have recently experienced a revival as a therapeutic approach.

Though generally non-cytolytic in cell culture, also arenaviruses such as lymphocytic choriomeningitis virus (LCMV), Junin virus (primary isolates and attenuated vaccine strains), Amapari virus, Tacaribe virus and Tamiami virus have long been shown to exhibit anti-tumor effects in various models (Kelly et al., Mol Ther. 2007 April; 15(4):651-9; Molomut et al., Nature. 1965 Dec. 4; 208(5014):948-50; Molomut et al., Cancer Immunol Immunother. 1984; 17(1):56-61; Rankin et al., Cancer Biol Ther. 2003 November-December; 2(6):687-93; Schadler et al., Cancer Res. 2014 Apr. 15; 74(8):2171-81; Mettler et al., Infect Immun. 1982 July; 37(1):23-7). Furthermore, a recent report has emphasized that therapeutically administered arenaviruses can replicate in cancer cells and induces tumor regression by enhancing local immune response (Kalkavan et al., Nat. Commun. 2017 Mar. 1; 8:14447).

However, in spite of encouraging data, existing approaches show clear limitations in efficacy, especially in the treatment of advanced cancers. Moreover, certain viruses entail risks when used as oncolytic agents. Specifically in immunocompromised patients, uncontrolled virus replication bears the potential for significant side effects potentially including life-threatening disease. Therefore, new and better treatment options are urgently required to achieve more effective and sustained tumor control, ideally on the basis of specific immunity, while minimizing the risk for side effects.

### 3. SUMMARY OF THE INVENTION

Provided herein are methods and compositions for treating a solid tumor using an arenavirus particle comprising a nucleotide sequence encoding a tumor antigen, tumor associated antigen or antigenic fragment thereof. Also provided herein are methods and compositions for treating a solid tumor using a first arenavirus particle and a second arenavirus particle comprising a nucleotide sequence encoding a tumor antigen, tumor associated antigen or antigenic fragment thereof.

Provided herein are kits comprising an arenavirus particle comprising a nucleotide sequence encoding a tumor antigen, tumor associated antigen or antigenic fragment thereof and an injection apparatus. Also, in certain embodiments, provided herein are kits comprising a first and second arenavirus particle, wherein the second arenavirus particle comprises a nucleotide sequence encoding a tumor antigen, tumor associated antigen or antigenic fragment thereof.

3.1 Methods for Treating a Solid Tumor with an Arenavirus Particle

Provided herein are methods for treating a solid tumor in a subject comprising injecting an arenavirus particle directly into the tumor (i.e., intratumoral) wherein the arenavirus particle expresses a tumor antigen or tumor-associated antigen or antigenic fragment thereof. In certain embodiments, said arenavirus particle is engineered to contain an arenavirus genomic segment comprising at least one arenavirus ORF in a position other than the wild-type position of said ORF. In certain embodiments, said arenavirus particle is replication competent. In certain embodiments, said arenavirus particle is tri-segmented. In specific embodiments, said tri-segmented genome comprises one L segment and two S segments. In specific embodiments, propagation of said arenavirus particle does not result in a replication-competent bi-segmented viral particle. In specific embodiments, propagation of said arenavirus particle does not result in a replication-competent bi-segmented viral particle after 70 days of persistent infection in mice lacking type I interferon receptor, type II interferon receptor and RAG1 and having been infected with 10⁴ PFU of said arenavirus particle. In specific embodiments, one of said two S segments is an S segment, wherein the ORF encoding the GP is under control of an arenavirus 3′ UTR. In specific embodiments, the arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments, the arenavirus particle is derived from LCMV, JUNV, or PICV. In specific embodiments, said arenavirus particle is derived from LCMV. In more specific embodiments, said LCMV is MP strain, WE strain, Armstrong strain, or Armstrong Clone 13 strain. In specific embodiments, said LCMV is Clone 13 strain with a glycoprotein (GP) from the WE strain. In specific embodiments, said arenavirus particle is derived from JUNV. In more specific embodiments, said JUNV is JUNV vaccine Candid #1 strain, or JUNV vaccine XJ Clone 3 strain. In specific embodiments, said arenavirus particle is derived from PICV. In more specific embodiments, said PICV is strain Munchique CoAn4763 isolate P18, or P2 strain.

In certain embodiments, the arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In specific embodiments, said tumor antigen or tumor associated antigen is selected from the group consisting of artificial fusion protein of HPV16 E7 and E6 proteins, HPV E6, HPV E7, GP100, TRP1, and TRP2. In certain embodiments, the arenavirus particle comprises a nucleotide sequence encoding two, three, four, five, six, seven, eight, nine, ten or more tumor antigens or tumor associated antigens or antigenic fragments thereof.

In certain embodiments, the methods herein further comprise administering a chemotherapeutic agent to said subject. In specific embodiments, said chemotherapeutic agent is cyclophosphamide. In specific embodiments, said arenavirus particle and said chemotherapeutic agent are co-administered simultaneously to the subject. In specific embodiments, said arenavirus particle is administered to the subject prior to administration of said chemotherapeutic agent. In specific embodiments, said arenavirus particle is administered to the subject after administration of said chemotherapeutic agent.

In certain embodiments, said subject is suffering from, is susceptible to, or is at risk for melanoma. In certain embodiments, provided herein are methods for curing, preventing, delaying the occurrence of or preventing the occurrence of a solid tumor in said subject. In certain embodiments, provided herein are methods for curing, preventing, delaying the occurrence of or preventing the occurrence of melanoma in said subject.

In certain embodiments, the methods described herein further comprise administering an immune checkpoint inhibitor to the subject. In specific embodiments, the immune checkpoint inhibitor is an anti-PD-1 antibody. In specific embodiments, the immune checkpoint inhibitor is an anti-PD-L1 antibody. In specific embodiments, said arenavirus particle and said immune checkpoint inhibitor are co-administered simultaneously. In specific embodiments, said arenavirus particle is administered prior to administration of said immune checkpoint inhibitor. In specific embodiments, said arenavirus particle is administered after administration of said immune checkpoint inhibitor.

In certain embodiments, the arenavirus particle comprises a first nucleotide sequence encoding a first human papillomavirus (HPV) antigen. In specific embodiments, the first nucleotide sequence further encodes a second HPV antigen. In specific embodiments, the first HPV antigen is selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof. In specific embodiments, the first and the second HPV antigens are selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof, and wherein the first and the second antigen are not the same.

In certain embodiments, said method comprises injecting a first arenavirus particle, and, after a period of time, injecting a second arenavirus particle. In certain embodiments, said first and second arenavirus particles are identical. In certain embodiments, said first and second arenavirus particles are not identical. In certain embodiments, said method comprises injecting said arenavirus particle(s) two, three, four, or five times.

In certain embodiments, the period of time between injecting a first arenavirus particle and injecting a second arenavirus particle is less than 21 days, including but not limited to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days. In certain embodiments, the period of time between injecting a first arenavirus particle and injecting a second arenavirus particle is greater than 21 days, including but not limited to 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 60 days, 70 days, 80 days, 90 days, or 100 days.

In certain embodiments of the methods provided herein, said step of injecting comprises injecting the same arenavirus particle multiple times. In certain embodiments of the methods provided herein, said step of injecting comprises injecting arenavirus particles derived from the same arenavirus, but expressing different tumor antigens or tumor-associated antigens or antigenic fragments thereof. In certain embodiments of the methods provided herein, said step of injecting comprises injecting arenavirus particles derived from different arenaviruses, but expressing the same tumor antigen or tumor-associated antigen or antigenic fragment thereof. In certain embodiments of the methods provided herein, said step of injecting comprises injecting arenavirus particles derived from different arenaviruses and expressing different tumor antigens or tumor-associated antigens or antigenic fragments thereof. In certain embodiments of the methods provided herein, a first arenavirus particle is administered systemically to the subject prior to said step of injecting. In certain embodiments of the methods provided herein, a second arenavirus particle is administered systemically to the subject after said step of injecting.

In certain embodiments, said systemically administered first and/or second arenavirus particle is replication-deficient. In certain embodiments, said systemically administered first and/or second arenavirus particle is engineered to contain an arenavirus genomic segment comprising at least one arenavirus ORF in a position other than the wild-type position of said ORF. In certain embodiments, said systemically administered first and/or second arenavirus particle is replication competent. In certain embodiments, the genome of said systemically administered first and/or second arenavirus particle is tri-segmented. In specific embodiments, said tri-segmented genome comprises one L segment and two S segments. In specific embodiments, said systemically administered first and/or second arenavirus particle does not result in a replication-competent bi-segmented viral particle. In certain embodiments, propagation of said systemically administered first and/or second arenavirus particle does not result in a replication-competent bi-segmented viral particle after 70 days of persistent infection in mice lacking type I interferon receptor, type II interferon receptor and RAG1 and having been infected with 10⁴ PFU of said arenavirus particle. In specific embodiments, one of said two S segments is an S segment, wherein the ORF encoding the GP is under control of an arenavirus 3′ UTR. In specific embodiments, the first and/or second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments of the methods provided herein, said systemically administered first and/or second arenavirus particle is derived from LCMV, JUNV, or PICV. In certain embodiments, said systemically administered first and/or second arenavirus particle is derived from LCMV. In specific embodiments, said LCMV is MP strain, WE strain, Armstrong strain, or Armstrong Clone 13 strain. In specific embodiments, said LCMV is Clone 13 strain with a glycoprotein (GP) from the WE strain. In certain embodiments, said systemically administered first and/or second arenavirus particle is derived from JUNV. In specific embodiments, said JUNV is JUNV vaccine Candid #1 strain, or JUNV vaccine XJ Clone 3 strain. In certain embodiments, said systemically administered first and/or second arenavirus particle is derived from PICV. In specific embodiments, said PICV is strain Munchique CoAn4763 isolate P18, or P2 strain.

In certain embodiments of the methods provided herein, the systemically administered first and/or second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In specific embodiments, said tumor antigen or tumor associated antigen is selected from the group consisting of artificial fusion protein of HPV16 E7 and E6 proteins, HPV E6, HPV E7, GP100, TRP1, and TRP2. In certain embodiments, the systemically administered first and/or second arenavirus particle comprises a nucleotide sequence encoding two, three, four, five, six, seven, eight, nine, ten or more tumor antigens or tumor associated antigens or antigenic fragments thereof.

In certain embodiments of the methods provided herein, the method further comprises administering a chemotherapeutic agent to said subject. In specific embodiments, said chemotherapeutic agent is cyclophosphamide. In certain embodiments, said systemically administered first and/or second arenavirus particle and said chemotherapeutic agent are co-administered simultaneously to the subject. In certain embodiments, said systemically administered first and/or second arenavirus particle is administered to the subject prior to administration of said chemotherapeutic agent. In certain embodiments, said systemically administered first and/or second arenavirus particle is administered to the subject after administration of said chemotherapeutic agent. In certain embodiments, said subject is suffering from, is susceptible to, or is at risk for melanoma.

In certain embodiments of the methods provided herein, the method further comprises administering an immune checkpoint inhibitor to the subject. In specific embodiments, the immune checkpoint inhibitor is an anti-PD-1 antibody. In specific embodiments, the immune checkpoint inhibitor is an anti-PD-L1 antibody. In certain embodiments, said systemically administered first and/or second arenavirus particle and said immune checkpoint inhibitor are co-administered simultaneously. In certain embodiments, said systemically administered first and/or second arenavirus particle is administered prior to administration of said immune checkpoint inhibitor. In certain embodiments, said systemically administered first and/or second arenavirus particle is administered after administration of said immune checkpoint inhibitor.

In certain embodiments of the methods provided herein, the systemically administered first and/or second arenavirus particle comprises a first nucleotide sequence encoding a first human papillomavirus (HPV) antigen. In certain embodiments, the first nucleotide sequence further encodes a second HPV antigen. In specific embodiments, the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

In specific embodiments, the systemically administered first and the second HPV antigens are selected from the group consisting of:
(v) an HPV16 protein E6, or an antigenic fragment thereof;
(vi) an HPV16 protein E7, or an antigenic fragment thereof;
(vii) an HPV18 protein E6, or an antigenic fragment thereof; and
(viii) an HPV18 protein E7, or an antigenic fragment thereof, wherein the first and the second antigen are not the same.

3.2 Kits for Treating a Solid Tumor with an Arenavirus Particle

Provided herein are kits comprising a container and instructions for use, wherein said container comprises an arenavirus particle in a pharmaceutical composition suitable for injection directly into a solid tumor, wherein said kit further comprises an injection apparatus suitable for performing an injection directly into a solid tumor, wherein said arenavirus particle expresses a tumor antigen or tumor-associated antigen or antigenic fragment thereof. In certain embodiments, said arenavirus particle is engineered to contain an arenavirus genomic segment comprising at least one arenavirus open reading frame (“ORF”) in a position other than the wild-type position of said ORF. In certain embodiments, said arenavirus particle is replication competent. In certain embodiments, said arenavirus particle is tri-segmented. In specific embodiments, said tri-segmented genome comprises one L segment and two S segments. In specific embodiments, propagation of said arenavirus particle does not result in a replication-competent bi-segmented viral particle. In specific embodiments, propagation of said arenavirus particle does not result in a replication-competent bi-segmented viral particle after 70 days of persistent infection in mice lacking type I interferon receptor, type II interferon receptor and RAG1 and having been infected with 10⁴ PFU of said arenavirus particle. In specific embodiments, one of said two S segments is an S segment, wherein the ORF encoding the GP is under control of an arenavirus 3′ UTR. In specific embodiments, the arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments, the arenavirus particle is derived from LCMV, JUNV, or PICV. In specific embodiments, said arenavirus particle is derived from LCMV. In more specific embodiments, said LCMV is MP strain, WE strain, Armstrong strain, or Armstrong Clone 13 strain. In specific embodiments, said LCMV is Clone 13 strain with a glycoprotein (GP) from the WE strain. In specific embodiments, said arenavirus particle is derived from JUNV. In more specific embodiments, said JUNV is JUNV vaccine Candid #1 strain, or JUNV vaccine XJ Clone 3 strain. In specific embodiments, said arenavirus particle is derived from PICV. In more specific embodiments, said PICV is strain Munchique CoAn4763 isolate P18, or P2 strain.

In certain embodiments, the arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In specific embodiments, said tumor antigen or tumor associated antigen is selected from the group consisting of artificial fusion protein of HPV16 E7 and E6 proteins, HPV E6, HPV E7, GP100, TRP1, and TRP2. In certain embodiments, the arenavirus particle comprises a nucleotide sequence encoding two, three, four, five, six, seven, eight, nine, ten or more tumor antigens or tumor associated antigens or antigenic fragments thereof.

In certain embodiments, the kits described herein further comprise a container comprising a chemotherapeutic agent. In specific embodiments, said chemotherapeutic agent is cyclophosphamide. In specific embodiments, said arenavirus particle and said chemotherapeutic agent are formulated for administration simultaneously to a subject. In specific embodiments, said arenavirus particle is formulated for administration to a subject prior to administration of said chemotherapeutic agent. In specific embodiments, said arenavirus particle is formulated for administration to a subject after administration of said chemotherapeutic agent.

In certain embodiments, said subject is suffering from, is susceptible to, or is at risk for melanoma.

In certain embodiments, the kits described herein further comprise a container comprising an immune checkpoint inhibitor. In specific embodiments, said immune checkpoint inhibitor is an anti-PD-1 antibody. In specific embodiments, said immune checkpoint inhibitor is an anti-PD-L1 antibody. In specific embodiments, said arenavirus particle and said immune checkpoint inhibitor are formulated for administration simultaneously to a subject. In specific embodiments, said arenavirus particle is formulated for administration to a subject prior to administration of said immune checkpoint inhibitor. In specific embodiments, said arenavirus particle is formulated for administration to a subject after administration of said immune checkpoint inhibitor.

In certain embodiments of the kits provided herein, the arenavirus particle comprises a first nucleotide sequence encoding a first human papillomavirus (HPV) antigen. In specific embodiments, the first nucleotide sequence further encodes a second HPV antigen. In specific embodiments, the first and the second HPV antigens are selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof, and wherein the first and the second antigen are not the same.

In certain embodiments, said kit comprises injecting a first arenavirus particle, and, after a period of time, injecting a second arenavirus particle. In certain embodiments, said first and second arenavirus particles are identical. In certain embodiments, said first and second arenavirus particles are not identical. In certain embodiments, said method comprises injecting said arenavirus particle(s) two, three, four, or five times.

In certain embodiments, the period of time between injecting a first arenavirus particle and injecting a second arenavirus particle is less than 21 days, including but not limited to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days. In certain embodiments, the period of time between injecting a first arenavirus particle and injecting a second arenavirus particle is greater than 21 days, including but not limited to 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 60 days, 70 days, 80 days, 90 days, or 100 days.

In certain embodiments, the kits described herein further comprise a container comprising a chemotherapeutic agent. In specific embodiments, said chemotherapeutic agent is cyclophosphamide. In specific embodiments, said first and/or second arenavirus particle and said chemotherapeutic agent are formulated for administration simultaneously to a subject. In specific embodiments, said first and/or second arenavirus particle is formulated for administration to a subject prior to administration of said chemotherapeutic agent. In specific embodiments, said first and/or second arenavirus particle is formulated for administration to a subject after administration of said chemotherapeutic agent.

In certain embodiments, the kits described herein further comprise a container comprising an immune checkpoint inhibitor. In specific embodiments, said immune checkpoint inhibitor is an anti-PD-1 antibody. In specific embodiments, said immune checkpoint inhibitor is an anti-PD-L1 antibody. In specific embodiments, said first and/or second arenavirus particle and said immune checkpoint inhibitor are formulated for administration simultaneously to a subject. In specific embodiments, said first and/or second arenavirus particle is formulated for administration to a subject prior to administration of said immune checkpoint inhibitor. In specific embodiments, said first and/or second arenavirus particle is formulated for administration to a subject after administration of said immune checkpoint inhibitor.

In certain embodiments of the kits provided herein, the first and/or second arenavirus particle comprises a first nucleotide sequence encoding a first human papillomavirus (HPV) antigen. In specific embodiments, the first nucleotide sequence further encodes a second HPV antigen. In specific embodiments, the first HPV antigen is selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof. In specific embodiments, the first and the second HPV antigens are selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof, and wherein the first and the second antigen are not the same.

In certain embodiments of the kits provided herein, the kit comprises multiple containers comprising the same arenavirus particle. In certain embodiments, the kit comprises multiple containers, comprising multiple arenavirus particles derived from the same arenavirus, but expressing different tumor antigens or tumor-associated antigens or antigenic fragments thereof. In certain embodiments, the kit comprises multiple containers, comprising multiple arenavirus particles derived from different arenaviruses, but expressing the same tumor antigen or tumor-associated antigen or antigenic fragment thereof. In certain embodiments, the kit comprises multiple containers, comprising multiple arenavirus particles derived from different arenaviruses and expressing different tumor antigens or tumor-associated antigens or antigenic fragments thereof.

In certain embodiments of the kits provided herein, the kit further comprises one or more arenavirus particles in a pharmaceutical composition suitable for intravenous administration. In certain embodiments, said one or more arenavirus particles in a pharmaceutical composition suitable for intravenous administration are engineered to contain an arenavirus genomic segment comprising at least one arenavirus ORF in a position other than the wild-type position of said ORF. In certain embodiments, said one or more arenavirus particles in a pharmaceutical composition suitable for intravenous administration are replication deficient. In certain embodiments, said one or more arenavirus particles in a pharmaceutical composition suitable for intravenous administration are replication competent.

In certain embodiments, the genome of said one or more arenavirus particles in a pharmaceutical composition suitable for intravenous administration is tri-segmented. In certain embodiments, said tri-segmented genome comprises one L segment and two S segments. In certain embodiments, propagation of said one or more arenavirus particles suitable for intravenous administration does not result in a replication-competent bi-segmented viral particle. In certain embodiments, propagation of said one or more arenavirus particles in a pharmaceutical composition suitable for intravenous administration does not result in a replication-competent bi-segmented viral particle after 70 days of persistent infection in mice lacking type I interferon receptor, type II interferon receptor and RAG1 and having been infected with 10⁴ PFU of said arenavirus particle. In certain embodiments, one of said two S segments is an S segment, wherein the ORF encoding the GP is under control of an arenavirus 3′ UTR. In certain embodiments, said one or more arenavirus particles in a pharmaceutical composition suitable for intravenous administration comprise two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments, said one or more arenavirus particles in a pharmaceutical composition suitable for intravenous administration are derived from LCMV, JUNV, or PICV. In certain embodiments, said one or more arenavirus particles in a pharmaceutical composition suitable for intravenous administration are derived from LCMV. In certain embodiments, said LCMV is MP strain, WE strain, Armstrong strain, or Armstrong Clone 13 strain. In certain embodiments, said LCMV is Clone 13 strain with a glycoprotein (GP) from the WE strain. In certain embodiments, said one or more arenavirus particles in a pharmaceutical composition suitable for intravenous administration are derived from JUNV. In certain embodiments, said JUNV is JUNV vaccine Candid #1 strain, or JUNV vaccine XJ Clone 3 strain. In certain embodiments, said one or more arenavirus particles in a pharmaceutical composition suitable for intravenous administration are derived from PICV. In certain embodiments, said PICV is strain Munchique CoAn4763 isolate P18, or P2 strain.

In certain embodiments, said one or more arenavirus particles in a pharmaceutical composition suitable for intravenous administration comprise a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A1, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In certain embodiments, said tumor antigen or tumor associated antigen is selected from the group consisting of artificial fusion protein of HPV16 E7 and E6 proteins, HPV E6, HPV E7, GP100, TRP1, and TRP2. In certain embodiments, said one or more arenavirus particles in a pharmaceutical composition suitable for intravenous administration comprise a nucleotide sequence encoding two, three, four, five, six, seven, eight, nine, ten or more tumor antigens or tumor associated antigens or antigenic fragments thereof.

In certain embodiments, said one or more arenavirus particles in a pharmaceutical composition suitable for intravenous administration comprise a first nucleotide sequence encoding a first human papillomavirus (HPV) antigen. In certain embodiments, the first nucleotide sequence further encodes a second HPV antigen. In certain embodiments, the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

In certain embodiments, the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof, and wherein the first and the second antigen are not the same.

In certain embodiments, said one or more arenavirus particles in a pharmaceutical composition suitable for intravenous administration are formulated for injection prior to said arenavirus particle in a pharmaceutical composition suitable for injection directly into a solid tumor. In certain embodiments, said one or more arenavirus particles in a pharmaceutical composition suitable for intravenous administration are formulated for injection subsequent to said arenavirus particle in a pharmaceutical composition suitable for injection directly into a solid tumor. In certain embodiments, said one or more arenavirus particles in a pharmaceutical composition suitable for intravenous administration are formulated for injection concurrently with said arenavirus particle in a pharmaceutical composition suitable for injection directly into a solid tumor. In certain embodiments, said kit further comprises an apparatus suitable for performing intravenous administration. In certain embodiments, said kit further comprises an injection apparatus suitable for performing an injection directly into a solid tumor.

3.3 Methods for Treating a Solid Tumor with a First and Second Arenavirus Particle

Provided herein are methods for treating a solid tumor comprising (a) administering a first arenavirus particle to the subject, wherein the first arenavirus particle does not express a tumor antigen or tumor-associated antigen or antigenic fragment thereof; and (b) administering a second arenavirus particle to the subject, wherein the second arenavirus particle expresses a tumor antigen or tumor-associated antigen or antigenic fragment thereof. In certain embodiments, the first and second arenavirus particles are injected directly into the tumor. In certain embodiments, the first arenavirus particle is administered intravenously and the second arenavirus particle is injected directly into the tumor. In certain embodiments, the first arenavirus particle is injected directly into the tumor and the second arenavirus particle is administered intravenously.

In certain embodiments, said first arenavirus particle is engineered to contain an arenavirus genomic segment comprising at least one arenavirus open reading frame (“ORF”) in a position other than the wild-type position of said ORF. In certain embodiments, said first arenavirus particle is replication competent. In certain embodiments, the genome of said first arenavirus particle is tri-segmented. In certain embodiments, said second arenavirus particle is engineered to contain an arenavirus genomic segment comprising: (i) a nucleotide sequence encoding a tumor antigen, tumor associated antigen or an antigenic fragment thereof; and (ii) at least one arenavirus ORF in a position other than the wild-type position. In certain embodiments, said second arenavirus particle is replication competent. In certain embodiments, the genome of said second arenavirus particle is tri-segmented. In specific embodiments, said tri-segmented genome comprises one L segment and two S segments. In specific embodiments, propagation of said first or second arenavirus particle does not result in a replication-competent bi-segmented viral particle. In specific embodiments, propagation of said first or second arenavirus particle does not result in a replication-competent bi-segmented viral particle after 70 days of persistent infection in mice lacking type I interferon receptor, type II interferon receptor and recombination activating gene 1 (RAG1) and having been infected with 10⁴ PFU of said first or second arenavirus particle. In specific embodiments, one of said two S segments is an S segment, wherein the ORF encoding the GP is under control of an arenavirus 3′ UTR. In specific embodiments, the second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF. In certain embodiments, said first arenavirus particle and said second arenavirus particle are derived from different arenavirus species.

In certain embodiments, said first and/or second arenavirus particle of the methods described herein is derived from lymphocytic choriomeningitis virus (“LCMV”), Junin virus (“JUNV”), or Pichinde virus (“PICV”). In specific embodiments, said first and/or second arenavirus particle is derived from LCMV. In more specific embodiments, said LCMV is MP strain, WE strain, Armstrong strain, or Armstrong Clone 13 strain. In more specific embodiments, said LCMV is Clone 13 strain with a glycoprotein (GP) from the WE strain. In specific embodiments, said first and/or second arenavirus particle is derived from JUNV. In more specific embodiments, said JUNV is JUNV vaccine Candid #1 strain, or JUNV vaccine XJ Clone 3 strain. In specific embodiments, said first and/or second arenavirus particle is derived from PICV. In more specific embodiments, said PICV is strain Munchique CoAn4763 isolate P18, or P2 strain.

In certain embodiments, the second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In specific embodiments, said tumor antigen or tumor associated antigen is selected from the group consisting of GP100, TRP1, and TRP2. In certain embodiments, the second arenavirus particle comprises a nucleotide sequence encoding two, three, four, five, six, seven, eight, nine, ten or more tumor antigens or tumor associated antigens or antigenic fragments thereof.

In certain embodiments, the methods provided herein further comprise administering a chemotherapeutic agent to said subject. In specific embodiments, said chemotherapeutic agent is cyclophosphamide. In specific embodiments, said first or second arenavirus particle and said chemotherapeutic agent are co-administered simultaneously to the subject. In specific embodiments, said first and second arenavirus particles are administered to the subject prior to administration of said chemotherapeutic agent. In specific embodiments, said first and second arenavirus particles are administered to the subject after administration of said chemotherapeutic agent.

In certain embodiments, said subject is suffering from, is susceptible to, or is at risk for melanoma.

In certain embodiments, the methods provided herein further comprise administering an immune checkpoint inhibitor to the subject. In specific embodiments, the immune checkpoint inhibitor is an anti-PD-1 antibody. In specific embodiments, the immune checkpoint inhibitor is an anti-PD-L1 antibody. In specific embodiments, said first or second arenavirus particle and said immune checkpoint inhibitor are co-administered simultaneously. In specific embodiments, said first and/or second arenavirus particles are administered prior to administration of said immune checkpoint inhibitor. In specific embodiments, said first and/or second arenavirus particles are administered after administration of said immune checkpoint inhibitor.

In certain embodiments, the second arenavirus particle comprises a first nucleotide sequence encoding a first human papillomavirus (HPV) antigen. In certain embodiments, the first nucleotide sequence further encodes a second HPV antigen. In certain embodiments, the first HPV antigen is selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof. In certain embodiments, the first and the second HPV antigens are selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof; and wherein the first and the second antigen are not the same.

In certain embodiments, said first and second arenavirus particles are injected concurrently. In certain embodiments, said first and second arenavirus particles are part of the same composition. In certain embodiments, said first arenavirus particle is injected prior to said second arenavirus particle. In certain embodiments, said first arenavirus particle is injected subsequent to said second arenavirus particle.

In certain embodiments of the methods provided herein, said step of administering said first arenavirus particle comprises administering the same arenavirus particle multiple times. In certain embodiments, said step of administering said first arenavirus particle comprises administering one or more arenavirus particles derived from different arenaviruses.

In certain embodiments of the methods provided herein, said step of administering said second arenavirus particle comprises administering the same arenavirus particle multiple times. In certain embodiments, said step of administering said second arenavirus particle comprises administering one or more arenavirus particles derived from the same arenavirus, but expressing different tumor antigens or tumor-associated antigens or antigenic fragments thereof. In certain embodiments, said step of administering said second arenavirus particle comprises administering one or more arenavirus particles derived from different arenaviruses, but expressing the same tumor antigen or tumor-associated antigen or antigenic fragment thereof. In certain embodiments, said step of administering said second arenavirus particle comprises administering one or more arenavirus particles derived from different arenaviruses and expressing different tumor antigens or tumor-associated antigens or antigenic fragments thereof.

### 3.4 Kits for Treating a Solid Tumor with a First and Second Arenavirus Particle

Provided herein are kits comprising two or more containers and instructions for use, wherein one of said containers comprises a first arenavirus particle in a pharmaceutical composition suitable for injection directly into a solid tumor or suitable for intravenous administration and another of said containers comprises a second arenavirus particle in a pharmaceutical composition suitable for injection directly into a solid tumor or suitable for intravenous administration, and wherein said first arenavirus particle does not express a tumor antigen or tumor-associated antigen or antigenic fragment thereof and said second arenavirus particle expresses a tumor antigen or tumor-associated antigen or antigenic fragment thereof. In certain embodiments, the first and second arenavirus particles are in a pharmaceutical composition suitable for injection directly into a solid tumor. In certain embodiments, the first arenavirus particle is in a pharmaceutical composition suitable for intravenous administration and the second arenavirus particle is in a pharmaceutical composition suitable for injection directly into a solid tumor. In certain embodiments, the first arenavirus particle is in a pharmaceutical composition suitable for injection directly into a solid tumor and the second arenavirus particle is in a pharmaceutical composition suitable for intravenous administration.

In certain embodiments, said first arenavirus particle is engineered to contain an arenavirus genomic segment comprising at least one arenavirus open reading frame (“ORF”) in a position other than the wild-type position of said ORF. In certain embodiments, said first arenavirus particle is replication competent. In certain embodiments, the genome of said first arenavirus particle is tri-segmented. In certain embodiments, said second arenavirus particle is engineered to contain an arenavirus genomic segment comprising: (i) a nucleotide sequence encoding a tumor antigen, tumor associated antigen or an antigenic fragment thereof; and (ii) at least one arenavirus ORF in a position other than the wild-type position. In certain embodiments, said second arenavirus particle is replication competent. In certain embodiments, the genome of said second arenavirus particle is tri-segmented. In specific embodiments, said tri-segmented genome comprises one L segment and two S segments. In specific embodiments, propagation of said first or second arenavirus particle does not result in a replication-competent bi-segmented viral particle. In specific embodiments, propagation of said first or second arenavirus particle does not result in a replication-competent bi-segmented viral particle after 70 days of persistent infection in mice lacking type I interferon receptor, type II interferon receptor and RAG1 and having been infected with 10⁴ PFU of said first or second arenavirus particle. In specific embodiments, one of said two S segments is an S segment, wherein the ORF encoding the GP is under control of an arenavirus 3′ UTR. In specific embodiments, the second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF. In certain embodiments, said first arenavirus particle and said second arenavirus particle are derived from different arenavirus species.

In certain embodiments, said first and/or second arenavirus particle of the methods described herein is derived from lymphocytic choriomeningitis virus (“LCMV”), Junin virus (“JUNV”), or Pichinde virus (“PICV”). In specific embodiments, said first and/or second arenavirus particle is derived from LCMV. In more specific embodiments, said LCMV is MP strain, WE strain, Armstrong strain, or Armstrong Clone 13 strain. In more specific embodiments, said LCMV is Clone 13 strain with a glycoprotein (GP) from the WE strain. In specific embodiments, said first and/or second arenavirus particle is derived from JUNV. In more specific embodiments, said JUNV is JUNV vaccine Candid #1 strain, or JUNV vaccine XJ Clone 3 strain. In specific embodiments, said first and/or second arenavirus particle is derived from PICV. In more specific embodiments, said PICV is strain Munchique CoAn4763 isolate P18, or P2 strain.

In certain embodiments, the second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1. In specific embodiments, said tumor antigen or tumor associated antigen is selected from the group consisting of GP100, TRP1, and TRP2. In certain embodiments, the second arenavirus particle comprises a nucleotide sequence encoding two, three, four, five, six, seven, eight, nine, ten or more tumor antigens or tumor associated antigens or antigenic fragments thereof.

In certain embodiments, the kits described herein further comprise a container comprising a chemotherapeutic agent. In specific embodiments, said chemotherapeutic agent is cyclophosphamide. In specific embodiments, said first and/or second arenavirus particle and said chemotherapeutic agent are formulated for administration simultaneously to a subject. In specific embodiments, said first and/or second arenavirus particles are formulated for administration to a subject prior to administration of said chemotherapeutic agent. In specific embodiments, said first and/or second arenavirus particles are formulated for administration to a subject after administration of said chemotherapeutic agent.

In certain embodiments, the kits described herein further comprise a container comprising an immune checkpoint inhibitor. In specific embodiments, said immune checkpoint inhibitor is an anti-PD-1 antibody. In specific embodiments, said immune checkpoint inhibitor is an anti-PD-L1 antibody. In specific embodiments, said first and/or second arenavirus particle and said immune checkpoint inhibitor are formulated for administration simultaneously to a subject. In specific embodiments, said first and/or second arenavirus particles are formulated for administration to a subject prior to administration of said immune checkpoint inhibitor. In specific embodiments, said first and/or second arenavirus particles are formulated for administration to a subject after administration of said immune checkpoint inhibitor.

In certain embodiments, the second arenavirus particle comprises a first nucleotide sequence encoding a first human papillomavirus (HPV) antigen. In specific embodiments, the first nucleotide sequence further encodes a second HPV antigen. In certain embodiments, the first nucleotide sequence further encodes a second HPV antigen. In certain embodiments, the first HPV antigen is selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof. In certain embodiments, the first and the second HPV antigens are selected from the group consisting of: (i) an HPV16 protein E6, or an antigenic fragment thereof; (ii) an HPV16 protein E7, or an antigenic fragment thereof; (iii) an HPV18 protein E6, or an antigenic fragment thereof; and (iv) an HPV18 protein E7, or an antigenic fragment thereof; and wherein the first and the second antigen are not the same.

In certain embodiments, said first and second arenavirus particles are formulated for concurrent injection directly into the solid tumor. In certain embodiments, said first arenavirus particle is formulated for injection prior to said second arenavirus particle. In certain embodiments, said first arenavirus particle is formulated for injection subsequent to said second arenavirus particle.

In certain embodiments, the kits described herein further comprise an apparatus suitable for performing intravenous administration. In certain embodiments, the kits described herein further comprise an injection apparatus suitable for performing an injection directly into a solid tumor.

In certain embodiments, the kits described herein comprise multiple containers comprising the same first arenavirus particle. In certain embodiments, the kits described herein comprise multiple containers comprising multiple first arenavirus particles derived from different arenaviruses. In certain embodiments, the kits described herein comprise multiple containers comprising the same second arenavirus particle. In certain embodiments, the kits described herein comprise multiple containers comprising multiple second arenavirus particles derived from the same arenavirus, but expressing different tumor antigens or tumor-associated antigens or antigenic fragments thereof. In certain embodiments, the kits described herein comprise multiple containers comprising multiple second arenavirus particles derived from different arenaviruses, but expressing the same tumor antigen or tumor-associated antigen or antigenic fragment thereof. In certain embodiments, the kits described herein comprise multiple containers comprising multiple second arenavirus particles derived from different arenaviruses and expressing different tumor antigens or tumor-associated antigens or antigenic fragments thereof.

### 3.5 Conventions and Abbreviations

| Abbreviation | Convention |
|---|---|
| APC | Antigen presenting cell |
| C-cell | Complementing cell line |
| CD4 | Cluster of differentiation 4 |
| CD8 | Cluster of differentiation 8 |
| CMI | cell-mediated immunity |
| GP | Glycoprotein |
| GS-plasmid | Plasmid expressing genome segments |
| IGR | Intergenic region |
| i.t. | Intratumoral |
| i.v. | Intravenous |
| JUNV | Junin virus |
| L protein | RNA-dependent RNA polymerase |
| L segment | Long segment |
| LCMV | Lymphocytic choriomeningitis virus |
| MHC | Major Histocompatibility Complex |
| NP | Nucleoprotein |
| ORF | Open reading frame |
| PICV | Pichinde virus |
| S segment | Short segment |
| TF-plasmid | Plasmid expressing transacting factors |
| UTR | Untranslated region |
| Z protein | Matrix protein Z |

### 4. BRIEF DESCRIPTION OF THE FIGURES

FIG. 1: Schematic representation of the genomic organization of bi- and tri-segmented LCMV. The bi-segmented genome of wild-type LCMV consists of one S segment encoding the GP and NP and one L segment encoding the Z protein and the L protein (i). Both segments are flanked by the respective 5′ and 3′ UTRs. The genome of recombinant tri-segmented LCMV (r3LCMV) consists of one L and two S segments with one position where to insert a gene of interest (here GFP, which can alternatively be a tumor antigen, tumor associated antigen or antigenic fragment thereof as described herein) into each one of the S segments. r3LCMV-GFP^{natural} (nat) has all viral genes in their natural position (ii), whereas the GP ORF in r3LCMV-GFP^{artificial} (art) is artificially juxtaposed to and expressed under control of the 3′ UTR (iii).

FIG. 2: Comparison of the antitumoral effects of r3LCMV-E7E6 and r3LCMV-GFP, respectively, after intratumoral or systemic administration. (A) Schematic representation of the experimental design described in Example 2. (B) Tumor growth after tumor challenge. (C) Log-rank Kaplan-Meier plot showing the overall survival of the indicated groups. ****Statistically significant (P<0.0001). The tumor volume was calculated according to the formula V=0.5 L×W² where L (length) and W (width) are the long and short diameters of the tumor, respectively. Measurements for each group are included in the plot until >50% of mice per group were sacrificed. Statistically significant differences (*P<0.05, **P<0.005) were determined by comparing tumor volume in the control group (buffer or r3LCMV-GFP) with r3LCMV-E7E6 treated groups until day 32 by Two-way ANOVA. A significant difference was also observed at the time points day 40, 42, 44, 46, and 48 between r3LCMV-E7E6 intravenous (i.v.) and intratumoral (i.t.) administration by Two-way ANOVA.

FIG. 3: Comparison of the antitumoral effects of (i) r3PICV-E7E6 and r3PICV-GFP, respectively, after intratumoral or systemic administration, (ii) r3LCMV-E7E6 and r3PICV-E7E6 and their respective wild-type virus counterparts, and (iii) prime-boost combinations using r3LCMV-E7E6 and r3PICV-E7E6. (A) Schematic representation of the experimental design described in Example 4. (B) Tumor growth after tumor challenge. Subcutaneous tumor growth was monitored every second day starting on day 4 post tumor inoculation. The animals were sacrificed upon reaching the final tumor size of ˜20 mm in diameter. The tumor volume was calculated according to the formula V=0.5 L×W² where L (length) and W (width) are the long and short diameters of the tumor, respectively. (Some tumor bearing mice with defined clinical signs (e.g., ulceration of the tumor or massive body weight loss) had to be sacrificed before reaching the final tumor size according to animal welfare regulations). Measurements for each group are included in the plot until >50% of mice per group were sacrificed. (C) Overall survival of the indicated groups shown by Log-rank Kaplan-Meier plot.

FIG. 4: The antitumoral effect of intratumoral compared to systemic administration of a tri-segmented, replication-competent arenavirus vector expressing the melanoma antigen Trp2, i.e., r3LCMV-Trp2, in tumor bearing mice was evaluated in the B16F10 mouse melanoma model, as described in Example 6. (A) Tumor growth after tumor challenge, and (B) animal survival, were monitored over time. Surviving mice immunized intratumorally with r3LCMV-Trp2 developed autoimmune-related depigmentation at the site of the injection (FIG. 4(C), red arrow) indicating a strong induction of anti-melanocyte directed CD8+ T cell responses.

FIG. 5: Long-time surviving mice from Example 6, i.e., mice cured of B16F10 tumors, acquired tumor-specific immune protection and were protected against re-challenge with B16F10 melanoma cells.

FIG. 6: The antitumoral effect after intratumoral administration of a tri-segmented, replication-competent arenavirus vector expressing either an irrelevant reporter antigen (i.e., r3LCMV-GFP) or the melanoma antigen Trp2 (i.e., r3LCMV-Trp2) were compared in tumor bearing mice in the B16F10 mouse melanoma model, as described in Example 7. Intratumoral administration of r3LCMV-GFP and r3LCMV-Trp2 delayed tumor growth compared to the untreated control animals. However, after initial delayed growth, tumors in mice treated with r3LCMV-GFP increased again and at growth rates comparable to that observed in the control group. In contrast, mice treated with r3LCMV-Trp2 showed a clear and sustained reduction in tumor progression compared to the r3LCMV-GFP or control group.

FIG. 7: Schematic representation of the genomic organization of bi- and tri-segmented lymphocytic choriomeningitis virus (LCMV) and Pichinde virus (PICV). The bi-segmented genome of wild-type LCMV and PICV consists of one S segment encoding the GP and NP and one L segment encoding the Z protein and the L protein. Both segments are flanked by the respective 5′ and 3′ UTRs. The genome of recombinant tri-segmented LCMV (r3LCMV) and recombinant tri-segmented PICV (r3PICV) consists of one L and two S segments with one position where to insert a gene of interest (here GFP, HPV16 E7E6, Trp2 or alternatively any other tumor antigen, tumor associated antigen or antigenic fragment thereof as described herein) into each one of the S segments. In all cases the GP ORF is artificially juxtaposed to and expressed under control of the 3′ UTR.

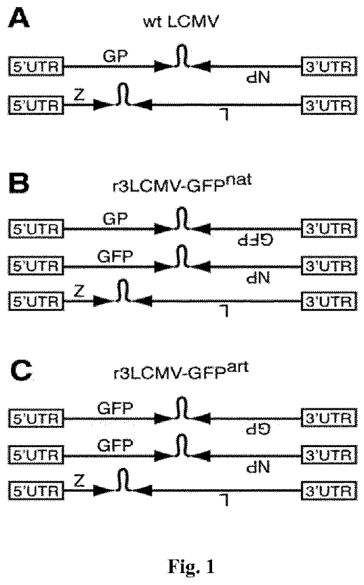

### 5. DETAILED DESCRIPTION OF THE INVENTION

Provided herein are methods and compositions for treating a solid tumor using an arenavirus particle comprising a nucleotide sequence encoding a tumor antigen, tumor associated antigen or antigenic fragment thereof by directly injecting the arenavirus particle into the tumor (i.e., intratumorally). Such methods may further comprise administering the same or different arenavirus particle systemically, for example, intravenously. Also provided herein are methods and compositions for treating a solid tumor using a first arenavirus particle and a second arenavirus particle, wherein the second arenavirus particle comprises a nucleotide sequence encoding a tumor antigen, tumor associated antigen or antigenic fragment thereof, wherein the first and/or second arenavirus particle is injected directly into the tumor.

Provided herein are kits comprising an arenavirus particle comprising a nucleotide sequence encoding a tumor antigen, tumor associated antigen or antigenic fragment thereof and an injection apparatus. Also, in certain embodiments, provided herein are kits comprising a first and second arenavirus particle, wherein the second arenavirus particle comprises a nucleotide sequence encoding a tumor antigen, tumor associated antigen or antigenic fragment thereof.

In certain embodiments, arenavirus particles comprising a nucleotide sequence encoding a tumor antigen, tumor associated antigen or an antigenic fragment thereof can be used as immunotherapies for treating a solid tumor. Such solid tumors may be the result of a neoplastic disease, such as cancer. The term “neoplastic” or “neoplasm” refers to an abnormal new growth of cells or tissue. This abnormal new growth can form a mass, also known as a tumor or neoplasia. A neoplasm includes a benign neoplasm, an in situ neoplasm, a malignant neoplasm, and a neoplasm of uncertain or unknown behavior.

Provided herein are combination treatments for the treatment of solid tumors. Specifically, such combination treatments comprise administering arenavirus particles or viral vectors that comprise a nucleotide sequence encoding one or more tumor antigens, tumor associated antigens or antigenic fragments thereof, optionally in combination with arenavirus particles or viral vectors that do not comprise a nucleotide sequence encoding a foreign antigen. In certain embodiments, said arenavirus particles or viral vectors that do not comprise a nucleotide sequence encoding a foreign antigen comprise a nucleotide comprising a deleted or inactivated viral ORF. In certain embodiments, said arenavirus particles or viral vectors that do not comprise a nucleotide sequence encoding a foreign antigen comprise a nucleotide wherein the UTR is directly fused to the IGR. In certain embodiments, said arenavirus particles or viral vectors that do not comprise a nucleotide sequence encoding a foreign antigen comprise a nucleotide comprising an ORF for a marker, such as GFP. In certain embodiments, said arenavirus particles or viral vectors that do not comprise a nucleotide sequence encoding a foreign antigen comprise a nucleotide comprising a heterologous non-coding sequence. Detailed descriptions of the arenaviruses provided herein, including the nucleotide sequences encoding a tumor antigen, tumor associated antigen or antigenic fragment thereof can be found in Sections 5.1, 5.2, and 5.3. Arenaviruses comprising an open reading frame at a non-natural position are described in Section 5.1. Tri-segmented arenaviruses are described in Section 5.2. Tumor antigens that can be used with the present methods and compositions can be found in Section 5.3. Additionally, methods for generation of arenavirus particles or viral vectors for use in the methods and compositions described herein are described in more detail in Section 5.4.

In addition to administering arenavirus particles or viral vectors to a subject, the immunotherapies for treating a solid tumor provided herein can include a chemotherapeutic agent. “Chemotherapeutic agents” are cytotoxic anti-cancer agents, and can be categorized by their mode of activity within a cell, for example, at what stage they affect the cell cycle (e.g., a mitosis inhibitor). Alternatively, chemotherapeutic agents can be characterized based on ability to cross-link DNA, to intercalate into DNA, or to induce chromosomal aberrations by affecting nucleic acid synthesis (e.g., alkylating agents), among other mechanisms of action. Chemotherapeutic agents can also be characterized based on chemical components or structure (e.g., platinum-based therapeutics). Thus, in certain embodiments, provided herein are methods and compositions for treating a solid tumor using an arenavirus particle or viral vector comprising a nucleotide sequence encoding a tumor antigen, tumor associated antigen or antigenic fragment thereof and a chemotherapeutic agent. Thus, in certain embodiments, provided herein are methods for treating a solid tumor using an arenavirus particle or viral vector comprising a nucleotide sequence encoding a tumor antigen, tumor associated antigen or antigenic fragment thereof, and a chemotherapeutic agent. Also, in certain embodiments, provided herein are compositions comprising an arenavirus particle or viral vector comprising a nucleotide sequence encoding a tumor antigen, tumor associated antigen or antigenic fragment thereof, and a chemotherapeutic agent. In certain embodiments, the arenavirus particle or viral vector provided herein is engineered to contain an arenavirus genomic segment having a nucleotide sequence encoding a tumor antigen, tumor associated antigen or antigenic fragment thereof and at least one arenavirus open reading frame (“ORF”) in a position other than the wild-type position of the ORF. In certain embodiments, the arenavirus particle provided herein is a tri-segmented arenavirus particle or viral vector, which is replication-competent. In still other embodiments, the tri-segmented arenavirus particle or viral vector provided herein, when propagated, does not result in a replication-competent bi-segmented viral particle. Methods and compositions for using an arenavirus particle or viral vector and a chemotherapeutic agent provided herein are described in more detail in Sections 5.6 and 5.7.

In addition to administering arenavirus particles or viral vectors to a subject with or without a chemotherapeutic agent, the immunotherapies for treating a solid tumor provided herein can also include an immune checkpoint modulator. The term “immune checkpoint modulator” (also referred to as a “checkpoint modulator” or as a “checkpoint regulator”) refers to a molecule or to a compound that modulates (e.g., totally or partially reduces, inhibits, interferes with, activates, stimulates, increases, reinforces or supports) the function of one or more checkpoint molecules. Thus, an immune checkpoint modulator may be an immune checkpoint inhibitor or an immune checkpoint activator.

An “immune checkpoint inhibitor” refers to a molecule that inhibits, decreases, or interferes with the activity of a negative checkpoint regulator. In certain embodiments, immune checkpoint inhibitors for use with the methods and compositions disclosed herein can inhibit the activity of a negative checkpoint regulator directly, or decrease the expression of a negative checkpoint regulator, or interfere with the interaction of a negative checkpoint regulator and a binding partner (e.g., a ligand). Immune checkpoint inhibitors for use with the methods and compositions disclosed herein include a protein, a polypeptide, a peptide, an antisense oligonucleotide, an antibody, an antibody fragment, or an inhibitory RNA molecule that targets the expression of a negative checkpoint regulator.

A “negative checkpoint regulator” refers to a molecule that down-regulates immune responses (e.g., T-cell activation) by delivery of a negative signal to T-cells following their engagement by ligands or counter-receptors. Exemplary functions of a negative-checkpoint regulator are to prevent out-of-proportion immune activation, minimize collateral damage, and/or maintain peripheral self-tolerance. In certain embodiments, a negative checkpoint regulator is a ligand or receptor expressed by an antigen presenting cell. In certain embodiments, a negative checkpoint regulator is a ligand or receptor expressed by a T-cell. In certain embodiments, a negative checkpoint regulator is a ligand or receptor expressed by both an antigen presenting cell and a T-cell.

### 5.1 Arenaviruses with an Open Reading Frame in a Non-Natural Position

In certain embodiments, arenaviruses with rearrangements of their ORFs and a nucleotide sequence encoding a tumor antigen, tumor associated antigen or an antigenic fragment thereof provided herein can be used with the methods and compositions provided herein. In certain embodiments, such arenaviruses are replication-competent and infectious. Thus, in certain embodiments, provided herein is an arenavirus genomic segment, wherein the arenavirus genomic segment is engineered to carry an arenavirus ORF in a position other than the position in which the respective gene is found in viruses isolated from the wild, such as LCMV-MP (referred to herein as “wild-type position”) of the ORF (i.e., a non-natural position) and a nucleotide sequence encoding a tumor antigen, tumor associated antigen or an antigenic fragment thereof provided herein.

In certain embodiments, arenaviruses with rearrangements of their ORFs and a nucleotide sequence not encoding a foreign antigen can be used with the methods and compositions provided herein. In certain embodiments, such arenaviruses are replication-competent and infectious. Thus, in certain embodiments, provided herein is an arenavirus genomic segment, wherein the arenavirus genomic segment is engineered to carry an arenavirus ORF in a position other than the position in which the respective gene is found in viruses isolated from the wild, such as LCMV-MP (referred to herein as “wild-type position”) of the ORF (i.e., a non-natural position). In certain embodiments, said arenavirus particles with rearrangements of their ORFs and a nucleotide sequence not encoding a foreign antigen comprise a nucleotide comprising a deleted or inactivated viral ORF. In specific embodiments, said arenavirus particles with rearrangements of their ORFs and a nucleotide sequence not encoding a foreign antigen comprise a nucleotide wherein the untranslated region (UTR) is fused directly to the intergenic region (IGR). In certain embodiments, said arenavirus particles with rearrangements of their ORFs and a nucleotide sequence not encoding a foreign antigen comprise a nucleotide comprising an ORF for a marker, such as GFP. In certain embodiments, said arenavirus particles with rearrangements of their ORFs and a nucleotide sequence not encoding a foreign antigen comprise a nucleotide comprising a heterologous non-coding sequence.

In certain embodiments, the constructs provided herein can have the GP ORF artificially juxtaposed to and expressed under control of the 3′ UTR. In certain embodiments, the arenaviruses described in WO/2016/075250 can be used and are referred to herein as r3LCMV-GFP^{artificial} (art). In certain embodiments, the arenaviruses described in WO/2017/0198726 can be used and are referred to herein as r3PICV-GFP^{artificial} (art).

The wild-type arenavirus genomic segments and ORFs are known in the art. In particular, the arenavirus genome consists of an S segment and an L segment. The S segment carries the ORFs encoding the GP and the NP. The L segment encodes the L protein and the Z protein. Both segments are flanked by the respective 5′ and 3′ UTRs.

In certain embodiments, an arenavirus genomic segment can be engineered to carry two or more arenavirus ORFs in a position other than the wild-type position. In other embodiments, the arenavirus genomic segment can be engineered to carry two arenavirus ORFs, or three arenavirus ORFs, or four arenavirus ORFs in a position other than the wild-type position.

In certain embodiments, an arenavirus genomic segment provided herein can be:
(i) an arenavirus S segment, wherein the ORF encoding the NP is under control of an arenavirus 5′ UTR;
(ii) an arenavirus S segment, wherein the ORF encoding the Z protein is under control of an arenavirus 5′ UTR;
(iii) an arenavirus S segment, wherein the ORF encoding the L protein is under control of an arenavirus 5′ UTR;
(iv) an arenavirus S segment, wherein the ORF encoding the GP is under control of an arenavirus 3′ UTR;
(v) an arenavirus S segment, wherein the ORF encoding the L protein is under control of an arenavirus 3′ UTR;
(vi) an arenavirus S segment, wherein the ORF encoding the Z protein is under control of an arenavirus 3′ UTR;
(vii) an arenavirus L segment, wherein the ORF encoding the GP is under control of an arenavirus 5′ UTR;
(viii) an arenavirus L segment, wherein the ORF encoding the NP is under control of an arenavirus 5′ UTR;
(ix) an arenavirus L segment, wherein the ORF encoding the L protein is under control of an arenavirus 5′ UTR;
(x) an arenavirus L segment, wherein the ORF encoding the GP is under control of an arenavirus 3′ UTR;
(xi) an arenavirus L segment, wherein the ORF encoding the NP is under control of an arenavirus 3′ UTR; and
(xii) an arenavirus L segment, wherein the ORF encoding the Z protein is under control of an arenavirus 3′ UTR.

In certain embodiments, the ORF that is in the non-natural position of the arenavirus genomic segment described herein can be under the control of an arenavirus 3′ UTR or an arenavirus 5′ UTR. In more specific embodiments, the arenavirus 3′ UTR is the 3′ UTR of the arenavirus S segment. In another specific embodiment, the arenavirus 3′ UTR is the 3′UTR of the arenavirus L segment. In more specific embodiments, the arenavirus 5′ UTR is the 5′ UTR of the arenavirus S segment. In other specific embodiments, the 5′ UTR is the 5′ UTR of the L segment.

In other embodiments, the ORF that is in the non-natural position of the arenavirus genomic segment described herein can be under the control of the arenavirus conserved terminal sequence element (the 5′- and 3′-terminal 19-20-nt regions) (see e.g., Perez & de la Torre, 2003, J Virol. 77(2): 1184-1194).

In certain embodiments, the ORF that is in the non-natural position of the arenavirus genomic segment can be under the control of the promoter element of the 5′ UTR (see e.g., Albarino et al., 2011, J Virol., 85(8):4020-4). In another embodiment, the ORF that is in the non-natural position of the arenavirus genomic segment can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment or the L segment. In another specific embodiment, the promoter element of the 3′ UTR is the 3′ UTR the promoter element of the S segment or the L segment.

In certain embodiments, the ORF that is in the non-natural position of the arenavirus genomic segment can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the arenavirus S segment or L segment. In more specific embodiments, the truncated 5′ UTR is the 5′ UTR of the arenavirus S segment or L segment.

Also provided herein, is an arenavirus particle comprising a first genomic segment that has been engineered to carry an ORF in a position other than the wild-type position of the ORF and a second arenavirus genomic segment so that the arenavirus particle comprises an S segment and an L segment. In specific embodiments, the ORF in a position other than the wild-type position of the ORF is one of the arenavirus ORFs.

In certain specific embodiments, the arenavirus particle can comprise a full complement of all four arenavirus ORFs. In specific embodiments, the second arenavirus genomic segment has been engineered to carry an ORF in a position other than the wild-type position of the ORF. In another specific embodiment, the second arenavirus genomic segment can be the wild-type genomic segment (i.e., comprises the ORFs on the segment in the wild-type position).

In certain embodiments, the first arenavirus genomic segment is an L segment and the second arenavirus genomic segment is an S segment. In other embodiments, the first arenavirus genomic segment is an S segment and the second arenavirus genomic segment is an L segment.

Non-limiting examples of the arenavirus particle comprising a genomic segment with an ORF in a position other than the wild-type position of the ORF and a second genomic segment are illustrated in Table 1.

TABLE 1
Arenavirus particle

| Position 1 | Position 2 | Position 3 | Position 4 |
|---|---|---|---|
| GP | NP | L | Z |
| GP | Z | L | NP |
| GP | Z | NP | L |
| GP | L | NP | Z |
| GP | L | Z | NP |
| NP | GP | L | Z |
| NP | GP | Z | L |
| NP | L | GP | Z |
| NP | L | Z | GP |
| NP | Z | GP | L |
| NP | Z | L | GP |
| Z | GP | L | NP |
| Z | GP | NP | L |
| Z | NP | GP | L |
| Z | NP | L | GP |
| Z | L | NP | GP |
| Z | L | GP | NP |
| L | NP | GP | Z |
| L | NP | Z | GP |
| L | GP | Z | NP |
| L | GP | NP | Z |
| L | Z | NP | GP |
| L | Z | GP | NP |

*Position 1 is under the control of an arenavirus S segment 5′ UTR; Position 2 is under the control of an arenavirus S segment 3′ UTR; Position 3 is under the control of an arenavirus L segment 5′ UTR; Position 4 is under the control of an arenavirus L segment 3′ UTR.

Also provided herein, is a cDNA of the arenavirus genomic segment engineered to carry an ORF in a position other than the wild-type position of the ORF and a nucleotide sequence encoding a tumor antigen, tumor associated antigen or an antigenic fragment thereof provided herein. In more specific embodiments, provided herein is a cDNA or a set of cDNAs of an arenavirus genome as set forth in Table 1.

In certain embodiments, a cDNA of the arenavirus genomic segment that is engineered to carry an ORF in a position other than the wild-type position of the ORF is part of or incorporated into a DNA expression vector. In a specific embodiment, a cDNA of the arenavirus genomic segment that is engineered to carry an ORF in a position other than the wild-type position of the ORF is part of or incorporated into a DNA expression vector that facilitates production of an arenavirus genomic segment as described herein. In another embodiment, a cDNA described herein can be incorporated into a plasmid. More detailed description of the cDNAs or nucleic acids and expression systems are provided is Section 5.5. Techniques for the production of a cDNA are routine and conventional techniques of molecular biology and DNA manipulation and production. Any cloning technique known to the skilled artesian can be used. Such as techniques are well known and are available to the skilled artesian in laboratory manuals such as, Sambrook and Russell, Molecular Cloning: A laboratory Manual, 3^{rd} edition, Cold Spring Harbor Laboratory N.Y. (2001).

In certain embodiments, the cDNA of the arenavirus genomic segment that is engineered to carry an ORF in a position other than the wild-type position of the ORF and a nucleotide sequence encoding a tumor antigen, tumor associated antigen or an antigenic fragment thereof provided herein is introduced (e.g., transfected) into a host cell. Thus, in some embodiments provided herein, is a host cell comprising a cDNA of the arenavirus genomic segment that is engineered to carry an ORF in a position other than the wild-type position of the ORF (i.e., a cDNA of the genomic segment) and a nucleotide sequence encoding a tumor antigen, tumor associated antigen or an antigenic fragment thereof provided herein. In other embodiments, the cDNA described herein is part of or can be incorporated into a DNA expression vector and introduced into a host cell. Thus, in some embodiments provided herein is a host cell comprising a cDNA described herein that is incorporated into a vector. In other embodiments, the arenavirus genomic segment described herein is introduced into a host cell.

In certain embodiments, described herein is a method of producing the arenavirus genomic segment comprising a nucleotide sequence encoding a tumor antigen, tumor associated antigen or an antigenic fragment thereof provided herein, wherein the method comprises transcribing the cDNA of the arenavirus genomic segment. In certain embodiments, a viral polymerase protein can be present during transcription of the arenavirus genomic segment in vitro or in vivo.

In certain embodiments transcription of the arenavirus genomic segment is performed using a bi-directional promoter. In other embodiments, transcription of the arenavirus genomic segment is performed using a bi-directional expression cassette (see e.g., Ortiz-Riaño et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively. In yet more specific embodiments the bi-directional expression cassette with pol-I and pol-II promoters read from opposite sides into the L segment and S segment

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promote or a T3 promoter.

In certain embodiments, the method of producing the arenavirus genomic segment can further comprise introducing into a host cell the cDNA of the arenavirus genomic segment comprising a nucleotide sequence encoding a tumor antigen, tumor associated antigen or an antigenic fragment thereof provided herein. In certain embodiments, the method of producing the arenavirus genomic segment can further comprise introducing into a host cell the cDNA of the arenavirus genomic segment comprising a nucleotide sequence encoding a tumor antigen, tumor associated antigen or an antigenic fragment thereof provided herein, wherein the host cell expresses all other components for production of the arenavirus genomic segment; and purifying the arenavirus genomic segment from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 5.5.

In certain embodiments, the arenavirus particle as described herein results in an infectious and replication competent arenavirus particle. In specific embodiments, the arenavirus particle described herein is attenuated. In a particular embodiment, the arenavirus particle is attenuated such that the virus remains, at least partially, able to spread and can replicate in vivo, but can only generate low viral loads resulting in subclinical levels of infection that are non-pathogenic. Such attenuated viruses can be used as an immunogenic composition. Provided herein, are immunogenic compositions that comprise an arenavirus with an ORF in a non-natural position as described in Section 5.7.

### 5.1.1 Replication-Defective Arenavirus Particle with an Open Reading Frame in a Non-Natural Position

In certain embodiments, replication-defective (e.g., replication-deficient) arenavirus particles with a nucleotide sequence encoding a tumor antigen, tumor associated antigen or an antigenic fragment thereof provided herein can be used with the methods and compositions provided herein. In specific embodiments, replication-defective arenavirus particles described herein are used with the methods and compositions provided herein in combination with replication-competent arenavirus particles described herein. In more specific embodiments, replication-defective arenavirus particles described herein are used with the methods and compositions provided herein in combination with replication-competent arenavirus particles described herein, wherein said replication-competent arenavirus particles are injected directly into a tumor in a subject.

In certain embodiments, provided herein is an arenavirus particle in which (i) an ORF is in a position other than the wild-type position of the ORF; and (ii) an ORF encoding GP, NP, Z protein, and L protein has been removed or functionally inactivated such that the resulting virus cannot produce further infectious progeny virus particles. An arenavirus particle comprising a genetically modified genome in which one or more ORFs has been deleted or functionally inactivated can be produced in complementing cells (i.e., cells that express the arenavirus ORF that has been deleted or functionally inactivated). The genetic material of the resulting arenavirus particle can be transferred upon infection of a host cell into the host cell, wherein the genetic material can be expressed and amplified. In addition, the genome of the genetically modified arenavirus particle described herein can encode a heterologous ORF from an organism other than an arenavirus particle.

In certain embodiments, an ORF of the arenavirus is deleted or functionally inactivated and replaced with a nucleotide sequence encoding a tumor antigen or tumor associated antigen as described herein. In a specific embodiment, the ORF that encodes the glycoprotein GP of the arenavirus is deleted or functionally inactivated. In certain embodiments, functional inactivation of a gene eliminates any translation product. In certain embodiments, functional inactivation refers to a genetic alteration that allows some translation, the translation product, however, is not longer functional and cannot replace the wild-type protein.

In certain embodiments, at least one of the four ORFs encoding GP, NP, Z protein, and L protein is removed and replaced with a nucleotide sequence encoding a tumor antigen, tumor associated antigen or an antigenic fragment thereof provided herein. In another embodiment, at least one ORF, at least two ORFs, at least three ORFs, or at least four ORFs encoding GP, NP, Z protein and L protein can be removed and replaced with a nucleotide sequence encoding a tumor antigen, tumor associated antigen or an antigenic fragment thereof provided herein. In specific embodiments, only one of the four ORFs encoding GP, NP, Z protein, and L protein is removed and replaced with a nucleotide sequence encoding a tumor antigen, tumor associated antigen or an antigenic fragment thereof provided herein. In more specific embodiments, the ORF that encodes GP of the arenavirus genomic segment is removed. In another specific embodiment, the ORF that encodes the NP of the arenavirus genomic segment is removed. In more specific embodiments, the ORF that encodes the Z protein of the arenavirus genomic segment is removed. In yet another specific embodiment, the ORF encoding the L protein is removed.

Thus, in certain embodiments, the arenavirus particle provided herein comprises a genomic segment that (i) is engineered to carry an ORF in a non-natural position; (ii) an ORF encoding GP, NP, Z protein, or L protein is removed; (iii) the ORF that is removed is replaced with a nucleotide sequence encoding a tumor antigen, tumor associated antigen or an antigenic fragment thereof provided herein.

In certain embodiments, the fragment of the tumor antigen or tumor associated antigen is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding an antigenic fragment provided herein is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the nucleotide sequence encoding an antigenic fragment provided herein is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence encoding a tumor antigen, tumor associated antigen or an antigenic fragment thereof provided herein.

In certain embodiments, the growth and infectivity of the arenavirus particle is not affected by the nucleotide sequence encoding a tumor antigen, tumor associated antigen or an antigenic fragment thereof provided herein.

Techniques known to one skilled in the art may be used to produce an arenavirus particle comprising an arenavirus genomic segment engineered to carry an arenavirus ORF in a position other than the wild-type position and a nucleotide sequence encoding a tumor antigen, tumor associated antigen or an antigenic fragment thereof provided herein. For example, reverse genetics techniques may be used to generate such arenavirus particle. In other embodiments, the replication-defective arenavirus particle (i.e., the arenavirus genomic segment engineered to carry an arenavirus ORF in a position other than the wild-type position, wherein an ORF encoding GP, NP, Z protein, L protein, has been deleted) can be produced in a complementing cell.

In certain embodiments, an arenavirus particle or arenavirus genomic segment provided herein comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof; Ubiquitin or a fragment thereof; Granulocyte-Macrophage Colony-Stimulating Factor (GM-CSF), or a fragment thereof; Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof; CD40 ligand or an antigenic fragment thereof; or Fms-related tyrosine kinase 3 (Flt3) ligand or an antigenic fragment thereof.

In certain embodiments, the arenavirus genomic segment or the arenavirus particle used according to the present application can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

In certain embodiments, the arenavirus particle as described herein is suitable for use as a vaccine and methods of using such arenavirus particle in a vaccination and treatment for a neoplastic disease, for example, cancer, is provided. More detailed description of the methods of using the arenavirus particle described herein is provided in Section 5.6

In certain embodiments, the arenavirus particle as described herein is suitable for use as a pharmaceutical composition and methods of using such arenavirus particle in a vaccination and treatment for a neoplastic disease, for example, cancer, is provided. More detailed description of the methods of using the arenavirus particle described herein is provided in Section 5.7.

### 5.2 Tri-Segmented Arenavirus Particle

Exemplary tri-segmented arenavirus particles are described, for example, International Patent Application Publication WO 2016/075250, which is incorporated by reference herein in its entirety.

In certain embodiments, tri-segmented arenavirus particles with rearrangements of their ORFs and a nucleotide sequence encoding a tumor antigen, tumor associated antigen or an antigenic fragment thereof provided herein can be used with the methods and compositions provided herein. In one aspect, provided herein is a tri-segmented arenavirus particle comprising one L segment and two S segments or two L segments and one S segment. In certain embodiments, the tri-segmented arenavirus particle does not recombine into a replication competent bi-segmented arenavirus particle. More specifically, in certain embodiments, two of the genomic segments (e.g., the two S segments or the two L segments, respectively) cannot recombine in a way to yield a single viral segment that could replace the two parent segments. In specific embodiments, the tri-segmented arenavirus particle comprises an ORF in a position other than the wild-type position of the ORF and a nucleotide sequence encoding a tumor antigen, tumor associated antigen or an antigenic fragment thereof provided herein. In yet another specific embodiment, the tri-segmented arenavirus particle comprises all four arenavirus ORFs. Thus, in certain embodiments, the tri-segmented arenavirus particle is replication competent and infectious. In other embodiments, the tri-segmented arenavirus particle lacks one of the four arenavirus ORFs. Thus, in certain embodiments, the tri-segmented arenavirus particle is infectious but unable to produce further infectious progeny in non-complementing cells.

In certain embodiments, tri-segmented arenavirus particles with rearrangements of their ORFs comprising a nucleotide sequence not encoding a foreign antigen can be used with the methods and compositions provided herein. In specific embodiments, the tri-segmented arenavirus particle comprises an ORF in a position other than the wild-type position of the ORF and a nucleotide sequence comprising a deleted or inactivated viral ORF. In specific embodiments, the tri-segmented arenavirus particle comprises an ORF in a position other than the wild-type position of the ORF and a nucleotide sequence wherein the untranslated region (UTR) is fused directly to the intergenic region (IGR). In specific embodiments, the tri-segmented arenavirus particle comprises an ORF in a position other than the wild-type position of the ORF and a nucleotide sequence comprising an ORF for a marker, such as GFP. In specific embodiments, the tri-segmented arenavirus particle comprises an ORF in a position other than the wild-type position of the ORF and a nucleotide sequence comprising a heterologous non-coding sequence. In yet another specific embodiment, the tri-segmented arenavirus particle comprises all four arenavirus ORFs. Thus, in certain embodiments, the tri-segmented arenavirus particle is replication competent and infectious. In other embodiments, the tri-segmented arenavirus particle lacks one of the four arenavirus ORFs. Thus, in certain embodiments, the tri-segmented arenavirus particle is infectious but unable to produce further infectious progeny in non-complementing cells.

In certain embodiments, the ORF encoding GP, NP, Z protein, or the L protein of the tri-segmented arenavirus particle described herein can be under the control of an arenavirus 3′ UTR or an arenavirus 5′ UTR. In more specific embodiments, the tri-segmented arenavirus 3′ UTR is the 3′ UTR of an arenavirus S segment(s). In another specific embodiment, the tri-segmented arenavirus 3′ UTR is the 3′ UTR of a tri-segmented arenavirus L segment(s). In more specific embodiments, the tri-segmented arenavirus 5′ UTR is the 5′ UTR of an arenavirus S segment(s). In other specific embodiments, the 5′ UTR is the 5′ UTR of the L segment(s).

In other embodiments, the ORF encoding GP, NP, Z protein, or the L protein of tri-segmented arenavirus particle described herein can be under the control of the arenavirus conserved terminal sequence element (the 5′- and 3′-terminal 19-20-nt regions) (see e.g., Perez & de la Torre, 2003, J Virol. 77(2): 1184-1194).

In certain embodiments, the ORF encoding GP, NP, Z protein or the L protein of the tri-segmented arenavirus particle can be under the control of the promoter element of the 5′ UTR (see e.g., Albarino et al., 2011, J Virol., 85(8):4020-4). In another embodiment, the ORF encoding GP, NP Z protein, L protein of the tri-segmented arenavirus particle can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment(s) or the L segment(s). In another specific embodiment, the promoter element of the 3′ UTR is the 3′ UTR the promoter element of the S segment(s) or the L segment(s).

In certain embodiments, the ORF that encoding GP, NP, Z protein or the L protein of the tri-segmented arenavirus particle can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the arenavirus S segment or L segment. In more specific embodiments, the truncated 5′ UTR is the 5′ UTR of the arenavirus S segment(s) or L segment(s).

Also provided herein, is a cDNA of the tri-segmented arenavirus particle comprising a nucleotide sequence encoding a tumor antigen, tumor associated antigen or an antigenic fragment thereof provided herein. In more specific embodiments, provided herein is a DNA nucleotide sequence or a set of DNA nucleotide sequences encoding a tri-segmented arenavirus particle as set forth in Table 2 or Table 3.

In certain embodiments, the nucleic acids encoding the tri-segmented arenavirus genome are part of or incorporated into one or more DNA expression vectors. In a specific embodiment, nucleic acids encoding the genome of the tri-segmented arenavirus particle are part of or incorporated into one or more DNA expression vectors that facilitate production of a tri-segmented arenavirus particle as described herein. In another embodiment, a cDNA described herein can be incorporated into a plasmid. More detailed description of the cDNAs and expression systems are provided is Section 5.5. Techniques for the production of a cDNA and routine and conventional techniques of molecular biology and DNA manipulation and production, including any cloning technique known to the skilled artisan can be used. Such techniques are well known and are available to the skilled artesian in laboratory manuals such as, Sambrook and Russell, Molecular Cloning: A laboratory Manual, 3^{rd} edition, Cold Spring Harbor Laboratory N.Y. (2001).

In certain embodiments, the cDNA of the tri-segmented arenavirus comprising a nucleotide sequence encoding a tumor antigen, tumor associated antigen or an antigenic fragment thereof provided herein is introduced (e.g., transfected) into a host cell. Thus, in some embodiments provided herein, is a host cell comprising a cDNA of the tri-segmented arenavirus particle (i.e., a cDNA of the genomic segments of the tri-segmented arenavirus particle) and a nucleotide sequence encoding a tumor antigen, tumor associated antigen or an antigenic fragment thereof provided herein. In other embodiments, the cDNA described herein that is part of or can be incorporated into a DNA expression vector and introduced into a host cell. Thus, in some embodiments provided herein is a host cell comprising a cDNA described herein that is incorporated into a vector. In other embodiments, the tri-segmented arenavirus genomic segments (i.e., the L segment and/or S segment or segments) described herein is introduced into a host cell.

In certain embodiments, described herein is a method of producing the tri-segmented arenavirus particle, wherein the method comprises transcribing the cDNA of the tri-segmented arenavirus particle comprising a nucleotide sequence encoding a tumor antigen, tumor associated antigen or an antigenic fragment thereof provided herein. In certain embodiments, a viral polymerase protein can be present during transcription of the tri-segmented arenavirus particle in vitro or in vivo. In certain embodiments, transcription of the arenavirus genomic segment is performed using a bi-directional promoter.

In other embodiments, transcription of the arenavirus genomic segment is performed using a bi-directional expression cassette (see e.g., Ortiz-Riaño et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

In certain embodiments, the method of producing the tri-segmented arenavirus particle can further comprise introducing into a host cell the cDNA of the tri-segmented arenavirus particle comprising a nucleotide sequence encoding a tumor antigen, tumor associated antigen or an antigenic fragment thereof provided herein. In certain embodiments, the method of producing the tri-segmented arenavirus particle can further comprise introducing into a host cell the cDNA of the tri-segmented arenavirus particle that comprises a nucleotide sequence encoding a tumor antigen, tumor associated antigen or an antigenic fragment thereof provided herein, wherein the host cell expresses all other components for production of the tri-segmented arenavirus particle; and purifying the tri-segmented arenavirus particle from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 5.5.

In certain embodiments, the tri-segmented arenavirus particle as described herein results in an infectious and replication competent arenavirus particle. In specific embodiments, the arenavirus particle described herein is attenuated. In a particular embodiment, the tri-segmented arenavirus particle is attenuated such that the virus remains, at least partially, replication-competent and can replicate in vivo, but can only generate low viral loads resulting in subclinical levels of infection that are non-pathogenic. Such attenuated viruses can be used as an immunogenic composition.

In certain embodiments, the tri-segmented arenavirus particle has the same tropism as the bi-segmented arenavirus particle.

Also provided herein, are compositions that comprise the tri-segmented arenavirus particle as described in Section 5.6 and 5.7.

### 5.2.1 Tri-Segmented Arenavirus Particle Comprising One L Segment and Two S Segments

In one aspect, provided herein is a tri-segmented arenavirus particle comprising one L segment and two S segments. In certain embodiments, propagation of the tri-segmented arenavirus particle comprising one L segment and two S segments does not result in a replication-competent bi-segmented viral particle. In specific embodiments, propagation of the tri-segmented arenavirus particle comprising one L segment and two S segments does not result in a replication-competent bi-segmented viral particle after at least 10 days, at least 20 days, at least 30 days, at least 40 days, at least 50 days, at least 60 days, at least 70 days, at least 80 days, at least 90 days, or at least 100 days of persistent infection in mice lacking type I interferon receptor, type II interferon receptor and recombination activating gene (RAG1), and having been infected with 10⁴ PFU of the tri-segmented arenavirus particle (see Section 5.8.14). In other embodiments, propagation of the tri-segmented arenavirus particle comprising one L segment and two S segments does not result in a replication-competent bi-segmented viral particle after at least 10 passages, at least 20 passages, at least 30 passages, at least 40 passages, or at least 50 passages.

The tri-segmented arenavirus particle with all viral genes in their respective wild-type position is known in the art (e.g., Emonet et al., 2011 J. Virol., 85(4):1473; Popkin et al., 2011, J. Virol, 85(15):7928). In particular, the tri-segmented arenavirus genome consists of one L segment and two S segments, in which a nucleotide sequence encoding a tumor antigen, tumor associated antigen or an antigenic fragment thereof provided herein is inserted into one position on each S segment. More specifically, one S segment encodes GP and a tumor antigen, tumor associated antigen or an antigenic fragment thereof, respectively. The other S segment encodes a tumor antigen, a tumor associated antigen or an antigenic fragment thereof and NP, respectively. The L segment encodes the L protein and Z protein. All segments are flanked by the respective 5′ and 3′ UTRs.

In certain embodiments, inter-segmental recombination of the two S segments of the tri-segmented arenavirus particle, provided herein, that unities the two arenaviral ORFs on one instead of two separate segments results in a non functional promoter (i.e., a genomic segment of the structure: 5′ UTR----------5′ UTR or a 3′ UTR----------3′ UTR), wherein each UTR forming one end of the genome is an inverted repeat sequence of the other end of the same genome.

In certain embodiments, the tri-segmented arenavirus particle comprising one L segment and two S segments has been engineered to carry an arenavirus ORF in a position other than the wild-type position of the ORF and a nucleotide sequence encoding a tumor antigen, tumor associated antigen or an antigenic fragment thereof provided herein. In other embodiments, the tri-segmented arenavirus particle comprising one L segment and two S segments has been engineered to carry two arenavirus ORFs, or three arenavirus ORFs, or four arenavirus ORFs, or five arenavirus ORFs, or six arenavirus ORFs in a position other than the wild-type position. In specific embodiments, the tri-segmented arenavirus particle comprising one L segment and two S segments comprises a full complement of all four arenavirus ORFs. Thus, in some embodiments, the tri-segmented arenavirus particle is an infectious and replication competent tri-segmented arenavirus particle. In specific embodiments, the two S segments of the tri-segmented arenavirus particle have been engineered to carry one of their ORFs in a position other than the wild-type position. In more specific embodiments, the two S segments comprise a full complement of the S segment ORFs. In certain specific embodiments, the L segment has been engineered to carry an ORF in a position other than the wild-type position or the L segment can be the wild-type genomic segment.

In certain embodiments, one of the two S segments can be:
(i) an arenavirus S segment, wherein the ORF encoding the Z protein is under control of an arenavirus 5′ UTR;
(ii) an arenavirus S segment, wherein the ORF encoding the L protein is under control of an arenavirus 5′ UTR;
(iii) an arenavirus S segment, wherein the ORF encoding the NP is under control of an arenavirus 5′ UTR;
(iv) an arenavirus S segment, wherein the ORF encoding the GP is under control of an arenavirus 3′ UTR;
(v) an arenavirus S segment, wherein the ORF encoding the L is under control of an arenavirus 3′ UTR; and
(vi) an arenavirus S segment, wherein the ORF encoding the Z protein is under control of an arenavirus 3′ UTR.

In certain embodiments, the tri-segmented arenavirus particle comprising one L segment and two S segments can comprise a duplicate ORF (i.e., two wild-type S segment ORFs e.g., GP or NP). In specific embodiments, the tri-segmented arenavirus particle comprising one L segment and two S segments can comprise one duplicate ORF (e.g., (GP, GP)) or two duplicate ORFs (e.g., (GP, GP) and (NP, NP)).

Table 2A, below, is an illustration of the genome organization of a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2A
Tri-segmented arenavirus particle comprising
one L segment and two S segments

| Position 1 | Position 2 | Position 3 | Position 4 | Position 5 | Position 6 |
|---|---|---|---|---|---|
| *ORF | GP | *ORF | NP | Z | L |
| *ORF | NP | *ORF | GP | Z | L |
| *ORF | NP | *ORF | GP | L | Z |
| *ORF | NP | *ORF | Z | L | GP |
| *ORF | NP | Z | GP | *ORF | Z |
| *ORF | NP | Z | GP | Z | *ORF |
| *ORF | NP | *ORF | L | Z | GP |
| *ORF | L | *ORF | NP | Z | GP |
| *ORF | L | Z | NP | *ORF | GP |
| *ORF | L | *ORF | GP | Z | NP |
| *ORF | L | Z | GP | *ORF | NP |
| *ORF | Z | L | NP | *ORF | GP |
| *ORF | Z | *ORF | GP | L | NP |
| *ORF | Z | L | GP | *ORF | NP |
| L | GP | *ORF | NP | *ORF | Z |
| L | GP | *ORF | *ORF | Z | NP |
| L | GP | *ORF | Z | *ORF | NP |
| L | *ORF | Z | GP | *ORF | NP |
| L | GP | *ORF | NP | *ORF | Z |
| L | GP | *ORF | Z | *ORF | NP |
| L | GP | Z | NP | *ORF | *ORF |
| L | GP | Z | NP | *ORF | *ORF |
| L | *ORF | Z | NP | *ORF | GP |
| L | NP | *ORF | Z | *ORF | GP |
| L | NP | Z | *ORF | GP | *ORF |
| L | *ORF | Z | *ORF | GP | NP |
| L | NP | Z | GP | *ORF | *ORF |
| L | NP | *ORF | Z | *ORF | GP |
| L | *ORF | Z | NP | *ORF | GP |
| L | Z | *ORF | GP | *ORF | NP |
| L | Z | *ORF | NP | *ORF | GP |
| Z | GP | *ORF | NP | *ORF | L |
| Z | GP | *ORF | *ORF | L | NP |
| Z | GP | *ORF | L | *ORF | NP |
| Z | *ORF | L | GP | *ORF | NP |
| Z | GP | *ORF | NP | *ORF | L |
| Z | GP | *ORF | L | *ORF | NP |
| Z | GP | L | NP | *ORF | *ORF |
| Z | GP | L | NP | *ORF | *ORF |
| Z | *ORF | L | NP | *ORF | GP |
| Z | NP | *ORF | *ORF | L | GP |
| Z | NP | *ORF | GP | *ORF | L |
| Z | NP | *ORF | *ORF | L | GP |
| Z | NP | *ORF | L | *ORF | GP |
| Z | NP | L | GP | *ORF | *ORF |
| Z | *ORF | L | GP | *ORF | NP |
| Z | NP | *ORF | GP | *ORF | L |
| Z | NP | *ORF | L | *ORF | GP |
| Z | *ORF | L | NP | *ORF | GP |
| Z | L | *ORF | GP | *ORF | NP |

Position 1 is under the control of an arenavirus S segment 5′ UTR; Position 2 is under the control of an arenavirus S segment 3′ UTR; Position 3 is under the control of an arenavirus S segment 5′ UTR; Position 4 under the control of an arenavirus S segment 3′ UTR; Position 5 is under the control of an arenavirus L segment 5′ UTR; Position 6 is under the control of an arenavirus L segment 3′ UTR.
*ORF indicates that a nucleotide sequence encoding a tumor antigen, tumor associated antigen or an antigenic fragment thereof provided herein has been inserted.

In certain embodiments, the IGR between position one and position two can be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus S segment IGR; the IGR between position two and three can be an arenavirus S segment IGR; and the IGR between the position five and six can be an arenavirus L segment IGR. In certain embodiments, other combinations are also possible. For example, a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, intersegmental recombination of an S segment and an L segment in the tri-segmented arenavirus particle comprising one L segment and two S segments, restores a functional segment with two viral genes on only one segment instead of two separate segments. In other embodiments, intersegmental recombination of an S segment and an L segment in the tri-segmented arenavirus particle comprising one L segment and two S segments does not result in a replication-competent bi-segmented viral particle.

Table 2B, below, is an illustration of the genome organization of a tri-segmented arenavirus particle comprising one L segment and two S segments, wherein intersegmental recombination of an S segment and an L segment in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2B
Tri-segmented arenavirus particle comprising
one L segment and two S segments

| Position 1 | Position 2 | Position 3 | Position 4 | Position 5 | Position 6 |
|---|---|---|---|---|---|
| L | GP | *ORF | NP | Z | *ORF |
| L | GP | Z | *ORF | *ORF | NP |
| L | GP | *ORF | NP | Z | *ORF |
| L | GP | Z | *ORF | *ORF | NP |
| L | NP | *ORF | GP | Z | *ORF |
| L | NP | Z | *ORF | *ORF | GP |
| L | NP | *ORF | GP | Z | *ORF |
| L | NP | Z | *ORF | *ORF | GP |
| Z | GP | *ORF | NP | L | *ORF |
| Z | GP | L | *ORF | *ORF | NP |
| Z | GP | *ORF | NP | L | *ORF |
| Z | NP | L | *ORF | *ORF | GP |
| Z | NP | *ORF | GP | L | *ORF |
| Z | NP | L | *ORF | *ORF | GP |

Position 1 is under the control of an arenavirus S segment 5′ UTR; Position 2 is under the control of an arenavirus S segment 3′ UTR; Position 3 is under the control of an arenavirus S segment 5′ UTR; Position 4 under the control of an arenavirus S segment 3′ UTR; Position 5 is under the control of an arenavirus L segment 5′ UTR; Position 6 is under the control of an arenavirus L segment 3′ UTR.
*ORF indicates that a nucleotide sequence encoding a tumor antigen, tumor associated antigen or an antigenic fragment thereof provided herein has been inserted.

In certain embodiments, the IGR between position one and position two can be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus S segment IGR; the IGR between position two and three can be an arenavirus S segment IGR; and the IGR between the position five and six can be an arenavirus L segment IGR. In certain embodiments, other combinations are also possible. For example, a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, one of skill in the art could construct an arenavirus genome with an organization as illustrated in Table 2A or 2B and as described herein, and then use an assay as described in Section 5.8 to determine whether the tri-segmented arenavirus particle is genetically stable, i.e., does not result in a replication-competent bi-segmented viral particle as discussed herein.

### 5.2.2 Tri-Segmented Arenavirus Particle Comprising Two L Segments and One S Segment

In one aspect, provided herein is a tri-segmented arenavirus particle comprising two L segments and one S segment. In certain embodiments, propagation of the tri-segmented arenavirus particle comprising two L segments and one S segment does not result in a replication-competent bi-segmented viral particle. In specific embodiments, propagation of the tri-segmented arenavirus particle comprising two L segments and one S segment does not result in a replication-competent bi-segmented viral particle after at least 10 days, at least 20 days, at least 30 days, at least 40 days, or at least 50 days, at least 60 days, at least 70 days, at least 80 days, at least 90 days, at least 100 days of persistent in mice lacking type I interferon receptor, type II interferon receptor and recombination activating gene (RAG1), and having been infected with 10⁴ PFU of the tri-segmented arenavirus particle (see Section 5.8.14). In other embodiments, propagation of the tri-segmented arenavirus particle comprising two L segments and one S segment does not result in a replication-competent bi-segmented viral particle after at least 10 passages, 20 passages, 30 passages, 40 passages, or 50 passages.

In certain embodiments, inter-segmental recombination of the two L segments of the tri-segmented arenavirus particle, provided herein, that unities the two arenaviral ORFs on one instead of two separate segments results in a non functional promoter (i.e., a genomic segment of the structure: 5′ UTR---------5′ UTR or a 3′ UTR------------3′ UTR), wherein each UTR forming one end of the genome is an inverted repeat sequence of the other end of the same genome.

In certain embodiments, the tri-segmented arenavirus particle comprising two L segments and one S segment has been engineered to carry an arenavirus ORF in a position other than the wild-type position of the ORF and a nucleotide sequence encoding a tumor antigen, tumor associated antigen or an antigenic fragment thereof provided herein. In other embodiments, the tri-segmented arenavirus particle comprising two L segments and one S segment has been engineered to carry two arenavirus ORFs, or three arenavirus ORFs, or four arenavirus ORFs, or five arenavirus ORFs, or six arenavirus ORFs in a position other than the wild-type position. In specific embodiments, the tri-segmented arenavirus particle comprising two L segments and one S segment comprises a full complement of all four arenavirus ORFs. Thus, in some embodiments, the tri-segmented arenavirus particle is an infectious and replication competent tri-segmented arenavirus particle. In specific embodiments, the two L segments of the tri-segmented arenavirus particle have been engineered to carry one of their ORFs in a position other than the wild-type position. In more specific embodiments, the two L segments comprise a full complement of the L segment ORFs. In certain specific embodiments, the S segment has been engineered to carry one of their ORFs in a position other than the wild-type position or the S segment can be the wild-type genomic segment.

In certain embodiments, one of the two L segments can be:
(i) an L segment, wherein the ORF encoding the GP is under control of an arenavirus 5′ UTR;
(i) an L segment, wherein the ORF encoding NP is under control of an arenavirus 5′ UTR;
(ii) an L segment, wherein the ORF encoding the L protein is under control of an arenavirus 5′ UTR;
(iii) an L segment, wherein the ORF encoding the GP is under control of an arenavirus 3′ UTR;
(iv) an L segment, wherein the ORF encoding the NP is under control of an arenavirus 3′ UTR; and
(v) an L segment, wherein the ORF encoding the Z protein is under control of an arenavirus 3′ UTR.

In certain embodiments, the tri-segmented arenavirus particle comprising one L segment and two S segments can comprise a duplicate ORF (i.e., two wild-type L segment ORFs e.g., Z protein or L protein). In specific embodiments, the tri-segmented arenavirus particle comprising two L segments and one S segment can comprise one duplicate ORF (e.g., (Z protein, Z protein)) or two duplicate ORFs (e.g., (Z protein, Z protein) and (L protein, L protein)).

Table 3, below, is an illustration of the genome organization of a tri-segmented arenavirus particle comprising two L segments and one S segment, wherein intersegmental recombination of the two L segments in the tri-segmented arenavirus genome does not result in a replication-competent bi-segmented viral particle and abrogates arenaviral promoter activity (i.e., the S segment is made up of two 3′UTRs instead of a 3′ UTR and a 5′ UTR). Based on Table 3 similar combinations could be predicted for generating an arenavirus particle made up of two 5′ UTRs instead of a 3′ UTR and a 5′ UTR.

TABLE 3
Tri-segmented arenavirus particle comprising
two L segments and one S segment

| Position 1 | Position 2 | Position 3 | Position 4 | Position 5 | Position 6 |
|---|---|---|---|---|---|
| ORF* | Z | ORF* | L | NP | GP |
| ORF* | Z | ORF* | L | GP | NP |
| ORF* | Z | GP | L | ORF* | NP |
| ORF* | Z | ORF* | GP | NP | L |
| ORF* | Z | GP | ORF* | NP | L |
| ORF* | Z | NP | ORF* | GP | L |
| ORF* | ORF* | NP | Z | GP | L |
| ORF* | Z | GP | NP | ORF* | L |
| ORF* | Z | NP | GP | ORF* | L |
| ORF* | L | ORF* | Z | NP | GP |
| ORF* | L | ORF* | Z | GP | NP |
| ORF* | L | ORF* | GP | NP | Z |
| ORF* | L | GP | Z | ORF* | NP |
| ORF* | L | ORF* | GP | NP | Z |
| ORF* | L | NP | Z | ORF* | GP |
| ORF* | L | GP | NP | ORF* | Z |
| ORF* | L | NP | GP | ORF* | Z |
| ORF* | GP | ORF* | L | NP | Z |
| ORF* | GP | NP | L | ORF* | Z |
| ORF* | GP | ORF* | Z | NP | L |
| ORF* | GP | NP | Z | ORF* | L |
| ORF* | NP | ORF* | L | GP | Z |
| ORF* | NP | GP | L | ORF* | Z |
| ORF* | NP | GP | Z | ORF* | L |
| ORF* | NP | ORF* | Z | GP | L |
| ORF* | L | ORF* | Z | NP | GP |
| ORF* | L | ORF* | Z | GP | NP |
| ORF* | L | ORF* | NP | GP | Z |
| ORF* | L | ORF* | GP | NP | Z |
| ORF* | L | NP | Z | ORF* | GP |
| ORF* | Z | ORF* | GP | NP | L |
| ORF* | Z | GP | L | ORF* | NP |
| ORF* | Z | NP | GP | ORF* | L |
| ORF* | Z | GP | NP | ORF* | L |
| ORF* | GP | ORF* | L | NP | Z |
| ORF* | GP | ORF* | L | Z | NP |
| ORF* | GP | ORF* | Z | GP | L |
| ORF* | GP | NP | L | ORF* | Z |
| GP | L | ORF* | Z | ORF* | NP |
| GP | L | ORF* | NP | ORF* | Z |
| GP | Z | ORF* | L | ORF* | NP |
| GP | Z | ORF* | L | ORF* | NP |
| GP | Z | ORF* | NP | ORF* | L |
| GP | NP | ORF* | Z | ORF* | L |
| NP | L | ORF* | Z | ORF* | GP |
| NP | L | ORF* | GP | ORF* | Z |
| NP | L | ORF* | Z | ORF* | GP |

*Position 1 is under the control of an arenavirus L segment 5′ UTR; position 2 is under the control of an arenavirus L segment 3′ UTR; position 3 is under the control of an arenavirus L segment 5′ UTR; position 4 is under the control of an arenavirus L segment 3′ UTR; position 5 is under the control of an arenavirus S segment 5′ UTR; position 6 is under the control of an arenavirus S segment 3′ UTR.
*ORF indicates that a nucleotide sequence encoding a tumor antigen, tumor associated antigen or an antigenic fragment thereof provided herein has been inserted.

In certain embodiments, the IGR between position one and position two cab be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus L segment IGR; the IGR between position two and three can be an arenavirus L segment IGR; and the IGR between the position five and six can be an arenavirus S segment IGR. In certain embodiments, other combinations are also possible.

In certain embodiments, intersegmental recombination of an L segment and an S segment from the tri-segmented arenavirus particle comprising two L segments and one S segment restores a functional segment with two viral genes on only one segment instead of two separate segments. In other embodiments, intersegmental recombination of an L segment and an S segment in the tri-segmented arenavirus particle comprising two L segments and one S segment does not result in a replication-competent bi-segmented viral particle.

Table 3B, below, is an illustration of the genome organization of a tri-segmented arenavirus particle comprising two L segments and one S segment, wherein intersegmental recombination of an L segment and an S segment in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 3B
Tri-segmented arenavirus particle comprising
two L segments and one S segment

| Position 1 | Position 2 | Position 3 | Position 4 | Position 5 | Position 6 |
|---|---|---|---|---|---|
| NP | Z | *ORF | GP | L | *ORF |
| NP | Z | GP | *ORF | *ORF | L |
| NP | Z | *ORF | GP | L | *ORF |
| NP | Z | GP | *ORF | *ORF | L |
| NP | L | *ORF | GP | Z | *ORF |
| NP | L | GP | *ORF | *ORF | Z |
| NP | L | *ORF | GP | Z | *ORF |
| NP | L | GP | *ORF | *ORF | Z |
| GP | Z | *ORF | NP | L | *ORF |
| GP | Z | NP | *ORF | *ORF | L |
| GP | Z | *ORF | NP | L | *ORF |
| GP | L | NP | *ORF | *ORF | Z |
| GP | L | *ORF | NP | Z | *ORF |
| GP | L | NP | *ORF | *ORF | Z |

*Position 1 is under the control of an arenavirus L segment 5′ UTR; position 2 is under the control of an arenavirus L segment 3′ UTR; position 3 is under the control of an arenavirus L segment 5′ UTR; position 4 is under the control of an arenavirus L segment 3′ UTR; position 5 is under the control of an arenavirus S segment 5′ UTR; position 6 is under the control of an arenavirus S segment 3′ UTR.
*ORF indicates that a nucleotide sequence encoding a tumor antigen, tumor associated antigen or an antigenic fragment thereof provided herein has been inserted.

In certain embodiments, the IGR between position one and position two cab be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus L segment IGR; the IGR between position two and three can be an arenavirus L segment IGR; and the IGR between the position five and six can be an arenavirus S segment IGR. In certain embodiments, other combinations are also possible.

In certain embodiments, one of skill in the art could construct an arenavirus genome with an organization as illustrated in Table 3A or 3B and as described herein, and then use an assay as described in Section 5.8 to determine whether the tri-segmented arenavirus particle is genetically stable, i.e., does not result in a replication-competent bi-segmented viral particle as discussed herein.

### 5.2.3 Replication-Defective Tri-Segmented Arenavirus Particle

In certain embodiments, tri-segmented replication-defective (e.g., replication-deficient) arenavirus particles with a nucleotide sequence encoding a tumor antigen, tumor associated antigen or an antigenic fragment thereof provided herein can be used with the methods and compositions provided herein. In specific embodiments, tri-segmented replication-defective arenavirus particles described herein are used with the methods and compositions provided herein in combination with replication-competent arenavirus particles described herein. In more specific embodiments, tri-segmented replication-defective arenavirus particles described herein are used with the methods and compositions provided herein in combination with replication-competent arenavirus particles described herein, wherein said replication-competent arenavirus particles are injected directly into a tumor in a subject.

In certain embodiments, provided herein is a tri-segmented arenavirus particle in which (i) an ORF is in a position other than the wild-type position of the ORF; and (ii) an ORF encoding GP, NP, Z protein, or L protein has been removed or functionally inactivated such that the resulting virus cannot produce further infectious progeny virus particles (i.e., is replication defective). In certain embodiments, the third arenavirus segment can be an S segment. In other embodiments, the third arenavirus segment can be an L segment. In more specific embodiments, the third arenavirus segment can be engineered to carry an ORF in a position other than the wild-type position of the ORF or the third arenavirus segment can be the wild-type arenavirus genomic segment. In yet more specific embodiments, the third arenavirus segment lacks an arenavirus ORF encoding GP, NP, Z protein, or the L protein.

In certain embodiments, a tri-segmented genomic segment could be an S or an L segment hybrid (i.e., a genomic segment that can be a combination of the S segment and the L segment). In other embodiments, the hybrid segment is an S segment comprising an L segment IGR. In another embodiment, the hybrid segment is an L segment comprising an S segment IGR. In other embodiments, the hybrid segment is an S segment UTR with and L segment IGR. In another embodiment, the hybrid segment is an L segment UTR with an S segment IGR. In specific embodiments, the hybrid segment is an S segment 5′ UTR with an L segment IGR or an S segment 3′ UTR with an L segment IGR. In other specific embodiments, the hybrid segment is an L segment 5′ UTR with an S segment IGR or an L segment 3′ UTR with an S segment IGR.

A tri-segmented arenavirus particle comprising a genetically modified genome in which one or more ORFs has been deleted or functionally inactivated can be produced in complementing cells (i.e., cells that express the arenavirus ORF that has been deleted or functionally inactivated). The genetic material of the resulting arenavirus particle can be transferred upon infection of a host cell into the host cell, wherein the genetic material can be expressed and amplified. In addition, the genome of the genetically modified arenavirus particle described herein can include a nucleotide sequence encoding a tumor antigen, tumor associated antigen or an antigenic fragment thereof provided herein.

In certain embodiments, at least one of the four ORFs encoding GP, NP, Z protein, and L protein is removed and replaced with a nucleotide sequence encoding a tumor antigen, tumor associated antigen or an antigenic fragment thereof provided herein. In another embodiment, at least one ORF, at least two ORFs, at least three ORFs, or at least four ORFs encoding GP, NP, Z protein and L protein can be removed and replaced with a nucleotide sequence encoding a tumor antigen, tumor associated antigen or an antigenic fragment thereof provided herein. In specific embodiments, only one of the four ORFs encoding GP, NP, Z protein, and L protein is removed and replaced with a nucleotide sequence encoding a tumor antigen, tumor associated antigen or an antigenic fragment thereof provided herein. In more specific embodiments, the ORF that encodes GP of the arenavirus genomic segment is removed. In another specific embodiment, the ORF that encodes the NP of the arenavirus genomic segment is removed. In more specific embodiments, the ORF that encodes the Z protein of the arenavirus genomic segment is removed. In yet another specific embodiment, the ORF encoding the L protein is removed.

In certain embodiments, provided herein is a tri-segmented arenavirus particle comprising one L segment and two S segments in which (i) an ORF is in a position other than the wild-type position of the ORF; and (ii) an ORF encoding GP or NP has been removed or functionally inactivated, such that the resulting virus is replication-defective and not infectious. In a specific embodiment, one ORF is removed and replaced with a nucleotide sequence encoding a tumor antigen, tumor associated antigen or an antigenic fragment thereof provided herein. In another specific embodiment, two ORFs are removed and replaced with a nucleotide sequence encoding a tumor antigen, tumor associated antigen or an antigenic fragment thereof provided herein. In other specific embodiments, three ORFs are removed and replaced with a nucleotide sequence encoding a tumor antigen, tumor associated antigen or an antigenic fragment thereof provided herein. In specific embodiments, the ORF encoding GP is removed and replaced with a nucleotide sequence encoding a tumor antigen, tumor associated antigen or an antigenic fragment thereof provided herein. In other specific embodiments, the ORF encoding NP is removed and replaced with a nucleotide sequence encoding a tumor antigen, tumor associated antigen or an antigenic fragment thereof provided herein. In yet more specific embodiments, the ORF encoding NP and the ORF encoding GP are removed and replaced with one or two nucleotide sequences encoding tumor antigens, tumor associated antigens or antigenic fragments thereof provided herein. Thus, in certain embodiments the tri-segmented arenavirus particle comprises (i) one L segment and two S segments; (ii) an ORF in a position other than the wild-type position of the ORF; (iii) one or more nucleotide sequences encoding tumor antigens, tumor associated antigens or an antigenic fragments thereof provided herein.

In certain embodiments, provided herein is a tri-segmented arenavirus particle comprising two L segments and one S segment in which (i) an ORF is in a position other than the wild-type position of the ORF; and (ii) an ORF encoding the Z protein, and/or the L protein has been removed or functionally inactivated, such that the resulting virus replication-defective and not infectious. In a specific embodiment, one ORF is removed and replaced with a nucleotide sequence encoding a tumor antigen, tumor associated antigen or an antigenic fragment thereof provided herein. In another specific embodiment, two ORFs are removed and replaced with a nucleotide sequence encoding a tumor antigen, tumor associated antigen or an antigenic fragment thereof provided herein. In specific embodiments, the ORF encoding the Z protein is removed and replaced with a nucleotide sequence encoding a tumor antigen, tumor associated antigen or an antigenic fragment thereof provided herein. In other specific embodiments, the ORF encoding the L protein is removed and replaced with a nucleotide sequence encoding a tumor antigen, tumor associated antigen or an antigenic fragment thereof provided herein. In yet more specific embodiments, the ORF encoding the Z protein and the ORF encoding the L protein is removed and replaced with a nucleotide sequence encoding a tumor antigen, tumor associated antigen or an antigenic fragment thereof provided herein. Thus, in certain embodiments the tri-segmented arenavirus particle comprises (i) two L segments and one S segment; (ii) an ORF in a position other than the wild-type position of the ORF; (iii) a nucleotide sequence encoding a tumor antigen, tumor associated antigen or an antigenic fragment thereof provided herein.

Thus, in certain embodiments, the tri-segmented arenavirus particle provided herein comprises a tri-segmented arenavirus particle (i.e., one L segment and two S segments or two L segments and one S segment) that i) is engineered to carry an ORF in a non-natural position; ii) an ORF encoding GP, NP, Z protein, or L protein is removed); iii) the ORF that is removed is replaced with one or more nucleotide sequences encoding tumor antigens, tumor associated antigens or antigenic fragments thereof provided herein.

In certain embodiments, the nucleotide sequence encoding an antigenic fragment provided herein is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the nucleotide sequence encoding an antigenic fragment provided herein is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence encoding a tumor antigen, tumor associated antigen or an antigenic fragment thereof provided herein.

Any nucleotide sequence encoding a tumor antigen, tumor associated antigen or an antigenic fragment thereof provided herein may be included in the tri-segmented arenavirus particle. In one embodiment, a nucleotide sequence encoding a tumor antigen, tumor associated antigen or an antigenic fragment thereof provided herein is capable of eliciting an immune response.

In certain embodiments, the growth and infectivity of the arenavirus particle is not affected by the nucleotide sequence encoding a tumor antigen, tumor associated antigen or an antigenic fragment thereof provided herein.

Techniques known to one skilled in the art may be used to produce an arenavirus particle comprising an arenavirus genomic segment engineered to carry an arenavirus ORF in a position other than the wild-type position and a nucleotide sequence encoding a tumor antigen, tumor associated antigen or an antigenic fragment thereof provided herein. For example, reverse genetics techniques may be used to generate such arenavirus particle. In other embodiments, the replication-defective arenavirus particle (i.e., the arenavirus genomic segment engineered to carry an arenavirus ORF in a position other than the wild-type position, wherein an ORF encoding GP, NP, Z protein, L protein, has been deleted) can be produced in a complementing cell.

In certain embodiments, a tri-segmented arenavirus particle provided herein comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof; Ubiquitin or a fragment thereof; Granulocyte-Macrophage Colony-Stimulating Factor (GM-CSF), or a fragment thereof; Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof; CD40 ligand or an antigenic fragment thereof; or Fms-related tyrosine kinase 3 (Flt3) ligand or an antigenic fragment thereof.

Arenaviruses for use with the methods and compositions provided herein can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

In certain embodiments, the tri-segmented arenavirus particle as described herein is suitable for use as a vaccine and methods of using such arenavirus particle in a vaccination and treatment for a neoplastic disease, for example, cancer, is provided. More detailed description of the methods of using the arenavirus particle described herein is provided in Section 5.6

In certain embodiments, the tri-segmented arenavirus particle as described herein is suitable for use as a pharmaceutical composition and methods of using such arenavirus particle in a vaccination and treatment for a neoplastic disease, for example, cancer, is provided. More detailed description of the methods of using the arenavirus particle described herein is provided in Section 5.7.

### 5.3 Tumor Antigens, Tumor Associated Antigens and Antigenic Fragments

In certain embodiments, arenavirus particles with nucleotide sequence encoding a tumor antigen, tumor associated antigen or an antigenic fragment thereof provided herein can be used with the methods and compositions provided herein. In certain embodiments, arenavirus particles with nucleotide sequence encoding a tumor antigen, tumor associated antigen or an antigenic fragment thereof provided herein can be used with the methods and compositions provided herein in combination with arenavirus particles with nucleotide sequence not encoding a foreign antigen. In certain embodiments, a tumor antigen or tumor associated antigen for use with the methods and compositions described herein is an immunogenic protein expressed in or on a neoplastic cell or tumor, such as a cancer cell or malignant tumor. In certain embodiments, a tumor antigen or tumor associated antigen for use with the methods and compositions described herein is a non-specific, mutant, overexpressed or abnormally expressed protein, which can be present on both a neoplastic cell or tumor and a normal cell or tissue. In certain embodiments, a tumor antigen or tumor associated antigen for use with the methods and compositions described herein is a tumor-specific antigen which is restricted to tumor cells. In certain embodiments, a tumor antigen for use with the methods and compositions described herein is a cancer-specific antigen which is restricted to cancer cells.

In certain embodiments, a tumor antigen or tumor associated antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, the tumor antigen or tumor associated antigen for use with the methods and compositions described herein includes antigens from neoplastic diseases including acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In certain embodiments, the tumor antigen or tumor associated antigen for use with the methods and compositions disclosed herein includes oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A1, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

In certain embodiments, the tumor antigen or tumor associated antigen is a neoantigen. A “neoantigen,” as used herein, means an antigen that arises by mutation in a tumor cell and such an antigen is not generally expressed in normal cells or tissue. Without being bound by theory, because healthy tissues generally do not posses these antigens, neoantigens represent a preferred target. Additionally, without being bound by theory, in the context of the present invention, since the T cells that recognize the neoantigen may not have undergone negative thymic selection, such cells can have high avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen.

In certain embodiments, the tumor antigen or tumor associated antigen can be an antigen ortholog, e.g., a mammalian (i.e., non-human primate, pig, dog, cat, or horse) to a human tumor antigen or tumor associated antigen.

In certain embodiments, an antigenic fragment of a tumor antigen or tumor associated antigen described herein is encoded by the nucleotide sequence included within the arenavirus. In certain embodiments, a fragment is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding antigenic fragment of a tumor antigen or tumor associated antigen is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the heterologous ORF is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the heterologous ORF encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence of a tumor antigen or tumor associated antigen.

In certain embodiments, the arenavirus genomic segment, the arenavirus particle or the tri-segmented arenavirus particle can comprise one or more nucleotide sequences encoding tumor antigens, tumor associated antigens, or antigenic fragments thereof. In other embodiments, the arenavirus genomic segment, the arenavirus particle or the tri-segmented arenavirus particle can comprise at least one nucleotide sequence encoding a tumor antigen, tumor associated antigen, or antigenic fragment thereof, at least two nucleotide sequences encoding tumor antigens, tumor associated antigens, or antigenic fragments thereof, at least three nucleotide sequences encoding tumor antigens, tumor associated antigens, or antigenic fragments thereof, or more nucleotide sequences encoding tumor antigens, tumor associated antigens, or antigenic fragments thereof.

In certain embodiments, an arenavirus particle comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof; Ubiquitin or a fragment thereof; Granulocyte-Macrophage Colony-Stimulating Factor (GM-CSF), or a fragment thereof; Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof; CD40 ligand or an antigenic fragment thereof; or Fms-related tyrosine kinase 3 (Flt3) ligand or an antigenic fragment thereof.

In certain embodiments, an arenavirus particle provided herein comprises a genomic segment that a) has a removal or functional inactivation of an ORF that is present in the wild-type form of the genomic segment; and b) encodes (either in sense or antisense): (i) one or more tumor antigen, tumor associated antigen or an antigenic fragment thereof provided herein, and (ii) one or more immunomodulatory peptide, polypeptide or protein provided herein.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on the same position of the viral genome. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on different positions of the viral genome.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated via a spacer sequence. In certain embodiments, the sequence encoding the tumor antigen, tumor associated antigen or an antigenic fragment thereof provided herein, and the nucleotide sequence encoding the immunomodulatory peptide, polypeptide or protein provided herein, are separated by an internal ribosome entry site, or a sequence encoding a protease cleavage site. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated by a nucleotide sequence encoding a linker or a self-cleaving peptide. Any linker peptide or self-cleaving peptide known to the skilled artisan can be used with the compositions and methods provided herein. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein, are directly fused together. In certain embodiments, the tumor antigen, tumor associated antigen or an antigenic fragment thereof provided herein, and the immunomodulatory peptide, polypeptide or protein provided herein, are fused together via a peptide linker. In certain embodiments, the tumor antigen, tumor associated antigen or an antigenic fragment thereof provided herein, and the immunomodulatory peptide, polypeptide or protein provided herein are separated from each other via a self-cleaving peptide. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein are expressed on the same arenavirus particle. In certain embodiments, the tumor antigen, tumor associated antigen or an antigenic fragment thereof provided herein, and the immunomodulatory peptide, polypeptide or protein provided herein are expressed on different arenavirus particles. In certain embodiments, the tumor antigen, tumor associated antigen or an antigenic fragment thereof provided herein, and the immunomodulatory peptide, polypeptide or protein provided herein are expressed on different viruses of the same strain. In certain embodiments, the tumor antigen, tumor associated antigen or an antigenic fragment thereof provided herein, and the immunomodulatory peptide, polypeptide or protein provided herein are expressed on different viruses of different strains.

In certain embodiments, an arenavirus particle generated to encode one or more tumor antigens, tumor associated antigens or antigenic fragments thereof comprises one or more nucleotide sequences encoding tumor antigens, tumor associated antigens or antigenic fragments thereof provided herein. In specific embodiments the tumor antigens, tumor associated antigens or antigenic fragments thereof provided herein are separated by various one or more linkers, spacers, or cleavage sites as described herein.

### 5.4 Generation of an Arenavirus Particle and a Tri-Segmented Arenavirus Particle

Generally, arenavirus particles for use in the methods and compositions provided herein can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below. The genome of the viruses can be modified as described herein.

### 5.4.1 Non-Natural Position Open Reading Frame

The generation of an arenavirus particle comprising a genomic segment that has been engineered to carry a viral ORF in a position other than the wild-type position of the ORF and a nucleotide sequence encoding a tumor antigen, tumor associated antigen or antigenic fragment thereof can be recombinantly produced by any reverse genetic techniques known to one skilled in the art.

### (i) Infectious and Replication Competent Arenavirus Particle

In certain embodiments, the method of generating the arenavirus particle comprises (i) transfecting into a host cell the cDNA of the first arenavirus genomic segment; (ii) transfecting into a host cell the cDNA of the second arenavirus genomic segment; (iii) transfecting into a host cell plasmids expressing the arenavirus' minimal trans-acting factors NP and L; (iv) maintaining the host cell under conditions suitable for virus formation; and (v) harvesting the arenavirus particle. In certain more specific embodiments, the cDNA is comprised in a plasmid.

Once generated from cDNA, arenavirus particles (e.g., infectious and replication competent) can be propagated. In certain embodiments, the arenavirus particle can be propagated in any host cell that allows the virus to grow to titers that permit the uses of the virus as described herein. In one embodiment, the host cell allows the arenavirus particle to grow to titers comparable to those determined for the corresponding wild-type.

In certain embodiments, the arenavirus particle may be propagated in host cells. Specific examples of host cells that can be used include BHK-21, HEK 293, VERO or other. In a specific embodiment, the arenavirus particle may be propagated in a cell line.

In certain embodiments, the host cells are kept in culture and are transfected with one or more plasmid(s). The plasmid(s) express the arenavirus genomic segment(s) to be generated under control of one or more expression cassettes suitable for expression in mammalian cells, e.g., consisting of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that direct intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, the arenavirus genomic segments are under the control of a promoter. Typically, RNA polymerase I-driven expression cassettes, RNA polymerase II-driven cassettes or T7 bacteriophage RNA polymerase driven cassettes can be used. In certain embodiments, the plasmid(s) encoding the arenavirus genomic segments can be the same, i.e., the genome sequence and transacting factors can be transcribed by a promoter from one plasmid. Specific examples of promoters include an RNA polymerase I promoter, an RNA polymerase II promoter, an RNA polymerase III promoter, a T7 promoter, an SP6 promoter or a T3 promoter.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of a host cell with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

For recovering the arenavirus particle described herein, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous ORF, wherein a plasmid encoding the genomic segment is modified to incorporated a heterologous ORF. The heterologous ORF can be incorporated into the plasmid using restriction enzymes.

### (ii) Infectious, Replication-Defective Arenavirus Particle

Infectious, replication-defective arenavirus particles can be rescued as described above. However, once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the EF1alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids can be of two types: i) two plasmids, referred to as TF-plasmids for expressing intracellularly in C-cells the minimal transacting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, poll and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, poll and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient arenavirus expressing an antigen. When used for expression of an antigen in cultured cells, the following two procedures can be used:

i) The cell type of interest is infected with the arenavirus vector preparation described herein at a multiplicity of infection (MOI) of one or more, e.g., two, three or four, resulting in production of the antigen in all cells already shortly after infection.

ii) Alternatively, a lower MOI can be used and individual cell clones can be selected for their level of virally driven antigen expression. Subsequently individual clones can be expanded infinitely owing to the non-cytolytic nature of arenavirus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of antigen using infectious, replication-deficient arenaviruses as vectors may be considered.

### 5.4.2 Generation of a Tri-Segmented Arenavirus Particle

A tri-segmented arenavirus particle comprising a nucleotide sequence encoding a tumor antigen, tumor associated antigen or antigenic fragment thereof can be recombinantly produced by reverse genetic techniques known in the art, for example as described by Emonet et al., 2008, PNAS, 106(9):3473-3478; Popkin et al., 2011, J. Virol., 85 (15):7928-7932, which are incorporated by reference herein. The generation of the tri-segmented arenavirus particle provided herein can be modified as described in Section 5.2.

### (i) Infectious and Replication Competent Tri-Segmented Arenavirus Particle

In certain embodiments, the method of generating the tri-segmented arenavirus particle comprises (i) transfecting into a host cell the cDNAs of the one L segment and two S segments or two L segments and one S segment; (ii) transfecting into a host cell plasmids expressing the arenavirus' minimal trans-acting factors NP and L; (iii) maintaining the host cell under conditions suitable for virus formation; and (iv) harvesting the arenavirus particle.

Once generated from cDNA, the tri-segmented arenavirus particle (i.e., infectious and replication competent) can be propagated. In certain embodiments tri-segmented arenavirus particle can be propagated in any host cell that allows the virus to grow to titers that permit the uses of the virus as described herein. In one embodiment, the host cell allows the tri-segmented arenavirus particle to grow to titers comparable to those determined for the corresponding wild-type.

In certain embodiments, the tri-segmented arenavirus particle may be propagated in host cells. Specific examples of host cells that can be used include BHK-21, HEK 293, VERO or other. In a specific embodiment, the tri-segmented arenavirus particle may be propagated in a cell line.

In certain embodiments, the host cells are kept in culture and are transfected with one or more plasmid(s). The plasmid(s) express the arenavirus genomic segment(s) to be generated under control of one or more expression cassettes suitable for expression in mammalian cells, e.g., consisting of a polymerase I promoter and terminator.

In specific embodiments, the host cells are kept in culture and are transfected with one or more plasmid(s). The plasmid(s) express the viral gene(s) to be generated under control of one or more expression cassettes suitable for expression in mammalian cells, e.g., consisting of a polymerase I promoter and terminator.

Plasmids that can be used for generating the tri-segmented arenavirus comprising one L segment and two S segments can include: i) two plasmids each encoding the S genome segment e.g., pol-I S, ii) a plasmid encoding the L genome segment e.g., pol-I L. Plasmids needed for the tri-segmented arenavirus comprising two L segments and one S segments are: i) two plasmids each encoding the L genome segment e.g., pol-L, ii) a plasmid encoding the S genome segment e.g., pol-I S.

In certain embodiments, plasmids encoding an arenavirus polymerase that direct intracellular synthesis of the viral L and S segments can be incorporated into the transfection mixture. For example, a plasmid encoding the L protein and a plasmid encoding NP (pC-L and pC-NP, respectively). The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In addition, the plasmid(s) features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of BHK-21 cells with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, poll and polII promoters from one plasmid.

For recovering the arenavirus the tri-segmented arenavirus vector, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

In certain embodiments, expression of a nucleotide sequence encoding a tumor antigen, tumor associated antigen or antigenic fragment thereof is provided, wherein a plasmid encoding the genomic segment is modified to incorporated a nucleotide sequence encoding a tumor antigen, tumor associated antigen or antigenic fragment thereof. The nucleotide sequence encoding a tumor antigen, tumor associated antigen or antigenic fragment thereof can be incorporated into the plasmid using restriction enzymes.

### (ii) Infectious, Replication-Defective Tri-Segmented Arenavirus Particle

Infectious, replication-defective tri-segmented arenavirus particles can be rescued as described above. However, once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a mammalian cell line such as BHK-21, HEK 293, VERO or other (here BHK-21 will be taken as an example) with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the CMV or EF 1 alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids of two types can be used: i) two plasmids, referred to as TF-plasmids for expressing intracellularly in C-cells the minimal transacting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, poll and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, poll and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient tri-segmented arenavirus expressing an antigen. When used for expression of a CMV antigen in cultured cells, the following two procedures can be used:

i) The cell type of interest is infected with the arenavirus vector preparation described herein at a multiplicity of infection (MOI) of one or more, e.g., two, three or four, resulting in production of the tumor antigen, tumor associated antigen, or antigenic fragment thereof in all cells already shortly after infection.

ii) Alternatively, a lower MOI can be used and individual cell clones can be selected for their level of virally driven expression of a tumor antigen, tumor associated antigen or antigenic fragment thereof. Subsequently individual clones can be expanded infinitely owing to the non-cytolytic nature of arenavirus vectors. Irrespective of the approach, the tumor antigen, tumor associated antigen or antigenic fragment thereof can subsequently be collected (and purified) either from the culture supernatant or from the cells themselves, depending on the properties of the tumor antigen, tumor associated antigen or antigenic fragment produced. However, the invention is not limited to these two strategies, and other ways of driving expression of tumor antigen, tumor associated antigen or antigenic fragment thereof using infectious, replication-deficient arenaviruses as vectors may be considered.

### 5.5 Nucleic Acids, Vector Systems and Cell Lines

In certain embodiments, provided herein are cDNAs comprising or consisting of the arenavirus genomic segment or the tri-segmented arenavirus particle as described herein, which can be used with the methods and compositions provided herein.

### 5.5.1 Non-natural Position Open Reading Frame

In one embodiment, provided herein are nucleic acids that encode an arenavirus genomic segment as described in Section 5.1. In more specific embodiments, provided herein is a DNA nucleotide sequence or a set of DNA nucleotide sequences as set forth in Table 1. Host cells that comprise such nucleic acids are also provided Section 5.1.

In specific embodiments, provided herein is a cDNA of the arenavirus genomic segment engineered to carry an ORF in a position other than the wild-type position of the ORF and a nucleotide sequence encoding a tumor antigen, tumor associated antigen or antigenic fragment thereof, wherein the arenavirus genomic segment encodes a heterologous ORF as described in Section 5.1

In one embodiment, provided herein is a DNA expression vector system that encodes the arenavirus genomic segment engineered to carry an ORF in a position other than the wild-type position of the ORF and a nucleotide sequence encoding a tumor antigen, tumor associated antigen or antigenic fragment thereof. Specifically, provided herein is a DNA expression vector system wherein one or more vectors encodes two arenavirus genomic segments, namely, an L segment and an S segment, of an arenavirus particle described herein. Such a vector system can encode a nucleotide sequence encoding a tumor antigen, tumor associated antigen or antigenic fragment thereof.

In another embodiment, provided herein is a cDNA of the arenavirus S segment that has been engineered to carry an ORF in a position other than the wild-type position and a nucleotide sequence encoding a tumor antigen, tumor associated antigen or antigenic fragment thereof that is part of or incorporated into a DNA expression system. In other embodiments, provided herein is a cDNA of the arenavirus L segment that has been engineered to carry an ORF in a position other than the wild-type position and a nucleotide sequence encoding a tumor antigen, tumor associated antigen or antigenic fragment thereof that is part of or incorporated into a DNA expression system. In certain embodiments, is a cDNA of the arenavirus genomic segment that has been engineered to carry (i) an ORF in a position other than the wild-type position of the ORF; and (ii) and ORF encoding GP, NP, Z protein, or L protein has been removed and replaced with a nucleotide sequence encoding a tumor antigen, tumor associated antigen or antigenic fragment thereof.

In certain embodiments, the cDNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In specific embodiments, the cDNA is derived from LCMV Clone 13. In other specific embodiments, the cDNA is derived from LCMV MP strain.

In certain embodiments, the vector generated to encode an arenavirus particle or a tri-segmented arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein LCMV MP strain.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the arenavirus genomic segment that has been engineered to carry an ORF in a position other than the wild-type position of the ORF and a nucleotide sequence encoding a tumor antigen, tumor associated antigen or antigenic fragment thereof. In some embodiments, the cell comprises the S segment and/or the L segment.

### 5.5.2 Tri-segmented Arenavirus Particle

In one embodiment, provided herein are nucleic acids that encode a tri-segmented arenavirus particle as described in Section 5.2. In more specific embodiments, provided herein is a DNA nucleotide sequence or a set of DNA nucleotide sequences, for example, as set forth in Table 2 or Table 3. Host cells that comprise such nucleic acids are also provided Section 5.2.

In specific embodiments, provided herein is a cDNA consisting of a cDNA of the tri-segmented arenavirus particle that has been engineered to carry an ORF in a position other than the wild-type position of the ORF. In other embodiments, is a cDNA of the tri-segmented arenavirus particle that has been engineered to (i) carry an arenavirus ORF in a position other than the wild-type position of the ORF; and (ii) wherein the tri-segmented arenavirus particle encodes a heterologous ORF as described in Section 5.2.

In one embodiment, provided herein is a DNA expression vector system that together encode the tri-segmented arenavirus particle comprising a nucleotide sequence encoding a tumor antigen, tumor associated antigen or antigenic fragment thereof as described herein. Specifically, provided herein is a DNA expression vector system wherein one or more vectors encode three arenavirus genomic segments, namely, one L segment and two S segments or two L segments and one S segment of a tri-segmented arenavirus particle described herein. Such a vector system can encode a tumor antigen, tumor associated antigen or antigenic fragment thereof.

In another embodiment, provided herein is a cDNA of the arenavirus S segment(s) that has been engineered to carry an ORF in a position other than the wild-type position and a nucleotide sequence encoding a tumor antigen, tumor associated antigen or antigenic fragment thereof that is part of or incorporated into a DNA expression system. In other embodiments, a cDNA of the arenavirus L segment(s) that has been engineered to carry an ORF in a position other than the wild-type position and a nucleotide sequence encoding a tumor antigen, tumor associated antigen or antigenic fragment thereof that is part of or incorporated into a DNA expression system. In certain embodiments, is a cDNA of the tri-segmented arenavirus particle that has been engineered to carry (i) an ORF in a position other than the wild-type position of the ORF; and (ii) an ORF encoding GP, NP, Z protein, or L protein has been removed and replaced with a nucleotide sequence encoding a tumor antigen, tumor associated antigen or antigenic fragment thereof.

In certain embodiments, the cDNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In specific embodiments, the cDNA is derived from LCMV Clone 13. In other specific embodiments, the cDNA is derived from LCMV MP strain.

In certain embodiments, the vector generated to encode an arenavirus particle or a tri-segmented arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein LCMV MP strain.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the tri-segmented arenavirus particle. In some embodiments, the cell comprises the S segment and/or the L segment.

### 5.6 Methods of Use

Vaccines have been successful for preventing and/or treating infectious diseases, such as those for polio virus and measles. However, therapeutic immunization in the setting of established, chronic disease, including cancer has been less successful. The ability to generate one or more arenavirus particles to be injected directly into a solid tumor represents a novel strategy.

In certain embodiments, provided herein are methods of treating a solid tumor in a subject. Such methods can include administering to a subject in need thereof an arenavirus particle provided herein. In certain embodiments, the arenavirus particle used in the methods is a tri-segmented arenavirus particle provided herein, including a replication-competent tri-segmented arenavirus particle. Thus, in certain embodiments, a tri-segmented arenavirus particle used in the methods is replication-competent, wherein the arenavirus particle is engineered to contain a genome comprising: (1) a nucleotide sequence encoding a tumor antigen, tumor associated antigen or an antigenic fragment thereof; (2) the ability to amplify and express its genetic information in infected cells; and (3) the ability to produce further infectious progeny particles in normal, not genetically engineered cells.

Provided herein are methods for treating a solid tumor in a subject comprising injecting an arenavirus particle directly into the tumor wherein the arenavirus particle expresses a tumor antigen or tumor-associated antigen or antigenic fragment thereof. In certain embodiments, injecting comprises multiple administrations of the same arenavirus particle. In certain embodiments, injecting comprises multiple administrations of arenavirus particles derived from the same arenavirus (that is, with the same backbone), but expressing different tumor antigens or tumor-associated antigens or antigenic fragments thereof. In certain embodiments, injecting comprises multiple administrations of arenavirus particles derived from different arenaviruses (that is, with different backbones), but expressing the same tumor antigen or tumor-associated antigen or antigenic fragment thereof. In certain embodiments, injecting comprises multiple administrations of arenavirus particles derived from different arenaviruses (that is, with different backbones), and expressing different tumor antigens or tumor-associated antigens or antigenic fragments thereof.

In other embodiments, provided herein are methods for treating a solid tumor in a subject comprising injecting an arenavirus particle directly into the tumor wherein the arenavirus particle expresses a tumor antigen or tumor-associated antigen or antigenic fragment thereof, further comprising systemically administering a first arenavirus particle prior said injecting. In certain embodiments, systemically administering comprises multiple administrations of the same arenavirus particle. In certain embodiments, systemically administering a first arenavirus particle comprises multiple administrations of arenavirus particles derived from the same arenavirus (that is, with the same backbone), but expressing different tumor antigens or tumor-associated antigens or antigenic fragments thereof. In certain embodiments, systemically administering a first arenavirus particle comprises multiple administrations of arenavirus particles derived from different arenaviruses (that is, with different backbones), but expressing the same tumor antigen or tumor-associated antigen or antigenic fragment thereof. In certain embodiments, systemically administering a first arenavirus particle comprises multiple administrations of arenavirus particles derived from different arenaviruses (that is, with different backbones), and expressing different tumor antigens or tumor-associated antigens or antigenic fragments thereof.

In other embodiments, provided herein are methods for treating a solid tumor in a subject comprising injecting an arenavirus particle directly into the tumor wherein the arenavirus particle expresses a tumor antigen or tumor-associated antigen or antigenic fragment thereof, further comprising systemically administering a second arenavirus particle after said injecting. In certain embodiments, systemically administering comprises multiple administrations of the same arenavirus particle. In certain embodiments, systemically administering a second arenavirus particle comprises multiple administrations of arenavirus particles derived from the same arenavirus (that is, with the same backbone), but expressing different tumor antigens or tumor-associated antigens or antigenic fragments thereof. In certain embodiments, systemically administering a second arenavirus particle comprises multiple administrations of arenavirus particles derived from different arenaviruses (that is, with different backbones), but expressing the same tumor antigen or tumor-associated antigen or antigenic fragment thereof. In certain embodiments, systemically administering a second arenavirus particle comprises multiple administrations of arenavirus particles derived from different arenaviruses (that is, with different backbones), and expressing different tumor antigens or tumor-associated antigens or antigenic fragments thereof.

In certain embodiments, provided herein are methods for treating a solid tumor in a subject comprising (a) administering a first arenavirus particle to a subject, wherein the first arenavirus particle does not express a tumor antigen or tumor-associated antigen or antigenic fragment thereof; and (b) administering a second arenavirus particle to a subject, wherein the second arenavirus particle expresses a tumor antigen or tumor-associated antigen or antigenic fragment thereof. In certain embodiments, administering comprises multiple administrations of the same arenavirus particle. In certain embodiments, administering a first arenavirus particle comprises multiple administrations of arenavirus particles derived from different arenaviruses (that is, with different backbones). In certain embodiments, administering a second arenavirus particle comprises multiple administrations of arenavirus particles derived from the same arenavirus (that is, with the same backbone), but expressing different tumor antigens or tumor-associated antigens or antigenic fragments thereof. In certain embodiments, administering a second arenavirus particle comprises multiple administrations of arenavirus particles derived from different arenaviruses (that is, with different backbones), but expressing the same tumor antigen or tumor-associated antigen or antigenic fragment thereof. In certain embodiments, administering a second arenavirus particle comprises multiple administrations of arenavirus particles derived from different arenaviruses (that is, with different backbones), and expressing different tumor antigens or tumor-associated antigens or antigenic fragments thereof.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof; and (b) injecting a second arenavirus particle directly into the tumor, wherein the second arenavirus particle expresses a tumor antigen or tumor-associated antigen or antigenic fragment thereof.

In another embodiment, provided herein are methods for treating a solid tumor in a subject comprising (a) intravenously administering a first arenavirus particle to the subject, wherein the first arenavirus particle does not express a tumor antigen or tumor-associated antigen or antigenic fragment thereof; and (b) injecting a second arenavirus particle directly into the tumor, wherein the second arenavirus particle expresses a tumor antigen or tumor-associated antigen or antigenic fragment thereof.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof; and (b) intravenously administering a second arenavirus particle to the subject, wherein the second arenavirus particle expresses a tumor antigen or tumor-associated antigen or antigenic fragment thereof.

In certain embodiments, the first arenavirus particle does not express a foreign antigen. In certain embodiments, the first arenavirus particle comprises a nucleotide comprising a deleted or inactivated viral ORF. In certain embodiments, the first arenavirus particle comprises a nucleotide wherein the UTR is directly fused to the IGR. In certain embodiments, the first arenavirus particle comprises a nucleotide comprising an ORF for a marker, such as GFP. In certain embodiments, the first arenavirus particle comprises a nucleotide comprising a heterologous non-coding sequence.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof; and (b) administering a second arenavirus particle to the subject, wherein the second arenavirus particle expresses a tumor antigen or tumor-associated antigen or antigenic fragment thereof. In certain embodiments, the first arenavirus particle does not express a foreign antigen. In certain embodiments, the first arenavirus particle comprises a nucleotide comprising a deleted or inactivated viral ORF. In certain embodiments, the first arenavirus particle comprises a nucleotide wherein the UTR is directly fused to the IGR. In certain embodiments, the first arenavirus particle comprises a nucleotide comprising an ORF for a marker, such as GFP. In certain embodiments, the first arenavirus particle comprises a nucleotide comprising a heterologous non-coding sequence. In specific embodiments, the second arenavirus particle is replication-competent. In specific embodiments, the second arenavirus particle is replication-defective. In certain embodiments, the second arenavirus particle is tri-segmented. In specific embodiments, the second arenavirus particle is tri-segmented and replication-competent. In specific embodiments, the second arenavirus particle is tri-segmented and replication-defective.

In another embodiment, provided herein are methods for treating a solid tumor in a subject comprising (a) injecting a first arenavirus particle directly into the tumor, wherein the first arenavirus particle is replication-competent and does not express a tumor antigen or tumor-associated antigen or antigenic fragment thereof; and (b) administering a second arenavirus particle to the subject, wherein the second arenavirus particle expresses a tumor antigen or tumor-associated antigen or antigenic fragment thereof. In certain embodiments, the first arenavirus particle does not express a foreign antigen. In certain embodiments, the first arenavirus particle comprises a nucleotide comprising a deleted or inactivated viral ORF. In certain embodiments, the first arenavirus particle comprises a nucleotide wherein the UTR is directly fused to the IGR. In certain embodiments, the first arenavirus particle comprises a nucleotide comprising an ORF for a marker, such as GFP. In certain embodiments, the first arenavirus particle comprises a nucleotide comprising a heterologous non-coding sequence.

In another embodiment, provided herein are methods for treating a solid tumor in a subject comprising (a) injecting a first arenavirus particle directly into the tumor, wherein the first arenavirus particle is replication-competent and expresses a tumor antigen or tumor-associated antigen or antigenic fragment thereof; and (b) administering a second arenavirus particle to the subject, wherein the second arenavirus particle expresses a tumor antigen or tumor-associated antigen or antigenic fragment thereof. In certain embodiments, the first arenavirus particle is tri-segmented. In specific embodiments, the second arenavirus particle is replication-competent. In specific embodiments, the second arenavirus particle is replication-defective. In certain embodiments, the second arenavirus particle is tri-segmented. In specific embodiments, the second arenavirus particle is tri-segmented and replication-competent. In specific embodiments, the second arenavirus particle is tri-segmented and replication-defective.

In one embodiment, provided herein are methods of treating a solid tumor in a subject comprising administering to the subject one or more arenavirus particles expressing a tumor antigen, tumor associated antigen or an antigenic fragment thereof as provided herein or a composition thereof, optionally in combination with one or more arenavirus particles that do not express a foreign antigen. In a specific embodiment, a method for treating a solid tumor described herein comprises administering to a subject in need thereof a therapeutically effective amount of one or more arenavirus particles expressing a tumor antigen, tumor associated antigen or an antigenic fragment thereof provided herein or a composition thereof, optionally in combination with one or more arenavirus particles that do not express a foreign antigen. The subject can be a mammal, such as but not limited to a human, a mouse, a rat, a guinea pig, a domesticated animal, such as, but not limited to, a cow, a horse, a sheep, a pig, a goat, a cat, a dog, a hamster, a donkey. In a specific embodiment, the subject is a human.

In another embodiment, provided herein are methods for inducing an immune response against a solid tumor cell in a subject comprising administering to the subject an arenavirus particle expressing a tumor antigen, tumor associated antigen or an antigenic fragment thereof provided herein, or a composition thereof, optionally in combination with one or more arenavirus particles that do not express a foreign antigen.

In another embodiment, the subjects having a solid tumor to whom an arenavirus particle expressing a tumor antigen, tumor associated antigen or an antigenic fragment thereof provided herein, or a composition thereof is administered, optionally in combination with one or more arenavirus particles that do not express a foreign antigen, have, are susceptible to, or are at risk for a neoplastic disease.

In another embodiment, the subjects having a solid tumor to whom an arenavirus particle expressing a tumor antigen, tumor associated antigen or an antigenic fragment thereof provided herein, or a composition thereof is administered, optionally in combination with one or more arenavirus particles that do not express a foreign antigen, have, are susceptible to, or are at risk for development of a neoplastic disease, such as cancer, or exhibit a pre-cancerous tissue lesion. In another specific embodiment, the subjects to whom arenavirus particle expressing a tumor antigen, tumor associated antigen or an antigenic fragment thereof provided herein, or a composition thereof is administered, optionally in combination with one or more arenavirus particles that do not express a foreign antigen, are diagnosed with a neoplastic disease, such as cancer, or exhibit a pre-cancerous tissue lesion.

In another embodiment, the subjects having a solid tumor to whom an arenavirus particle expressing a tumor antigen, tumor associated antigen or an antigenic fragment thereof provided herein, or a composition thereof, is administered, optionally in combination with one or more arenavirus particles that do not express a foreign antigen, are suffering from, are susceptible to, or are at risk for, a neoplastic disease selected from, but not limited to, acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to a subject of any age group having a solid tumor and suffering from, susceptible to, or at risk for a neoplastic disease. In a specific embodiment, an arenavirus particle expressing a tumor antigen, tumor associated antigen or an antigenic fragment thereof provided herein, or a composition thereof, is administered, optionally in combination with one or more arenavirus particles that do not express a foreign antigen, to a subject having a solid tumor with a compromised immune system, a pregnant subject, a subject undergoing an organ or bone marrow transplant, a subject taking immunosuppressive drugs, a subject undergoing hemodialysis, a subject who has cancer, or a subject who is suffering from, are susceptible to, or are at risk for a neoplastic disease. In a more specific embodiment, an arenavirus particle expressing a tumor antigen, tumor associated antigen or an antigenic fragment thereof provided herein, or a composition thereof, provided herein is administered, optionally in combination with one or more arenavirus particles that do not express a foreign antigen, to a subject who is a child of 0, 1, 2, 3, 4, 5, 6, 7, 8, 9, 10, 11, 12, 13, 14, 15, 16, or 17 years of age suffering from, are susceptible to, or are at risk for a neoplastic disease. In yet another specific embodiment, an arenavirus particle expressing a tumor antigen, tumor associated antigen or an antigenic fragment thereof provided herein, or a composition thereof, is administered, optionally in combination with one or more arenavirus particles that do not express a foreign antigen, to a subject who is an infant suffering from, is susceptible to, or is at risk for a neoplastic disease. In yet another specific embodiment, an arenavirus particle expressing a tumor antigen, tumor associated antigen or an antigenic fragment thereof provided herein, or a composition thereof, is administered, optionally in combination with one or more arenavirus particles that do not express a foreign antigen, to a subject who is an infant of 0, 1, 2, 3, 4, 5, 6, 7, 8, 9, 10, 11, or 12 months of age suffering from, is susceptible to, or is at risk for a neoplastic disease. In yet another specific embodiment, an arenavirus particle expressing a tumor antigen, tumor associated antigen or an antigenic fragment thereof provided herein, or a composition thereof, is administered, optionally in combination with one or more arenavirus particles that do not express a foreign antigen, to an elderly subject who is suffering from, is susceptible to, or is at risk for a neoplastic disease. In a more specific embodiment, an arenavirus particle expressing a tumor antigen, tumor associated antigen or an antigenic fragment thereof provided herein, or a composition thereof, is administered, optionally in combination with one or more arenavirus particles that do not express a foreign antigen, to a subject who is a senior subject of 65, 66, 67, 68, 69, 70, 71, 72, 73, 74, 75, 76, 77, 78, 79, 80, 81, 82, 83, 84, 85, 86, 87, 88, 89, or 90 years of age. Provided herein is a method for preventing a cancer in a subject susceptible to, or is at risk for a neoplastic disease.

In another embodiment, an arenavirus particle expressing a tumor antigen, tumor associated antigen or an antigenic fragment thereof provided herein, or a composition thereof, provided herein is administered, optionally in combination with one or more arenavirus particles that do not express a foreign antigen, to subjects with a heightened risk of cancer metastasis. In a specific embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to subjects in the neonatal period with a neonatal and therefore immature immune system.

In another embodiment, an arenavirus particle expressing a tumor antigen, tumor associated antigen or an antigenic fragment thereof provided herein, or a composition thereof, provided herein is administered, optionally in combination with one or more arenavirus particles that do not express a foreign antigen, to a subject having grade 0 (i.e., in situ neoplasm), grade 1, grade 2, grade 3 or grade 4 cancer or a subcategory thereof, such as grade 3A, 3B, or 3C, or an equivalent thereof.

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to a subject having cancer at a Tumor, Node, Metastasis (TNM) stage of any combination selected from Tumor T1, T2, T3, and T4, and Node N0, N1, N2, or N3, and Metastasis M0 and M1.

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., T0, T is, or T1-4), state of metastasis (e.g., M0, M1), number of observable tumors, node involvement (e.g., NO, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measureable by percent, can be at least 5, 10, 15, 20, 25, 30, 40, 50, 60, 70, 80, or 90% (e.g., survival, or volume or linear dimensions of a tumor).

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to a subject having a dormant cancer (e.g., the subject is in remission). Thus, provided herein is a method for preventing reactivation of a cancer. Also provided herein are methods for reducing the frequency of reoccurrence of a cancer.

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to a subject having a recurrent a cancer.

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to a subject with a genetic predisposition for a cancer. In another embodiment, an arenavirus particle expressing a tumor antigen, tumor associated antigen or an antigenic fragment thereof provided herein, or a composition thereof, is administered to a subject with risk factors. Exemplary risk factors include aging, tobacco, sun exposure, radiation exposure, chemical exposure, family history, alcohol, poor diet, lack of physical activity, or being overweight.

In another embodiment, an arenavirus particle expressing a tumor antigen, tumor associated antigen or an antigenic fragment thereof provided herein, or a composition thereof is administered, optionally in combination with one or more arenavirus particles that do not express a foreign antigen, to subjects who suffer from one or more types of cancers. In other embodiments, any type of neoplastic disease, such as cancer, that is susceptible to treatment with the compositions described herein might be targeted.

In another embodiment, administering an arenavirus particle expressing a tumor antigen, tumor associated antigen or an antigenic fragment thereof provided or a composition thereof, optionally in combination with one or more arenavirus particles that do not express a foreign antigen, to subjects confer cell-mediated immunity (CMI) against a neoplastic cell or tumor, such as a cancer cell or tumor. Without being bound by theory, in another embodiment, an arenavirus particle expressing a tumor antigen, tumor associated antigen or an antigenic fragment thereof provided or a composition thereof, optionally in combination with one or more arenavirus particles that do not express a foreign antigen, infects and expresses antigens of interest in antigen presenting cells (APC) of the host (e.g., macrophages) for direct presentation of antigens on Major Histocompatibility Complex (MHC) class I and II. In another embodiment, administering an arenavirus particle expressing a tumor antigen, tumor associated antigen or an antigenic fragment thereof provided herein, or a composition thereof, optionally in combination with one or more arenavirus particles that do not express a foreign antigen, to subjects induces plurifunctional IFN-γ and TNF-α co-producing cancer-specific CD4+ and CD8+ T cell responses (IFN-γ is produced by CD4+ and CD8+ T cells and TNF-α is produced by CD4+ T cells) of high magnitude to treat a neoplastic disease.

In another embodiment, administering an arenavirus particle expressing a tumor antigen, tumor associated antigen or an antigenic fragment thereof provided herein, or a composition thereof, optionally in combination with one or more arenavirus particles that do not express a foreign antigen, increases or improves one or more clinical outcome for cancer treatment. Non-limiting examples of such outcomes are overall survival, progression-free survival, time to progression, time to treatment failure, event-free survival, time to next treatment, overall response rate and duration of response. The increase or improvement in one or more of the clinical outcomes can be by at least about 10%, at least about 20%, at least about 25%, at least about 30%, at least about 35%, at least about 40%, at least about 50%, at least about 60%, at least about 70%, at least about 80%, at least about 90%, or more, compared to a patient or group of patients having the same neoplastic disease in the absence of such treatment.

Changes in cell-mediated immunity (CMI) response function against a neoplastic cell or tumor, including a cancer cell or tumor, induced by administering an arenavirus particle expressing a tumor antigen, tumor associated antigen or an antigenic fragment thereof provided herein, or a composition thereof, in subjects can be measured by any assay known to the skilled artisan including, but not limited to flow cytometry (see, e.g., Perfetto S. P. et al., Nat Rev Immun. 2004; 4(8):648-55), lymphocyte proliferation assays (see, e.g., Bonilla F. A. et al., Ann Allergy Asthma Immunol. 2008; 101:101-4; and Hicks M. J. et al., Am J Clin Pathol. 1983; 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C. C. et al., J Immunol Methods. 1983; 65:109-121; and Hutchings P. R., et al., J Immunol Methods. 1989; 120:1-8), or Natural killer cell cytotoxicity assays (see, e.g., Bonilla F. A. et al., Ann Allergy Asthma Immunol. 2005 May; 94(5 Suppl 1):S1-63).

Chemotherapeutic agents described herein administered in combination with an arenavirus particle expressing a tumor antigen, tumor associated antigen or an antigenic fragment thereof provided herein, or a composition thereof, optionally in combination with one or more arenavirus particles that do not express a foreign antigen, can be alkylating agents (e.g., cyclophosphamide), platinum-based therapeutics, antimetabolites, topoisomerase inhibitors, cytotoxic antibiotics, intercalating agents, mitosis inhibitors, taxanes, or combinations of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide.

Immune checkpoint modulators described herein administered in combination with an arenavirus particle expressing a tumor antigen, tumor associated antigen or an antigenic fragment thereof provided herein, or a composition thereof, optionally in combination with one or more arenavirus particles that do not express a foreign antigen, can be immune checkpoint inhibitors that inhibit, decrease or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, one or more arenavirus particles provided herein, or a composition thereof, are preferably administered via intratumoral injection, that is, directly into the tumor. In certain embodiments, such intratumoral injection is administered via multiple injections (e.g., at least 2, 3, 4, 5, 6, 7, 8, 9, 10, 12, 14, 16, 18, 20, 25, 30, 40, 45, or 50 injections). In certain embodiments, said multiple injections administer different arenavirus particles, for example, a first arenavirus particle that does not express a foreign antigen and a second arenavirus particle that expresses a tumor antigen, tumor associated antigen or an antigenic fragment thereof provided herein.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, in two or more separate injections over a 1-hour period, 2-hour period, 3-hour period, 6-hour period, a 12-hour period, a 24-hour period, or a 48-hour period.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, in two or more separate injections over a 3-day period, a 5-day period, a 1-week period, a 2-week period, a 3-week period, a 4-week period, or a 12-week period.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, in two or more separate injections over a 6-month period, a 12-month period, a 24-month period, or a 48-month period.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected time, and a second dose at least 2 hours after the first dose. In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, a second dose at least 2 hours after the first dose, and a third dose 6 hours after the first dose.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, and a second dose at least 2 days after the first dose. In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, a second dose at least 2 days after the first dose, and a third dose 6 days after the first dose.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, and a second dose at least 2 weeks after the first dose. In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, a second dose at least 2 weeks after the first dose, and a third dose 6 weeks after the first dose.

In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, and a second dose at least 2 months after the first dose. In certain embodiments, the one or more arenavirus particles expressing a tumor antigen, tumor associated antigen or an antigenic fragment thereof provided herein, or a composition thereof, are administered, optionally in combination with one or more arenavirus particles that do not express a foreign antigen, with a first dose at an elected date, a second dose at least 2 months after the first dose, and a third dose 6 months after the first dose.

In certain embodiments, one or more arenavirus particles provided herein, or a composition thereof, are administered via peritumoral injection.

In certain embodiments, one or more arenavirus particles provided herein, or a composition thereof are administered, optionally in combination with one or more arenavirus particles that do not express a foreign antigen, via intratumoral injection in combination with a second set of one or more arenavirus particles provided herein administered via another method. In certain embodiments, the second set of one or more arenavirus particles provided herein are administered systemically, for example, intravenously. In certain embodiments, one or more arenavirus particles provided herein that do not express a foreign antigen are administered intratumorally in combination with one or more arenavirus particles expressing a tumor antigen, tumor associated antigen or an antigenic fragment thereof provided herein, administered systemically, for example, intravenously.

In certain embodiments, the methods further comprise co-administration of the arenavirus particle provided herein and another agent, such as a chemotherapeutic agent or an immune checkpoint modulator. In certain embodiments, the co-administration is simultaneous. In another embodiment, the arenavirus particle is administered prior to administration of the other agent. In other embodiments, the arenavirus particle is administered after administration of the other agent. In certain embodiments, the interval between administration of the arenavirus particle and the other agent is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the arenavirus particle and the other agent is about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the arenavirus particle and the other agent is about 1 month, about 2 months, about 3 months, about 4 months, about 5 months, or about 6 months. In some embodiments, the method further includes administering at least one additional therapy.

In embodiments wherein two arenavirus particles are administered in a treatment regime, administration may be at molar ratios ranging from about 1:1 to 1:1000, in particular including: 1:1 ratio, 1:2 ratio, 1:5 ratio, 1:10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio. In certain embodiments, one arenavirus particle that does not express a foreign antigen is administered in combination with a second arenavirus particle expressing a tumor antigen, tumor associated antigen or an antigenic fragment thereof provided herein.

In certain embodiments, provided herein is a method of treating solid tumor wherein a first arenavirus particle is administered first as a “prime,” and a second arenavirus particle is administered as a “boost.” The first and the second arenavirus particles can express the same or different tumor antigens, tumor associated antigens or antigenic fragments thereof, or the first or second arenavirus particle does not express a foreign antigen. Alternatively, or additionally, some certain embodiments, the “prime” and “boost” administration are performed with an arenavirus particle derived from different species. In certain specific embodiments, the “prime” administration is performed with an arenavirus particle derived from LCMV, and the “boost” is performed with an arenavirus particle derived from Junin virus. In certain specific embodiments, the “prime” administration is performed with an arenavirus particle derived from Junin virus, and the “boost” is performed with an arenavirus particle derived from LCMV.

In certain embodiments, administering a first arenavirus particle expressing a tumor antigen, tumor associated antigen or antigenic fragment thereof, followed by administering a second arenavirus particle expressing a tumor antigen, tumor associated antigen or antigenic fragment thereof results in a greater antigen specific CD8+ T cell response than administering a single arenavirus particle expressing a tumor antigen, tumor associated antigen or antigenic fragment thereof. In certain embodiments, said first or second arenavirus particle does not express a foreign antigen. In certain embodiments, the antigen specific CD8+ T cell count increases by 50%, 100%, 150% or 200% after the second administration compared to the first administration. In certain embodiments, administering a third arenavirus particle expressing a tumor antigen, tumor associated antigen or antigenic fragment thereof results in a greater antigen specific CD8+ T cell response than administering two consecutive arenavirus particles expressing a tumor antigen, tumor associated antigen or antigenic fragment thereof. In certain embodiments, the antigen specific CD8+ T cell count increases by about 50%, about 100%, about 150%, about 200% or about 250% after the third administration compared to the first administration.

In certain embodiments, provided herein are methods for treating a solid tumor comprising administering two or more arenavirus particles, wherein the two or more arenavirus particles are homologous, and wherein the time interval between each administration is about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, administering a first arenavirus particle expressing a tumor antigen, tumor associated antigen or antigenic fragment thereof and a second, heterologous, arenavirus particle expressing a tumor antigen, tumor associated antigen or antigenic fragment thereof elicits a greater CD8+ T cell response than administering a first arenavirus particle expressing a tumor antigen, tumor associated antigen or antigenic fragment thereof and a second, homologous, arenavirus particle expressing a tumor antigen, tumor associated antigen or antigenic fragment thereof. In certain embodiments, said first or second arenavirus particle does not express a foreign antigen.

### 5.7 Compositions, Administration, and Dosage

In certain embodiments, immunogenic compositions (e.g., vaccine formulations), and pharmaceutical compositions comprising an arenavirus particle provided herein can be used with the methods and compositions provided herein. Such vaccines, immunogenic compositions and pharmaceutical compositions can be formulated according to standard procedures in the art.

In another embodiment, provided herein are compositions comprising an arenavirus particle described herein. Such compositions can be used in methods of treating a solid tumor. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the treatment of a neoplastic disease a subject (e.g., human subject). In other embodiments, the vaccine, immunogenic composition or pharmaceutical composition are suitable for veterinary and/or human administration.

In certain embodiments, provided herein are immunogenic compositions comprising an arenavirus particle (or a combination of different arenavirus particles) as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an arenavirus particle, but when the compound is administered alone does not generate an immune response to the arenavirus particle. In some embodiments, the adjuvant generates an immune response to the arenavirus particle and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions comprise the arenavirus particles described herein alone or together with a pharmaceutically acceptable carrier. Suspensions or dispersions of genetically engineered arenavirus particles, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2-8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenavirus particles. Unit dose forms for parenteral administration are, for example, ampoules or vials, e.g., vials containing from about 10³ to 10¹⁰ focus forming units or 10⁵ to 10¹⁵ physical particles of genetically engineered arenavirus particles.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous, intramuscular or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

The dosage of the active ingredient depends upon the type of vaccination and upon the subject, and their age, weight, individual condition, the individual pharmacokinetic data, and the mode of administration.

In certain embodiments, the compositions can be administered to the patient in a single dosage comprising a therapeutically effective amount of the arenavirus particle and, optionally, a therapeutically effective amount of another agent. In some embodiments, the arenavirus particle can be administered to the patient in a single dose comprising an arenavirus particle, optionally with another agent, in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus particle or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

In certain embodiments, the vaccine, immunogenic composition, or pharmaceutical composition comprising an arenavirus particle can be used as a live vaccination. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 101² pfu, and can be administered to a subject once, twice, three or more times with intervals as often as needed. In another embodiment, a live arenavirus is formulated such that a 0.2-mL dose contains 10^{6.5}-10^{7.5} fluorescent focal units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus

Also provided are processes and uses of an arenavirus particle for the manufacture of vaccines in the form of pharmaceutical preparations, which comprise the arenavirus particle as an active ingredient. Still further provided is a combination of an arenavirus particle provided herein and a second agent for use in the treatment of a neoplastic disease described herein. In certain embodiments, the combination is in the same pharmaceutical composition. In certain embodiments, the combination is not in the same pharmaceutical composition, such as when the arenavirus particle and the second agent are to be separately administered. The pharmaceutical compositions of the present application are prepared in a manner known per se, for example by means of conventional mixing and/or dispersing processes.

Also provided herein are kits that can be used to perform the methods described herein. In certain embodiments, the kit provided herein can include one or more containers. These containers can hold for storage the compositions (e.g., pharmaceutical, immunogenic or vaccine composition) provided herein. Also included in the kit are instructions for use. These instructions describe, in sufficient detail, a treatment protocol for using the compositions contained therein. For example, the instructions can include dosing and administration instructions as provided herein for the methods of treating a neoplastic disease.

In certain embodiments, a kit provided herein includes containers that each contains the active ingredients for performing the methods described herein.

### 5.8 Assays

### 5.8.1 Arenavirus Detection Assays

The skilled artesian could detect an arenavirus genomic segment or tri-segmented arenavirus particle, as described herein using techniques known in the art. For example, RT-PCR can be used with primers that are specific to an arenavirus to detect and quantify an arenavirus genomic segment that has been engineered to carry an ORF in a position other than the wild-type position of the ORF or a tri-segmented arenavirus particle. Western blot, ELISA, radioimmunoassay, immunoprecipitation, immunocytochemistry, or immunocytochemistry in conjunction with FACS can be used to quantify the gene products of the arenavirus genomic segment or tri-segmented arenavirus particle.

### 5.8.2 Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells, e.g., MC57 cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and thus countable using antibodies against LCMV-NP or another protein expressed by the arenavirus particle or the tri-segmented arenavirus particle and a HRP-based color reaction. The titer of a virus/vector can be calculated in focus-forming units per milliliter (FFU/mL).

### 5.8.3 Growth of an Arenavirus Particle

Growth of an arenavirus particle described herein can be assessed by any method known in the art or described herein (e.g., cell culture). Viral growth may be determined by inoculating serial dilutions of an arenavirus particle described herein into cell cultures (e.g., Vero cells or BHK-21 cells). After incubation of the virus for a specified time, the virus is isolated using standard methods.

### 5.8.4 Serum ELISA

Determination of the humoral immune response upon vaccination of animals (e.g., mice, guinea pigs) can be done by antigen-specific serum ELISAs (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

Immunocapture ELISA (IC-ELISA) may also be performed (see Shanmugham et al., 2010, Clin. Vaccine Immunol. 17(8):1252-1260), wherein the capture agents are cross-linked to beads.

### 5.8.5 Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

### 5.8.6 Plaque Reduction Assay

In brief, plaque reduction (neutralization) assays for LCMV can be performed by use of a replication-competent or -deficient LCMV that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples. qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### 5.8.7 Neutralization Assay in Guinea Pig Lung Fibroblast (GPL) Cells

In brief, serial dilutions of test and control (pre-vaccination) sera were prepared in GPL complete media with supplemental rabbit serum (1%) as a source of exogenous complement. The dilution series spanned 1:40 through 1:5120. Serum dilutions were incubated with eGFP tagged virus (100-200 pfu per well) for 30 min at 37° C., and then transferred to 12-well plates containing confluent GPL cells. Samples were processed in triplicate. After 2 hours incubation at 37° C. the cells were washed with PBS, re-fed with GPL complete media and incubated at 37° C./5% C02 for 5 days. Plaques were visualized by fluorescence microscopy, counted, and compared to control wells. That serum dilution resulting in a 50% reduction in plaque number compared to controls was designated as the neutralizing titer.

### 5.8.8 Western Blotting

Infected cells grown in tissue culture flasks or in suspension are lysed at indicated time points post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogen's iBlot Gel transfer Device and visualized by Ponceau staining. Finally, the preparations are probed with a primary antibodies directed against proteins of interest and alkaline phosphatase conjugated secondary antibodies followed by staining with 1-Step NBT/BCIP solution (INVITROGEN).

### 5.8.9 MHC-Peptide Multimer Staining Assay for Detection of Antigen-Specific CD8+ T-Cell Proliferation

Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for a defined antigen specificity and MHC haplotype of T-cells (typically fluorescently labeled). The tetramer is then detected by flow cytometry via the fluorescent label.

### 5.8.10 ELISPOT Assay for Detection of Antigen-Specific CD4+ T-Cell Proliferation

Any assay known to the skilled artisan can be used to test antigen-specific CD4+ T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Briefly, the assay comprises the following steps: An immunospot plate is coated with an anti-cytokine antibody. Cells are incubated in the immunospot plate. Cells secrete cytokines and are then washed off. Plates are then coated with a second biotyinlated-anticytokine antibody and visualized with an avidin-HRP system.

### 5.8.11 Intracellular Cytokine Assay for Detection of Functionality of CD8+ and CD4+ T-Cell Responses

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After a defined period of incubation, typically 5 hours, a washing steps follows, and antibodies to other cellular markers can be added to the cells. Cells are then fixed and permeabilized. The fluorochrome-conjugated anti-cytokine antibodies are added and the cells can be analyzed by flow cytometry.

### 5.8.12 Assay for Confirming Replication-Deficiency of Viral Vectors

Any assay known to the skilled artisan that determines concentration of infectious and replication-competent virus particles can also be used to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32: Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

### 5.8.13 Assay for Expression of Viral Antigen

Any assay known to the skilled artisan can be used for measuring expression of viral antigens. For example, FFU assays can be performed. For detection, mono- or polyclonal antibody preparation(s) against the respective viral antigens are used (transgene-specific FFU).

### 5.8.14 Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice can be used to investigate recombination and infectivity of an arenavirus particle are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal model. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene stability.

### 6. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 7. SEQUENCES

The sequences in Table 4 are illustrative amino acid sequences and nucleotide sequences that can be used with the methods and compositions described herein. In some instances a DNA sequence is used to describe the RNA sequence of a viral genomic segment. The RNA sequence can be readily deduced from the DNA sequence.

TABLE 4

| SEQ | | |
|---|---|---|
| ID | | |
| NO. | Description | Sequence |
| 1 | Lymphocytic | GCGCACCGGGGATCCTAGGCGTTTAGTTGCGCTGTTTGGTTGCACAACT |
| | choriomeningitis | TTCTTCGTGAGGCTGTCAGAAGTGGACCTGGCTGATAGCGATGGGTCAA |
| | virus clone 13 | GGCAAGTCCAGAGAGGAGAAAGGCACCAATAGTACAAACAGGGCCGAAA |
| | segment L, complete | TCCTACCAGATACCACCTATCTTGGCCCTTTAAGCTGCAAATCTTGCTG |
| | sequence (GenBank: | GCAGAAATTTGACAGCTTGGTAAGATGCCATGACCACTACCTTTGCAGG |
| | DQ361066.1) | CACTGTTTAAACCTTCTGCTGTCAGTATCCGACAGGTGTCCTCTTTGTA |
| | (The genomic | AATATCCATTACCAACCAGATTGAAGATATCAACAGCCCCAAGCTCTCC |
| | segment is RNA, the | ACCTCCCTACGAAGAGTAACACCGTCCGGCCCCGGCCCCGACAAACAGC |
| | sequence in SEQ ID | CCAGCACAAGGGAACCGCACGTCaCCCAACGCACACAGACACAGCACCC |
| | NO: 1 is shown for | AACACAGAACACGCACACACACACACACACACACCCACACGCACGCGCC |
| | DNA; however, | CCCACCACCGGGGGGCGCCCCCCCCCGGGGGGCGGCCCCCCGGGAGCCC |
| | exchanging all | GGGCGGAGCCCCACGGAGATGCCCATCAGTCGATGTCCTCGGCCACCGA |
| | thymidines (“T”) in | CCCGCCcAGCCAATCGTCGCAGGACCTCCCCTTGAGTCTAAACCTGCCC |
| | SEQ ID NO: 1 for | CCCACTgTTTCATACATCAAAGTGCTCCTAGATTTGCTAAAACAAAGTC |
| | uridines (“U”) | TGCAATCCTTAAAGGCGAACCAGTCTGGCAAAAGCGACAGTGGAATCAG |
| | provides the RNA | CAGAATAGATCTGTCTATACATAGTTCCTGGAGGATTACACTTATCTCT |
| | sequence.) | GAACCCAACAAATGTTCACCAGTTCTGAATCGATGCAGGAAGAGGTTCC |
| | | CAAGGACATCACTAATCTTTTCATAGCCCTCAAGTCCTGCTAGAAAGAC |
| | | TTTCATGTCCTTGGTCTCCAGCTTCACAATGATATTTTGGACAAGGTTT |
| | | CTTCCTTCAAAAAGGGCACCCATCTTTACAGTCAGTGGCACAGGCTCCC |
| | | ACTCAGGTCCAACTCTCTCAAAGTCAATAGATCTAATCCCATCCAGTAT |
| | | TCTTTTGGAGCCCAACAACTCAAGCTCAAGAGAATCACCAAGTATCAAG |
| | | GGATCTTCCATGTAATCCTCAAACTCTTCAGATCTGATATCAAAGACAC |
| | | CATCGTTCACCTTGAAGACAGAGTCTGTCCTCAGTAAGTGGAGGCATTC |
| | | ATCCAACATTCTTCTATCTATCTCACCCTTAAAGAGGTGAGAGCATGAT |
| | | AAAAGTTCAGCCACACCTGGATTCTGTAATTGGCACCTAACCAAGAATA |
| | | TCAATGAAAATTTCCTTAAACAGTCAGTATTATTCTGATTGTGCGTAAA |
| | | GTCCACTGAAATTGAAAACTCCAATACCCCTTTTGTGTAGTTGAGCATG |
| | | TAGTCCCACAGATCCTTTAAGGATTTAAATGCCTTTGGGTTTGTCAGGC |
| | | CCTGCCTAATCAACATGGCAGCATTACACACAACATCTCCCATTCGGTA |
| | | AGAGAACCACCCAAAACCAAACTGCAAATCATTCCTAAACATAGGCCTC |
| | | TCCACATTTTTGTTCACCACCTTTGAGACAAATGATTGAAAGGGGCCCA |
| | | GTGCCTCAGCACCATCTTCAGATGGCATCATTTCTTTATGAGGGAACCA |
| | | TGAAAAATTGCCTAATGTCCTGGTTGTTGCAACAAATTCTCGAACAAAT |
| | | GATTCAAAATACACCTGTTTTAAGAAGTTCTTGCAGACATCCCTCGTGC |
| | | TAACAACAAATTCATCAACCAGACTGGAGTCAGATCGCTGATGAGAATT |
| | | GGCAAGGTCAGAAAACAGAACAGTGTAATGTTCATCCCTTTTCCACTTA |
| | | ACAACATGAGAAATGAGTGACAAGGATTCTGAGTTAATATCAATTAAAA |
| | | CACAGAGGTCAAGGAATTTAATTCTGGGACTCCACCTCATGTTTTTTGA |
| | | GCTCATGTCAGACATAAATGGAAGAAGCTGATCCTCAAAGATCTTGGGA |
| | | TATAGCCGCCTCACAGATTGAATCACTTGGTTCAAATTCACTTTGTCCT |
| | | CCAGTAGCCTTGAGCTCTCAGGCTTTCTTGCTACATAATCACATGGGTT |
| | | TAAGTGCTTAAGAGTTAGGTTCTCACTGTTATTCTTCCCTTTGGTCGGT |
| | | TCTGCTAGGACCCAAACACCCAACTCAAAAGAGTTGCTCAATGAAATAC |
| | | AAATGTAGTCCCAAAGAAGAGGCCTTAAAAGGCATATATGATCACGGTG |
| | | GGCTTCTGGATGAGACTGTTTGTCACAAATGTACAGCGTTATACCATCC |
| | | CGATTGCAAACTCTTGTCACATGATCATCTGTGGTTAGATCCTCAAGCA |
| | | GCTTTTTGATATACAGATTTTCCCTATTTTTGTTTCTCACACACCTGCT |
| | | TCCTAGAGTTTTGCAAAGGCCTATAAAGCCAGATGAGATACAACTCTGG |
| | | AAAGCTGACTTGTTGATTGCTTCTGACAGCAGCTTCTGTGCACCCCTTG |
| | | TGAATTTACTACAAAGTTTGTTCTGGAGTGTCTTGATCAATGATGGGAT |
| | | TCTTTCCTCTTGGAAAGTCATCACTGATGGATAAACCACCTTTTGTCTT |
| | | AAAACCATCCTTAATGGGAACATTTCATTCAAATTCAACCAGTTAACAT |
| | | CTGCTAACTGATTCAGATCTTCTTCAAGACCGAGGAGGTCTCCCAATTG |
| | | AAGAATGGCCTCCtTTTTATCTCTGTTAAATAGGTCTAAGAAAAATTCT |
| | | TCATTAAATTCACCATTTTTGAGCTTATGATGCAGTTTCCTTACAAGCT |
| | | TTCTTACAACCTTTGTTTCATTAGGACACAGTTCCTCAATGAGTCTTTG |
| | | TATTCTGTAACCTCTAGAACCATCCAGCCAATCTTTCACATCAGTGTTG |
| | | GTATTCAGTAGAAATGGATCCAAAGGGAAATTGGCATACTTTAGGAGGT |
| | | CCAGTGTTCTCCTTTGGATACTATTAACTAGGGAGACTGGGACGCCATT |
| | | TGCGATGGCTTGATCTGCAATTGTATCTATTGTTTCACAAAGTTGATGT |
| | | GGCTCTTTACACTTGACATTGTGTAGCGCTGCAGATACAAACTTTGTGA |
| | | GAAGAGGGACTTCCTCCCCCCATACATAGAATCTAGATTTAAATTCTGC |
| | | AGCGAACCTCCCAGCCACACTTTTTGGGCTGATAAATTTGTTTAACAAG |
| | | CCGCTCAGATGAGATTGGAATTCCAACAGGACAAGGACTTCCTCCGGAT |
| | | CACTTACAACCAGGTCACTCAGCCTCCTATCAAATAAAGTGATCTGATC |
| | | ATCACTTGATGTGTAAGCCTCTGGTCTTTCGCCAAAGATAACACCAATG |
| | | CAGTAGTTGATGAACCTCTCGCTAAGCAAACCATAGAAGTCAGAAGCAT |
| | | TATGCAAGATTCCCTGCCCCATATCAATAAGGCTGGATATATGGGATGG |
| | | CACTATCCCCATTTCAAAATATTGTCTGAAAATTCTCTCAGTAACAGTT |
| | | GTTTCTGAACCCCTGAGAAGTTTTAGCTTCGACTTGACATATGATTTCA |
| | | TCATTGCATTCACAACAGGAAAGGGGACCTCGACAAGCTTATGCATGTG |
| | | CCAAGTTAACAAAGTGCTAACATGATCTTTCCCGGAACGCACATACTGG |
| | | TCATCACCTAGTTTGAGATTTTGTAGAAACATTAAGAACAAAAATGGGC |
| | | ACATCATTGGTCCCCATTTGCTGTGATCCATACTATAGTTTAAGAACCC |
| | | TTCCCGCACATTGATAGTCATTGACAAGATTGCATTTTCAAATTCCTTA |
| | | TCATTGTTTAAACAGGAGCCTGAAAAGAAACTTGAAAAAGACTCAAAAT |
| | | AATCTTCTATTAACCTTGTGAACATTTTTGTCCTCAAATCTCCAATATA |
| | | GAGTTCTCTATTTCCCCCAACCTGCTCTTTATAAGATAGTGCAAATTTC |
| | | AGCCTTCCAGAGTCAGGACCTACTGAGGTGTATGATGTTGGTGATTCTT |
| | | CTGAGTAGAAGCACAGATTTTTCAAAGCAGCACTCATACATTgTGTCAA |
| | | CGACAGAGCTTTACTAAGGGACTCAGAATTACTTTCCCTCTCACTGATT |
| | | CTCACGTCTTCTTCCAGTTTGTCCCAGTCAAATTTGAAATTCAAGCCTT |
| | | GCCTTTGCATATGCCTGTATTTCCCTGAGTACGCATTTGCATTCATTTG |
| | | CAACAGAATCATCTTCATGCAAGAAAACCAATCATTCTCAGAAAAGAAC |
| | | TTTCTACAAAGGTTTTTTGCCATCTCATCGAGGCCACACTGATCTTTAA |
| | | TGACTGAGGTGAAATACAAAGGTGACAGCTCTGTGGAACCCTCAACAGC |
| | | CTCACAGATAAATTTCATGTCATCATTGGTTAGACATGATGGGTCAAAG |
| | | TCTTCTACTAAATGGAAAGATATTTCTGACAAGATAACTTTTCTTAAGT |
| | | GAGCCATCTTCCCTGTTAGAATAAGCTGTAAATGATGTAGTCCTTTTGT |
| | | ATTTGTAAGTTTTTCTCCATCTCCTTTGTCATTGGCCCTCCTACCTCTT |
| | | CTGTACCGTGCTATTGTGGTGTTGACCTTTTCTTCGAGACTTTTGAAGA |
| | | AGCTTGTCTCTTCTTCTCCATCAAAACATATTTCTGCCAGGTTGTCTTC |
| | | CGATCTCCCTGTCTCTTCTCCCTTGGAACCGATGACCAATCTAGAGACT |
| | | AACTTGGAAACTTTATATTCATAGTCTGAGTGGCTCAACTTATACTTTT |
| | | GTTTTCTTACGAAACTCTCCGTAATTTGACTCACAGCACTAACAAGCAA |
| | | TTTGTTAAAGTCATATTCCAGAAGTCGTTCTCCATTTAGATGCTTATTA |
| | | ACCACCACACTTTTGTTACTAGCAAGATCTAATGCTGTCGCACATCCAG |
| | | AGTTAGTCATGGGATCTAGGCTGTTTAGCTTCTTCTCTCCTTTGAAAAT |
| | | TAAAGTGCCGTTGTTAAATGAAGACACCATTAGGCTAAAGGCTTCCAGA |
| | | TTAACACCTGGAGTTGTATGCTGACAGTCAATTTCTTTACTAGTGAATC |
| | | TCTTCATTTGCTCATAGAACACACATTCTTCCTCAGGAGTGATTGCTTC |
| | | CTTGGGGTTGACAAAAAAACCAAATTGACTTTTGGGCTCAAAGAACTTT |
| | | TCAAAACATTTTATCTGATCTGTTAGCCTGTCAGGGGTCTCCTTTGTGA |
| | | TCAAATGACACAGGTATGACACATTCAACATAAATTTAAATTTTGCACT |
| | | CAACAACACCTTCTCACCAGTACCAAAAATAGTTTTTATTAGGAATCTA |
| | | AGCAGCTTATACACCACCTTCTCAGCAGGTGTGATCAGATCCTCCCTCA |
| | | ACTTATCCATTAATGATGTAGATGAAAAATCTGACACTATTGCCATCAC |
| | | CAAATATCTGACACTCTGTACCTGCTTTTGATTTCTCTTTGTTGGGTTG |
| | | GTGAGCATTAGCAACAATAGGGTCCTCAGTGCAACCTCAATGTCGGTGA |
| | | GACAGTCTTTCAAATCAGGACATGATCTAATCCATGAAATCATGATGTC |
| | | TATCATATTGTATAAGACCTCATCTGAAAAAATTGGTAAAAAGAACCTT |
| | | TTAGGATCTGCATAGAAGGAAATTAAATGACCATCCGGGCCTTGTATGG |
| | | AGTAGCACCTTGAAGATTCTCCAGTCTTCTGGTATAATAGGTGGTATTC |
| | | TTCAGAGTCCAGTTTTATTACTTGGCAAAACACTTCTTTGCATTCTACC |
| | | ACTTGATATCTCACAGACCCTATTTGATTTTGCCTTAGTCTAGCAACTG |
| | | AGCTAGTTTTCATACTGTTTGTTAAGGCCAGACAAACAGATGATAATCT |
| | | TCTCAGGCTCTGTATGTTCTTCAGCTGCTCTGTGCTGGGTTGGAAATTG |
| | | TAATCTTCAAACTTCGTATAATACATTATCGGGTGAGCTCCAATTTTCA |
| | | TAAAGTTCTCAAATTCAGTGAATGGTATGTGGCATTCTTGCTCAAGGTG |
| | | TTCAGACAGTCCGTAATGCTCGAAACTCAGTCCCACCACTAACAGGCAT |
| | | TTTTGAATTTTTGCAATGAACTCACTAATAGAtGCCCTAAACAATTCCT |
| | | CAAAAGACACCTTTCTAAACACCTTTGACTTTTTTCTATTCCTCAAAAG |
| | | TCTAATGAACTCCTCTTTAGTGCTGTGAAAGCTTACCAGCCTATCATTC |
| | | ACACTACTATAGCAACAACCCACCCAGTGTTTATCATTTTTTAACCCTT |
| | | TGAATTTCGACTGTTTTATCAATGAGGAAAGACACAAAACATCCAGATT |
| | | TAACAACTGTCTCCTTCTAGTATTCAACAGTTTCAAACTCTTGACTTTG |
| | | TTTAACATAGAGAGGAGCCTCTCATATTCAGTGCTAGTCTCACTTCCCC |
| | | TTTCGTGCCCATGGGTCTCTGCAGTTATGAATCTCATCAAAGGACAGGA |
| | | TTCGACTGCCTCCCTGCTTAATGTTAAGATATCATCACTATCAGCAAGG |
| | | TTTTCATAGAGCTCAGAGAATTCCTTGATCAAGCCTTCAGGGTTTACTT |
| | | TCTGAAAGTTTCTCTTTAATTTCCCACTTTCTAAATCTCTTCTAAACCT |
| | | GCTGAAAAGAGAGTTTATTCCAAAAACCACATCATCACAGCTCATGTTG |
| | | GGGTTGATGCCTTCGTGGCACATCCTCATAATTTCATCATTGTGAGTTG |
| | | ACCTCGCATCTTTCAGAATTTTCATAGAGTCCATACCGGAGCGCTTGTC |
| | | GATAGTAGTCTTCAGGGACTCACAGAGTCTAAAATATTCAGACTCTTCA |
| | | AAGACTTTCTCATTTTGGTTAGAATACTCCAAAAGTTTGAATAAAAGGT |
| | | CTCTAAATTTGAAGTTTGCCCACTCTGGCATAAAACTATTATCATAATC |
| | | ACAACGACCATCTACTATTGGAACTAATGTGACACCCGCAACAGCAAGG |
| | | TCTTCCCTGATGCATGCCAATTTGTTAGTGTCCTCTATAAATTTCTTCT |
| | | CAAAACTGGCTGGaGtGCTCCTAACAAAACACTCAAGAAGAATGAGAGA |
| | | ATTGTCTATCAGCTTGTAACCATCAGGAATGATAAGTGGTAGTCCTGGG |
| | | CATACAATTCCAGACTCCACCAAAATTGTTTCCACAGACTTATCGTCGT |
| | | GGTTGTGTGTGCAGCCACTCTTGTCTGCACTGTCTATTTCAATGCAGCG |
| | | TGACAGCAACTTGAGTCCCTCAATCAGAACCATTCTGGGTTCCCTTTGT |
| | | CCCAGAAAGTTGAGTTTCTGCCTTGACAACCTCTCATCCTGTTCTATAT |
| | | AGTTTAAACATAACTCTCTCAATTCTGAGATGATTTCATCCATTGCGCA |
| | | TCAAAAAGCCTAGGATCCTCGGTGCG |
| 2 | Lymphocytic | CGCACCGGGGATCCTAGGCTTTTTGGATTGCGCTTTCCTC |
| | choriomeningitis | TAGATCAACTGGGTGTCAGGCCCTATCCTACAGAAGGATG |
| | virus segment S, | GGTCAGATTGTGACAATGTTTGAGGCTCTGCCTCACATCA |
| | complete sequence | TCGATGAGGTGATCAACATTGTCATTATTGTGCTTATCGT |
| | (The genomic | GATCACGGGTATCAAGGCTGTCTACAATTTTGCCACCTGT |
| | segment is RNA, the | GGGATATTCGCATTGATCAGTTTCCTACTTCTGGCTGGCA |
| | sequence in SEQ ID | GGTCCTGTGGCATGTACGGTCTTAAGGGACCCGACATTTA |
| | NO: 2 is shown for | CAAAGGAGTTTACCAATTTAAGTCAGTGGAGTTTGATATG |
| | DNA; however, | TCACATCTGAACCTGACCATGCCCAACGCATGTTCAGCCA |
| | exchanging all | ACAACTCCCACCATTACATCAGTATGGGGACTTCTGGACT |
| | thymidines (“T”) in | AGAATTGACCTTCACCAATGATTCCATCATCAGTCACAAC |
| | SEQ ID NO: 2 for | TTTTGCAATCTGACCTCTGCCTTCAACAAAAAGACCTTTG |
| | uridines (“U”) | ACCACACACTCATGAGTATAGTTTCGAGCCTACACCTCAG |
| | provides the RNA | TATCAGAGGGAACTCCAACTATAAGGCAGTATCCTGCGAC |
| | sequence.) | TTCAACAATGGCATAACCATCCAATACAACTTGACATTCT |
| | | CAGATCGACAAAGTGCTCAGAGCCAGTGTAGAACCTTCAG |
| | | AGGTAGAGTCCTAGATATGTTTAGAACTGCCTTCGGGGGG |
| | | AAATACATGAGGAGTGGCTGGGGCTGGACAGGCTCAGATG |
| | | GCAAGACCACCTGGTGTAGCCAGACGAGTTACCAATACCT |
| | | GATTATACAAAATAGAACCTGGGAAAACCACTGCACATAT |
| | | GCAGGTCCTTTTGGGATGTCCAGGATTCTCCTTTCCCAAG |
| | | AGAAGACTAAGTTCTTCACTAGGAGACTAGCGGGCACATT |
| | | CACCTGGACTTTGTCAGACTCTTCAGGGGTGGAGAATCCA |
| | | GGTGGTTATTGCCTGACCAAATGGATGATTCTTGCTGCAG |
| | | AGCTTAAGTGTTTCGGGAACACAGCAGTTGCGAAATGCAA |
| | | TGTAAATCATGATGCCGAATTCTGTGACATGCTGCGACTA |
| | | ATTGACTACAACAAGGCTGCTTTGAGTAAGTTCAAAGAGG |
| | | ACGTAGAATCTGCCTTGCACTTATTCAAAACAACAGTGAA |
| | | TTCTTTGATTTCAGATCAACTACTGATGAGGAACCACTTG |
| | | AGAGATCTGATGGGGGTGCCATATTGCAATTACTCAAAGT |
| | | TTTGGTACCTAGAACATGCAAAGACCGGCGAAACTAGTGT |
| | | CCCCAAGTGCTGGCTTGTCACCAATGGTTCTTACTTAAAT |
| | | GAGACCCACTTCAGTGATCAAATCGAACAGGAAGCCGATA |
| | | ACATGATTACAGAGATGTTGAGGAAGGATTACATAAAGAG |
| | | GCAGGGGAGTACCCCCCTAGCATTGATGGACCTTCTGATG |
| | | TTTTCCACATCTGCATATCTAGTCAGCATCTTCCTGCACC |
| | | TTGTCAAAATACCAACACACAGGCACATAAAAGGTGGCTC |
| | | ATGTCCAAAGCCACACCGATTAACCAACAAAGGAATTTGT |
| | | AGTTGTGGTGCATTTAAGGTGCCTGGTGTAAAAACCGTCT |
| | | GGAAAAGACGCTGAAGAACAGCGCCTCCCTGACTCTCCAC |
| | | CTCGAAAGAGGTGGAGAGTCAGGGAGGCCCAGAGGGTCTT |
| | | AGAGTGTCACAACATTTGGGCCTCTAAAAATTAGGTCATG |
| | | TGGCAGAATGTTGTGAACAGTTTTCAGATCTGGGAGCCTT |
| | | GCTTTGGAGGCGCTTTCAAAAATGATGCAGTCCATGAGTG |
| | | CACAGTGCGGGGTGATCTCTTTCTTCTTTTTGTCCCTTAC |
| | | TATTCCAGTATGCATCTTACACAACCAGCCATATTTGTCC |
| | | CACACTTTGTCTTCATACTCCCTCGAAGCTTCCCTGGTCA |
| | | TTTCAACATCGATAAGCTTAATGTCCTTCCTATTCTGTGA |
| | | GTCCAGAAGCTTTCTGATGTCATCGGAGCCTTGACAGCTT |
| | | AGAACCATCCCCTGCGGAAGAGCACCTATAACTGACGAGG |
| | | TCAACCCGGGTTGCGCATTGAAGAGGTCGGCAAGATCCAT |
| | | GCCGTGTGAGTACTTGGAATCTTGCTTGAATTGTTTTTGA |
| | | TCAACGGGTTCCCTGTAAAAGTGTATGAACTGCCCGTTCT |
| | | GTGGTTGGAAAATTGCTATTTCCACTGGATCATTAAATCT |
| | | ACCCTCAATGTCAATCCATGTAGGAGCGTTGGGGTCAATT |
| | | CCTCCCATGAGGTCTTTTAAAAGCATTGTCTGGCTGTAGC |
| | | TTAAGCCCACCTGAGGTGGACCTGCTGCTCCAGGCGCTGG |
| | | CCTGGGTGAATTGACTGCAGGTTTCTCGCTTGTGAGATCA |
| | | ATTGTTGTGTTTTCCCATGCTCTCCCCACAATCGATGTTC |
| | | TACAAGCTATGTATGGCCATCCTTCACCTGAAAGGCAAAC |
| | | TTTATAGAGGATGTTTTCATAAGGGTTCCTGTCCCCAACT |
| | | TGGTCTGAAACAAACATGTTGAGTTTTCTCTTGGCCCCGA |
| | | GAACTGCCTTCAAGAGGTCCTCGCTGTTGCTTGGCTTGAT |
| | | CAAAATTGACTCTAACATGTTACCCCCATCCAACAGGGCT |
| | | GCCCCTGCCTTCACGGCAGCACCAAGACTAAAGTTATAGC |
| | | CAGAAATGTTGATGCTGGACTGCTGTTCAGTGATGACCCC |
| | | CAGAACTGGGTGCTTGTCTTTCAGCCTTTCAAGATCATTA |
| | | AGATTTGGATACTTGACTGTGTAAAGCAAGCCAAGGTCTG |
| | | TGAGCGCTTGTACAACGTCATTGAGCGGAGTCTGTGACTG |
| | | TTTGGCCATACAAGCCATAGTTAGACTTGGCATTGTGCCA |
| | | AATTGATTGTTCAAAAGTGATGAGTCTTTCACATCCCAAA |
| | | CTCTTACCACACCACTTGCACCCTGCTGAGGCTTTCTCAT |
| | | CCCAACTATCTGTAGGATCTGAGATCTTTGGTCTAGTTGC |
| | | TGTGTTGTTAAGTTCCCCATATATACCCCTGAAGCCTGGG |
| | | GCCTTTCAGACCTCATGATCTTGGCCTTCAGCTTCTCAAG |
| | | GTCAGCCGCAAGAGACATCAGTTCTTCTGCACTGAGCCTC |
| | | CCCACTTTCAAAACATTCTTCTTTGATGTTGACTTTAAAT |
| | | CCACAAGAGAATGTACAGTCTGGTTGAGACTTCTGAGTCT |
| | | CTGTAGGTCTTTGTCATCTCTCTTTTCCTTCCTCATGATC |
| | | CTCTGAACATTGCTGACCTCAGAGAAGTCCAACCCATTCA |
| | | GAAGGTTGGTTGCATCCTTAATGACAGCAGCCTTCACATC |
| | | TGATGTGAAGCTCTGCAATTCTCTTCTCAATGCTTGCGTC |
| | | CATTGGAAGCTCTTAACTTCCTTAGACAAGGACATCTTGT |
| | | TGCTCAATGGTTTCTCAAGACAAATGCGCAATCAAATGCC |
| | | TAGGATCCACTGTGCG |
| 3 | Lymphocytic | GCGCACCGGGGATCCTAGGCTTTTTGGATTGCGCTTTCCT |
| | choriomeningitis | CTAGATCAACTGGGTGTCAGGCCCTATCCTACAGAAGGAT |
| | virus clone 13 | GGGTCAGATTGTGACAATGTTTGAGGCTCTGCCTCACATC |
| | segment S, complete | ATCGATGAGGTGATCAACATTGTCATTATTGTGCTTATCG |
| | sequence (GenBank: | TGATCACGGGTATCAAGGCTGTCTACAATTTTGCCACCTG |
| | DQ361065.2) | TGGGATATTCGCATTGATCAGTTTCCTACTTCTGGCTGGC |
| | (The genomic | AGGTCCTGTGGCATGTACGGTCTTAAGGGACCCGACATTT |
| | segment is RNA, the | ACAAAGGAGTTTACCAATTTAAGTCAGTGGAGTTTGATAT |
| | sequence in SEQ ID | GTCACATCTGAACCTGACCATGCCCAACGCATGTTCAGCC |
| | NO: 3 is shown for | AACAACTCCCACCATTACATCAGTATGGGGACTTCTGGAC |
| | DNA; however, | TAGAATTGACCTTCACCAATGATTCCATCATCAGTCACAA |
| | exchanging all | CTTTTGCAATCTGACCTCTGCCTTCAACAAAAAGACCTTT |
| | thymidines (“T”) in | GACCACACACTCATGAGTATAGTTTCGAGCCTACACCTCA |
| | SEQ ID NO: 3 for | GTATCAGAGGGAACTCCAACTATAAGGCAGTATCCTGCGA |
| | uridines (“U”) | CTTCAACAATGGCATAACCATCCAATACAACTTGACATTC |
| | provides the RNA | TCAGATGCACAAAGTGCTCAGAGCCAGTGTAGAACCTTCA |
| | sequence.) | GAGGTAGAGTCCTAGATATGTTTAGAACTGCCTTCGGGGG |
| | | GAAATACATGAGGAGTGGCTGGGGCTGGACAGGCTCAGAT |
| | | GGCAAGACCACCTGGTGTAGCCAGACGAGTTACCAATACC |
| | | TGATTATACAAAATAGAACCTGGGAAAACCACTGCACATA |
| | | TGCAGGTCCTTTTGGGATGTCCAGGATTCTCCTTTCCCAA |
| | | GAGAAGACTAAGTTCCTCACTAGGAGACTAGCGGGCACAT |
| | | TCACCTGGACTTTGTCAGACTCTTCAGGGGTGGAGAATCC |
| | | AGGTGGTTATTGCCTGACCAAATGGATGATTCTTGCTGCA |
| | | GAGCTTAAGTGTTTCGGGAACACAGCAGTTGCGAAATGCA |
| | | ATGTAAATCATGATGAAGAATTCTGTGACATGCTGCGACT |
| | | AATTGACTACAACAAGGCTGCTTTGAGTAAGTTCAAAGAG |
| | | GACGTAGAATCTGCCTTGCACTTATTCAAAACAACAGTGA |
| | | ATTCTTTGATTTCAGATCAACTACTGATGAGGAACCACTT |
| | | GAGAGATCTGATGGGGGTGCCATATTGCAATTACTCAAAG |
| | | TTTTGGTACCTAGAACATGCAAAGACCGGCGAAACTAGTG |
| | | TCCCCAAGTGCTGGCTTGTCACCAATGGTTCTTACTTAAA |
| | | TGAGACCCACTTCAGTGACCAAATCGAACAGGAAGCCGAT |
| | | AACATGATTACAGAGATGTTGAGGAAGGATTACATAAAGA |
| | | GGCAGGGGAGTACCCCCCTAGCATTGATGGACCTTCTGAT |
| | | GTTTTCCACATCTGCATATCTAGTCAGCATCTTCCTGCAC |
| | | CTTGTCAAAATACCAACACACAGGCACATAAAAGGTGGCT |
| | | CATGTCCAAAGCCACACCGATTAACCAACAAAGGAATTTG |
| | | TAGTTGTGGTGCATTTAAGGTGCCTGGTGTAAAAACCGTC |
| | | TGGAAAAGACGCTGAAGAACAGCGCCTCCCTGACTCTCCA |
| | | CCTCGAAAGAGGTGGAGAGTCAGGGAGGCCCAGAGGGTCT |
| | | TAGAGTGTCACAACATTTGGGCCTCTAAAAATTAGGTCAT |
| | | GTGGCAGAATGTTGTGAACAGTTTTCAGATCTGGGAGCCT |
| | | TGCTTTGGAGGCGCTTTCAAAAATGATGCAGTCCATGAGT |
| | | GCACAGTGCGGGGTGATCTCTTTCTTCTTTTTGTCCCTTA |
| | | CTATTCCAGTATGCATCTTACACAACCAGCCATATTTGTC |
| | | CCACACTTTGTCTTCATACTCCCTCGAAGCTTCCCTGGTC |
| | | ATTTCAACATCGATAAGCTTAATGTCCTTCCTATTCTGTG |
| | | AGTCCAGAAGCTTTCTGATGTCATCGGAGCCTTGACAGCT |
| | | TAGAACCATCCCCTGCGGAAGAGCACCTATAACTGACGAG |
| | | GTCAACCCGGGTTGCGCATTGAAGAGGTCGGCAAGATCCA |
| | | TGCCGTGTGAGTACTTGGAATCTTGCTTGAATTGTTTTTG |
| | | ATCAACGGGTTCCCTGTAAAAGTGTATGAACTGCCCGTTC |
| | | TGTGGTTGGAAAATTGCTATTTCCACTGGATCATTAAATC |
| | | TACCCTCAATGTCAATCCATGTAGGAGCGTTGGGGTCAAT |
| | | TCCTCCCATGAGGTCTTTTAAAAGCATTGTCTGGCTGTAG |
| | | CTTAAGCCCACCTGAGGTGGACCTGCTGCTCCAGGCGCTG |
| | | GCCTGGGTGAATTGACTGCAGGTTTCTCGCTTGTGAGATC |
| | | AATTGTTGTGTTTTCCCATGCTCTCCCCACAATCGATGTT |
| | | CTACAAGCTATGTATGGCCATCCTTCACCTGAAAGGCAAA |
| | | CTTTATAGAGGATGTTTTCATAAGGGTTCCTGTCCCCAAC |
| | | TTGGTCTGAAACAAACATGTTGAGTTTTCTCTTGGCCCCG |
| | | AGAACTGCCTTCAAGAGGTCCTCGCTGTTGCTTGGCTTGA |
| | | TCAAAATTGACTCTAACATGTTACCCCCATCCAACAGGGC |
| | | TGCCCCTGCCTTCACGGCAGCACCAAGACTAAAGTTATAG |
| | | CCAGAAATGTTGATGCTGGACTGCTGTTCAGTGATGACCC |
| | | CCAGAACTGGGTGCTTGTCTTTCAGCCTTTCAAGATCATT |
| | | AAGATTTGGATACTTGACTGTGTAAAGCAAGCCAAGGTCT |
| | | GTGAGCGCTTGTACAACGTCATTGAGCGGAGTCTGTGACT |
| | | GTTTGGCCATACAAGCCATAGTTAGACTTGGCATTGTGCC |
| | | AAATTGATTGTTCAAAAGTGATGAGTCTTTCACATCCCAA |
| | | ACTCTTACCACACCACTTGCACCCTGCTGAGGCTTTCTCA |
| | | TCCCAACTATCTGTAGGATCTGAGATCTTTGGTCTAGTTG |
| | | CTGTGTTGTTAAGTTCCCCATATATACCCCTGAAGCCTGG |
| | | GGCCTTTCAGACCTCATGATCTTGGCCTTCAGCTTCTCAA |
| | | GGTCAGCCGCAAGAGACATCAGTTCTTCTGCACTGAGCCT |
| | | CCCCACTTTCAAAACATTCTTCTTTGATGTTGACTTTAAA |
| | | TCCACAAGAGAATGTACAGTCTGGTTGAGACTTCTGAGTC |
| | | TCTGTAGGTCTTTGTCATCTCTCTTTTCCTTCCTCATGAT |
| | | CCTCTGAACATTGCTGACCTCAGAGAAGTCCAACCCATTC |
| | | AGAAGGTTGGTTGCATCCTTAATGACAGCAGCCTTCACAT |
| | | CTGATGTGAAGCTCTGCAATTCTCTTCTCAATGCTTGCGT |
| | | CCATTGGAAGCTCTTAACTTCCTTAGACAAGGACATCTTG |
| | | TTGCTCAATGGTTTCTCAAGACAAATGCGCAATCAAATGC |
| | | CTAGGATCCACTGTGCG |
| 4 | Lymphocytic | GCGCACCGGGGATCCTAGGCATTTTTGTTGCGCATTTTGT |
| | choriomeningitis | TGTGTTATTTGTTGCACAGCCCTTCATCGTGGGACCTTCA |
| | strain MP segment | CAAACAAACCAAACCACCAGCCATGGGCCAAGGCAAGTCC |
| | L, complete | AAAGAGGGAAGGGATGCCAGCAATACGAGCAGAGCTGAAA |
| | sequence | TTCTGCCAGACACCACCTATCTCGGACCTCTGAACTGCAA |
| | (The genomic | GTCATGCTGGCAGAGATTTGACAGTTTAGTCAGATGCCAT |
| | segment is RNA, the | GACCACTATCTCTGCAGACACTGCCTGAACCTCCTGCTGT |
| | sequence in SEQ ID | CAGTCTCCGACAGGTGCCCTCTCTGCAAACATCCATTGCC |
| | NO: 4 is shown for | AACCAAACTGAAAATATCCACGGCCCCAAGCTCTCCACCC |
| | DNA; however, | CCTTACGAGGAGTGACGCCCCGAGCCCCAACACCGACACA |
| | exchanging all | AGGAGGCCACCAACACAACGCCCAACACGGAACACACACA |
| | thymidines (“T”) in | CACACACCCACACACACATCCACACACACGCGCCCCCACA |
| | SEQ ID NO: 4 for | ACGGGGGCGCCCCCCCGGGGGTGGCCCCCCGGGTGCTCGG |
| | uridines (“U”) | GCGGAGCCCCACGGAGAGGCCAATTAGTCGATCTCCTCGA |
| | provides the RNA | CCACCGACTTGGTCAGCCAGTCATCACAGGACTTGCCCTT |
| | sequence.) | AAGTCTGTACTTGCCCACAACTGTTTCATACATCACCGTG |
| | | TTCTTTGACTTACTGAAACATAGCCTACAGTCTTTGAAAG |
| | | TGAACCAGTCAGGCACAAGTGACAGCGGTACCAGTAGAAT |
| | | GGATCTATCTATACACAACTCTTGGAGAATTGTGCTAATT |
| | | TCCGACCCCTGTAGATGCTCACCAGTTCTGAATCGATGTA |
| | | GAAGAAGGCTCCCAAGGACGTCATCAAAATTTCCATAACC |
| | | CTCGAGCTCTGCCAAGAAAACTCTCATATCCTTGGTCTCC |
| | | AGTTTCACAACGATGTTCTGAACAAGGCTTCTTCCCTCAA |
| | | AAAGAGCACCCATTCTCACAGTCAAGGGCACAGGCTCCCA |
| | | TTCAGGCCCAATCCTCTCAAAATCAAGGGATCTGATCCCG |
| | | TCCAGTATTTTCCTTGAGCCTATCAGCTCAAGCTCAAGAG |
| | | AGTCACCGAGTATCAGGGGGTCCTCCATATAGTCCTCAAA |
| | | CTCTTCAGACCTAATGTCAAAAACACCATCGTTCACCTTG |
| | | AAGATAGAGTCTGATCTCAACAGGTGGAGGCATTCGTCCA |
| | | AGAACCTTCTGTCCACCTCACCTTTAAAGAGGTGAGAGCA |
| | | TGATAGGAACTCAGCTACACCTGGACCTTGTAACTGGCAC |
| | | TTCACTAAAAAGATCAATGAAAACTTCCTCAAACAATCAG |
| | | TGTTATTCTGGTTGTGAGTGAAATCTACTGTAATTGAGAA |
| | | CTCTAGCACTCCCTCTGTATTATTTATCATGTAATCCCAC |
| | | AAGTTTCTCAAAGACTTGAATGCCTTTGGATTTGTCAAGC |
| | | CTTGTTTGATTAGCATGGCAGCATTGCACACAATATCTCC |
| | | CAATCGGTAAGAGAACCATCCAAATCCAAATTGCAAGTCA |
| | | TTCCTAAACATGGGCCTCTCCATATTTTTGTTCACTACTT |
| | | TTAAGATGAATGATTGGAAAGGCCCCAATGCTTCAGCGCC |
| | | ATCTTCAGATGGCATCATGTCTTTATGAGGGAACCATGAA |
| | | AAACTTCCTAGAGTTCTGCTTGTTGCTACAAATTCTCGTA |
| | | CAAATGACTCAAAATACACTTGTTTTAAAAAGTTTTTGCA |
| | | GACATCCCTTGTACTAACGACAAATTCATCAACAAGGCTT |
| | | GAGTCAGAGCGCTGATGGGAATTTACAAGATCAGAAAATA |
| | | GAACAGTGTAGTGTTCGTCCCTCTTCCACTTAACTACATG |
| | | AGAAATGAGCGATAAAGATTCTGAATTGATATCGATCAAT |
| | | ACGCAAAGGTCAAGGAATTTGATTCTGGGACTCCATCTCA |
| | | TGTTTTTTGAGCTCATATCAGACATGAAGGGAAGCAGCTG |
| | | ATCTTCATAGATTTTAGGGTACAATCGCCTCACAGATTGG |
| | | ATTACATGGTTTAAACTTATCTTGTCCTCCAGTAGCCTTG |
| | | AACTCTCAGGCTTCCTTGCTACATAATCACATGGGTTCAA |
| | | GTGCTTGAGGCTTGAGCTTCCCTCATTCTTCCCTTTCACA |
| | | GGTTCAGCTAAGACCCAAACACCCAACTCAAAGGAATTAC |
| | | TCAGTGAGATGCAAATATAGTCCCAAAGGAGGGGCCTCAA |
| | | GAGACTGATGTGGTCGCAGTGAGCTTCTGGATGACTTTGC |
| | | CTGTCACAAATGTACAACATTATGCCATCATGTCTGTGGA |
| | | TTGCTGTCACATGCGCATCCATAGCTAGATCCTCAAGCAC |
| | | TTTTCTAATGTATAGATTGTCCCTATTTTTATTTCTCACA |
| | | CATCTACTTCCCAAAGTTTTGCAAAGACCTATAAAGCCTG |
| | | ATGAGATGCAACTTTGAAAGGCTGACTTATTGATTGCTTC |
| | | TGACAGCAACTTCTGTGCACCTCTTGTGAACTTACTGCAG |
| | | AGCTTGTTCTGGAGTGTCTTGATTAATGATGGGATTCTTT |
| | | CCTCTTGGAAAGTCATTACTGATGGATAAACCACTTTCTG |
| | | CCTCAAGACCATTCTTAATGGGAACAACTCATTCAAATTC |
| | | AGCCAATTTATGTTTGCCAATTGACTTAGATCCTCTTCGA |
| | | GGCCAAGGATGTTTCCCAACTGAAGAATGGCTTCCTTTTT |
| | | ATCCCTATTGAAGAGGTCTAAGAAGAATTCTTCATTGAAC |
| | | TCACCATTCTTGAGCTTATGATGTAGTCTCCTTACAAGCC |
| | | TTCTCATGACCTTCGTTTCACTAGGACACAATTCTTCAAT |
| | | AAGCCTTTGGATTCTGTAACCTCTAGAGCCATCCAACCAA |
| | | TCCTTGACATCAGTATTAGTGTTAAGCAAAAATGGGTCCA |
| | | AGGGAAAGTTGGCATATTTTAAGAGGTCTAATGTTCTCTT |
| | | CTGGATGCAGTTTACCAATGAAACTGGAACACCATTTGCA |
| | | ACAGCTTGATCGGCAATTGTATCTATTGTTTCACAGAGTT |
| | | GGTGTGGCTCTTTACACTTAACGTTGTGTAATGCTGCTGA |
| | | CACAAATTTTGTTAAAAGTGGGACCTCTTCCCCCCACACA |
| | | TAAAATCTGGATTTAAATTCTGCAGCAAATCGCCCCACCA |
| | | CACTTTTCGGACTGATGAACTTGTTAAGCAAGCCACTCAA |
| | | ATGAGAATGAAATTCCAGCAATACAAGGACTTCCTCAGGG |
| | | TCACTATCAACCAGTTCACTCAATCTCCTATCAAATAAGG |
| | | TGATCTGATCATCACTTGATGTGTAAGATTCTGGTCTCTC |
| | | ACCAAAAATGACACCGATACAATAATTAATGAATCTCTCA |
| | | CTGATTAAGCCGTAAAAGTCAGAGGCATTATGTAAGATTC |
| | | CCTGTCCCATGTCAATGAGACTGCTTATATGGGAAGGCAC |
| | | TATTCCTAATTCAAAATATTCTCGAAAGATTCTTTCAGTC |
| | | ACAGTTGTCTCTGAACCCCTAAGAAGTTTCAGCTTTGATT |
| | | TGATATATGATTTCATCATTGCATTCACAACAGGAAAAGG |
| | | GACCTCAACAAGTTTGTGCATGTGCCAAGTTAATAAGGTG |
| | | CTGATATGATCCTTTCCGGAACGCACATACTGGTCATCAC |
| | | CCAGTTTGAGATTTTGAAGGAGCATTAAAAACAAAAATGG |
| | | GCACATCATTGGCCCCCATTTGCTATGATCCATACTGTAG |
| | | TTCAACAACCCCTCTCGCACATTGATGGTCATTGATAGAA |
| | | TTGCATTTTCAAATTCTTTGTCATTGTTTAAGCATGAACC |
| | | TGAGAAGAAGCTAGAAAAAGACTCAAAATAATCCTCTATC |
| | | AATCTTGTAAACATTTTTGTTCTCAAATCCCCAATATAAA |
| | | GTTCTCTGTTTCCTCCAACCTGCTCTTTGTATGATAACGC |
| | | AAACTTCAACCTTCCGGAATCAGGACCAACTGAAGTGTAT |
| | | GACGTTGGTGACTCCTCTGAGTAAAAACATAAATTCTTTA |
| | | AAGCAGCACTCATGCATTTTGTCAATGATAGAGCCTTACT |
| | | TAGAGACTCAGAATTACTTTCCCTTTCACTAATTCTAACA |
| | | TCTTCTTCTAGTTTGTCCCAGTCAAACTTGAAATTCAGAC |
| | | CTTGTCTTTGCATGTGCCTGTATTTCCCTGAGTATGCATT |
| | | TGCATTCATTTGCAGTAGAATCATTTTCATACACGAAAAC |
| | | CAATCACCCTCTGAAAAAAACTTCCTGCAGAGGTTTTTTG |
| | | CCATTTCATCCAGACCACATTGTTCTTTGACAGCTGAAGT |
| | | GAAATACAATGGTGACAGTTCTGTAGAAGTTTCAATAGCC |
| | | TCACAGATAAATTTCATGTCATCATTGGTGAGACAAGATG |
| | | GGTCAAAATCTTCCACAAGATGAAAAGAAATTTCTGATAA |
| | | GATGACCTTCCTTAAATATGCCATTTTACCTGACAATATA |
| | | GTCTGAAGGTGATGCAATCCTTTTGTATTTTCAAACCCCA |
| | | CCTCATTTTCCCCTTCATTGGTCTTCTTGCTTCTTTCATA |
| | | CCGCTTTATTGTGGAGTTGACCTTATCTTCTAAATTCTTG |
| | | AAGAAACTTGTCTCTTCTTCCCCATCAAAGCATATGTCTG |
| | | CTGAGTCACCTTCTAGTTTCCCAGCTTCTGTTTCTTTAGA |
| | | GCCGATAACCAATCTAGAGACCAACTTTGAAACCTTGTAC |
| | | TCGTAATCTGAGTGGTTCAATTTGTACTTCTGCTTTCTCA |
| | | TGAAGCTCTCTGTGATCTGACTCACAGCACTAACAAGCAA |
| | | TTTGTTAAAATCATACTCTAGGAGCCGTTCCCCATTTAAA |
| | | TGTTTGTTAACAACCACACTTTTGTTGCTGGCAAGGTCTA |
| | | ATGCTGTTGCACACCCAGAGTTAGTCATGGGATCCAAGCT |
| | | ATTGAGCCTCTTCTCCCCTTTGAAAATCAAAGTGCCATTG |
| | | TTGAATGAGGACACCATCATGCTAAAGGCCTCCAGATTGA |
| | | CACCTGGGGTTGTGCGCTGACAGTCAACTTCTTTCCCAGT |
| | | GAACTTCTTCATTTGGTCATAAAAAACACACTCTTCCTCA |
| | | GGGGTGATTGACTCTTTAGGGTTAACAAAGAAGCCAAACT |
| | | CACTTTTAGGCTCAAAGAATTTCTCAAAGCATTTAATTTG |
| | | ATCTGTCAGCCTATCAGGGGTTTCCTTTGTGATTAAATGA |
| | | CACAGGTATGACACATTCAACATGAACTTGAACTTTGCGC |
| | | TCAACAGTACCTTTTCACCAGTCCCAAAAACAGTTTTGAT |
| | | CAAAAATCTGAGCAATTTGTACACTACTTTCTCAGCAGGT |
| | | GTGATCAAATCCTCCTTCAACTTGTCCATCAATGATGTGG |
| | | ATGAGAAGTCTGAGACAATGGCCATCACTAAATACCTAAT |
| | | GTTTTGAACCTGTTTTTGATTCCTCTTTGTTGGGTTGGTG |
| | | AGCATGAGTAATAATAGGGTTCTCAATGCAATCTCAACAT |
| | | CATCAATGCTGTCCTTCAAGTCAGGACATGATCTGATCCA |
| | | TGAGATCATGGTGTCAATCATGTTGTGCAACACTTCATCT |
| | | GAGAAGATTGGTAAAAAGAACCTTTTTGGGTCTGCATAAA |
| | | AAGAGATTAGATGGCCATTGGGACCTTGTATAGAATAACA |
| | | CCTTGAGGATTCTCCAGTCTTTTGATACAGCAGGTGATAT |
| | | TCCTCAGAGTCCAATTTTATCACTTGGCAAAATACCTCTT |
| | | TACATTCCACCACTTGATACCTTACAGAGCCCAATTGGTT |
| | | TTGTCTTAATCTAGCAACTGAACTTGTTTTCATACTGTTT |
| | | GTCAAAGCTAGACAGACAGATGACAATCTTTTCAAACTAT |
| | | GCATGTTCCTTAATTGTTCCGTATTAGGCTGGAAATCATA |
| | | ATCTTCAAACTTTGTATAATACATTATAGGATGAGTTCCG |
| | | GACCTCATGAAATTCTCAAACTCAATAAATGGTATGTGGC |
| | | ACTCATGCTCAAGATGTTCAGACAGACCATAGTGCCCAAA |
| | | ACTAAGTCCCACCACTGACAAGCACCTTTGAACTTTTAAA |
| | | ATGAACTCATTTATGGATGTTCTAAACAAATCCTCAAGAG |
| | | ATACCTTTCTATACGCCTTTGACTTTCTCCTGTTCCTTAG |
| | | AAGTCTGATGAACTCTTCCTTGGTGCTATGAAAGCTCACC |
| | | AACCTATCATTCACACTCCCATAGCAACAACCAACCCAGT |
| | | GCTTATCATTTTTTGACCCTTTGAGTTTAGACTGTTTGAT |
| | | CAACGAAGAGAGACACAAGACATCCAAATTCAGTAACTGT |
| | | CTCCTTCTGGTGTTCAATAATTTTAAACTTTTAACTTTGT |
| | | TCAACATAGAGAGGAGCCTCTCATACTCAGTGCTAGTCTC |
| | | ACTTCCTCTCTCATAACCATGGGTATCTGCTGTGATAAAT |
| | | CTCATCAAAGGACAGGATTCAACTGCCTCCTTGCTTAGTG |
| | | CTGAAATGTCATCACTGTCAGCAAGAGTCTCATAAAGCTC |
| | | AGAGAATTCCTTAATTAAATTTCCGGGGTTGATTTTCTGA |
| | | AAACTCCTCTTGAGCTTCCCAGTTTCCAAGTCTCTTCTAA |
| | | ACCTGCTGTAAAGGGAGTTTATGCCAAGAACCACATCATC |
| | | GCAGTTCATGTTTGGGTTGACACCATCATGGCACATTTTC |
| | | ATAATTTCATCATTGTGAAATGATCTTGCATCTTTCAAGA |
| | | TTTTCATAGAGTCTATACCGGAACGCTTATCAACAGTGGT |
| | | CTTGAGAGATTCGCAAAGTCTGAAGTACTCAGATTCCTCA |
| | | AAGACTTTCTCATCTTGGCTAGAATACTCTAAAAGTTTAA |
| | | ACAGAAGGTCTCTGAACTTGAAATTCACCCACTCTGGCAT |
| | | AAAGCTGTTATCATAATCACACCGACCATCCACTATTGGG |
| | | ACCAATGTGATACCCGCAATGGCAAGGTCTTCTTTGATAC |
| | | AGGCTAGTTTATTGGTGTCCTCTATAAATTTCTTCTCAAA |
| | | ACTAGCTGGTGTGCTTCTAACGAAGCACTCAAGAAGAATG |
| | | AGGGAATTGTCAATCAGTTTATAACCATCAGGAATGATCA |
| | | AAGGCAGTCCCGGGCACACAATCCCAGACTCTATTAGAAT |
| | | TGCCTCAACAGATTTATCATCATGGTTGTGTATGCAGCCG |
| | | CTCTTGTCAGCACTGTCTATCTCTATACAACGCGACAAAA |
| | | GTTTGAGTCCCTCTATCAATACCATTCTGGGTTCTCTTTG |
| | | CCCTAAAAAGTTGAGCTTCTGCCTTGACAACCTCTCATCT |
| | | TGTTCTATGTGGTTTAAGCACAACTCTCTCAACTCCGAAA |
| | | TAGCCTCATCCATTGCGCATCAAAAAGCCTAGGATCCTCG |
| | | GTGCG |
| 5 | Lymphocytic | CGCACCGGGGATCCTAGGCTTTTTGGATTGCGCTTTCCTC |
| | choriomeningitis | AGCTCCGTCTTGTGGGAGAATGGGTCAAATTGTGACGATG |
| | strain MP segment | TTTGAGGCTCTGCCTCACATCATTGATGAGGTCATTAACA |
| | S, complete | TTGTCATTATCGTGCTTATTATCATCACGAGCATCAAAGC |
| | sequence | TGTGTACAATTTCGCCACCTGCGGGATACTTGCATTGATC |
| | (The genomic | AGCTTTCTTTTTCTGGCTGGCAGGTCCTGTGGAATGTATG |
| | segment is RNA, the | GTCTTGATGGGCCTGACATTTACAAAGGGGTTTACCGATT |
| | sequence in SEQ ID | CAAGTCAGTGGAGTTTGACATGTCTTACCTTAACCTGACG |
| | NO: 5 is shown for | ATGCCCAATGCATGTTCGGCAAACAACTCCCATCATTATA |
| | DNA; however, | TAAGTATGGGGACTTCTGGATTGGAGTTAACCTTCACAAA |
| | exchanging all | TGACTCCATCATCACCCACAACTTTTGTAATCTGACTTCC |
| | thymidines (“T”) in | GCCCTCAACAAGAGGACTTTTGACCACACACTTATGAGTA |
| | SEQ ID NO: 5 for | TAGTCTCAAGTCTGCACCTCAGCATTAGAGGGGTCCCCAG |
| | uridines (“U”) | CTACAAAGCAGTGTCCTGTGATTTTAACAATGGCATCACT |
| | provides the RNA | ATTCAATACAACCTGTCATTTTCTAATGCACAGAGCGCTC |
| | sequence.) | TGAGTCAATGTAAGACCTTCAGGGGGAGAGTCCTGGATAT |
| | | GTTCAGAACTGCTTTTGGAGGAAAGTACATGAGGAGTGGC |
| | | TGGGGCTGGACAGGTTCAGATGGCAAGACTACTTGGTGCA |
| | | GCCAGACAAACTACCAATATCTGATTATACAAAACAGGAC |
| | | TTGGGAAAACCACTGCAGGTACGCAGGCCCTTTCGGAATG |
| | | TCTAGAATTCTCTTCGCTCAAGAAAAGACAAGGTTTCTAA |
| | | CTAGAAGGCTTGCAGGCACATTCACTTGGACTTTATCAGA |
| | | CTCATCAGGAGTGGAGAATCCAGGTGGTTACTGCTTGACC |
| | | AAGTGGATGATCCTCGCTGCAGAGCTCAAGTGTTTTGGGA |
| | | ACACAGCTGTTGCAAAGTGCAATGTAAATCATGATGAAGA |
| | | GTTCTGTGATATGCTACGACTGATTGATTACAACAAGGCT |
| | | GCTTTGAGTAAATTCAAAGAAGATGTAGAATCCGCTCTAC |
| | | ATCTGTTCAAGACAACAGTGAATTCTTTGATTTCTGATCA |
| | | GCTTTTGATGAGAAATCACCTAAGAGACTTGATGGGAGTG |
| | | CCATACTGCAATTACTCGAAATTCTGGTATCTAGAGCATG |
| | | CAAAGACTGGTGAGACTAGTGTCCCCAAGTGCTGGCTTGT |
| | | CAGCAATGGTTCTTATTTGAATGAAACCCATTTCAGCGAC |
| | | CAAATTGAGCAGGAAGCAGATAATATGATCACAGAAATGC |
| | | TGAGAAAGGACTACATAAAAAGGCAAGGGAGTACCCCTCT |
| | | AGCCTTGATGGATCTATTGATGTTTTCTACATCAGCATAT |
| | | TTGATCAGCATCTTTCTGCATCTTGTGAGGATACCAACAC |
| | | ACAGACACATAAAGGGCGGCTCATGCCCAAAACCACATCG |
| | | GTTAACCAGCAAGGGAATCTGTAGTTGTGGTGCATTTAAA |
| | | GTACCAGGTGTGGAAACCACCTGGAAAAGACGCTGAACAG |
| | | CAGCGCCTCCCTGACTCACCACCTCGAAAGAGGTGGTGAG |
| | | TCAGGGAGGCCCAGAGGGTCTTAGAGTGTTACGACATTTG |
| | | GACCTCTGAAGATTAGGTCATGTGGTAGGATATTGTGGAC |
| | | AGTTTTCAGGTCGGGGAGCCTTGCCTTGGAGGCGCTTTCA |
| | | AAGATGATACAGTCCATGAGTGCACAGTGTGGGGTGACCT |
| | | CTTTCTTTTTCTTGTCCCTCACTATTCCAGTGTGCATCTT |
| | | GCATAGCCAGCCATATTTGTCCCAGACTTTGTCCTCATAT |
| | | TCTCTTGAAGCTTCTTTAGTCATCTCAACATCGATGAGCT |
| | | TAATGTCTCTTCTGTTTTGTGAATCTAGGAGTTTCCTGAT |
| | | GTCATCAGATCCCTGACAACTTAGGACCATTCCCTGTGGA |
| | | AGAGCACCTATTACTGAAGATGTCAGCCCAGGTTGTGCAT |
| | | TGAAGAGGTCAGCAAGGTCCATGCCATGTGAGTATTTGGA |
| | | GTCCTGCTTGAATTGTTTTTGATCAGTGGGTTCTCTATAG |
| | | AAATGTATGTACTGCCCATTCTGTGGCTGAAATATTGCTA |
| | | TTTCTACCGGGTCATTAAATCTGCCCTCAATGTCAATCCA |
| | | TGTAGGAGCGTTAGGGTCAATACCTCCCATGAGGTCCTTC |
| | | AGCAACATTGTTTGGCTGTAGCTTAAGCCCACCTGAGGTG |
| | | GGCCCGCTGCCCCAGGCGCTGGTTTGGGTGAGTTGGCCAT |
| | | AGGCCTCTCATTTGTCAGATCAATTGTTGTGTTCTCCCAT |
| | | GCTCTCCCTACAACTGATGTTCTACAAGCTATGTATGGCC |
| | | ACCCCTCCCCTGAAAGACAGACTTTGTAGAGGATGTTCTC |
| | | GTAAGGATTCCTGTCTCCAACCTGATCAGAAACAAACATG |
| | | TTGAGTTTCTTCTTGGCCCCAAGAACTGCTTTCAGGAGAT |
| | | CCTCACTGTTGCTTGGCTTAATTAAGATGGATTCCAACAT |
| | | GTTACCCCCATCTAACAAGGCTGCCCCTGCTTTCACAGCA |
| | | GCACCGAGACTGAAATTGTAGCCAGATATGTTGATGCTAG |
| | | ACTGCTGCTCAGTGATGACTCCCAAGACTGGGTGCTTGTC |
| | | TTTCAGCCTTTCAAGGTCACTTAGGTTCGGGTACTTGACT |
| | | GTGTAAAGCAGCCCAAGGTCTGTGAGTGCTTGCACAACGT |
| | | CATTGAGTGAGGTTTGTGATTGTTTGGCCATACAAGCCAT |
| | | TGTTAAGCTTGGCATTGTGCCGAATTGATTGTTCAGAAGT |
| | | GATGAGTCCTTCACATCCCAGACCCTCACCACACCATTTG |
| | | CACTCTGCTGAGGTCTCCTCATTCCAACCATTTGCAGAAT |
| | | CTGAGATCTTTGGTCAAGCTGTTGTGCTGTTAAGTTCCCC |
| | | ATGTAGACTCCAGAAGTTAGAGGCCTTTCAGACCTCATGA |
| | | TTTTAGCCTTCAGTTTTTCAAGGTCAGCTGCAAGGGACAT |
| | | CAGTTCTTCTGCACTAAGCCTCCCTACTTTTAGAACATTC |
| | | TTTTTTGATGTTGACTTTAGGTCCACAAGGGAATACACAG |
| | | TTTGGTTGAGGCTTCTGAGTCTCTGTAAATCTTTGTCATC |
| | | CCTCTTCTCTTTCCTCATGATCCTCTGAACATTGCTCACC |
| | | TCAGAGAAGTCTAATCCATTCAGAAGGCTGGTGGCATCCT |
| | | TGATCACAGCAGCTTTCACATCTGATGTGAAGCCTTGAAG |
| | | CTCTCTCCTCAATGCCTGGGTCCATTGAAAGCTTTTAACT |
| | | TCTTTGGACAGAGACATTTTGTCACTCAGTGGATTTCCAA |
| | | GTCAAATGCGCAATCAAAATGCCTAGGATCCACTGTGCG |
| 6 | Amino acid sequence | MSLSKEVKSFQWTQALRRELQGFTSDVKAAVIKDATSLLN |
| | of the NP protein | GLDFSEVSNVQRIMRKEKRDDKDLQRLRSLNQTVYSLVDL |
| | of the MP strain of | KSTSKKNVLKVGRLSAEELMSLAADLEKLKAKIMRSERPL |
| | LCMV | TSGVYMGNLTAQQLDQRSQILQMVGMRRPQQSANGVVRVW |
| | | DVKDSSLLNNQFGTMPSLTMACMAKQSQTSLNDVVQALTD |
| | | LGLLYTVKYPNLSDLERLKDKHPVLGVITEQQSSINISGY |
| | | NFSLGAAVKAGAALLDGGNMLESILIKPSNSEDLLKAVLG |
| | | AKKKLNMFVSDQVGDRNPYENILYKVCLSGEGWPYIACRT |
| | | SVVGRAWENTTIDLTNERPMANSPKPAPGAAGPPQVGLSY |
| | | SQTMLLKDLMGGIDPNAPTWIDIEGRFNDPVEIAIFQPQN |
| | | GQYIHFYREPTDQKQFKQDSKYSHGMDLADLFNAQPGLTS |
| | | SVIGALPQGMVLSCQGSDDIRKLLDSQNRRDIKLIDVEMT |
| | | KEASREYEDKVWDKYGWLCKMHTGIVRDKKKKEVTPHCAL |
| | | MDCIIFESASKARLPDLKTVHNILPHDLIFRGPNVVTL |
| 7 | Amino acid sequence | MGQIVTMFEALPHIIDEVINIVIIVLIIITSIKAVYNFAT |
| | of the GP protein | CGILALISFLFLAGRSCGMYGLDGPDIYKGVYRFKSVEFD |
| | of the MP strain of | MSYLNLTMPNACSANNSHHYISMGTSGLELTFTNDSIITH |
| | LCMV | NFCNLTSALNKRTFDHTLMSIVSSLHLSIRGVPSYKAVSC |
| | | DFNNGITIQYNLSFSNAQSALSQCKTFRGRVLDMFRTAFG |
| | | GKYMRSGWGWTGSDGKTTWCSQTNYQYLIIQNRTWENHCR |
| | | YAGPFGMSRILFAQEKTRFLTRRLAGTFTWTLSDSSGVEN |
| | | PGGYCLTKWMILAAELKCFGNTAVAKCNVNHDEEFCDMLR |
| | | LIDYNKAALSKFKEDVESALHLFKTTVNSLISDQLLMRNH |
| | | LRDLMGVPYCNYSKFWYLEHAKTGETSVPKCWLVSNGSYL |
| | | NETHFSDQIEQEADNMITEMLRKDYIKRQGSTPLALMDLL |
| | | MFSTSAYLISIFLHLVRIPTHRHIKGGSCPKPHRLTSKGI |
| | | CSCGAFKVPGVETTWKRR |
| 8 | amino acid sequence | MDEAISELRELCLNHIEQDERLSRQKLNFLGQREPRMVLI |
| | of the L protein of | EGLKLLSRCIEIDSADKSGCIHNHDDKSVEAILIESGIVC |
| | the MP strain of | PGLPLIIPDGYKLIDNSLILLECFVRSTPASFEKKFIEDT |
| | LCMV | NKLACIKEDLAIAGITLVPIVDGRCDYDNSFMPEWVNFKF |
| | | RDLLFKLLEYSSQDEKVFEESEYFRLCESLKTTVDKRSGI |
| | | DSMKILKDARSFHNDEIMKMCHDGVNPNMNCDDVVLGINS |
| | | LYSRFRRDLETGKLKRSFQKINPGNLIKEFSELYETLADS |
| | | DDISALSKEAVESCPLMRFITADTHGYERGSETSTEYERL |
| | | LSMLNKVKSLKLLNTRRRQLLNLDVLCLSSLIKQSKLKGS |
| | | KNDKHWVGCCYGSVNDRLVSFHSTKEEFIRLLRNRRKSKA |
| | | YRKVSLEDLFRTSINEFILKVQRCLSVVGLSFGHYGLSEH |
| | | LEHECHIPFIEFENFMRSGTHPIMYYTKFEDYDFQPNTEQ |
| | | LRNMHSLKRLSSVCLALTNSMKTSSVARLRQNQLGSVRYQ |
| | | VVECKEVFCQVIKLDSEEYHLLYQKTGESSRCYSIQGPNG |
| | | HLISFYADPKRFFLPIFSDEVLHNMIDTMISWIRSCPDLK |
| | | DSIDDVEIALRTLLLLMLTNPTKRNQKQVQNIRYLVMAIV |
| | | SDFSSTSLMDKLKEDLITPAEKVVYKLLRFLIKTVFGTGE |
| | | KVLLSAKFKFMLNVSYLCHLITKETPDRLTDQIKCFEKFF |
| | | EPKSEFGFFVNPKESITPEEECVFYDQMKKFTGKEVDCQR |
| | | TTPGVNLEAFSMMVSSFNNGTLIFKGEKRLNSLDPMTNSG |
| | | CATALDLASNKSVVVNKHLNGERLLEYDFNKLLVSAVSQI |
| | | TESFMRKQKYKLNHSDYEYKVSKLVSRLVIGSKETEAGKL |
| | | EGDSADICFDGEEETSFFKNLEDKVNSTIKRYERSKKTNE |
| | | GENEVGFENTKGLHHLQTILSGKMAYLRKVILSEISFHLV |
| | | EDFDPSCLTNDDMKFICEAIETSTELSPLYFTSAVKEQCG |
| | | LDEMAKNLCRKFFSEGDWFSCMKMILLQMNANAYSGKYRH |
| | | MQRQGLNFKFDWDKLEEDVRISERESNSESLSKALSLTKC |
| | | MSAALKNLCFYSEESPTSYTSVGPDSGRLKFALSYKEQVG |
| | | GNRELYIGDLRTKMFTRLIEDYFESFSSFFSGSCLNNDKE |
| | | FENAILSMTINVREGLLNYSMDHSKWGPMMCPFLFLMLLQ |
| | | NLKLGDDQYVRSGKDHISTLLTWHMHKLVEVPFPVVNAMM |
| | | KSYIKSKLKLLRGSETTVTERIFREYFELGIVPSHISSLI |
| | | DMGQGILHNASDFYGLISERFINYCIGVIFGERPESYTSS |
| | | DDQITLFDRRLSELVDSDPEEVLVLLEFHSHLSGLLNKFI |
| | | SPKSVVGRFAAEFKSRFYVWGEEVPLLTKFVSAALHNVKC |
| | | KEPHQLCETIDTIADQAVANGVPVSLVNCIQKRTLDLLKY |
| | | ANFPLDPFLLNTNTDVKDWLDGSRGYRIQRLIEELCPSET |
| | | KVMRRLVRRLHHKLKNGEFNEEFFLDLFNRDKKEAILQLG |
| | | NILGLEEDLSQLANINWLNLNELFPLRMVLRQKVVYPSVM |
| | | TFQEERIPSLIKTLQNKLCSKFTRGAQKLLSEAINKSAFQ |
| | | SCISSGFIGLCKTLGSRCVRNKNRDNLYIRKVLEDLAMDA |
| | | HVTAIHRHDGIMLYICDRQSHPEAHCDHISLLRPLLWDYI |
| | | CISLSNSFELGVWVLAEPVKGKNEGSSSLKHLNPCDYVAR |
| | | KPESSRLLEDKISLNHVIQSVRRLYPKIYEDQLLPFMSDM |
| | | SSKNMRWSPRIKFLDLCVLIDINSESLSLISHVVKWKRDE |
| | | HYTVLFSDLVNSHQRSDSSLVDEFVVSTRDVCKNFLKQVY |
| | | FESFVREFVATSRTLGSFSWFPHKDMMPSEDGAEALGPFQ |
| | | SFILKVVNKNMERPMFRNDLQFGFGWFSYRLGDIVCNAAM |
| | | LIKQGLTNPKAFKSLRNLWDYMINNTEGVLEFSITVDFTH |
| | | NQNNTDCLRKFSLIFLVKCQLQGPGVAEFLSCSHLFKGEV |
| | | DRRFLDECLHLLRSDSIFKVNDGVFDIRSEEFEDYMEDPL |
| | | ILGDSLELELIGSRKILDGIRSLDFERIGPEWEPVPLTVR |
| | | MGALFEGRSLVQNIVVKLETKDMRVFLAELEGYGNFDDVL |
| | | GSLLLHRFRTGEHLQGSEISTILQELCIDRSILLVPLSLV |
| | | PDWFTFKDCRLCFSKSKNTVMYETVVGKYRLKGKSCDDWL |
| | | TKSVVEEID |
| 9 | Amino acid sequence | MGQGKSKEGRDASNTSRAEILPDTTYLGPLNCKSCWQRFD |
| | of the Z protein of | SLVRCHDHYLCRHCLNLLLSVSDRCPLCKHPLPTKLKIST |
| | the MP strain of | APSSPPPYEE |
| | LCMV | |
| 10 | Junin virus | GCGCACCGGGGATCCTAGGCGTAACTTCATCATTAAAATCTCAGATTCT |
| | Candid#1 L segment | GCTCTGAGTGTGACTTACTGCGAAGAGGCAGACAAATGGGCAACTGCAA |
| | | CGGGGCATCCAAGTCTAACCAGCCAGACTCCTCAAGAGCCACACAGCCA |
| | | GCCGCAGAATTTAGGAGGGTAGCTCACAGCAGTCTATATGGTAGATATA |
| | | ACTGTAAGTGCTGCTGGTTTGCTGATACCAATTTGATAACCTGTAATGA |
| | | TCACTACCTTTGTTTAAGGTGCCATCAGGGTATGTTAAGGAATTCAGAT |
| | | CTCTGCAATATCTGCTGGAAGCCCCT |
| | | GCCCACCACAATCACAGTACCGGTGGAGCCAACAGCACCACCACCATAG |
| | | GCAGACTGCACAGGGTCAGACCCGACCCCCCGGGGGGCCCCCATGGGGA |
| | | CCCCCCGTGGGGGAACCCCGGGGGTGATGCGCCATTAGTCAATGTCTTT |
| | | GATCTCGACTTTGTGCTTCAGTGGCCTGCATGTCACCCCTTTCAATCTG |
| | | AACTGCCCTTGGGGATCTGATATCAGCAGGTCATTTAAAGATCT |
| | | GCTGAATGCCACCTTGAAATTTGAGAATTCCAACCAGTCACCAAATTTA |
| | | TCAAGTGAACGGATCAACTGCTCTTTGTGTA |
| | | GATCATAAACGAGGACAAAGTCCTCTTGCTGAAATAATATTGTTTGTGA |
| | | TGTTGTTTTTAGATAAGGCCATAGTTGGCTT |
| | | AATAAGGTTTCCACACTATCAATGTCCTCTAGTGCTCCAATTGCCTTGA |
| | | CTATGACATCCCCAGACAACTCAACTCTATA |
| | | TGTTGACAACCTTTCATTACCTCTGTAAAAGATACCCTCTTTCAAGACA |
| | | AGAGGTTCTCCTGGGTTATCTGGCCCAATGA |
| | | GGTCATATGCATACTTGTTACTTAGTTCAGAATAAAAGTCACCAAAGTT |
| | | GAACTTAACATGGCTCAGAATATTGTCATCA |
| | | TTTGTCGCAGCGTAGCCTGCATCAATAAACAAGCCAGCTAGGTCAAAGC |
| | | TCTCATGGCCTGTGAACAATGGTAGGCTAGC |
| | | GATAACCAGTGCACCATCCAACAATGAGTGGCTTCCCTCAGACCCAGAA |
| | | ACACATTGACTCATTGCATCCACATTCAGCT |
| | | CTAATTCAGGGGTACCGACATCATCCACTCCTAGTGAACTGACAATGGT |
| | | GTAACTGTACACCATCTTTCTTCTAAGTTTA |
| | | AATTTTGTCGAAACTCGTGTGTGTTCTACTTGAATGATCAATTTTAGTT |
| | | TCACAGCTTCTTGGCAAGCAACATTGCGCAA |
| | | CACAGTGTGCAGGTCCATCATGTCTTCCTGAGGCAACAAGGAGATGTTG |
| | | TCAACAGAGACACCCTCAAGGAAAACCTTGA |
| | | TATTATCAAAGCTAGAAACTACATAACCCATTGCAATGTCTTCAACAAA |
| | | CATTGCTCTTGATACTTTATTATTCCTAACT |
| | | GACAAGGTAAAATCTGTGAGTTCAGCTAGATCTACTTGACTGTCATCTT |
| | | CTAGATCTAGAACTTCATTGAACCAAAAGAA |
| | | GGATTTGAGACACGATGTTGACATGACTAGTGGGTTTATCATCGAAGAT |
| | | AAGACAACTTGCACCATGAAGTTCCTGCAAA |
| | | CTTGCTGTGGGCTGATGCCAACTTCCCAATTTGTATACTCTGACTGTCT |
| | | AACATGGGCTGAAGCGCAATCACTCTGTTTC |
| | | ACAATATAAACATTATTATCTCTTACTTTCAATAAGTGACTTATAATCC |
| | | CTAAGTTTTCATTCATCATGTCTAGAGCCAC |
| | | ACAGACATCTAGAAACTTGAGTCTTCCACTATCCAAAGATCTGTTCACT |
| | | TGAAGATCATTCATAAAGGGTGCCAAATGTT |
| | | CTTCAAATAGTTTGGGGTAATTTCTTCGTATAGAATGCAATACATGGTT |
| | | CATGCCTAATTGGTCTTCTATCTGTCGTACT |
| | | GCTTTGGGTTTAACAGCCCAGAAGAAATTCTTATTACATAAGACCAGAG |
| | | GGGCCTGTGGACTCTTAATAGCAGAAAACAC |
| | | CCACTCCCCTAACTCACAGGCATTTGTCAGCACCAAAGAGAAGTAATCC |
| | | CACAAAATTGGTTTAGAAAATTGGTTAACTT |
| | | CTTTAAGTGATTTTTGACAGTAAATAACTTTAGGCTTTCTCTCACAAAT |
| | | TCCACAAAGACATGGCATTATTCGAGTAAAT |
| | | ATGTCCTTTATATACAGAAATCCGCCTTTACCATCCCTAACACACTTAC |
| | | TCCCCATACTCTTACAAAACCCAATGAAGCC |
| | | TGAGGCAACAGAAGACTGAAATGCAGATTTGTTGATTGACTCTGCCAAG |
| | | ATCTTCTTCACGCCTTTTGTGAAATTTCTTG |
| | | ACAGCCTGGACTGTATTGTCCTTATCAATGTTGGCATCTCTTCTTTCTC |
| | | TAACACTCTTCGACTTGTCATGAGTTTGGTC |
| | | CTCAAGACCAACCTCAAGTCCCCAAAGCTCGCTAAATTGACCCATCTGT |
| | | AGTCTAGAGTTTGTCTGATTTCATCTTCACT |
| | | ACACCCGGCATATTGCAGGAATCCGGATAAAGCCTCATCCCCTCCCCTG |
| | | CTTATCAAGTTGATAAGGTTTTCCTCAAAGA |
| | | TTTTGCCTCTCTTAATGTCATTGAACACTTTCCTCGCGCAGTTCCTTAT |
| | | AAACATTGTCTCCTTATCATCAGAAAAAATA |
| | | GCTTCAATTTTCCTCTGTAGACGGTACCCTCTAGACCCATCAACCCAGT |
| | | CTTTGACATCTTGTTCTTCAATAGCTCCAAA |
| | | CGGAGTCTCTCTGTATCCAGAGTATCTAATCAATTGGTTGACTCTAATG |
| | | GAAATCTTTGACACTATATGAGTGCTAACCC |
| | | CATTAGCAATACATTGATCACAAATTGTGTCTATGGTCTCTGACAGTTG |
| | | TGTTGGAGTTTTACACTTAACGTTGTGTAGA |
| | | GCAGCAGACACAAACTTGGTGAGTAAAGGAGTCTCTTCACCCATGACAA |
| | | AAAATCTTGACTTAAACTCAGCAACAAAAGTTCCTATCACACTCTTTGG |
| | | GCTGATAAACTTGTTTAATTTAGAAGATAAGAATTCATGGAAGCACACC |
| | | ATTTCCAGCAGTT |
| | | CTGTCCTGTCTTGAAACTTTTCATCACTAAGGCAAGGAATTTTTATAAG |
| | | GCTAACCTGGTCATCGCTGGAGGTATAAGTG |
| | | ACAGGTATCACATCATACAATAAGTCAAGTGCATAACACAGAAATTGTT |
| | | CAGTAATTAGCCCATATAAATCTGATGTGTT |
| | | GTGCAAGATTCCCTGGCCCATGTCCAAGACAGACATTATATGGCTGGGG |
| | | ACCTGGTCCCTTGACTGCAGATACTGGTGAA |
| | | AAAACTCTTCACCAACACTAGTACAGTCACAACCCATTAAACCTAAAGA |
| | | TCTCTTCAATTTCCCTACACAGTAGGCTTCT |
| | | GCAACATTAATTGGAACTTCAACGACCTTATGAAGATGCCATTTGAGAA |
| | | TGTTCATTACTGGTTCAAGATTCACCTTTGT |
| | | TCTATCTCTGGGATTCTTCAATTCTAATGTGTACAAAAAAGAAAGGAAA |
| | | AGTGCTGGGCTCATAGTTGGTCCCCATTTGG |
| | | AGTGGTCATATGAACAGGACAAGTCACCATTGTTAACAGCCATTTTCAT |
| | | ATCACAGATTGCACGTTCGAATTCCTTTTCT |
| | | GAATTCAAGCATGTGTATTTCATTGAACTACCCACAGCTTCTGAGAAGT |
| | | CTTCAACTAACCTGGTCATCAGCTTAGTGTT |
| | | GAGGTCTCCCACATACAGTTCTCTATTTGAGCCAACCTGCTCCTTATAA |
| | | CTTAGTCCAAATTTCAAGTTCCCTGTATTTG |
| | | AGCTGATGCTTGTGAACTCTGTAGGAGAGTCGTCTGAATAGAAACATAA |
| | | ATTCCGTAGGGCTGCATTTGTAAAATAACTT |
| | | TTGTCTAGCTTATCAGCAATGGCTTCAGAATTGCTTTCCCTGGTACTAA |
| | | GCCGAACCTCATCCTTTAGTCTCAGAACTTC |
| | | ACTGGAAAAGCCCAATCTAGATCTACTTCTATGCTCATAACTACCCAAT |
| | | TTCTGATCATAATGTCCTTGAATTAAAAGAT |
| | | ACTTGAAGCATTCAAAGAATTCATCTTCTTGGTAGGCTATTGTTGTCAA |
| | | ATTTTTTAATAACAAACCCAAAGGGCAGATG |
| | | TCCTGCGGTGCTTCAAGAAAATAAGTCAATTTAAATGGAGATAGATAAA |
| | | CAGCATCACATAACTCTTTATACACATCAGA |
| | | CCTGAGCACATCTGGATCAAAATCCTTCACCTCATGCATTGACACCTCT |
| | | GCTTTAATCTCTCTCAACACTCCAAAAGGGG |
| | | CCCACAATGACTCAAGAGACTCTCGCTCATCAACAGATGGATTTTTTGA |
| | | TTTCAACTTGGTGATCTCAACTTTTGTCCCC |
| | | TCACTATTAGCCATCTTGGCTAGTGTCATTTGTACGTCATTTCTAATAC |
| | | CCTCAAAGGCCCTTACTTGATCCTCTGTTAA |
| | | ACTCTCATACATCACTGATAATTCTTCTTGATTGGTTCTGGTTCTTGAA |
| | | CCGGTGCTCACAAGACCTGTTAGATTTTTTA |
| | | ATATTAAGTAGTCCATGGAATCAGGATCAAGATTATACCTGCCTTTTGT |
| | | TTTAAACCTCTCAGCCATAGTAGAAACGCAT |
| | | GTTGAAACAAGTTTCTCCTTATCATAAACAGAAAGAATATTTCCAAGTT |
| | | CGTCGAGCTTGGGGATTACCACACTTTTATT |
| | | GCTTGACAGATCCAGAGCTGTGCTAGTGATGTTAGGCCTGTAGGGATTG |
| | | CTTTTCAGTTCACCTGTAACTTTAAGTCTTC |
| | | CTCTATTGAAGAGAGAAATGCAGAAGGACAAAATCTCTTTACACACTCC |
| | | TGGAATTTGAGTATCTGAGGAAGTCTTAGCC |
| | | TCTTTGGAAAAGAATCTGTCCAATCCTCTTATCATGGTGTCCTCTTGTT |
| | | CCAGTGTTAGACTCCCACTTAGAGGGGGGTT |
| | | TACAACAACACAATCAAACTTGACTTTGGGCTCAATAAACTTCTCAAAA |
| | | CACTTTATTTGATCTGTCAGGCGATCAGGTG |
| | | TCTCTTTGGTTACCAAGTGACACAGATAACTAACATTTAATAGATATTT |
| | | AAACCTTCTTGCAAAGTAAAGATCTGCATCT |
| | | TCCCCTTCACCCAAAATTGTCTGGAAAAGTTCCACAGCCATCCTCTGAA |
| | | TCAGCACCTCTGATCCAGACATGCAGTCGAC |
| | | CCTTAACTTTGACATCAAATCCACATGATGGATTTGATTTGCATATGCC |
| | | ATCAAGAAATATCTTAGACCTTGTAAAAATG |
| | | TCTGGTTCCTTTTGGAAGGGGAACAGAGTACAGCTAACACTAACAATCT |
| | | TAATATTGGCCTTGTCATTGTCATGAGTTCG |
| | | TGGCTAAAATCCAACCAGCTGGTCATTTCCTCACACATTTCAATTAACA |
| | | CATCCTCCGAAAATATAGGCAGGAAAAATCT |
| | | CTTTGGATCACAGTAAAAAGAGCCTTGTTCTTCCAATACCCCATTGATG |
| | | GATAGATAGATAGAATAGCACCTTGACTTCT |
| | | CACCTGTTTTTTGGTAAAACAAGAGACCAAATGTATTCTTTGTCAGATG |
| | | AAATCTTTGTACATAACACTCTCTTAGTCTA |
| | | ACATTCCCAAAATATCTAGAATACTCTCTTTCATTGATTAACAATCGGG |
| | | AGGAAAATGATGTCTTCATCGAGTTGACCAA |
| | | TGCAAGGGAAATGGAGGACAAAATCCTAAATAATTTCTTCTGCTCACCT |
| | | TCCACTAAGCTGCTGAATGGCTGATGTCTAC |
| | | AGATTTTCTCAAATTCCTTGTTAATAGTATATCTCATCACTGGTCTGTC |
| | | AGAAACAAGTGCCTGAGCTAAAATCATCAAG |
| | | CTATCCATATCAGGGTGTTTTATTAGTTTTTCCAGCTGTGACCAGAGAT |
| | | CTTGATGAGAGTTCTTCAATGTTCTGGAACA |
| | | CGCTTGAACCCACTTGGGGCTGGTCATCAATTTCTTCCTTATTAGTTTA |
| | | ATCGCCTCCAGAATATCTAGAAGTCTGTCAT |
| | | TGACTAACATTAACATTTGTCCAACAACTATTCCCGCATTTCTTAACCT |
| | | TACAATTGCATCATCATGCGTTTTGAAAAGA |
| | | TCACAAAGTAAATTGAGTAAAACTAAGTCCAGAAACAGTAAAGTGTTTC |
| | | TCCTGGTGTTGAAAACTTTTAGACCTTTCAC |
| | | TTTGTTACACACGGAAAGGGCTTGAAGATAACACCTCTCTACAGCATCA |
| | | ATAGATATAGAATTCTCATCTGACTGGCTTT |
| | | CCATGTTGACTTCATCTATTGGATGCAATGCGATAGAGTAGACTACATC |
| | | CATCAACTTGTTTGCACAAAAAGGGCAGCTG |
| | | GGCACATCACTGTCTTTGTGGCTTCCTAATAAGATCAAGTCATTTATAA |
| | | GCTTAGACTTTTGTGAAAATTTGAATTTCCC |
| | | CAACTGCTTGTCAAAAATCTCCTTCTTAAACCAAAACCTTAACTTTATG |
| | | AGTTCTTCTCTTATGACAGATTCTCTAATGT |
| | | CTCCTCTAACCCCAACAAAGAGGGATTCATTTAACCTCTCATCATAACC |
| | | CAAAGAATTCTTTTTCAAGCATTCGATGTTT |
| | | TCTAATCCCAAGCTCTGGTTTTTTGTGTTGGACAAACTATGGATCAATC |
| | | GCTGGTATTCTTGTTCTTCAATATTAATCTC |
| | | TTGCATAAATTTTGATTTCTTTAGGATGTCGATCAGCAACCACCGAACT |
| | | CTTTCAACAACCCAATCAGCAAGGAATCTAT |
| | | TGCTGTAGCTAGATCTGCCATCAACCACAGGAACCAACGTAATCCCTGC |
| | | CCTTAGTAGGTCGGACTTTAGGTTTAAGAGC |
| | | TTTGACATGTCACTCTTCCATTTTCTCTCAAACTCATCAGGATTGACCC |
| | | TAACAAAGGTTTCCAATAGGATGAGTGTTTT |
| | | CCCTGTGAGTTTGAAGCCATCCGGAATGACTTTTGGAAGGGTGGGACAT |
| | | AGTATGCCATAGTCAGACAGGATCACATCAA |
| | | CAAACTTCTGATCTGAATTGATCTGACAGGCGTGTGCCTCACAGGACTC |
| | | AAGCTCTACTAAACTTGACAGAAGTTTGAAC |
| | | CCTTCCAACAACAGAGAGCTGGGGTGATGTTGAGATAAAAAGATGTCCC |
| | | TTTGGTATGCTAGCTCCTGTCTTTCTGGAAA |
| | | ATGCTTTCTAATAAGGCTTTTTATTTCATTTACTGATTCCTCCATGCTC |
| | | AAGTGCCGCCTAGGATCCTCGGTGCG |
| 11 | Junin virus | GCGCACCGGGGATCCTAGGCGATTTTGGTTACGCTATAATTGTAACTGT |
| | Candid#1 S segment | TTTCTGTTTGGACAACATCAAAAACATCCATTGCACAATGGGGCAGTTC |
| | | ATTAGCTTCATGCAAGAAATACCAACCTTTTTGCAGGAGGCTCTGAACA |
| | | TTGCTCTTGTTGC |
| | | AGTCAGTCTCATTGCCATCATTAAGGGTATAGTGAACTTGTACAAAAGT |
| | | GGTTTATTCCAATTCTTTGTATTCCTAGCGC |
| | | TTGCAGGAAGATCCTGCACAGAAGAAGCTTTCAAAATCGGACTGCACAC |
| | | TGAGTTCCAGACTGTGTCCTTCTCAATGGTG |
| | | GGTCTCTTTTCCAACAATCCACATGACCTACCTTTGTTGTGTACCTTAA |
| | | ACAAGAGCCATCTTTACATTAAGGGGGGCAA |
| | | TGCTTCATTTCAGATCAGCTTTGATGATATTGCAGTATTGTTGCCACAG |
| | | TATGATGTTATAATACAACATCCAGCAGATA |
| | | TGAGCTGGTGTTCCAAAAGTGATGATCAAATTTGGTTGTCTCAGTGGTT |
| | | CATGAATGCTGTGGGACATGATTGGCATCTA |
| | | GACCCACCATTTCTGTGTAGGAACCGTGCAAAGACAGAAGGCTTCATCT |
| | | TTCAAGTCAACACCTCCAAGACTGGTGTCAA |
| | | TGGAAATTATGCTAAGAAGTTTAAGACTGGCATGCATCATTTATATAGA |
| | | GAATATCCTGACCCTTGCTTGAATGGCAAAC |
| | | TGTGCTTAATGAAGGCACAACCTACCAGTTGGCCTCTCCAATGTCCACT |
| | | CGACCACGTTAACACATTACACTTCCTTACA |
| | | AGAGGTAAAAACATTCAACTTCCAAGGAGGTCCTTGAAAGCATTCTTCT |
| | | CCTGGTCTTTGACAGACTCATCCGGCAAGGA |
| | | TACCCCTGGAGGCTATTGTCTAGAAGAGTGGATGCTCGTAGCAGCCAAA |
| | | ATGAAGTGTTTTGGCAATACTGCTGTAGCAA |
| | | AATGCAATTTGAATCATGACTCTGAATTCTGTGACATGTTGAGGCTCTT |
| | | TGATTACAACAAAAATGCTATCAAAACCCTA |
| | | AATGATGAAACTAAGAAACAAGTAAATCTGATGGGGCAGACAATCAATG |
| | | CCCTGATATCTGACAATTTATTGATGAAAAA |
| | | CAAAATTAGGGAACTGATGAGTGTCCCTTACTGCAATTACACAAAATTT |
| | | TGGTATGTCAACCACACACTTTCAGGACAAC |
| | | ACTCATTACCAAGGTGCTGGTTAATAAAAAACAACAGCTATTTGAACAT |
| | | CTCTGACTTCCGTAATGACTGGATATTAGAA |
| | | AGTGACTTCTTAATTTCTGAAATGCTAAGCAAAGAGTATTCGGACAGGC |
| | | AGGGTAAAACTCCTTTGACTTTAGTTGACAT |
| | | CTGTATTTGGAGCACAGTATTCTTCACAGCGTCACTCTTCCTTCACTTG |
| | | GTGGGTATACCCTCCCACAGACACATCAGGG |
| | | GCGAAGCATGCCCTTTGCCACACAGGTTGAACAGCTTGGGTGGTTGCAG |
| | | ATGTGGTAAGTACCCCAATCTAAAGAAACCA |
| | | ACAGTTTGGCGTAGAGGACACTAAGACCTCCTGAGGGTCCCCACCAGCC |
| | | CGGGCACTGCCCGGGCTGGTGTGGCCCCCCAGTCCGCGGCCTGGCCGCG |
| | | GACTGGGGAGGCACTGCTTACAGTGCATAGGCTGCCTTCGGGAGGAACA |
| | | GCAAGCTCGGTGGTAATAGAGGTGTAGGTTCCTCCTCATAGAGCTTCCC |
| | | ATCTAGCACTGACTGAAACATTATGCAGTCTAGCAGAGCACAGTGTGGT |
| | | TCACTGGAGGCCAACTTGAAGGGAGTATCCTTTTCCCTCTTTTTCTTAT |
| | | TGACAACCACTCCATTGTGATATTTG |
| | | CATAAGTGACCATATTTCTCCCAGACCTGTTGATCAAACTGCCTGGCTT |
| | | GTTCAGATGTGAGCTTAACATCAACCAGTTT |
| | | AAGATCTCTTCTTCCATGGAGGTCAAACAACTTCCTGATGTCATCGGAT |
| | | CCTTGAGTAGTCACAACCATGTCTGGAGGCA |
| | | GCAAGCCGATCACGTAACTAAGAACTCCTGGCATTGCATCTTCTATGTC |
| | | CTTCATTAAGATGCCGTGAGAGTGTCTGCTA |
| | | CCATTTTTAAACCCTTTCTCATCATGTGGTTTTCTGAAGCAGTGAATGT |
| | | ACTGCTTACCTGCAGGTTGGAATAATGCCAT |
| | | CTCAACAGGGTCAGTGGCTGGTCCTTCAATGTCGAGCCAAAGGGTGTTG |
| | | GTGGGGTCGAGTTTCCCCACTGCCTCTCTGA |
| | | TGACAGCTTCTTGTATCTCTGTCAAGTTAGCCAATCTCAAATTCTGACC |
| | | GTTTTTTTCCGGCTGTCTAGGACCAGCAACT |
| | | GGTTTCCTTGTCAGATCAATACTTGTGTTGTCCCATGACCTGCCTGTGA |
| | | TTTGTGATCTAGAACCAATATAAGGCCAACC |
| | | ATCGCCAGAAAGACAAAGTTTGTACAAAAGGTTTTCATAAGGATTTCTA |
| | | TTGCCTGGTTTCTCATCAATAAACATGCCTT |
| | | CTCTTCGTTTAACCTGAATGGTTGATTTTATGAGGGAAGAGAAGTTTTC |
| | | TGGGGTGACTCTGATTGTTTCCAACATGTTT |
| | | CCACCATCAAGAATAGATGCTCCAGCCTTTACTGCAGCTGAAAGACTGA |
| | | AGTTGTAACCAGAAATATTGATGGAGCTTTC |
| | | ATCTTTAGTCACAATCTGAAGGCAGTCATGTTCCTGAGTCAGTCTGTCA |
| | | AGGTCACTTAAGTTTGGATACTTCACAGTGT |
| | | ATAGAAGCCCAAGTGAGGTTAAAGCTTGTATGACACTGTTCATTGTCTC |
| | | ACCTCCTTGAACAGTCATGCATGCAATTGTC |
| | | AATGCAGGAACAGAGCCAAACTGATTGTTTAGCTTTGAAGGGTCTTTAA |
| | | CATCCCATATCCTCACCACACCATTTCCCCC |
| | | AGTCCCTTGCTGTTGAAATCCCAGTGTTCTCAATATCTCTGATCTTTTA |
| | | GCAAGTTGTGACTGGGACAAGTTACCCATGT |
| | | AAACCCCCTGAGAGCCTGTCTCTGCTCTTCTTATCTTGTTTTTTAATTT |
| | | CTCAAGGTCAGACGCCAACTCCATCAGTTCA |
| | | TCCCTCCCCAGATCTCCCACCTTGAAAACTGTGTTTCGTTGAACACTCC |
| | | TCATGGACATGAGTCTGTCAACCTCTTTATT |
| | | CAGGTCCCTCAACTTGTTGAGGTCTTCTTCCCCCTTTTTAGTCTTTCTG |
| | | AGTGCCCGCTGCACCTGTGCCACTTGGTTGA |
| | | AGTCGATGCTGTCAGCAATTAGCTTGGCGTCCTTCAAAACATCTGACTT |
| | | GACAGTCTGAGTGAATTGGCTCAAACCTCTC |
| | | CTTAAGGACTGAGTCCATCTAAAGCTTGGAACCTCCTTGGAGTGTGCCA |
| | | TGCCAGAAGTTCTGGTGATTTTGATCTAGAA |
| | | TAGAGTTGCTCAGTGAAAGTGTTAGACACTATGCCTAGGATCCACTGTG |
| | | CG |
| 12 | Amino acid sequence | MSLSKEVKSFQWTQALRRELQSFTSDVKAAVIKDATNLLNGLDFSEVSN |
| | of the NP protein | VQRIMRKEKRDDKDLQRLRSLNQTVHSLVDLKSTSKKNVLKVGRLSAEE |
| | of the Clone 13 | LMSLAADLEKLKAKIMRSERPQASGVYMGNLTTQQLDQRSQILQIVGMR |
| | strain of LCMV | KPQQGASGVVRVWDVKDSSLLNNQFGTMPSLTMACMAKQSQTPLNDVVQ |
| | (GenBank Accession | ALTDLGLLYTVKYPNLNDLERLKDKHPVLGVITEQQSSINISGYNFSLG |
| | No. ABC96002.1; | AAVKAGAALLDGGNMLESILIKPSNSEDLLKAVLGAKRKLNMFVSDQVG |
| | GI: 86440166) | DRNPYENILYKVCLSGEGWPYIACRTSIVGRAWENTTIDLTSEKPAVNS |
| | | PRPAPGAAGPPQVGLSYSQTMLLKDLMGGIDPNAPTWIDIEGRFNDPVE |
| | | IAIFQPQNGQFIHFYREPVDQKQFKQDSKYSHGMDLADLFNAQPGLTSS |
| | | VIGALPQGMVLSCQGSDDIRKLLDSQNRKDIKLIDVEMTREASREYEDK |
| | | VWDKYGWLCKMHTGIVRDKKKKEITPHCALMDCIIFESASKARLPDLKT |
| | | VHNILPHDLIFRGPNVVTL |
| 13 | Amino acid sequence | MGQIVTMFEALPHIIDEVINIVIIVLIVITGIKAVYNFATCGIFALISF |
| | of the GP protein | LLLAGRSCGMYGLKGPDIYKGVYQFKSVEFDMSHLNLTMPNACSANNSH |
| | of the Clone 13 | HYISMGTSGLELTFTNDSIISHNFCNLTSAFNKKTFDHTLMSIVSSLHL |
| | strain of LCMV | SIRGNSNYKAVSCDFNNGITIQYNLTFSDAQSAQSQCRTFRGRVLDMFR |
| | (GenBank Accession | TAFGGKYMRSGWGWTGSDGKTTWCSQTSYQYLIIQNRTWENHCTYAGPF |
| | No. ABC96001.2; | GMSRILLSQEKTKFLTRRLAGTFTWTLSDSSGVENPGGYCLTKWMILAA |
| | GI: 116563462) | ELKCFGNTAVAKCNVNHDEEFCDMLRLIDYNKAALSKFKEDVESALHLF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLVSIFLHLVKIPTHRHIKGGSCPKPHRLTNKGICSCGAFKVPG |
| | | VKTVWKRR |
| 14 | amino acid sequence | MDEIISELRELCLNYIEQDERLSRQKLNFLGQREPRMVLIEGLKLLSRC |
| | of the L protein of | IEIDSADKSGCTHNHDDKSVETILVESGIVCPGLPLIIPDGYKLIDNSL |
| | the Clone 13 strain | ILLECFVRSTPASFEKKFIEDTNKLACIREDLAVAGVTLVPIVDGRCDY |
| | of LCMV | DNSFMPEWANFKFRDLLFKLLEYSNQNEKVFEESEYFRLCESLKTTIDK |
| | (GenBank Accession | RSGMDSMKILKDARSTHNDEIMRMCHEGINPNMSCDDVVFGINSLFSRF |
| | No. ABC96004.1; | RRDLESGKLKRNFQKVNPEGLIKEFSELYENLADSDDILTLSREAVESC |
| | GI: 86440169) | PLMRFITAETHGHERGSETSTEYERLLSMLNKVKSLKLLNTRRRQLLNL |
| | | DVLCLSSLIKQSKFKGLKNDKHWVGCCYSSVNDRLVSFHSTKEEFIRLL |
| | | RNRKKSKVFRKVSFEELFRASISEFIAKIQKCLLVVGLSFEHYGLSEHL |
| | | EQECHIPFTEFENFMKIGAHPIMYYTKFEDYNFQPSTEQLKNIQSLRRL |
| | | SSVCLALTNSMKTSSVARLRQNQIGSVRYQVVECKEVFCQVIKLDSEEY |
| | | HLLYQKTGESSRCYSIQGPDGHLISFYADPKRFFLPIFSDEVLYNMIDI |
| | | MISWIRSCPDLKDCLTDIEVALRTLLLLMLTNPTKRNQKQVQSVRYLVM |
| | | AIVSDFSSTSLMDKLREDLITPAEKVVYKLLRFLIKTIFGTGEKVLLSA |
| | | KFKFMLNVSYLCHLITKETPDRLTDQIKCFEKFFEPKSQFGFFVNPKEA |
| | | ITPEEECVFYEQMKRFTSKEIDCQHTTPGVNLEAFSLMVSSFNNGTLIF |
| | | KGEKKLNSLDPMTNSGCATALDLASNKSVVVNKHLNGERLLEYDFNKLL |
| | | VSAVSQITESFVRKQKYKLSHSDYEYKVSKLVSRLVIGSKGEETGRSED |
| | | NLAEICFDGEEETSFFKSLEEKVNTTIARYRRGRRANDKGDGEKLTNTK |
| | | GLHHLQLILTGKMAHLRKVILSEISFHLVEDFDPSCLTNDDMKFICEAV |
| | | EGSTELSPLYFTSVIKDQCGLDEMAKNLCRKFFSENDWFSCMKMILLQM |
| | | NANAYSGKYRHMQRQGLNFKFDWDKLEEDVRISERESNSESLSKALSLT |
| | | QCMSAALKNLCFYSEESPTSYTSVGPDSGRLKFALSYKEQVGGNRELYI |
| | | GDLRTKMFTRLIEDYFESFSSFFSGSCLNNDKEFENAILSMTINVREGF |
| | | LNYSMDHSKWGPMMCPFLELMFLQNLKLGDDQYVRSGKDHVSTLLTWHM |
| | | HKLVEVPFPVVNAMMKSYVKSKLKLLRGSETTVTERIFRQYFEMGIVPS |
| | | HISSLIDMGQGILHNASDFYGLLSERFINYCIGVIFGERPEAYTSSDDQ |
| | | ITLFDRRLSDLVVSDPEEVLVLLEFQSHLSGLLNKFISPKSVAGRFAAE |
| | | FKSRFYVWGEEVPLLTKFVSAALHNVKCKEPHQLCETIDTIADQAIANG |
| | | VPVSLVNSIQRRTLDLLKYANFPLDPFLLNTNTDVKDWLDGSRGYRIQR |
| | | LIEELCPNETKVVRKLVRKLHHKLKNGEFNEEFFLDLFNRDKKEAILQL |
| | | GDLLGLEEDLNQLADVNWLNLNEMFPLRMVLRQKVVYPSVMTFQEERIP |
| | | SLIKTLQNKLCSKFTRGAQKLLSEAINKSAFQSCISSGFIGLCKTLGSR |
| | | CVRNKNRENLYIKKLLEDLTTDDHVTRVCNRDGITLYICDKQSHPEAHR |
| | | DHICLLRPLLWDYICISLSNSFELGVWVLAEPTKGKNNSENLTLKHLNP |
| | | CDYVARKPESSRLLEDKVNLNQVIQSVRRLYPKIFEDQLLPFMSDMSSK |
| | | NMRWSPRIKFLDLCVLIDINSESLSLISHVVKWKRDEHYTVLFSDLANS |
| | | HQRSDSSLVDEFVVSTRDVCKNFLKQVYFESFVREFVATTRTLGNFSWF |
| | | PHKEMMPSEDGAEALGPFQSFVSKVVNKNVERPMFRNDLQFGFGWFSYR |
| | | MGDVVCNAAMLIRQGLTNPKAFKSLKDLWDYMLNYTKGVLEFSISVDFT |
| | | HNQNNTDCLRKFSLIFLVRCQLQNPGVAELLSCSHLFKGEIDRRMLDEC |
| | | LHLLRTDSVFKVNDGVFDIRSEEFEDYMEDPLILGDSLELELLGSKRIL |
| | | DGIRSIDFERVGPEWEPVPLTVKMGALFEGRNLVQNIIVKLETKDMKVF |
| | | LAGLEGYEKISDVLGNLFLHRFRTGEHLLGSEISVILQELCIDRSILLI |
| | | PLSLLPDWFAFKDCRLCFSKSRSTLMYETVGGRFRLKGRSCDDWLGGSV |
| | | AEDID |
| 15 | Amino acid | MGQGKSREEKGTNSTNRAEILPDTTYLGPLSCKSCWQKFDSLVRCHDHY |
| | sequence of the Z | LCRHCLNLLLSVSDRCPLCKYPLPTRLKISTAPSSPPPYEE |
| | protein of the | |
| | Clone 13 strain of | |
| | LCMV | |
| | (GenBank Accession | |
| | No. ABC96003.1; | |
| | GI: 86440168) | |
| 16 | Amino acid sequence | MGQIVTMFEALPHIIDEVINIVIIVLIIITSIKAVYNFATCGILALVSF |
| | of the GP protein | LFLAGRSCGMYGLNGPDIYKGVYQFKSVEFDMSHLNLTMPNACSANNSH |
| | of the WE strain of | HYISMGSSGLELTFTNDSILNHNFCNLTSAFNKKTFDHTLMSIVSSLHL |
| | LCMV | SIRGNSNHKAVSCDFNNGITIQYNLSFSDPQSAISQCRTFRGRVLDMFR |
| | | TAFGGKYMRSGWGWAGSDGKTTWCSQTSYQYLIIQNRTWENHCRYAGPF |
| | | GMSRILFAQEKTKFLTRRLAGTFTWTLSDSSGVENPGGYCLTKWMILAA |
| | | ELKCFGNTAVAKCNVNHDEEFCDMLRLIDYNKAALSKFKQDVESALHVF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLISIFLHLVKIPTHRHIKGGSCPKPHRLTNKGICSCGAFKVPG |
| | | VKTIWKRR |
| 17 | WE specific primer | 5′AATCGTCTCTAAGGATGGGTCAGATTGTGACAATG-3′ |
| 18 | WE specific fusion- | 5′AATCGTCTCTAAGGATGGGTCAGATTGTGACAATG-3′ |
| | primer carrying an | |
| | overhang | |
| | complementary to | |
| | the WE-specific | |
| | primer | |
| 19 | WE specific primer | 5′CTCGGTGATCATGTTATCTGCTTCTTGTTCGATTTGA-3′ |
| 20 | WE specific fusion- | 5′AATCGTCTCTTTCTTTATCTCCTCTTCCAGATGG-3′ |
| | primer | |
| | complementary to | |
| | the WE-sequence | |
| 21 | Primer specific for | 5′-GGCTCCCAGATCTGAAAACTGTT-3′ |
| | LCMV NP | |
| 22 | NP- and GP-specific | 5′-GCTGGCTTGTCACTAATGGCTC-3′ |
| | primers; NP- | |
| | specific: same as | |
| | in RT reaction, GP- | |
| | specific: 5′ | |
| 23 | Lymphocytic | GCGCACCGGGGATCCTAGGCTTTTTGGATTGCGCTTTCCTCTAGATCAA |
| | choriomeningitis | CTGGGTGTCAGGCCCTATCCTACAGAAGGATGGGTCAGATTGTGACAAT |
| | virus clone 13 | GTTTGAGGCTTTGCCTCACATCATTGATGAGGTCATCAACATTGTCATT |
| | wildtype - Segment | ATTGTGCTCATTATAATCACGAGCATCAAAGCTGTGTACAATTTCGCCA |
| | S with WE - GP | CCTGTGGGATATTAGCACTGGTCAGCTTCCTTTTTTTGGCTGGTAGGTC |
| | (The genomic | CTGTGGCATGTACGGCCTTAATGGTCCCGACATCTATAAAGGGGTTTAC |
| | segment is RNA, the | CAGTTCAAATCAGTGGAGTTTGATATGTCTCACTTAAATCTGACGATGC |
| | sequence in SEQ ID | CCAATGCGTGCTCAGCCAACAACTCTCATCACTACATCAGTATGGGAAG |
| | NO: 23 is shown | CTCTGGACTGGAGCTAACTTTCACTAACGACTCCATCCTTAATCACAAT |
| | for DNA; however, | TTTTGCAACTTAACCTCCGCTTTCAACAAAAAGACTTTTGACCATACAC |
| | exchanging all | TCATGAGTATAGTCTCGAGTCTGCACCTCAGTATTAGAGGGAATTCCAA |
| | thymidines (“T”) in | CCACAAAGCAGTGTCTTGTGATTTTAACAATGGCATCACCATTCAATAC |
| | SEQ ID NO: 23 for | AACTTGTCATTTTCGGACCCACAGAGCGCTATAAGCCAGTGTAGGACTT |
| | uridines (“U”) | TCAGAGGTAGAGTCTTGGACATGTTTAGAACTGCCTTTGGAGGAAAATA |
| | provides the RNA | CATGAGAAGTGGCTGGGGCTGGGCAGGTTCAGATGGCAAGACCACTTGG |
| | sequence.) | TGCAGCCAAACAAGCTATCAGTACCTAATCATACAAAACAGGACTTGGG |
| | | AAAACCACTGTAGATATGCAGGCCCTTTTGGGATGTCTAGAATCCTCTT |
| | | TGCTCAGGAAAAGACAAAGTTTCTCACTAGGAGACTTGCAGGCACATTC |
| | | ACCTGGACCCTGTCAGACTCCTCAGGAGTAGAAAATCCAGGTGGTTATT |
| | | GCCTGACCAAATGGATGATCCTTGCTGCAGAGCTCAAATGTTTTGGGAA |
| | | TACAGCTGTTGCAAAATGTAATGTCAATCATGATGAAGAGTTCTGTGAC |
| | | ATGCTACGACTAATTGATTACAACAAGGCCGCCCTGAGTAAGTTCAAGC |
| | | AAGATGTAGAGTCTGCCTTGCATGTATTCAAAACAACAGTAAATTCTCT |
| | | GATTTCCGATCAGCTGTTGATGAGGAATCATCTAAGAGATCTAATGGGG |
| | | GTACCATACTGTAATTACTCAAAGTTCTGGTATCTGGAACATGCTAAGA |
| | | CTGGTGAGACTAGTGTACCCAAGTGCTGGCTTGTCACTAATGGCTCCTA |
| | | CTTGAATGAGACCCACTTTAGTGATCAAATCGAACAAGAAGCAGATAAC |
| | | ATGATCACAGAGATGTTGAGGAAGGACTACATAAAAAGACAAGGGAGTA |
| | | CTCCTTTAGCCTTAATGGATCTTTTGATGTTTTCAACATCAGCATATCT |
| | | AATCAGCATCTTTCTGCATCTTGTGAAGATACCAACACATAGACACATA |
| | | AAGGGCGGTTCATGTCCAAAGCCACACCGCTTGACCAACAAGGGGATCT |
| | | GTAGTTGTGGTGCATTCAAGGTGCCTGGTGTAAAAACTATCTGGAAAAG |
| | | ACGCTGAAGAACAGCGCCTCCCTGACTCTCCACCTCGAAAGAGGTGGAG |
| | | AGTCAGGGAGGCCCAGAGGGTCTTAGAGTGTCACAACATTTGGGCCTCT |
| | | AAAAATTAGGTCATGTGGCAGAATGTTGTGAACAGTTTTCAGATCTGGG |
| | | AGCCTTGCTTTGGAGGCGCTTTCAAAAATGATGCAGTCCATGAGTGCAC |
| | | AGTGCGGGGTGATCTCTTTCTTCTTTTTGTCCCTTACTATTCCAGTATG |
| | | CATCTTACACAACCAGCCATATTTGTCCCACACTTTaTCTTCATACTCC |
| | | CTCGAAGCTTCCCTGGTCATTTCAACATCGATAAGCTTAATGTCCTTCC |
| | | TATTtTGTGAGTCCAGAAGCTTTCTGATGTCATCGGAGCCTTGACAGCT |
| | | TAGAACCATCCCCTGCGGAAGAGCACCTATAACTGACGAGGTCAACCCG |
| | | GGTTGCGCATTGAAGAGGTCGGCAAGATCCATGCCGTGTGAGTACTTGG |
| | | AATCTTGCTTGAATTGTTTTTGATCAACGGGTTCCCTGTAAAAGTGTAT |
| | | GAACTGCCCGTTCTGTGGTTGGAAAATTGCTATTTCCACTGGATCATTA |
| | | AATCTACCCTCAATGTCAATCCATGTAGGAGCGTTGGGGTCAATTCCTC |
| | | CCATGAGGTCTTTTAAAAGCATTGTCTGGCTGTAGCTTAAGCCCACCTG |
| | | AGGTGGACCTGCTGCTCCAGGCGCTGGCCTGGGTGAgTTGACTGCAGGT |
| | | TTCTCGCTTGTGAGATCAATTGTTGTGTTTTCCCATGCTCTCCCCACAA |
| | | TCGATGTTCTACAAGCTATGTATGGCCATCCTTCACCTGAAAGGCAAAC |
| | | TTTATAGAGGATGTTTTCATAAGGGTTCCTGTCCCCAACTTGGTCTGAA |
| | | ACAAACATGTTGAGTTTTCTCTTGGCCCCGAGAACTGCCTTCAAGAGaT |
| | | CCTCGCTGTTGCTTGGCTTGATCAAAATTGACTCTAACATGTTACCCCC |
| | | ATCCAACAGGGCTGCCCCTGCCTTCACGGCAGCACCAAGACTAAAGTTA |
| | | TAGCCAGAAATGTTGATGCTGGACTGCTGTTCAGTGATGACCCCCAGAA |
| | | CTGGGTGCTTGTCTTTCAGCCTTTCAAGATCATTAAGATTTGGATACTT |
| | | GACTGTGTAAAGCAAGCCAAGGTCTGTGAGCGCTTGTACAACGTCATTG |
| | | AGCGGAGTCTGTGACTGTTTGGCCATACAAGCCATAGTTAGACTTGGCA |
| | | TTGTGCCAAATTGATTGTTCAAAAGTGATGAGTCTTTCACATCCCAAAC |
| | | TCTTACCACACCACTTGCACCCTGCTGAGGCTTTCTCATCCCAACTATC |
| | | TGTAGGATCTGAGATCTTTGGTCTAGTTGCTGTGTTGTTAAGTTCCCCA |
| | | TATATACCCCTGAAGCCTGGGGCCTTTCAGACCTCATGATCTTGGCCTT |
| | | CAGCTTCTCAAGGTCAGCCGCAAGAGACATCAGTTCTTCTGCACTGAGC |
| | | CTCCCCACTTTCAAAACATTCTTCTTTGATGTTGACTTTAAATCCACAA |
| | | GAGAATGTACAGTCTGGTTGAGACTTCTGAGTCTCTGTAGGTCTTTGTC |
| | | ATCTCTCTTTTCCTTCCTCATGATCCTCTGAACATTGCTGACCTCAGAG |
| | | AAGTCCAACCCATTCAGAAGGTTGGTTGCATCCTTAATGACAGCAGCCT |
| | | TCACATCTGATGTGAAGCTCTGCAATTCTCTTCTCAATGCTTGCGTCCA |
| | | TTGGAAGCTCTTAACTTCCTTAGACAAGGACATCTTGTTGCTCAATGGT |
| | | TTCTCAAGACAAATGCGCAATCAAATGCCTAGGATCCACTGTGCG |
| 24 | Pichinde virus | GCGCACCGGGGATCCTAGGCATACCTTGGACGCGCATATTACTTGATCA |
| | wildtype - Segment | AAGATGGGACAAGTTGTGACTTTGATCCAGTCTATACCCGAAGTCCTGC |
| | S | AGGAGGTGTTCAATGTCGCCTTAATCATTGTCTCAACCCTATGCATCAT |
| | Reference Sequence | CAAAGGATTTGTCAATCTGATGAGATGTGGCCTATTCCAACTCATCACC |
| | GenBank: EF529746.1 | TTCCTCATTTTGGCTGGCAGAAGTTGTGATGGCATGATGATTGATAGGA |
| | (The genomic | GGCACAATCTCACCCACGTTGAGTTCAACCTCACAAGAATGTTTGACAA |
| | segment is RNA, the | CTTGCCACAATCATGTAGCAAGAACAACACACATCATTACTACAAAGGA |
| | sequence in SEQ ID | CCATCTAACACAACATGGGGAATTGAACTCACTTTGACAAACACATCCA |
| | NO: 24 is shown | TTGCAAATGAAACTACTGGAAACTTTTCCAACATCAGAAGCCTTGCATA |
| | for DNA; however, | TGGTAACATTAGTAATTGTGATAAGACAGAAGAAGCAGGTCACACATTA |
| | exchanging all | AAATGGTTGCTTAATGAGTTACACTTCAATGTGCTCCATGTCACTCGTC |
| | thymidines (“T”) in | ATGTAGGTGCCAGATGCAAAACAGTTGAGGGTGCTGGGGTGTTGATCCA |
| | SEQ ID NO: 24 for | GTACAACTTGACAGTTGGGGATAGAGGAGGTGAGGTTGGCAGACATCTT |
| | uridines (“U”) | ATTGCGTCGCTTGCTCAAATCATTGGGGACCCAAAAATTGCGTGGGTTG |
| | provides the RNA | GAAAATGTTTCAATAACTGTAGTGGAGGGTCTTGCAGACTAACAAACTG |
| | sequence.) | TGAAGGTGGGACACATTACAATTTCCTGATCATACAGAACACCACATGG |
| | | GAAAATCACTGTACATATACTCCaATGGCAACAATAAGGATGGCTCTCC |
| | | AAAAAACTGCTTATAGTTCTGTGAGCAGGAAACTCCTTGGCTTTTTCAC |
| | | TTGGGACTTGAGTGACTCTACTGGGCAACATGTCCCAGGTGGTTACTGT |
| | | TTGGAGCAATGGGCTATTGTTTGGGCTGGAATAAAATGTTTTGATAACA |
| | | CTGTGATGGCAAAATGCAACAAAGATCACAATGAAGAATTTTGCGATAC |
| | | GATGAGGTTATTTGATTTCAATCAGAATGCTATCAAAACCTTACAACTT |
| | | AATGTTGAGAATTCGTTGAATCTCTTTAAAAAGACTATCAACGGACTTA |
| | | TTTCTGACTCACTTGTGATTAGAAACAGTCTCAAACAGCTTGCCAAAAT |
| | | CCCTTATTGCAACTATACAAAATTTTGGTACATCAATGATACCATCACA |
| | | GGgAGACATTCTTTACCGCAGTGTTGGTTAGTTCACAATGGCTCGTACC |
| | | TCAATGAAACGCATTTTAAGAATGATTGGTTGTGGGAGAGCCAGAATCT |
| | | GTACAATGAAATGCTGATAAAAGAATATGAAGAAAGACAAGGTAAGACT |
| | | CCACTAGCATTGACAGACATTTGCTTCTGGTCTTTGGTGTTTTACACCA |
| | | TCACAGTGTTTCTCCACTTAGTTGGAATACCCACTCATAGGCACATCAT |
| | | TGGTGATGGCTGTCCGAAGCCACATAGGATTACTAGGAACTCTCTTTGC |
| | | AGCTGTGGGTATTATAAAATCCCAAAGAAACCCTACAAATGGGTGAGAC |
| | | TGGGTAAATAAGCCCTAGCCTCGACATGGGCCTCGACGTCACTCCCCAA |
| | | TAGGGGAGTGACGTCGAGGCCTCTGAGGACTTGAGCTCAGAGGTTGATC |
| | | AGATCTGTGTTGTTCCTGTACAGCGTGTCAATAGGCAAGCATCTCATCG |
| | | GCTTCTGGTCCCTAACCCAGCCTGTCACTGTTGCATCAAACATGATGGT |
| | | ATCAAGCAATGCACAGTGAGGATTCGCAGTGGTTTGTGCAGCCCCCTTC |
| | | TTCTTCTTCTTTATGACCAAACCTTTATGTTTGGTGCAGAGTAGATTGT |
| | | ATCTCTCCCAGATCTCATCCTCAAAGGTGCGTGCTTGCTCGGCACTGAG |
| | | TTTCACGTCAAGCACTTTTAAGTCTCTTCTCCCATGCATTTCGAACAAA |
| | | CTGATTATATCATCTGAACCTTGAGCAGTGAAAACCATGTTTTGAGGTA |
| | | AATGTCTGATGATTGAGGAAATCAGGCCTGGTTGGGCATCAGCCAAGTC |
| | | CTTTAAAAGgAGACCATGTGAGTACTTGCTTTGCTCTTTGAAGGACTTC |
| | | TCATCGTGGGGAAATCTGTAACAATGTATGTAGTTGCCCGTGTCAGGCT |
| | | GGTAGATGGCCATTTCCACCGGATCATTTGGTGTTCCTTCAATGTCAAT |
| | | CCATGTGGTAGCTTTTGAATCAAGCATCTGAATTGAGGACACAACAGTa |
| | | TCTTCTTTCTCCTTAGGGATTTGTTTAAGGTCCGGTGATCCTCCGTTTC |
| | | TTACTGGTGGCTGGATAGCACTCGGCTTCGAATCTAAATCTACAGTGGT |
| | | GTTATCCCAAGCCCTCCCTTGAACTTGAGACCTTGAGCCAATGTAAGGC |
| | | CAACCATCCCCTGAAAGACAAATCTTGTATAGTAAATTTTCATAAGGAT |
| | | TTCTCTGTCCGGGTGTAGTGCTCACAAACATACCTTCACGATTCTTTAT |
| | | TTGCAATAGACTCTTTATGAGAGTACTAAACATAGAAGGCTTCACCTGG |
| | | ATGGTCTCAAGCATATTGCCACCATCAATCATGCAAGCAGCTGCTTTGA |
| | | CTGCTGCAGACAAACTGAGATTGTACCCTGAGATGTTTATGGCTGATGG |
| | | CTCATTACTAATGATTTTTAGGGCACTGTGTTGCTGTGTGAGTTTCTCT |
| | | AGATCTGTCATGTTCGGGAACTTGACAGTGTAGAGCAAACCAAGTGCAC |
| | | TCAGCGCTTGGACAACATCATTAAGTTGTTCACCCCCTTGCTCAGTCAT |
| | | ACAAGCGATGGTTAAGGCTGGCATTGATCCAAATTGATTGATCAACAAT |
| | | GTATTATCCTTGATGTCCCAGATCTTCACAACCCCATCTCTGTTGCCTG |
| | | TGGGTCTAGCATTAGCGAACCCCATTGAGCGAAGGATTTCGGCTCTTTG |
| | | TTCCAACTGAGTGTTTGTGAGATTGCCCCCATAAACACCAGGCTGAGAC |
| | | AAACTCTCAGTTCTAGTGACTTTCTTTCTTAACTTGTCCAAATCAGATG |
| | | CAAGCTCCATTAGCTCCTCTTTGGCTAAGCCTCCCACCTTAAGCACATT |
| | | GTCCCTCTGGATTGATCTCATATTCATCAGAGCATCAACCTCTTTGTTC |
| | | ATGTCTCTTAACTTGGTCAGATCAGAATCAGTCCTTTTATCTTTGCGCA |
| | | TCATTCTTTGAACTTGAGCAACTTTGTGAAAGTCAAGAGCAGATAACAG |
| | | TGCTCTTGTGTCCGACAACACATCAGCCTTCACAGGATGGGTCCAGTTG |
| | | GATAGACCCCTCCTAAGGGACTGTACCCAGCGGAATGATGGGATGTTGT |
| | | CAGACATTTTGGGGTTGTTTGCACTTCCTCCGAGTCAGTGAAGAAGTGA |
| | | ACGTACAGCGTGATCTAGAATCGCCTAGGATCCACTGTGCG |
| 25 | Pichinde virus | GCGCACCGGGGATCCTAGGCATCTTTGGGTCACGCTTCAAATTTGTCCA |
| | wildtype - Segment | ATTTGAACCCAGCTCAAGTCCTGGTCAAAACTTGGGATGGGACTCAGAT |
| | L | ATAGCAAAGAGGTCAGGAAGAGACATGGCGACGAAGATGTGGTGGGAAG |
| | Reference Sequence | GGTCCCCATGACCCTCAATCTACCACAGGGCCTGTATGGCAGGTTCAAC |
| | GenBank: EF529747.1 | TGCAAATCTTGCTGGTTCGTCAACAAAGGTCTCATCAGGTGCAAAGACC |
| | (The genomic | ACTATCTGTGTCTTGGGTGCTTAACCAAAATGCACTCCAGAGGCAATCT |
| | segment is RNA, the | CTGCGAGATATGCGGCCACTCACTGCCAACCAAGATGGAGTTCCTAGAA |
| | sequence in SEQ ID | AGCCCCTCTGCACCACCCTACGAGCCATAAACCAGGGCCCCTGGGCGCA |
| | NO: 25 is shown | CCCCCCTCCGGGGGTGCGCCCGGGGGCCCCCGGCCCCATGGGGCCGGTT |
| | for DNA; however, | GTTTACTCGATCTCCACTGACTCATTGTCCTCAAACAACTTTCGACACC |
| | exchanging all | TGATTCCCTTGATCTTGAAGGGTCCTGTCTCGTCTGCAATCATAACAGA |
| | thymidines (“T”) in | TCCTAGAGTCTTACTTCTTATTATACTAAAGTGACCACAATTCAACCAA |
| | SEQ ID NO: 25 for | TCTTTGGCATCATGCAACATGTGTTCAAACACTTCGGGGAAATTTTCAA |
| | uridines (“U”) | TCATGAGTCTTAAATCCTGCTCGTTCATACTTATTCCCTTGTTGTGAGA |
| | provides the RNA | CTGTGCACTTGAAAGGTACTGAAAAAGGTTGGCAATAAATCTTGGCCTT |
| | sequence.) | TTCTCAGGTTCTAATGCTTCCAGTGCAATGATGACCACCTTTGAGTCTA |
| | | AGTTCACTTCCAATCTAGAAACCACTCTGTTGCCCTCTTTGATCAACCC |
| | | ACCCTCTAAAATGAGGGGTTGCATCCCAACATCAGGACCAATCAACTTA |
| | | TAGGAAAATTTGTTTTTCAAATCCTTGAAACGATTTTTCAAATCTATTC |
| | | TCACCTTCTGGAACACAGTTGACCTTGACTTGAAGTGAATGTCTTGACC |
| | | TTCCAATAGATCATTGAAGTCTAGAACATCTTTTCCGTTGATGAGAGGA |
| | | TTCAGAACCAAAAGTGACACACCATCCAGACTTATGTGATTCCCGGAAG |
| | | ATTGAGAAACATAATACTCAACAGAATGGGGGTTCAACAATAGGTAACC |
| | | ATCAGAGTCCAATGAGTCCAGCAATGACTCCCTTTCAATAAGAAATCTT |
| | | AATTTTAATATGTAATTGGTAGACCTCTCATATCTAAATTTGTGGCTCA |
| | | CTCTCTTATGAGAAAATGTTAGGTTGAGCTCAATGGGAATGACCTCAGA |
| | | AGGTGATGCTAAAATGAGTTGTTCAATGTTCTCATAGTTATCTCTATTC |
| | | ACCCAGTCAAGTTCATTAATAAATACACTAATGTTCAAATTAACACAGG |
| | | ACAAAATCAGTTTGCTGCTTACAAAGCCAACATCCAAGTCATCCAGATT |
| | | CATTGTCCTAGAAGTGTTATTCTTTTTGCAGTCACAAATGAACTGGGTT |
| | | AATTGTTTCAGATCATGTTGTGCATTGTTTGGCAACAATTCAAGCTCAC |
| | | CAAACCAAAAATATTTCTTGAACTGAGATGTTGACATAATCACAGGCAC |
| | | CAACATTGACTCAAACAAAATCTGTATCAAGAAATTTGTGCACACTTCT |
| | | TCTGGTTCAAGGTTGAATCCTCTCTCCAGTGGATGAGACTCTCTGCTAT |
| | | GGGACATTGCAAGCTCATTTTGCTTTACAATATACAATTCTTCTCTGCG |
| | | ATGTTTTATAATATGACTAACAATACCAAGACATTCTGATGTTATATCA |
| | | ATTGCCACACAAAGGTCTAAGAACTTTATCCTCTGAACCCATGATAGCC |
| | | TCAGCATATTCAAATCAGACAGGAAAGGGGATATGTGTTCATCAAATAG |
| | | TGTAGGGAAGTTCCTCCTGATTGAGTAAAGTATGTGGTTGATGCCCACC |
| | | TTGTCCTCAAGCTCAGAATGTGTGCTTGGTTTTATTGGCCAGAAGTGAT |
| | | TGGGATTGTTTAGGTGAGTGACTATCTTGGGTACTTCAGCTTTTTGAAA |
| | | CACCCAGTTACCCAACTCGCAAGCATTGGTTAACACAAGAGCAAAATAA |
| | | TCCCAAATTAAGGGTCTGGAGTACTCACTTACTTCACCAAGTGCTGCTT |
| | | TACAATAAACACCTTTGCGCTGATTACAAAAGTGACAATCACGGTGTAA |
| | | GATAATCTTGCTTGTAATATCCCTGATATACTTAAATCCTCCTTTCCCa |
| | | TCTCTTACACATTTTGAGCCCATACTTTTGCAAACTCCTATGAATCCTG |
| | | ATGCTATGCTGCTCTGAAAAGCTGATTTGTTGATAGCATCAGCCAAAAT |
| | | CTTCTTAGCCCCTCTGACATAGTTCTTTGATAATTTGGACTGTACGGAT |
| | | TTGACAAGACTGGGTATTTCTTCTCGCTGCACAGTTCTTGTTGTGCTCA |
| | | TTAACTTAGTACGAAGCACCAATCTGAGATCACCATGAACCCTTAAATT |
| | | TAACCACCTAATATTAAGAGCATCCTCAATAGCCTCAGTCTCGACATCA |
| | | CAAGTCTCTAATAACTGTTTTAAGCAGTCATCCGGTGATTGCTGAAGAG |
| | | TTGTTACAATATAACTTTCTTCCAGGGCTCCAGACTGTATTTTGTAAAA |
| | | TATTTTCCTGCATGCCTTTCTGATTATTGAAAGTAGCAGATCATCAGGA |
| | | AATAGTGTCTCAATTGATCGCTGAAGTCTGTACCCTCTCGACCCATTAA |
| | | CCCAATCGAGTACATCCATTTCTTCCAGGCACAAAAATGGATCATTTGG |
| | | AAACCCACTATAGATTATCATGCTATTTGTTCGTTTTGCAATGGCCCCT |
| | | ACAACCTCTATTGACACCCCGTTAGCAACACATTGGTCCAGTATTGTGT |
| | | CAATTGTATCTGCTTGCTGATTGGGTGCTTTAGCCTTTATGTTGTGTAG |
| | | AGCTGCAGCAACAAACTTTGTAAGGAGGGGGACTTCTTGTGACCAAATG |
| | | AAGAATCTCGATTTGAACTCACTTGCAAAGGTCCCCACAACTGTTTTAG |
| | | GGCTCACAAACTTGTTGAGTTTGTCTGATAGAAAGTAGTGAAACTCCAT |
| | | ACAGTCCAATACCAATTCAACATTCAACTCATCTCTGTCCTTAAATTTG |
| | | AAACCCTCATTCAAGGATAACATGATCTCATCATCACTCGAAGTATATG |
| | | AGATGAACCGTGCTCCATAACAAAGCTCCAATGCGTAATTGATGAACTG |
| | | CTCAGTGATTAGACCATATAAGTCAGAGGTGTTGTGTAGGATGCCCTGA |
| | | CCCATATCTAAGACTGAAGAGATGTGTGATGGTACCTTGCCCTTCTCAA |
| | | AGTACCCAAACATAAATTCCTCTGCAATTGTGCACCCCCCTTTATCCAT |
| | | CATACCCAACCCCCTTTTCAAGAAACCTTTCATGTATGCCTCAACGACA |
| | | TTGAAGGGCACTTCCACCATCTTGTGAATGTGCCATAGCAATATGTTGA |
| | | TGACTGCAGCATTGGGAACTTCTGACCCATCTTTGAGTTTGAACTCAAG |
| | | ACCTTTTAATAATGCGGCAAAGATAACCGGCGACATGTGTGGCCCCCAT |
| | | TTTGAATGGTCCATTGACACCGCAAGACCACTTTGCCTAACAACTGACT |
| | | TCATGTCTAATAATGCTCTCTCAAACTCTTTCTCGTTGTTCAGACAAGT |
| | | ATACCTCATGTTTTGCATAAGGGATTCAGAGTAATCCTCAATGAGTCTG |
| | | GTTGTGAGTTTAGTATTTAAATCACCGACATAAAGCTCCCTGTTGCCAC |
| | | CCACCTGTTCTTTATAAGAAAGACCAAATTTCAATCTCCCTACATTGGT |
| | | GGATACACCAGACCTCTCTGTGGGAGACTCATCTGAATAGAAACAGAGA |
| | | TTTCGTAAGGATGAGTTGGTAAAAAAGCTTTGATCCAATCTTTTAGCTA |
| | | TCGATTCAGAATTGCTCTCTCTTGAGCTTATACGTGATGTCTCTCTAAT |
| | | TTGTAGTGCTGCATCTGTGAACCCAAGTCTGCTTCTACTTTTGTGATCA |
| | | TATCTTCCGACTCGATTATCATAATCGCTTGCAATGAGAATGTATTTAA |
| | | AGCACTCAAAATAATCAGCTTCTTTGTACGCCTTCAATGTGAGGTTCTT |
| | | TATTAAAAACTCCAGAGGACACGGATTCATTAGTCTGTCTGCAAAGTAC |
| | | ACTGATCTAGCAGTGACATCCTCATAGATCAAGTTTACAAGATCCTCAT |
| | | ACACTTCTGCTGAAAACAGGCTGTAATCAAAATCCTTTACATCATGAAG |
| | | TGAAGTCTCTCTTTTGATGACAACCATTGTCGATTTGGGCCATAATCTC |
| | | TCTAGTGGACATGAAGTCTTAAGGTTGGTTTTGACATTGGTGTCAACCT |
| | | TAGACAATACTTTTGCAACTCTGGTCTCAATTTCTTTAAGACAGTCACC |
| | | CTGATCTTCTGATAGTAACTCTTCAACTCCATCAGGCTCTATTGACTCC |
| | | TTTTTTATTTGGATCAATGATGACAACCTCTTCAGAATCTTGAAATTTA |
| | | CCTCCTTTGGATCtAACTTGTATTTACCCTTAGTTTTGAAATGTTCAAT |
| | | CATTTCCACAACAACAGCAGACACAATGGAAGAGTAATCATATTCAGTG |
| | | ATGACCTCACCAACTTCATTGAGTTTTGGAACCACCACACTTTTGTTGC |
| | | TGGACATATCCAAGGCTGTACTTGTGAAGGAGGGAGTCATAGGGTCACA |
| | | AGGAAGCAGGGGTTTCACTTCCAATGAGCTACTGTTAAATAGTGATAGA |
| | | CAAACACTAAGTACATCCTTATTCAACCCCGGCCTTCCCTCACATTTGG |
| | | ATTCCAGCTTTTTACCAAGTAGTCTCTCTATATCATGCACCATCTTCTC |
| | | TTCTTCCTCAGTAGGAAGTTCCATACTATTAGAAGGGTTGACCAAGACT |
| | | GAATCAAACTTTAACTTTGGTTCCAAGAACTTCTCAAAACATTTGATTT |
| | | GATCAGTTAATCTATCAGGGGTTTCTTTGGTTATAAAATGGCATAAATA |
| | | GGAGACATTCAAAACAAACTTAAAGATCTTAGCCATATCTTCCTCTCTG |
| | | GAGTTGCTGAGTACCAGAAGTATCAAATCATCAATAAGCATTGCTGTCT |
| | | GCCATTCTGAAGGTGTTAGCATAACGACTTTCAATTTCTCAAACAATTC |
| | | TTTAAAATGAACTTCATTTACAAAGGCCATAATGTAATATCTAAAGCCT |
| | | TGCAAGTAAACTTGAATACGCTTGGAAGGGGTGCACAGTATGCAGAGAA |
| | | TAAGTCGTCTGAGTAAATCAGAAACAGAATCCAAGAGGGGTTGGGACAT |
| | | AAAGTCCAACCAGGATAACATCTCCACACAAGTCCTTTGAATCACATCT |
| | | GCACTAAAGATCGGTAAGAAAAATCTCTTGGGATCACAGTAAAAAGACG |
| | | CTTTTGTTTCATACAAACCCCCACTTTTGGATCTATAAGCAACAGCATA |
| | | ACACCTGGACCTCTCCCCTGTCTTCTGGTACAGTAGTGTGAGAGAACCT |
| | | CCTTCTCCAAATCGCTGGAAGAAAACTTCGTCACAGTAAACCTTCCCAT |
| | | AAAACTCATCAGCATTGTTCACCTTCATCTTAGGAACTGCTGCTGTCTT |
| | | CATGCTATTAATGAGTGACAAACTCAAACTTGACAATGTTTTCAGCAAT |
| | | TCCTCAAACTCACTTTCGCCCATGATGGTATAATCAGGCTGCCCTCTTC |
| | | CTGGCCTACCCCCACACATACACTGTGACTTTGTCTTGTATTGAAGACA |
| | | GGGTTTAGCACCCCATTCATCTAACACTGATGTTTTCAGATTGAAGTAA |
| | | TATTCAACATCAGGTTCCCGTAGAAGAGGGAGAATGTCATCAAGGGGAA |
| | | GTTCACCACAGACCGAGCTCAGTCTCTTCTTAGCCTTCTCTAACCAGTT |
| | | GGGGTTTTTAATGAATTTTTTAGTGATTTGTTCCATCAGGAAGTCGACA |
| | | TTAATCAACCTGTCATTTACAGACGGTAACCCTTGCATTAGGAGCACCT |
| | | CTCTGAACACAGCACCTGGAGAAGACTTGTCCAAGTCACACAAAATGTT |
| | | GTACATGATAAGGTCCAGAACCAACATGGTGTTCCTCCTTGTGTTAAAA |
| | | ACCTTTTGAGACTTAATTTTGTTGCATATTGAAAGTACTCTAAAATATT |
| | | CTCTGCTTTCAGTTGATGAATGCTTGACCTCAGATTGCCTGAGTTGGCC |
| | | TATTATGCCCAAAATGTGTACTGAGCAAAACTCACATAATCTGATTTCT |
| | | GATTTAGGTACATCTTTGACAGAACATTGGATAAATTCATGGTTCTGAA |
| | | GTCTAGAAATCATATCTTCCCTATCTGTAGCCTGCAGTTTCCTATCGAG |
| | | TTGACCAGCAAGTTGCAACATTTTAAATTGCTGAAAGATTTCCATGATT |
| | | TTTGTTCTACATTGATCTGTTGTCAGTTTATTATTAATGCCAGACATTA |
| | | ATGCCTTTTCCAACCTCACTTTGTAAGGAAGTCCCCTTTCCTTTACAGC |
| | | AAGTAGTGACTCCAGACCGAGACTCTGATTTTCTAAGGATGAGAGGGAA |
| | | CTTATAAGGCGTTCGTACTCCAACTCCTCAACTTCTTCACCAGATGTCC |
| | | TTAATCCATCCATGAGTTTTAAAAGCAACCACCGAAGTCTCTCTACCAC |
| | | CCAATCAGGAACAAATTCTACATAATAACTGGATCTACCGTCAATAACA |
| | | GGTACTAAGGTTATGTTCTGTCTCTTGAGATCAGAACTAAGCTGCAACA |
| | | GCTTCAAAAAGTCCTGGTTGTATTTCTTCTCAAATGCTTCTTGACTGGT |
| | | CCTCACAAACACTTCCAAAAGAATGAGGACATCTCCAACCATACAGTAA |
| | | CCATCTGGTGTAACATCCGGCAATGTAGGACATGTTACTCTCAACTCCC |
| | | TAAGGATAGCATTGACAGTCATCTTTGTGTTGTGTTTGCAGGAGTGTTT |
| | | CTTGCATGAATCCACTTCCACTAGCATGGACAAAAGCTTCAGGCCCTCT |
| | | ATCGTGATGGCCCTATCTTTGACTTGTGCAAGAACGTTGTTTTTCTGTT |
| | | CAGATAGCTCTTCCCATTCGGGAACCCATTTTCTGACTATGTCTTTAAG |
| | | TTCGAAAACGTATTCCTCCATGATCAAGAAATGCCTAGGATCCTCGGTG |
| | | CG |
| 26 | Genomic sequence of | gcgcaccggggatcCTAGGCTTTTTGGATTGCGCTTTCCTCTAGATCAA |
| | LCMV vector | CTGGGTGTCAGGCCCTATCCTACAGAAGGATGCATGGTGACACCCCCAC |
| | (r3LCMV) encoding | CCTGCATGAGTACATGCTGGACCTGCAGCCAGAGACCACAGACCTGTAT |
| | HPV16 E7E6 fusion S | GGCTATGGCCAGCTGAATGACAGCAGTGAGGAAGAGGATGAGATTGATG |
| | Segment 1 | GGCCAGCAGGCCAGGCAGAACCTGACAGAGCCCACTACAACATTGTCAC |
| | (containing NP) | CTTCTGCTGCAAGTGTGACAGCACCCTGAGACTGTGTGTGCAGAGCACC |
| | | CATGTGGACATCAGAACCCTGGAAGACCTGCTGATGGGCACCCTGGGCA |
| | | TTGTGGGCCCCATCTGCTCCCAGAAGCCCCACCAGAAAAGAACTGCCAT |
| | | GTTCCAGGACCCCCAGGAGAGGCCCAGAAAGCTGCCCCAGCTCTGCACA |
| | | GAGCTGCAGACCACCATCCATGACATCATCCTGGAATGTGTCTACTGCA |
| | | AGCAGCAGCTGCTGAGGAGAGAGGTGTATGACTTTGCCTTCAGGGACCT |
| | | GTGCATTGTGTACAGGGATGGCAACCCCTATGCTGTGGGGGACAAGTGC |
| | | CTCAAGTTCTACAGCAAGATCAGTGAGTACAGGCACTACTGCTACAGCC |
| | | TGTATGGCACCACCCTGGAACAGCAGTACAACAAGCCCCTGTGTGACCT |
| | | CCTGATCAGATGCATCAATGGCCAGAAACCCCTCTGCCCTGAGGAAAAG |
| | | CAGAGACACCTGGACAAGAAGCAGAGGTTCCACAACATCAGAGGCAGGT |
| | | GGACAGGCAGATGCATGAGCTGCTGCAGAAGCAGCAGAACCAGAAGAGA |
| | | GACCCAGCTGTGAAGAACAGCGCCTCCCTGACTCTCCACCTCGAAAGAG |
| | | GTGGAGAGTCAGGGAGGCCCAGAGGGTCTTAGAGTGTCACAACATTTGG |
| | | GCCTCTAAAAATTAGGTCATGTGGCAGAATGTTGTGAACAGTTTTCAGA |
| | | TCTGGGAGCCTTGCTTTGGAGGCGCTTTCAAAAATGATGCAGTCCATGA |
| | | GTGCACAGTGCGGGGTGATCTCTTTCTTCTTTTTGTCCCTTACTATTCC |
| | | AGTATGCATCTTACACAACCAGCCATATTTGTCCCACACTTTaTCTTCA |
| | | TACTCCCTCGAAGCTTCCCTGGTCATTTCAACATCGATAAGCTTAATGT |
| | | CCTTCCTATTtTGTGAGTCCAGAAGCTTTCTGATGTCATCGGAGCCTTG |
| | | ACAGCTTAGAACCATCCCCTGCGGAAGAGCACCTATAACTGACGAGGTC |
| | | AACCCGGGTTGCGCATTGAAGAGGTCGGCAAGATCCATGCCGTGTGAGT |
| | | ACTTGGAATCTTGCTTGAATTGTTTTTGATCAACGGGTTCCCTGTAAAA |
| | | GTGTATGAACTGCCCGTTCTGTGGTTGGAAAATTGCTATTTCCACTGGA |
| | | TCATTAAATCTACCCTCAATGTCAATCCATGTAGGAGCGTTGGGGTCAA |
| | | TTCCTCCCATGAGGTCTTTTAAAAGCATTGTCTGGCTGTAGCTTAAGCC |
| | | CACCTGAGGTGGACCTGCTGCTCCAGGCGCTGGCCTGGGTGAgTTGACT |
| | | GCAGGTTTCTCGCTTGTGAGATCAATTGTTGTGTTTTCCCATGCTCTCC |
| | | CCACAATCGATGTTCTACAAGCTATGTATGGCCATCCTTCACCTGAAAG |
| | | GCAAACTTTATAGAGGATGTTTTCATAAGGGTTCCTGTCCCCAACTTGG |
| | | TCTGAAACAAACATGTTGAGTTTTCTCTTGGCCCCGAGAACTGCCTTCA |
| | | AGAGaTCCTCGCTGTTGCTTGGCTTGATCAAAATTGACTCTAACATGTT |
| | | ACCCCCATCCAACAGGGCTGCCCCTGCCTTCACGGCAGCACCAAGACTA |
| | | AAGTTATAGCCAGAAATGTTGATGCTGGACTGCTGTTCAGTGATGACCC |
| | | CCAGAACTGGGTGCTTGTCTTTCAGCCTTTCAAGATCATTAAGATTTGG |
| | | ATACTTGACTGTGTAAAGCAAGCCAAGGTCTGTGAGCGCTTGTACAACG |
| | | TCATTGAGCGGAGTCTGTGACTGTTTGGCCATACAAGCCATAGTTAGAC |
| | | TTGGCATTGTGCCAAATTGATTGTTCAAAAGTGATGAGTCTTTCACATC |
| | | CCAAACTCTTACCACACCACTTGCACCCTGCTGAGGCTTTCTCATCCCA |
| | | ACTATCTGTAGGATCTGAGATCTTTGGTCTAGTTGCTGTGTTGTTAAGT |
| | | TCCCCATATATACCCCTGAAGCCTGGGGCCTTTCAGACCTCATGATCTT |
| | | GGCCTTCAGCTTCTCAAGGTCAGCCGCAAGAGACATCAGTTCTTCTGCA |
| | | CTGAGCCTCCCCACTTTCAAAACATTCTTCTTTGATGTTGACTTTAAAT |
| | | CCACAAGAGAATGTACAGTCTGGTTGAGACTTCTGAGTCTCTGTAGGTC |
| | | TTTGTCATCTCTCTTTTCCTTCCTCATGATCCTCTGAACATTGCTGACC |
| | | TCAGAGAAGTCCAACCCATTCAGAAGGTTGGTTGCATCCTTAATGACAG |
| | | CAGCCTTCACATCTGATGTGAAGCTCTGCAATTCTCTTCTCAATGCTTG |
| | | CGTCCATTGGAAGCTCTTAACTTCCTTAGACAAGGACATCTTGTTGCTC |
| | | AATGGTTTCTCAAGACAAATGCGCAATCAAATGCctaggatccactgtg |
| | | cg |
| 27 | Genomic sequence of | gcgcaccggggatcCTAGGCTTTTTGGATTGCGCTTTCCTCTAGATCAA |
| | LCMV vector | CTGGGTGTCAGGCCCTATCCTACAGAAGGATGCATGGTGACACCCCCAC |
| | (r3LCMV) encoding | CCTGCATGAGTACATGCTGGACCTGCAGCCAGAGACCACAGACCTGTAT |
| | HPV16 E7E6 fusion S | GGCTATGGCCAGCTGAATGACAGCAGTGAGGAAGAGGATGAGATTGATG |
| | Segment 2 | GGCCAGCAGGCCAGGCAGAACCTGACAGAGCCCACTACAACATTGTCAC |
| | (containing GP) | CTTCTGCTGCAAGTGTGACAGCACCCTGAGACTGTGTGTGCAGAGCACC |
| | | CATGTGGACATCAGAACCCTGGAAGACCTGCTGATGGGCACCCTGGGCA |
| | | TTGTGGGCCCCATCTGCTCCCAGAAGCCCCACCAGAAAAGAACTGCCAT |
| | | GTTCCAGGACCCCCAGGAGAGGCCCAGAAAGCTGCCCCAGCTCTGCACA |
| | | GAGCTGCAGACCACCATCCATGACATCATCCTGGAATGTGTCTACTGCA |
| | | AGCAGCAGCTGCTGAGGAGAGAGGTGTATGACTTTGCCTTCAGGGACCT |
| | | GTGCATTGTGTACAGGGATGGCAACCCCTATGCTGTGGGGGACAAGTGC |
| | | CTCAAGTTCTACAGCAAGATCAGTGAGTACAGGCACTACTGCTACAGCC |
| | | TGTATGGCACCACCCTGGAACAGCAGTACAACAAGCCCCTGTGTGACCT |
| | | CCTGATCAGATGCATCAATGGCCAGAAACCCCTCTGCCCTGAGGAAAAG |
| | | CAGAGACACCTGGACAAGAAGCAGAGGTTCCACAACATCAGAGGCAGGT |
| | | GGACAGGCAGATGCATGAGCTGCTGCAGAAGCAGCAGAACCAGAAGAGA |
| | | GACCCAGCTGTGAAGAACAGCGCCTCCCTGACTCTCCACCTCGAAAGAG |
| | | GTGGAGAGTCAGGGAGGCCCAGAGGGTCTCAGCGTCTTTTCCAGATAGT |
| | | TTTTACACCAGGCACCTTGAATGCACCACAACTACAGATCCCCTTGTTG |
| | | GTCAAGCGGTGTGGCTTTGGACATGAACCGCCCTTTATGTGTCTATGTG |
| | | TTGGTATCTTCACAAGATGCAGAAAGATGCTGATTAGATATGCTGATGT |
| | | TGAAAACATCAAAAGATCCATTAAGGCTAAAGGAGTACTCCCTTGTCTT |
| | | TTTATGTAGTCCTTCCTCAACATCTCTGTGATCATGTTATCTGCTTCTT |
| | | GTTCGATTTGATCACTAAAGTGGGTCTCATTCAAGTAGGAGCCATTAGT |
| | | GACAAGCCAGCACTTGGGTACACTAGTCTCACCAGTCTTAGCATGTTCC |
| | | AGATACCAGAACTTTGAGTAATTACAGTATGGTACCCCCATTAGATCTC |
| | | TTAGATGATTCCTCATCAACAGCTGATCGGAAATCAGAGAATTTACTGT |
| | | TGTTTTGAATACATGCAAGGCAGACTCTACATCTTGCTTGAACTTACTC |
| | | AGGGCGGCCTTGTTGTAATCAATTAGTCGTAGCATGTCACAGAACTCTT |
| | | CATCATGATTGACATTACATTTTGCAACAGCTGTATTCCCAAAACATTT |
| | | GAGCTCTGCAGCAAGGATCATCCATTTGGTCAGGCAATAACCACCTGGA |
| | | TTTTCTACTCCTGAGGAGTCTGACAGGGTCCAGGTGAATGTGCCTGCAA |
| | | GTCTCCTAGTGAGAAACTTTGTCTTTTCCTGAGCAAAGAGGATTCTAGA |
| | | CATCCCAAAAGGGCCTGCATATCTACAGTGGTTTTCCCAAGTCCTGTTT |
| | | TGTATGATTAGGTACTGATAGCTTGTTTGGCTGCACCAAGTGGTCTTGC |
| | | CATCTGAACCTGCCCAGCCCCAGCCACTTCTCATGTATTTTCCTCCAAA |
| | | GGCAGTTCTAAACATGTCCAAGACTCTACCTCTGAAAGTCCTACACTGG |
| | | CTTATAGCGCTCTGTGGGTCCGAAAATGACAAGTTGTATTGAATGGTGA |
| | | TGCCATTGTTAAAATCACAAGACACTGCTTTGTGGTTGGAATTCCCTCT |
| | | AATACTGAGGTGCAGACTCGAGACTATACTCATGAGTGTATGGTCAAAA |
| | | GTCTTTTTGTTGAAAGCGGAGGTTAAGTTGCAAAAATTGTGATTAAGGA |
| | | TGGAGTCGTTAGTGAAAGTTAGCTCCAGTCCAGAGCTTCCCATACTGAT |
| | | GTAGTGATGAGAGTTGTTGGCTGAGCACGCATTGGGCATCGTCAGATTT |
| | | AAGTGAGACATATCAAACTCCACTGATTTGAACTGGTAAACCCCTTTAT |
| | | AGATGTCGGGACCATTAAGGCCGTACATGCCACAGGACCTACCAGCCAA |
| | | AAAAAGGAAGCTGACCAGTGCTAATATCCCACAGGTGGCGAAATTGTAC |
| | | ACAGCTTTGATGCTCGTGATTATAATGAGCACAATAATGACAATGTTGA |
| | | TGACCTCATCAATGATGTGAGGCAAAGCCTCAAACATTGTCACAATCTG |
| | | ACCCATCTTGTTGCTCAATGGTTTCTCAAGACAAATGCGCAATCAAATG |
| | | Cctaggatccactgtgcg |
| 28 | Genomic sequence of | gcgcaccggggatcCTAGGCATACCTTGGACGCGCATATTACTTGATCA |
| | Pichinde vector | AAGATGCATGGTGACACCCCCACCCTGCATGAGTACATGCTGGACCTGC |
| | (r3PICV) encoding | AGCCAGAGACCACAGACCTGTATGGCTATGGCCAGCTGAATGACAGCAG |
| | HPV16 E7E6 fusion S | TGAGGAAGAGGATGAGATTGATGGGCCAGCAGGCCAGGCAGAACCTGAC |
| | Segment 1 | AGAGCCCACTACAACATTGTCACCTTCTGCTGCAAGTGTGACAGCACCC |
| | (containing NP) | TGAGACTGTGTGTGCAGAGCACCCATGTGGACATCAGAACCCTGGAAGA |
| | | CCTGCTGATGGGCACCCTGGGCATTGTGGGCCCCATCTGCTCCCAGAAG |
| | | CCCCACCAGAAAAGAACTGCCATGTTCCAGGACCCCCAGGAGAGGCCCA |
| | | GAAAGCTGCCCCAGCTCTGCACAGAGCTGCAGACCACCATCCATGACAT |
| | | CATCCTGGAATGTGTCTACTGCAAGCAGCAGCTGCTGAGGAGAGAGGTG |
| | | TATGACTTTGCCTTCAGGGACCTGTGCATTGTGTACAGGGATGGCAACC |
| | | CCTATGCTGTGGGGGACAAGTGCCTCAAGTTCTACAGCAAGATCAGTGA |
| | | GTACAGGCACTACTGCTACAGCCTGTATGGCACCACCCTGGAACAGCAG |
| | | TACAACAAGCCCCTGTGTGACCTCCTGATCAGATGCATCAATGGCCAGA |
| | | AACCCCTCTGCCCTGAGGAAAAGCAGAGACACCTGGACAAGAAGCAGAG |
| | | GTTCCACAACATCAGAGGCAGGTGGACAGGCAGATGCATGAGCTGCTGC |
| | | AGAAGCAGCAGAACCAGAAGAGAGACCCAGCTGTGAGCCCTAGCCTCGA |
| | | CATGGGCCTCGACGTCACTCCCCAATAGGGGAGTGACGTCGAGGCCTCT |
| | | GAGGACTTGAGCTCAGAGGTTGATCAGATCTGTGTTGTTCCTGTACAGC |
| | | GTGTCAATAGGCAAGCATCTCATCGGCTTCTGGTCCCTAACCCAGCCTG |
| | | TCACTGTTGCATCAAACATGATGGTATCAAGCAATGCACAGTGAGGATT |
| | | CGCAGTGGTTTGTGCAGCCCCCTTCTTCTTCTTCTTTATGACCAAACCT |
| | | TTATGTTTGGTGCAGAGTAGATTGTATCTCTCCCAGATCTCATCCTCAA |
| | | AGGTGCGTGCTTGCTCGGCACTGAGTTTCACGTCAAGCACTTTTAAGTC |
| | | TCTTCTCCCATGCATTTCGAACAAACTGATTATATCATCTGAACCTTGA |
| | | GCAGTGAAAACCATGTTTTGAGGTAAATGTCTGATGATTGAGGAAATCA |
| | | GGCCTGGTTGGGCATCAGCCAAGTCCTTTAAAAGgAGACCATGTGAGTA |
| | | CTTGCTTTGCTCTTTGAAGGACTTCTCATCGTGGGGAAATCTGTAACAA |
| | | TGTATGTAGTTGCCCGTGTCAGGCTGGTAGATGGCCATTTCCACCGGAT |
| | | CATTTGGTGTTCCTTCAATGTCAATCCATGTGGTAGCTTTTGAATCAAG |
| | | CATCTGAATTGAGGACACAACAGTaTCTTCTTTCTCCTTAGGGATTTGT |
| | | TTAAGGTCCGGTGATCCTCCGTTTCTTACTGGTGGCTGGATAGCACTCG |
| | | GCTTCGAATCTAAATCTACAGTGGTGTTATCCCAAGCCCTCCCTTGAAC |
| | | TTGAGACCTTGAGCCAATGTAAGGCCAACCATCCCCTGAAAGACAAATC |
| | | TTGTATAGTAAATTTTCATAAGGATTTCTCTGTCCGGGTGTAGTGCTCA |
| | | CAAACATACCTTCACGATTCTTTATTTGCAATAGACTCTTTATGAGAGT |
| | | ACTAAACATAGAAGGCTTCACCTGGATGGTCTCAAGCATATTGCCACCA |
| | | TCAATCATGCAAGCAGCTGCTTTGACTGCTGCAGACAAACTGAGATTGT |
| | | ACCCTGAGATGTTTATGGCTGATGGCTCATTACTAATGATTTTTAGGGC |
| | | ACTGTGTTGCTGTGTGAGTTTCTCTAGATCTGTCATGTTCGGGAACTTG |
| | | ACAGTGTAGAGCAAACCAAGTGCACTCAGCGCTTGGACAACATCATTAA |
| | | GTTGTTCACCCCCTTGCTCAGTCATACAAGCGATGGTTAAGGCTGGCAT |
| | | TGATCCAAATTGATTGATCAACAATGTATTATCCTTGATGTCCCAGATC |
| | | TTCACAACCCCATCTCTGTTGCCTGTGGGTCTAGCATTAGCGAACCCCA |
| | | TTGAGCGAAGGATTTCGGCTCTTTGTTCCAACTGAGTGTTTGTGAGATT |
| | | GCCCCCATAAACACCAGGCTGAGACAAACTCTCAGTTCTAGTGACTTTC |
| | | TTTCTTAACTTGTCCAAATCAGATGCAAGCTCCATTAGCTCCTCTTTGG |
| | | CTAAGCCTCCCACCTTAAGCACATTGTCCCTCTGGATTGATCTCATATT |
| | | CATCAGAGCATCAACCTCTTTGTTCATGTCTCTTAACTTGGTCAGATCA |
| | | GAATCAGTCCTTTTATCTTTGCGCATCATTCTTTGAACTTGAGCAACTT |
| | | TGTGAAAGTCAAGAGCAGATAACAGTGCTCTTGTGTCCGACAACACATC |
| | | AGCCTTCACAGGATGGGTCCAGTTGGATAGACCCCTCCTAAGGGACTGT |
| | | ACCCAGCGGAATGATGGGATGTTGTCAGACATTTTGGGGTTGTTTGCAC |
| | | TTCCTCCGAGTCAGTGAAGAAGTGAACGTACAGCGTGATCTAGAATCGC |
| | | ctaggatccactgtgcg |
| 29 | Genomic sequence of | gcgcaccggggatcCTAGGCATACCTTGGACGCGCATATTACTTGATCA |
| | Pichinde vector | AAGATGCATGGTGACACCCCCACCCTGCATGAGTACATGCTGGACCTGC |
| | (r3PICV) encoding | AGCCAGAGACCACAGACCTGTATGGCTATGGCCAGCTGAATGACAGCAG |
| | HPV16 E7E6 fusion S | TGAGGAAGAGGATGAGATTGATGGGCCAGCAGGCCAGGCAGAACCTGAC |
| | Segment 2 | AGAGCCCACTACAACATTGTCACCTTCTGCTGCAAGTGTGACAGCACCC |
| | (containing GP) | TGAGACTGTGTGTGCAGAGCACCCATGTGGACATCAGAACCCTGGAAGA |
| | | CCTGCTGATGGGCACCCTGGGCATTGTGGGCCCCATCTGCTCCCAGAAG |
| | | CCCCACCAGAAAAGAACTGCCATGTTCCAGGACCCCCAGGAGAGGCCCA |
| | | GAAAGCTGCCCCAGCTCTGCACAGAGCTGCAGACCACCATCCATGACAT |
| | | CATCCTGGAATGTGTCTACTGCAAGCAGCAGCTGCTGAGGAGAGAGGTG |
| | | TATGACTTTGCCTTCAGGGACCTGTGCATTGTGTACAGGGATGGCAACC |
| | | CCTATGCTGTGGGGGACAAGTGCCTCAAGTTCTACAGCAAGATCAGTGA |
| | | GTACAGGCACTACTGCTACAGCCTGTATGGCACCACCCTGGAACAGCAG |
| | | TACAACAAGCCCCTGTGTGACCTCCTGATCAGATGCATCAATGGCCAGA |
| | | AACCCCTCTGCCCTGAGGAAAAGCAGAGACACCTGGACAAGAAGCAGAG |
| | | GTTCCACAACATCAGAGGCAGGTGGACAGGCAGATGCATGAGCTGCTGC |
| | | AGAAGCAGCAGAACCAGAAGAGAGACCCAGCTGTGAGCCCTAGCCTCGA |
| | | CATGGGCCTCGACGTCACTCCCCAATAGGGGAGTGACGTCGAGGCCTCT |
| | | GAGGACTTGAGCTTATTTACCCAGTCTCACCCATTTGTAGGGTTTCTTT |
| | | GGGATTTTATAATACCCACAGCTGCAAAGAGAGTTCCTAGTAATCCTAT |
| | | GTGGCTTCGGACAGCCATCACCAATGATGTGCCTATGAGTGGGTATTCC |
| | | AACTAAGTGGAGAAACACTGTGATGGTGTAAAACACCAAAGACCAGAAG |
| | | CAAATGTCTGTCAATGCTAGTGGAGTCTTACCTTGTCTTTCTTCATATT |
| | | CTTTTATCAGCATTTCATTGTACAGATTCTGGCTCTCCCACAACCAATC |
| | | ATTCTTAAAATGCGTTTCATTGAGGTACGAGCCATTGTGAACTAACCAA |
| | | CACTGCGGTAAAGAATGTCTcCCTGTGATGGTATCATTGATGTACCAAA |
| | | ATTTTGTATAGTTGCAATAAGGGATTTTGGCAAGCTGTTTGAGACTGTT |
| | | TCTAATCACAAGTGAGTCAGAAATAAGTCCGTTGATAGTCTTTTTAAAG |
| | | AGATTCAACGAATTCTCAACATTAAGTTGTAAGGTTTTGATAGCATTCT |
| | | GATTGAAATCAAATAACCTCATCGTATCGCAAAATTCTTCATTGTGATC |
| | | TTTGTTGCATTTTGCCATCACAGTGTTATCAAAACATTTTATTCCAGCC |
| | | CAAACAATAGCCCATTGCTCCAAACAGTAACCACCTGGGACATGTTGCC |
| | | CAGTAGAGTCACTCAAGTCCCAAGTGAAAAAGCCAAGGAGTTTCCTGCT |
| | | CACAGAACTATAAGCAGTTTTTTGGAGAGCCATCCTTATTGTTGCCATt |
| | | GGAGTATATGTACAGTGATTTTCCCATGTGGTGTTCTGTATGATCAGGA |
| | | AATTGTAATGTGTCCCACCTTCACAGTTTGTTAGTCTGCAAGACCCTCC |
| | | ACTACAGTTATTGAAACATTTTCCAACCCACGCAATTTTTGGGTCCCCA |
| | | ATGATTTGAGCAAGCGACGCAATAAGATGTCTGCCAACCTCACCTCCTC |
| | | TATCCCCAACTGTCAAGTTGTACTGGATCAACACCCCAGCACCCTCAAC |
| | | TGTTTTGCATCTGGCACCTACATGACGAGTGACATGGAGCACATTGAAG |
| | | TGTAACTCATTAAGCAACCATTTTAATGTGTGACCTGCTTCTTCTGTCT |
| | | TATCACAATTACTAATGTTACCATATGCAAGGCTTCTGATGTTGGAAAA |
| | | GTTTCCAGTAGTTTCATTTGCAATGGATGTGTTTGTCAAAGTGAGTTCA |
| | | ATTCCCCATGTTGTGTTAGATGGTCCTTTGTAGTAATGATGTGTGTTGT |
| | | TCTTGCTACATGATTGTGGCAAGTTGTCAAACATTCTTGTGAGGTTGAA |
| | | CTCAACGTGGGTGAGATTGTGCCTCCTATCAATCATCATGCCATCACAA |
| | | CTTCTGCCAGCCAAAATGAGGAAGGTGATGAGTTGGAATAGGCCACATC |
| | | TCATCAGATTGACAAATCCTTTGATGATGCATAGGGTTGAGACAATGAT |
| | | TAAGGCGACATTGAACACCTCCTGCAGGACTTCGGGTATAGACTGGATC |
| | | AAAGTCACAACTTGTCCCATTTTGGGGTTGTTTGCACTTCCTCCGAGTC |
| | | AGTGAAGAAGTGAACGTACAGCGTGATCTAGAATCGCctaggatccact |
| | | gtgcg |
| 30 | Genomic sequence of | gCGCACCGGGGATCCTAGGCTTTTTGGATTGCGCTTTCCTCTAGATCAA |
| | LCMV vector | CTGGGTGTCAGGCCCTATCCTACAGAAGGATGGGCCTTGTGGGATGGGG |
| | (r3LCMV) encoding | GCTTCTGCTGGGTTGTCTGGGCTGTGGAATTCTGCTCAGAGCCAGGGCT |
| | TRP2 S Segment 1 | CAGTTTCCCAGAGTCTGCATGACCTTGGATGGGGTGCTGAACAAGGAAT |
| | (containing NP) | GCTGCCCCCCTCTGGGTCCAGAGGCAACCAACATCTGTGGATTTCTGGA |
| | | GGGCAGGGGGCAGTGTGCAGAGGTGCAAACAGACACCAGACCCTGGAGT |
| | | GGCCCTTACATCCTCAGAAACCAGGATGACAGGGAGCAATGGCCAAGAA |
| | | AATTCTTCAACAGGACATGCAAATGCACAGGAAACTTTGCTGGTTACAA |
| | | TTGTGGAGGCTGCAAGTTTGGCTGGACTGGCCCAGACTGCAACAGGAAG |
| | | AAGCCAGCCATCCTCAGAAGGAACATCCATTCCCTGACTGCCCAGGAGA |
| | | GGGAGCAGTTCTTGGGAGCCTTGGACCTGGCCAAGAAGAGCATCCATCC |
| | | AGACTATGTGATCACCACACAACACTGGCTGGGGCTGCTGGGACCCAAT |
| | | GGGACCCAGCCCCAGATTGCCAACTGCAGTGTGTATGACTTTTTTGTGT |
| | | GGCTCCATTATTATTCTGTGAGAGACACATTGTTGGGTCCAGGAAGACC |
| | | CTACAAGGCCATTGATTTCTCTCACCAAGGGCCTGCCTTTGTCACCTGG |
| | | CACAGGTACCATCTGTTGTGGCTGGAAAGAGAACTCCAGAGACTCACTG |
| | | GCAATGAGTCCTTTGCCTTGCCCTACTGGAACTTTGCAACTGGGAAGAA |
| | | TGAGTGTGATGTGTGCACAGATGAGCTGCTTGGAGCAGCAAGACAAGAT |
| | | GACCCAACACTGATCAGCAGGAACTCAAGATTCTCAACCTGGGAGATTG |
| | | TGTGTGACAGCTTGGATGACTACAACAGGAGGGTCACACTGTGCAATGG |
| | | AACCTATGAAGGTTTGCTGAGAAGAAACAAAGTGGGCAGAAACAATGAG |
| | | AAACTGCCAACCTTGAAAAATGTGCAAGATTGCCTGTCTCTCCAGAAGT |
| | | TTGACAGCCCTCCCTTCTTCCAGAACTCCACCTTCAGCTTCAGGAATGC |
| | | ACTGGAAGGGTTTGACAAAGCAGATGGAACACTGGACTCTCAAGTCATG |
| | | AACCTTCACAACTTGGCTCACTCCTTCCTGAATGGGACCAATGCCTTGC |
| | | CACACTCAGCAGCCAATGACCCTGTGTTTGTGGTCCTCCACTCTTTCAC |
| | | AGATGCCATCTTTGATGAGTGGCTGAAGAGAAACAACCCTTCCACAGAT |
| | | GCCTGGCCTCAGGAACTGGCACCCATTGGTCACAACAGAATGTACAACA |
| | | TGGTCCCCTTCTTCCCACCTGTGACCAATGAGGAGCTCTTCCTCACTGC |
| | | AGAGCAACTTGGCTACAATTATGCAGTTGATCTGTCAGAGGAAGAAGCT |
| | | CCAGTTTGGTCCACAACTCTCTCAGTGGTCATTGGAATCCTGGGAGCTT |
| | | TTGTCTTGCTCTTGGGGTTGCTGGCTTTTCTTCAATACAGAAGGCTGAG |
| | | GAAAGGCTATGCTCCCTTGATGGAGACAGGTCTCAGCAGCAAGAGATAC |
| | | ACAGAGGAAGCCTAGAGAACAGCGCCTCCCTGACTCTCCACCTCGAAAG |
| | | AGGTGGAGAGTCAGGGAGGCCCAGAGGGTCTTAGAGTGTCACAACATTT |
| | | GGGCCTCTAAAAATTAGGTCATGTGGCAGAATGTTGTGAACAGTTTTCA |
| | | GATCTGGGAGCCTTGCTTTGGAGGCGCTTTCAAAAATGATGCAGTCCAT |
| | | GAGTGCACAGTGCGGGGTGATCTCTTTCTTCTTTTTGTCCCTTACTATT |
| | | CCAGTATGCATCTTACACAACCAGCCATATTTGTCCCACACTTTaTCTT |
| | | CATACTCCCTCGAAGCTTCCCTGGTCATTTCAACATCGATAAGCTTAAT |
| | | GTCCTTCCTATTtTGTGAGTCCAGAAGCTTTCTGATGTCATCGGAGCCT |
| | | TGACAGCTTAGAACCATCCCCTGCGGAAGAGCACCTATAACTGACGAGG |
| | | TCAACCCGGGTTGCGCATTGAAGAGGTCGGCAAGATCCATGCCGTGTGA |
| | | GTACTTGGAATCTTGCTTGAATTGTTTTTGATCAACGGGTTCCCTGTAA |
| | | AAGTGTATGAACTGCCCGTTCTGTGGTTGGAAAATTGCTATTTCCACTG |
| | | GATCATTAAATCTACCCTCAATGTCAATCCATGTAGGAGCGTTGGGGTC |
| | | AATTCCTCCCATGAGGTCTTTTAAAAGCATTGTCTGGCTGTAGCTTAAG |
| | | CCCACCTGAGGTGGACCTGCTGCTCCAGGCGCTGGCCTGGGTGAgTTGA |
| | | CTGCAGGTTTCTCGCTTGTGAGATCAATTGTTGTGTTTTCCCATGCTCT |
| | | CCCCACAATCGATGTTCTACAAGCTATGTATGGCCATCCTTCACCTGAA |
| | | AGGCAAACTTTATAGAGGATGTTTTCATAAGGGTTCCTGTCCCCAACTT |
| | | GGTCTGAAACAAACATGTTGAGTTTTCTCTTGGCCCCGAGAACTGCCTT |
| | | CAAGAGaTCCTCGCTGTTGCTTGGCTTGATCAAAATTGACTCTAACATG |
| | | TTACCCCCATCCAACAGGGCTGCCCCTGCCTTCACGGCAGCACCAAGAC |
| | | TAAAGTTATAGCCAGAAATGTTGATGCTGGACTGCTGTTCAGTGATGAC |
| | | CCCCAGAACTGGGTGCTTGTCTTTCAGCCTTTCAAGATCATTAAGATTT |
| | | GGATACTTGACTGTGTAAAGCAAGCCAAGGTCTGTGAGCGCTTGTACAA |
| | | CGTCATTGAGCGGAGTCTGTGACTGTTTGGCCATACAAGCCATAGTTAG |
| | | ACTTGGCATTGTGCCAAATTGATTGTTCAAAAGTGATGAGTCTTTCACA |
| | | TCCCAAACTCTTACCACACCACTTGCACCCTGCTGAGGCTTTCTCATCC |
| | | CAACTATCTGTAGGATCTGAGATCTTTGGTCTAGTTGCTGTGTTGTTAA |
| | | GTTCCCCATATATACCCCTGAAGCCTGGGGCCTTTCAGACCTCATGATC |
| | | TTGGCCTTCAGCTTCTCAAGGTCAGCCGCAAGAGACATCAGTTCTTCTG |
| | | CACTGAGCCTCCCCACTTTCAAAACATTCTTCTTTGATGTTGACTTTAA |
| | | ATCCACAAGAGAATGTACAGTCTGGTTGAGACTTCTGAGTCTCTGTAGG |
| | | TCTTTGTCATCTCTCTTTTCCTTCCTCATGATCCTCTGAACATTGCTGA |
| | | CCTCAGAGAAGTCCAACCCATTCAGAAGGTTGGTTGCATCCTTAATGAC |
| | | AGCAGCCTTCACATCTGATGTGAAGCTCTGCAATTCTCTTCTCAATGCT |
| | | TGCGTCCATTGGAAGCTCTTAACTTCCTTAGACAAGGACATCTTGTTGC |
| | | TCAATGGTTTCTCAAGACAAATGCGCAATCAAATGCCTAGGATCCACTG |
| | | TGCG |
| 31 | Genomic sequence of | gCGCACAGTGGATCCTAGGCATTTGATTGCGCATTTGTCTTGAGAAACC |
| | LCMV vector | ATTGAGCAACAAGATGGGTCAGATTGTGACAATGTTTGAGGCTTTGCCT |
| | (r3LCMV) encoding | CACATCATTGATGAGGTCATCAACATTGTCATTATTGTGCTCATTATAA |
| | TRP2 S Segment 2 | TCACGAGCATCAAAGCTGTGTACAATTTCGCCACCTGTGGGATATTAGC |
| | (containing GP) | ACTGGTCAGCTTCCTTTTTTTGGCTGGTAGGTCCTGTGGCATGTACGGC |
| | | CTTAATGGTCCCGACATCTATAAAGGGGTTTACCAGTTCAAATCAGTGG |
| | | AGTTTGATATGTCTCACTTAAATCTGACGATGCCCAATGCGTGCTCAGC |
| | | CAACAACTCTCATCACTACATCAGTATGGGAAGCTCTGGACTGGAGCTA |
| | | ACTTTCACTAACGACTCCATCCTTAATCACAATTTTTGCAACTTAACCT |
| | | CCGCTTTCAACAAAAAGACTTTTGACCATACACTCATGAGTATAGTCTC |
| | | GAGTCTGCACCTCAGTATTAGAGGGAATTCCAACCACAAAGCAGTGTCT |
| | | TGTGATTTTAACAATGGCATCACCATTCAATACAACTTGTCATTTTCGG |
| | | ACCCACAGAGCGCTATAAGCCAGTGTAGGACTTTCAGAGGTAGAGTCTT |
| | | GGACATGTTTAGAACTGCCTTTGGAGGAAAATACATGAGAAGTGGCTGG |
| | | GGCTGGGCAGGTTCAGATGGCAAGACCACTTGGTGCAGCCAAACAAGCT |
| | | ATCAGTACCTAATCATACAAAACAGGACTTGGGAAAACCACTGTAGATA |
| | | TGCAGGCCCTTTTGGGATGTCTAGAATCCTCTTTGCTCAGGAAAAGACA |
| | | AAGTTTCTCACTAGGAGACTTGCAGGCACATTCACCTGGACCCTGTCAG |
| | | ACTCCTCAGGAGTAGAAAATCCAGGTGGTTATTGCCTGACCAAATGGAT |
| | | GATCCTTGCTGCAGAGCTCAAATGTTTTGGGAATACAGCTGTTGCAAAA |
| | | TGTAATGTCAATCATGATGAAGAGTTCTGTGACATGCTACGACTAATTG |
| | | ATTACAACAAGGCCGCCCTGAGTAAGTTCAAGCAAGATGTAGAGTCTGC |
| | | CTTGCATGTATTCAAAACAACAGTAAATTCTCTGATTTCCGATCAGCTG |
| | | TTGATGAGGAATCATCTAAGAGATCTAATGGGGGTACCATACTGTAATT |
| | | ACTCAAAGTTCTGGTATCTGGAACATGCTAAGACTGGTGAGACTAGTGT |
| | | ACCCAAGTGCTGGCTTGTCACTAATGGCTCCTACTTGAATGAGACCCAC |
| | | TTTAGTGATCAAATCGAACAAGAAGCAGATAACATGATCACAGAGATGT |
| | | TGAGGAAGGACTACATAAAAAGACAAGGGAGTACTCCTTTAGCCTTAAT |
| | | GGATCTTTTGATGTTTTCAACATCAGCATATCTAATCAGCATCTTTCTG |
| | | CATCTTGTGAAGATACCAACACATAGACACATAAAGGGCGGTTCATGTC |
| | | CAAAGCCACACCGCTTGACCAACAAGGGGATCTGTAGTTGTGGTGCATT |
| | | CAAGGTGCCTGGTGTAAAAACTATCTGGAAAAGACGCTGAGACCCTCTG |
| | | GGCCTCCCTGACTCTCCACCTCTTTCGAGGTGGAGAGTCAGGGAGGCGC |
| | | TGTTCTCTAGGCTTCCTCTGTGTATCTCTTGCTGCTGAGACCTGTCTCC |
| | | ATCAAGGGAGCATAGCCTTTCCTCAGCCTTCTGTATTGAAGAAAAGCCA |
| | | GCAACCCCAAGAGCAAGACAAAAGCTCCCAGGATTCCAATGACCACTGA |
| | | GAGAGTTGTGGACCAAACTGGAGCTTCTTCCTCTGACAGATCAACTGCA |
| | | TAATTGTAGCCAAGTTGCTCTGCAGTGAGGAAGAGCTCCTCATTGGTCA |
| | | CAGGTGGGAAGAAGGGGACCATGTTGTACATTCTGTTGTGACCAATGGG |
| | | TGCCAGTTCCTGAGGCCAGGCATCTGTGGAAGGGTTGTTTCTCTTCAGC |
| | | CACTCATCAAAGATGGCATCTGTGAAAGAGTGGAGGACCACAAACACAG |
| | | GGTCATTGGCTGCTGAGTGTGGCAAGGCATTGGTCCCATTCAGGAAGGA |
| | | GTGAGCCAAGTTGTGAAGGTTCATGACTTGAGAGTCCAGTGTTCCATCT |
| | | GCTTTGTCAAACCCTTCCAGTGCATTCCTGAAGCTGAAGGTGGAGTTCT |
| | | GGAAGAAGGGAGGGCTGTCAAACTTCTGGAGAGACAGGCAATCTTGCAC |
| | | ATTTTTCAAGGTTGGCAGTTTCTCATTGTTTCTGCCCACTTTGTTTCTT |
| | | CTCAGCAAACCTTCATAGGTTCCATTGCACAGTGTGACCCTCCTGTTGT |
| | | AGTCATCCAAGCTGTCACACACAATCTCCCAGGTTGAGAATCTTGAGTT |
| | | CCTGCTGATCAGTGTTGGGTCATCTTGTCTTGCTGCTCCAAGCAGCTCA |
| | | TCTGTGCACACATCACACTCATTCTTCCCAGTTGCAAAGTTCCAGTAGG |
| | | GCAAGGCAAAGGACTCATTGCCAGTGAGTCTCTGGAGTTCTCTTTCCAG |
| | | CCACAACAGATGGTACCTGTGCCAGGTGACAAAGGCAGGCCCTTGGTGA |
| | | GAGAAATCAATGGCCTTGTAGGGTCTTCCTGGACCCAACAATGTGTCTC |
| | | TCACAGAATAATAATGGAGCCACACAAAAAAGTCATACACACTGCAGTT |
| | | GGCAATCTGGGGCTGGGTCCCATTGGGTCCCAGCAGCCCCAGCCAGTGT |
| | | TGTGTGGTGATCACATAGTCTGGATGGATGCTCTTCTTGGCCAGGTCCA |
| | | AGGCTCCCAAGAACTGCTCCCTCTCCTGGGCAGTCAGGGAATGGATGTT |
| | | CCTTCTGAGGATGGCTGGCTTCTTCCTGTTGCAGTCTGGGCCAGTCCAG |
| | | CCAAACTTGCAGCCTCCACAATTGTAACCAGCAAAGTTTCCTGTGCATT |
| | | TGCATGTCCTGTTGAAGAATTTTCTTGGCCATTGCTCCCTGTCATCCTG |
| | | GTTTCTGAGGATGTAAGGGCCACTCCAGGGTCTGGTGTCTGTTTGCACC |
| | | TCTGCACACTGCCCCCTGCCCTCCAGAAATCCACAGATGTTGGTTGCCT |
| | | CTGGACCCAGAGGGGGGCAGCATTCCTTGTTCAGCACCCCATCCAAGGT |
| | | CATGCAGACTCTGGGAAACTGAGCCCTGGCTCTGAGCAGAATTCCACAG |
| | | CCCAGACAACCCAGCAGAAGCCCCCATCCCACAAGGCCCATCCTTCTGT |
| | | AGGATAGGGCCTGACACCCAGTTGATCTAGAGGAAAGCGCAATCCAAAA |
| | | AGCCTAGGATCCCCGGTGCG |
| 32 | Genomic sequence of | GCGCACCGGGGATCCTAGGCATACCTTGGACGCGCATATTACTTGATCA |
| | Pichinde vector | AAGATGGGCCTTGTGGGATGGGGGCTTCTGCTGGGTTGTCTGGGCTGTG |
| | (r3PICV) encoding | GAATTCTGCTCAGAGCCAGGGCTCAGTTTCCCAGAGTCTGCATGACCTT |
| | TRP2 S Segment 1 | GGATGGGGTGCTGAACAAGGAATGCTGCCCCCCTCTGGGTCCAGAGGCA |
| | (containing NP) | ACCAACATCTGTGGATTTCTGGAGGGCAGGGGGCAGTGTGCAGAGGTGC |
| | | AAACAGACACCAGACCCTGGAGTGGCCCTTACATCCTCAGAAACCAGGA |
| | | TGACAGGGAGCAATGGCCAAGAAAATTCTTCAACAGGACATGCAAATGC |
| | | ACAGGAAACTTTGCTGGTTACAATTGTGGAGGCTGCAAGTTTGGCTGGA |
| | | CTGGCCCAGACTGCAACAGGAAGAAGCCAGCCATCCTCAGAAGGAACAT |
| | | CCATTCCCTGACTGCCCAGGAGAGGGAGCAGTTCTTGGGAGCCTTGGAC |
| | | CTGGCCAAGAAGAGCATCCATCCAGACTATGTGATCACCACACAACACT |
| | | GGCTGGGGCTGCTGGGACCCAATGGGACCCAGCCCCAGATTGCCAACTG |
| | | CAGTGTGTATGACTTTTTTGTGTGGCTCCATTATTATTCTGTGAGAGAC |
| | | ACATTGTTGGGTCCAGGAAGACCCTACAAGGCCATTGATTTCTCTCACC |
| | | AAGGGCCTGCCTTTGTCACCTGGCACAGGTACCATCTGTTGTGGCTGGA |
| | | AAGAGAACTCCAGAGACTCACTGGCAATGAGTCCTTTGCCTTGCCCTAC |
| | | TGGAACTTTGCAACTGGGAAGAATGAGTGTGATGTGTGCACAGATGAGC |
| | | TGCTTGGAGCAGCAAGACAAGATGACCCAACACTGATCAGCAGGAACTC |
| | | AAGATTCTCAACCTGGGAGATTGTGTGTGACAGCTTGGATGACTACAAC |
| | | AGGAGGGTCACACTGTGCAATGGAACCTATGAAGGTTTGCTGAGAAGAA |
| | | ACAAAGTGGGCAGAAACAATGAGAAACTGCCAACCTTGAAAAATGTGCA |
| | | AGATTGCCTGTCTCTCCAGAAGTTTGACAGCCCTCCCTTCTTCCAGAAC |
| | | TCCACCTTCAGCTTCAGGAATGCACTGGAAGGGTTTGACAAAGCAGATG |
| | | GAACACTGGACTCTCAAGTCATGAACCTTCACAACTTGGCTCACTCCTT |
| | | CCTGAATGGGACCAATGCCTTGCCACACTCAGCAGCCAATGACCCTGTG |
| | | TTTGTGGTCCTCCACTCTTTCACAGATGCCATCTTTGATGAGTGGCTGA |
| | | AGAGAAACAACCCTTCCACAGATGCCTGGCCTCAGGAACTGGCACCCAT |
| | | TGGTCACAACAGAATGTACAACATGGTCCCCTTCTTCCCACCTGTGACC |
| | | AATGAGGAGCTCTTCCTCACTGCAGAGCAACTTGGCTACAATTATGCAG |
| | | TTGATCTGTCAGAGGAAGAAGCTCCAGTTTGGTCCACAACTCTCTCAGT |
| | | GGTCATTGGAATCCTGGGAGCTTTTGTCTTGCTCTTGGGGTTGCTGGCT |
| | | TTTCTTCAATACAGAAGGCTGAGGAAAGGCTATGCTCCCTTGATGGAGA |
| | | CAGGTCTCAGCAGCAAGAGATACACAGAGGAAGCCTAGGCCCTAGCCTC |
| | | GACATGGGCCTCGACGTCACTCCCCAATAGGGGAGTGACGTCGAGGCCT |
| | | CTGAGGACTTGAGCTCAGAGGTTGATCAGATCTGTGTTGTTCCTGTACA |
| | | GCGTGTCAATAGGCAAGCATCTCATCGGCTTCTGGTCCCTAACCCAGCC |
| | | TGTCACTGTTGCATCAAACATGATGGTATCAAGCAATGCACAGTGAGGA |
| | | TTCGCAGTGGTTTGTGCAGCCCCCTTCTTCTTCTTCTTTATGACCAAAC |
| | | CTTTATGTTTGGTGCAGAGTAGATTGTATCTCTCCCAGATCTCATCCTC |
| | | AAAGGTGCGTGCTTGCTCGGCACTGAGTTTCACGTCAAGCACTTTTAAG |
| | | TCTCTTCTCCCATGCATTTCGAACAAACTGATTATATCATCTGAACCTT |
| | | GAGCAGTGAAAACCATGTTTTGAGGTAAATGTCTGATGATTGAGGAAAT |
| | | CAGGCCTGGTTGGGCATCAGCCAAGTCCTTTAAAAGgAGACCATGTGAG |
| | | TACTTGCTTTGCTCTTTGAAGGACTTCTCATCGTGGGGAAATCTGTAAC |
| | | AATGTATGTAGTTGCCCGTGTCAGGCTGGTAGATGGCCATTTCCACCGG |
| | | ATCATTTGGTGTTCCTTCAATGTCAATCCATGTGGTAGCTTTTGAATCA |
| | | AGCATCTGAATTGAGGACACAACAGTaTCTTCTTTCTCCTTAGGGATTT |
| | | GTTTAAGGTCCGGTGATCCTCCGTTTCTTACTGGTGGCTGGATAGCACT |
| | | CGGCTTCGAATCTAAATCTACAGTGGTGTTATCCCAAGCCCTCCCTTGA |
| | | ACTTGAGACCTTGAGCCAATGTAAGGCCAACCATCCCCTGAAAGACAAA |
| | | TCTTGTATAGTAAATTTTCATAAGGATTTCTCTGTCCGGGTGTAGTGCT |
| | | CACAAACATACCTTCACGATTCTTTATTTGCAATAGACTCTTTATGAGA |
| | | GTACTAAACATAGAAGGCTTCACCTGGATGGTCTCAAGCATATTGCCAC |
| | | CATCAATCATGCAAGCAGCTGCTTTGACTGCTGCAGACAAACTGAGATT |
| | | GTACCCTGAGATGTTTATGGCTGATGGCTCATTACTAATGATTTTTAGG |
| | | GCACTGTGTTGCTGTGTGAGTTTCTCTAGATCTGTCATGTTCGGGAACT |
| | | TGACAGTGTAGAGCAAACCAAGTGCACTCAGCGCTTGGACAACATCATT |
| | | AAGTTGTTCACCCCCTTGCTCAGTCATACAAGCGATGGTTAAGGCTGGC |
| | | ATTGATCCAAATTGATTGATCAACAATGTATTATCCTTGATGTCCCAGA |
| | | TCTTCACAACCCCATCTCTGTTGCCTGTGGGTCTAGCATTAGCGAACCC |
| | | CATTGAGCGAAGGATTTCGGCTCTTTGTTCCAACTGAGTGTTTGTGAGA |
| | | TTGCCCCCATAAACACCAGGCTGAGACAAACTCTCAGTTCTAGTGACTT |
| | | TCTTTCTTAACTTGTCCAAATCAGATGCAAGCTCCATTAGCTCCTCTTT |
| | | GGCTAAGCCTCCCACCTTAAGCACATTGTCCCTCTGGATTGATCTCATA |
| | | TTCATCAGAGCATCAACCTCTTTGTTCATGTCTCTTAACTTGGTCAGAT |
| | | CAGAATCAGTCCTTTTATCTTTGCGCATCATTCTTTGAACTTGAGCAAC |
| | | TTTGTGAAAGTCAAGAGCAGATAACAGTGCTCTTGTGTCCGACAACACA |
| | | TCAGCCTTCACAGGATGGGTCCAGTTGGATAGACCCCTCCTAAGGGACT |
| | | GTACCCAGCGGAATGATGGGATGTTGTCAGACATTTTGGGGTTGTTTGC |
| | | ACTTCCTCCGAGTCAGTGAAGAAGTGAACGTACAGCGTGATCTAGAATC |
| | | GCCTAGGATCCACTGTGCG |
| 33 | Genomic sequence of | GCGCACCGGGGATCCTAGGCATACCTTGGACGCGCATATTACTTGATCA |
| | Pichinde vector | AAGATGGGCCTTGTGGGATGGGGGCTTCTGCTGGGTTGTCTGGGCTGTG |
| | (r3PICV) encoding | GAATTCTGCTCAGAGCCAGGGCTCAGTTTCCCAGAGTCTGCATGACCTT |
| | TRP2 S Segment 2 | GGATGGGGTGCTGAACAAGGAATGCTGCCCCCCTCTGGGTCCAGAGGCA |
| | (containing GP) | ACCAACATCTGTGGATTTCTGGAGGGCAGGGGGCAGTGTGCAGAGGTGC |
| | | AAACAGACACCAGACCCTGGAGTGGCCCTTACATCCTCAGAAACCAGGA |
| | | TGACAGGGAGCAATGGCCAAGAAAATTCTTCAACAGGACATGCAAATGC |
| | | ACAGGAAACTTTGCTGGTTACAATTGTGGAGGCTGCAAGTTTGGCTGGA |
| | | CTGGCCCAGACTGCAACAGGAAGAAGCCAGCCATCCTCAGAAGGAACAT |
| | | CCATTCCCTGACTGCCCAGGAGAGGGAGCAGTTCTTGGGAGCCTTGGAC |
| | | CTGGCCAAGAAGAGCATCCATCCAGACTATGTGATCACCACACAACACT |
| | | GGCTGGGGCTGCTGGGACCCAATGGGACCCAGCCCCAGATTGCCAACTG |
| | | CAGTGTGTATGACTTTTTTGTGTGGCTCCATTATTATTCTGTGAGAGAC |
| | | ACATTGTTGGGTCCAGGAAGACCCTACAAGGCCATTGATTTCTCTCACC |
| | | AAGGGCCTGCCTTTGTCACCTGGCACAGGTACCATCTGTTGTGGCTGGA |
| | | AAGAGAACTCCAGAGACTCACTGGCAATGAGTCCTTTGCCTTGCCCTAC |
| | | TGGAACTTTGCAACTGGGAAGAATGAGTGTGATGTGTGCACAGATGAGC |
| | | TGCTTGGAGCAGCAAGACAAGATGACCCAACACTGATCAGCAGGAACTC |
| | | AAGATTCTCAACCTGGGAGATTGTGTGTGACAGCTTGGATGACTACAAC |
| | | AGGAGGGTCACACTGTGCAATGGAACCTATGAAGGTTTGCTGAGAAGAA |
| | | ACAAAGTGGGCAGAAACAATGAGAAACTGCCAACCTTGAAAAATGTGCA |
| | | AGATTGCCTGTCTCTCCAGAAGTTTGACAGCCCTCCCTTCTTCCAGAAC |
| | | TCCACCTTCAGCTTCAGGAATGCACTGGAAGGGTTTGACAAAGCAGATG |
| | | GAACACTGGACTCTCAAGTCATGAACCTTCACAACTTGGCTCACTCCTT |
| | | CCTGAATGGGACCAATGCCTTGCCACACTCAGCAGCCAATGACCCTGTG |
| | | TTTGTGGTCCTCCACTCTTTCACAGATGCCATCTTTGATGAGTGGCTGA |
| | | AGAGAAACAACCCTTCCACAGATGCCTGGCCTCAGGAACTGGCACCCAT |
| | | TGGTCACAACAGAATGTACAACATGGTCCCCTTCTTCCCACCTGTGACC |
| | | AATGAGGAGCTCTTCCTCACTGCAGAGCAACTTGGCTACAATTATGCAG |
| | | TTGATCTGTCAGAGGAAGAAGCTCCAGTTTGGTCCACAACTCTCTCAGT |
| | | GGTCATTGGAATCCTGGGAGCTTTTGTCTTGCTCTTGGGGTTGCTGGCT |
| | | TTTCTTCAATACAGAAGGCTGAGGAAAGGCTATGCTCCCTTGATGGAGA |
| | | CAGGTCTCAGCAGCAAGAGATACACAGAGGAAGCCTAGGCCCTAGCCTC |
| | | GACATGGGCCTCGACGTCACTCCCCAATAGGGGAGTGACGTCGAGGCCT |
| | | CTGAGGACTTGAGCTTATTTACCCAGTCTCACCCATTTGTAGGGTTTCT |
| | | TTGGGATTTTATAATACCCACAGCTGCAAAGAGAGTTCCTAGTAATCCT |
| | | ATGTGGCTTCGGACAGCCATCACCAATGATGTGCCTATGAGTGGGTATT |
| | | CCAACTAAGTGGAGAAACACTGTGATGGTGTAAAACACCAAAGACCAGA |
| | | AGCAAATGTCTGTCAATGCTAGTGGAGTCTTACCTTGTCTTTCTTCATA |
| | | TTCTTTTATCAGCATTTCATTGTACAGATTCTGGCTCTCCCACAACCAA |
| | | TCATTCTTAAAATGCGTTTCATTGAGGTACGAGCCATTGTGAACTAACC |
| | | AACACTGCGGTAAAGAATGTCTcCCTGTGATGGTATCATTGATGTACCA |
| | | AAATTTTGTATAGTTGCAATAAGGGATTTTGGCAAGCTGTTTGAGACTG |
| | | TTTCTAATCACAAGTGAGTCAGAAATAAGTCCGTTGATAGTCTTTTTAA |
| | | AGAGATTCAACGAATTCTCAACATTAAGTTGTAAGGTTTTGATAGCATT |
| | | CTGATTGAAATCAAATAACCTCATCGTATCGCAAAATTCTTCATTGTGA |
| | | TCTTTGTTGCATTTTGCCATCACAGTGTTATCAAAACATTTTATTCCAG |
| | | CCCAAACAATAGCCCATTGCTCCAAACAGTAACCACCTGGGACATGTTG |
| | | CCCAGTAGAGTCACTCAAGTCCCAAGTGAAAAAGCCAAGGAGTTTCCTG |
| | | CTCACAGAACTATAAGCAGTTTTTTGGAGAGCCATCCTTATTGTTGCCA |
| | | TtGGAGTATATGTACAGTGATTTTCCCATGTGGTGTTCTGTATGATCAG |
| | | GAAATTGTAATGTGTCCCACCTTCACAGTTTGTTAGTCTGCAAGACCCT |
| | | CCACTACAGTTATTGAAACATTTTCCAACCCACGCAATTTTTGGGTCCC |
| | | CAATGATTTGAGCAAGCGACGCAATAAGATGTCTGCCAACCTCACCTCC |
| | | TCTATCCCCAACTGTCAAGTTGTACTGGATCAACACCCCAGCACCCTCA |
| | | ACTGTTTTGCATCTGGCACCTACATGACGAGTGACATGGAGCACATTGA |
| | | AGTGTAACTCATTAAGCAACCATTTTAATGTGTGACCTGCTTCTTCTGT |
| | | CTTATCACAATTACTAATGTTACCATATGCAAGGCTTCTGATGTTGGAA |
| | | AAGTTTCCAGTAGTTTCATTTGCAATGGATGTGTTTGTCAAAGTGAGTT |
| | | CAATTCCCCATGTTGTGTTAGATGGTCCTTTGTAGTAATGATGTGTGTT |
| | | GTTCTTGCTACATGATTGTGGCAAGTTGTCAAACATTCTTGTGAGGTTG |
| | | AACTCAACGTGGGTGAGATTGTGCCTCCTATCAATCATCATGCCATCAC |
| | | AACTTCTGCCAGCCAAAATGAGGAAGGTGATGAGTTGGAATAGGCCACA |
| | | TCTCATCAGATTGACAAATCCTTTGATGATGCATAGGGTTGAGACAATG |
| | | ATTAAGGCGACATTGAACACCTCCTGCAGGACTTCGGGTATAGACTGGA |
| | | TCAAAGTCACAACTTGTCCCATTTTGGGGTTGTTTGCACTTCCTCCGAG |
| | | TCAGTGAAGAAGTGAACGTACAGCGTGATCTAGAATCGCCTAGGATCCA |
| | | CTGTGCG |
| 34 | E7E6 Fusion protein | MHGDTPTLHEYMLDLQPETTDLYGYGQLNDSSEEEDEIDGPAGQAEPDR |
| | | AHYNIVTFCCKCDSTLRLCVQSTHVDIRTLEDLLMGTLGIVGPICSQKP |
| | | HQKRTAMFQDPQERPRKLPQLCTELQTTIHDIILECVYCKQQLLRREVY |
| | | DFAFRDLCIVYRDGNPYAVGDKCLKFYSKISEYRHYCYSLYGTTLEQQY |
| | | NKPLCDLLIRCINGQKPLCPEEKQRHLDKKQRFHNIRGRWTGRCMSCCR |
| | | SSRTRRETQL |
| 35 | murine TRP2 protein | MGLVGWGLLLGCLGCGILLRARAQFPRVCMTLDGVLNKECCPPLGPEAT |
| | (Reference Sequence | NICGFLEGRGQCAEVQTDTRPWSGPYILRNQDDREQWPRKFFNRTCKCT |
| | NM_010024) | GNFAGYNCGGCKFGWTGPDCNRKKPAILRRNIHSLTAQEREQFLGALDL |
| | | AKKSIHPDYVITTQHWLGLLGPNGTQPQIANCSVYDFFVWLHYYSVRDT |
| | | LLGPGRPYKAIDFSHQGPAFVTWHRYHLLWLERELQRLTGNESFALPYW |
| | | NFATGKNECDVCTDELLGAARQDDPTLISRNSRFSTWEIVCDSLDDYNR |
| | | RVTLCNGTYEGLLRRNKVGRNNEKLPTLKNVQDCLSLQKFDSPPFFQNS |
| | | TFSFRNALEGFDKADGTLDSQVMNLHNLAHSFLNGTNALPHSAANDPVF |
| | | VVLHSFTDAIFDEWLKRNNPSTDAWPQELAPIGHNRMYNMVPFFPPVTN |
| | | EELFLTAEQLGYNYAVDLSEEEAPVWSTTLSVVIGILGAFVLLLGLLAF |
| | | LQYRRLRKGYAPLMETGLSSKRYTEEA |
| 36 | GFP (reporter | MVSKGEELFTGVVPILVELDGDVNGHKFSVSGEGEGDATYGKLTLKFIC |
| | antigen) | TTGKLPVPWPTLVTTFTYGVQCFARYPDHMKQHDFFKSAMPEGYVQERT |
| | | IFFKDDGNYKTRAEVKFEGDTLVNRIELKGIDFKEDGNILGHKLEYNYN |
| | | SHKVYITADKQKNGIKVNFKTRHNIEDGSVQLADHYQQNTPIGDGPVLL |
| | | PDNHYLSTQSALSKDPNEKRDHMVLLEFVTAAGITLGMDELYK |
| 37 | LCMV cl13 | MSLSKEVKSFQWTQALRRELQSFTSDVKAAVIKDATNLLNGLDFSEVSN |
| | Nucleoprotein | VQRIMRKEKRDDKDLQRLRSLNQTVHSLVDLKSTSKKNVLKVGRLSAEE |
| | Sequence | LMSLAADLEKLKAKIMRSERPQASGVYMGNLTTQQLDQRSQILQIVGMR |
| | | KPQQGASGVVRVWDVKDSSLLNNQFGTMPSLTMACMAKQSQTPLNDVVQ |
| | | ALTDLGLLYTVKYPNLNDLERLKDKHPVLGVITEQQSSINISGYNFSLG |
| | | AAVKAGAALLDGGNMLESILIKPSNSEDLLKAVLGAKRKLNMFVSDQVG |
| | | DRNPYENILYKVCLSGEGWPYIACRTSIVGRAWENTTIDLTSEKPAVNS |
| | | PRPAPGAAGPPQVGLSYSQTMLLKDLMGGIDPNAPTWIDIEGRFNDPVE |
| | | IAIFQPQNGQFIHFYREPVDQKQFKQDSKYSHGMDLADLFNAQPGLTSS |
| | | VIGALPQGMVLSCQGSDDIRKLLDSQNRKDIKLIDVEMTREASREYEDK |
| | | VWDKYGWLCKMHTGIVRDKKKKEITPHCALMDCIIFESASKARLPDLKT |
| | | VHNILPHDLIFRGPNVVTL |
| 38 | LCMV cl13 | MGQIVTMFEALPHIIDEVINIVIIVLIVITGIKAVYNFATCGIFALISF |
| | Glycoprotein | LLLAGRSCGMYGLKGPDIYKGVYQFKSVEFDMSHLNLTMPNACSANNSH |
| | Sequence | HYISMGTSGLELTFTNDSIISHNFCNLTSAFNKKTFDHTLMSIVSSLHL |
| | | SIRGNSNYKAVSCDFNNGITIQYNLTFSDAQSAQSQCRTFRGRVLDMFR |
| | | TAFGGKYMRSGWGWTGSDGKTTWCSQTSYQYLIIQNRTWENHCTYAGPF |
| | | GMSRILLSQEKTKFLTRRLAGTFTWTLSDSSGVENPGGYCLTKWMILAA |
| | | ELKCFGNTAVAKCNVNHDEEFCDMLRLIDYNKAALSKFKEDVESALHLF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLVSIFLHLVKIPTHRHIKGGSCPKPHRLTNKGICSCGAFKVPG |
| | | VKTVWKRR |
| 39 | LCMV WE | MGQIVTMFEALPHIIDEVINIVIIVLIIITSIKAVYNFATCGILALVSF |
| | Glycoprotein | LFLAGRSCGMYGLNGPDIYKGVYQFKSVEFDMSHLNLTMPNACSANNSH |
| | Sequence | HYISMGSSGLELTFTNDSILNHNFCNLTSAFNKKTFDHTLMSIVSSLHL |
| | | SIRGNSNHKAVSCDFNNGITIQYNLSFSDPQSAISQCRTFRGRVLDMFR |
| | | TAFGGKYMRSGWGWAGSDGKTTWCSQTSYQYLIIQNRTWENHCRYAGPF |
| | | GMSRILFAQEKTKFLTRRLAGTFTWTLSDSSGVENPGGYCLTKWMILAA |
| | | ELKCFGNTAVAKCNVNHDEEFCDMLRLIDYNKAALSKFKQDVESALHVF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLISIFLHLVKIPTHRHIKGGSCPKPHRLTNKGICSCGAFKVPG |
| | | VKTIWKRR |
| 40 | LCMV cl13 | MDEIISELRELCLNYIEQDERLSRQKLNFLGQREPRMVLIEGLKLLSRC |
| | Polymerase Sequence | IEIDSADKSGCTHNHDDKSVETILVESGIVCPGLPLIIPDGYKLIDNSL |
| | | ILLECFVRSTPASFEKKFIEDTNKLACIREDLAVAGVTLVPIVDGRCDY |
| | | DNSFMPEWANFKFRDLLFKLLEYSNQNEKVFEESEYFRLCESLKTTIDK |
| | | RSGMDSMKILKDARSTHNDEIMRMCHEGINPNMSCDDVVFGINSLFSRF |
| | | RRDLESGKLKRNFQKVNPEGLIKEFSELYENLADSDDILTLSREAVESC |
| | | PLMRFITAETHGHERGSETSTEYERLLSMLNKVKSLKLLNTRRRQLLNL |
| | | DVLCLSSLIKQSKFKGLKNDKHWVGCCYSSVNDRLVSFHSTKEEFIRLL |
| | | RNRKKSKVFRKVSFEELFRASISEFIAKIQKCLLVVGLSFEHYGLSEHL |
| | | EQECHIPFTEFENFMKIGAHPIMYYTKFEDYNFQPSTEQLKNIQSLRRL |
| | | SSVCLALTNSMKTSSVARLRQNQIGSVRYQVVECKEVFCQVIKLDSEEY |
| | | HLLYQKTGESSRCYSIQGPDGHLISFYADPKRFFLPIFSDEVLYNMIDI |
| | | MISWIRSCPDLKDCLTDIEVALRTLLLLMLTNPTKRNQKQVQSVRYLVM |
| | | AIVSDFSSTSLMDKLREDLITPAEKVVYKLLRFLIKTIFGTGEKVLLSA |
| | | KFKFMLNVSYLCHLITKETPDRLTDQIKCFEKFFEPKSQFGFFVNPKEA |
| | | ITPEEECVFYEQMKRFTSKEIDCQHTTPGVNLEAFSLMVSSFNNGTLIF |
| | | KGEKKLNSLDPMTNSGCATALDLASNKSVVVNKHLNGERLLEYDFNKLL |
| | | VSAVSQITESFVRKQKYKLSHSDYEYKVSKLVSRLVIGSKGEETGRSED |
| | | NLAEICFDGEEETSFFKSLEEKVNTTIARYRRGRRANDKGDGEKLTNTK |
| | | GLHHLQLILTGKMAHLRKVILSEISFHLVEDFDPSCLTNDDMKFICEAV |
| | | EGSTELSPLYFTSVIKDQCGLDEMAKNLCRKFFSENDWFSCMKMILLQM |
| | | NANAYSGKYRHMQRQGLNFKFDWDKLEEDVRISERESNSESLSKALSLT |
| | | QCMSAALKNLCFYSEESPTSYTSVGPDSGRLKFALSYKEQVGGNRELYI |
| | | GDLRTKMFTRLIEDYFESFSSFFSGSCLNNDKEFENAILSMTINVREGF |
| | | LNYSMDHSKWGPMMCPFLFLMFLQNLKLGDDQYVRSGKDHVSTLLTWHM |
| | | HKLVEVPFPVVNAMMKSYVKSKLKLLRGSETTVTERIFRQYFEMGIVPS |
| | | HISSLIDMGQGILHNASDFYGLLSERFINYCIGVIFGERPEAYTSSDDQ |
| | | ITLFDRRLSDLVVSDPEEVLVLLEFQSHLSGLLNKFISPKSVAGRFAAE |
| | | FKSRFYVWGEEVPLLTKFVSAALHNVKCKEPHQLCETIDTIADQAIANG |
| | | VPVSLVNSIQRRTLDLLKYANFPLDPFLLNTNTDVKDWLDGSRGYRIQR |
| | | LIEELCPNETKVVRKLVRKLHHKLKNGEFNEEFFLDLFNRDKKEAILQL |
| | | GDLLGLEEDLNQLADVNWLNLNEMFPLRMVLRQKVVYPSVMTFQEERIP |
| | | SLIKTLQNKLCSKFTRGAQKLLSEAINKSAFQSCISSGFIGLCKTLGSR |
| | | CVRNKNRENLYIKKLLEDLTTDDHVTRVCNRDGITLYICDKQSHPEAHR |
| | | DHICLLRPLLWDYICISLSNSFELGVWVLAEPTKGKNNSENLTLKHLNP |
| | | CDYVARKPESSRLLEDKVNLNQVIQSVRRLYPKIFEDQLLPFMSDMSSK |
| | | NMRWSPRIKFLDLCVLIDINSESLSLISHVVKWKRDEHYTVLFSDLANS |
| | | HQRSDSSLVDEFVVSTRDVCKNFLKQVYFESFVREFVATTRTLGNFSWF |
| | | PHKEMMPSEDGAEALGPFQSFVSKVVNKNVERPMFRNDLQFGFGWFSYR |
| | | MGDVVCNAAMLIRQGLTNPKAFKSLKDLWDYMLNYTKGVLEFSISVDFT |
| | | HNQNNTDCLRKFSLIFLVRCQLQNPGVAELLSCSHLFKGEIDRRMLDEC |
| | | LHLLRTDSVFKVNDGVFDIRSEEFEDYMEDPLILGDSLELELLGSKRIL |
| | | DGIRSIDFERVGPEWEPVPLTVKMGALFEGRNLVQNIIVKLETKDMKVF |
| | | LAGLEGYEKISDVLGNLFLHRFRTGEHLLGSEISVILQELCIDRSILLI |
| | | PLSLLPDWFAFKDCRLCFSKSRSTLMYETVGGRFRLKGRSCDDWLGGSV |
| | | AEDID |
| 41 | LCMV cl13 Z protein | MGQGKSREEKGTNSTNRAEILPDTTYLGPLSCKSCWQKFDSLVRCHDHY |
| | Sequence | LCRHCLNLLLSVSDRCPLCKYPLPTRLKISTAPSSPPPYEE |
| 42 | Pichinde | MSDNIPSFRWVQSLRRGLSNWTHPVKADVLSDTRALLSALDFHKVAQVQ |
| | Nucleoprotein | RMMRKDKRTDSDLTKLRDMNKEVDALMNMRSIQRDNVLKVGGLAKEELM |
| | Sequence | ELASDLDKLRKKVTRTESLSQPGVYGGNLTNTQLEQRAEILRSMGFANA |
| | | RPTGNRDGVVKIWDIKDNTLLINQFGSMPALTIACMTEQGGEQLNDVVQ |
| | | ALSALGLLYTVKFPNMTDLEKLTQQHSALKIISNEPSAINISGYNLSLS |
| | | AAVKAAACMIDGGNMLETIQVKPSMFSTLIKSLLQIKNREGMFVSTTPG |
| | | QRNPYENLLYKICLSGDGWPYIGSRSQVQGRAWDNTTVDLDSKPSAIQP |
| | | PVRNGGSPDLKQIPKEKEDTVVSSIQMLDSKATTWIDIEGTPNDPVEMA |
| | | IYQPDTGNYIHCYRFPHDEKSFKEQSKYSHGLLLKDLADAQPGLISSII |
| | | RHLPQNMVFTAQGSDDIISLFEMHGRRDLKVLDVKLSAEQARTFEDEIW |
| | | ERYNLLCTKHKGLVIKKKKKGAAQTTANPHCALLDTIMFDATVTGWVRD |
| | | QKPMRCLPIDTLYRNNTDLINL |
| 43 | Pichinde | MGQVVTLIQSIPEVLQEVFNVALIIVSTLCIIKGFVNLMRCGLFQLITF |
| | Glycoprotein | LILAGRSCDGMMIDRRHNLTHVEFNLTRMFDNLPQSCSKNNTHHYYKGP |
| | Sequence | SNTTWGIELTLTNTSIANETTGNFSNIRSLAYGNISNCDKTEEAGHTLK |
| | | WLLNELHFNVLHVTRHVGARCKTVEGAGVLIQYNLTVGDRGGEVGRHLI |
| | | ASLAQIIGDPKIAWVGKCFNNCSGGSCRLTNCEGGTHYNFLIIQNTTWE |
| | | NHCTYTPMATIRMALQKTAYSSVSRKLLGFFTWDLSDSTGQHVPGGYCL |
| | | EQWAIVWAGIKCFDNTVMAKCNKDHNEEFCDTMRLFDFNQNAIKTLQLN |
| | | VENSLNLFKKTINGLISDSLVIRNSLKQLAKIPYCNYTKFWYINDTITG |
| | | RHSLPQCWLVHNGSYLNETHFKNDWLWESQNLYNEMLIKEYEERQGKTP |
| | | LALTDICFWSLVFYTITVFLHLVGIPTHRHIIGDGCPKPHRITRNSLCS |
| | | CGYYKIPKKPYKWVRLGK |
| 44 | Pichinde Polymerase | MEEYVFELKDIVRKWVPEWEELSEQKNNVLAQVKDRAITIEGLKLLSML |
| | Sequence | VEVDSCKKHSCKHNTKMTVNAILRELRVTCPTLPDVTPDGYCMVGDVLI |
| | | LLEVFVRTSQEAFEKKYNQDFLKLLQLSSDLKRQNITLVPVIDGRSSYY |
| | | VEFVPDWVVERLRWLLLKLMDGLRTSGEEVEELEYERLISSLSSLENQS |
| | | LGLESLLAVKERGLPYKVRLEKALMSGINNKLTTDQCRTKIMEIFQQFK |
| | | MLQLAGQLDRKLQATDREDMISRLQNHEFIQCSVKDVPKSEIRLCEFCS |
| | | VHILGIIGQLRQSEVKHSSTESREYFRVLSICNKIKSQKVFNTRRNTML |
| | | VLDLIMYNILCDLDKSSPGAVFREVLLMQGLPSVNDRLINVDFLMEQIT |
| | | KKFIKNPNWLEKAKKRLSSVCGELPLDDILPLLREPDVEYYFNLKTSVL |
| | | DEWGAKPCLQYKTKSQCMCGGRPGRGQPDYTIMGESEFEELLKTLSSLS |
| | | LSLINSMKTAAVPKMKVNNADEFYGKVYCDEVFFQRFGEGGSLTLLYQK |
| | | TGERSRCYAVAYRSKSGGLYETKASFYCDPKRFFLPIFSADVIQRTCVE |
| | | MLSWLDFMSQPLLDSVSDLLRRLILCILCTPSKRIQVYLQGFRYYIMAF |
| | | VNEVHFKELFEKLKVVMLTPSEWQTAMLIDDLILLVLSNSREEDMAKIF |
| | | KFVLNVSYLCHFITKETPDRLTDQIKCFEKFLEPKLKFDSVLVNPSNSM |
| | | ELPTEEEEKMVHDIERLLGKKLESKCEGRPGLNKDVLSVCLSLFNSSSL |
| | | EVKPLLPCDPMTPSFTSTALDMSSNKSVVVPKLNEVGEVITEYDYSSIV |
| | | SAVVVEMIEHFKTKGKYKLDPKEVNFKILKRLSSLIQIKKESIEPDGVE |
| | | ELLSEDQGDCLKEIETRVAKVLSKVDTNVKTNLKTSCPLERLWPKSTMV |
| | | VIKRETSLHDVKDFDYSLFSAEVYEDLVNLIYEDVTARSVYFADRLMNP |
| | | CPLEFLIKNLTLKAYKEADYFECFKYILIASDYDNRVGRYDHKSRSRLG |
| | | FTDAALQIRETSRISSRESNSESIAKRLDQSFFTNSSLRNLCFYSDESP |
| | | TERSGVSTNVGRLKFGLSYKEQVGGNRELYVGDLNTKLTTRLIEDYSES |
| | | LMQNMRYTCLNNEKEFERALLDMKSVVRQSGLAVSMDHSKWGPHMSPVI |
| | | FAALLKGLEFKLKDGSEVPNAAVINILLWHIHKMVEVPFNVVEAYMKGF |
| | | LKRGLGMMDKGGCTIAEEFMFGYFEKGKVPSHISSVLDMGQGILHNTSD |
| | | LYGLITEQFINYALELCYGARFISYTSSDDEIMLSLNEGFKFKDRDELN |
| | | VELVLDCMEFHYFLSDKLNKFVSPKTVVGTFASEFKSRFFIWSQEVPLL |
| | | TKFVAAALHNIKAKAPNQQADTIDTILDQCVANGVSIEVVGAIAKRTNS |
| | | MIIYSGFPNDPFLCLEEMDVLDWVNGSRGYRLQRSIETLFPDDLLLSII |
| | | RKACRKIFYKIQSGALEESYIVTTLQQSPDDCLKQLLETCDVETEAIED |
| | | ALNIRWLNLRVHGDLRLVLRTKLMSTTRTVQREEIPSLVKSVQSKLSKN |
| | | YVRGAKKILADAINKSAFQSSIASGFIGVCKSMGSKCVRDGKGGFKYIR |
| | | DITSKIILHRDCHFCNQRKGVYCKAALGEVSEYSRPLIWDYFALVLTNA |
| | | CELGNWVFQKAEVPKIVTHLNNPNHFWPIKPSTHSELEDKVGINHILYS |
| | | IRRNFPTLFDEHISPFLSDLNMLRLSWVQRIKFLDLCVAIDITSECLGI |
| | | VSHIIKHRREELYIVKQNELAMSHSRESHPLERGFNLEPEEVCTNFLIQ |
| | | ILFESMLVPVIMSTSQFKKYFWFGELELLPNNAQHDLKQLTQFICDCKK |
| | | NNTSRTMNLDDLDVGFVSSKLILSCVNLNISVFINELDWVNRDNYENIE |
| | | QLILASPSEVIPIELNLTFSHKRVSHKFRYERSTNYILKLRFLIERESL |
| | | LDSLDSDGYLLLNPHSVEYYVSQSSGNHISLDGVSLLVLNPLINGKDVL |
| | | DFNDLLEGQDIHFKSRSTVFQKVRIDLKNRFKDLKNKFSYKLIGPDVGM |
| | | QPLILEGGLIKEGNRVVSRLEVNLDSKVVIIALEALEPEKRPRFIANLF |
| | | QYLSSAQSHNKGISMNEQDLRLMIENFPEVFEHMLHDAKDWLNCGHFSI |
| | | IRSKTLGSVMIADETGPFKIKGIRCRKLFEDNESVEIE |
| 45 | Pichinde Z protein | MGLRYSKEVRKRHGDEDVVGRVPMTLNLPQGLYGRFNCKSCWFVNKGLI |
| | Sequence | RCKDHYLCLGCLTKMHSRGNLCEICGHSLPTKMEFLESPSAPPYEP |

### 8. EXAMPLES

All constructs used in the following examples have the GP ORF artificially juxtaposed to and expressed under control of the 3′ UTR.

### 8.1 Efficacy of Intratumoral Administration of Replication-Competent Arenavirus Vectors in the TC-1 Model

### 8.1.1 Example 1

The antitumoral effect of tri-segmented, replication-competent arenavirus vectors, e.g. r3LCMV, is analyzed in tumor bearing mice after intratumoral administration compared to peripheral administration.

Study Design

C57BL/6 mice are inoculated subcutaneously at the right flank with 1×10⁵ TC-1 cells in 0.1 ml of PBS for tumor development on day 1 (groups 1-8) or left untreated (group 9).

When tumors are palpable and reach a size suitable for intratumoral application (day ˜4), mice are either treated intratumorally with buffer (group 1), a high dose of a replication-competent arenavirus vector encoding an artificial fusion protein of HPV16 E6 and E7 proteins harboring 5 mutations abrogating the oncogenic potential of E6 and E7 (“r3LCMV-E7E6”) (group 2), a low dose of r3LCMV-E7E6 (group 3), a high dose of a replication-competent arenavirus vector expressing the reporter gene GFP (“r3LCMV-GFP”) (or analogous) as a vector control (group 4), a low dose of r3LCMV-GFP (or analogous) (group 5), or injected intravenously with buffer (group 6), r3LCMV-E7E6 (group 7), or r3LCMV-GFP (or analogous) (group 8). Tumor growth after tumor challenge as well as animal survival are monitored.

### 8.1.2 Example 2(a)

The antitumoral effect of tri-segmented, replication-competent lymphocytic choriomeningitis virus (r3LCMV) vector encoding an artificial fusion protein of HPV16 E6 and E7 proteins harboring five mutations abrogating the oncogenic potential of E6 and E7, i.e., r3LCMV-E7E6, was analyzed in tumor bearing mice in the TC-1 tumor model after intratumoral administration compared to intravenous administration.

Study Design:

TC-1 tumor bearing mice were treated intravenously (groups 1 to 3) or intratumorally (groups 4 to 6) with 1×10⁵ RCV FFU of r3LCMV-E7E6 (groups 1 and 4), 1×10⁵ RCV FFU of r3LCMV expressing the reporter gene GFP, i.e., r3LCMV-GFP (groups 2 and 5), or with buffer (control groups 3 and 6). Tumor growth as well as animal survival were monitored.

Eight weeks old female C57BL/6 mice were subcutaneously inoculated on day 0 with a single-cell suspension of 1×10⁵ cells of the TC-1 tumor cells in the right flank. When tumors were palpable (with a size suitable for intratumoral application, i.e., around 100 mm³), mice were randomized and injected intravenously with 1×10⁵ RCV FFU of r3LCMV-E7E6 (group 1), 1×10⁵ RCV FFU of r3LCMV expressing the reporter gene GFP, i.e., r3LCMV-GFP (group 2), buffer (group 3), or were treated intratumorally with 1×10⁵ RCV FFU of r3LCMV-E7E6 (group 4), 1×10⁵ RCV FFU of r3LCMV-GFP (group 5), or with buffer (group 6). Ten mice were considered for each group. Tumor size was measured every second day. Mice were sacrificed when the tumor reached a size of 20 mm in diameter. Animals with defined clinical signs (e.g., ulceration of the tumor or massive body weight loss) were euthanized regardless of tumor size in accordance with animal welfare regulations.

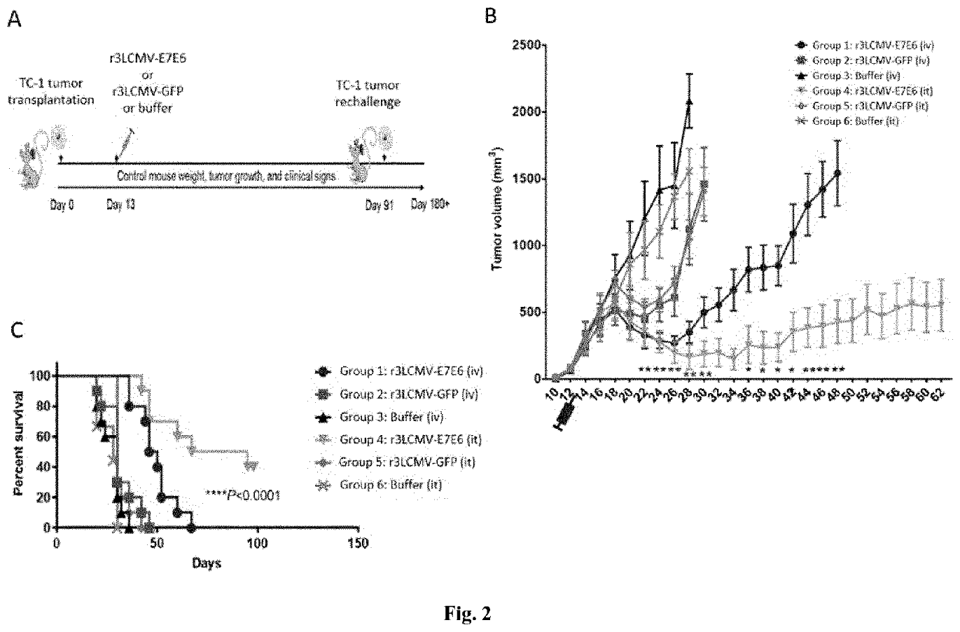

FIG. 2 provides (A) a schematic representation of the experimental design, and (B) tumor growth after tumor challenge. The tumor volume was calculated according to the formula V=0.5 L×W² where L (length) and W (width) are the long and short diameters of the tumor, respectively. Measurements for each group are included in the plot until >50% of mice per group were sacrificed. Statistically significant differences (*P<0.05, **P<0.005) were determined by comparing tumor volume in the control group (buffer or r3LCMV-GFP) with r3LCMV-E7E6 treated groups until day 32 by Two-way ANOVA. A significant difference was also observed at the time points day 40, 42, 44, 46, and 48 between r3LCMV-E7E6 i.v. and i.t. administration by Two-way ANOVA. (C) Overall survival. Log-rank Kaplan-Meier plot showing the overall survival of the indicated groups. ****Statistically significant (P<0.0001).

Respective results indicate that intratumoral as well as intravenous treatment with r3LCMV-E7E6 or r3LCMV-GFP vectors, but not buffer control, resulted in shrinkage of existing TC-1 tumors. However, tumors in mice treated with r3LCMV-GFP either by i.v. or i.t. administration increased again at similar growth rates as observed in buffer control groups, resulting in similar survival and tumor growth patterns. In contrast, mice treated intravenously or intratumorally with r3LCMV-E7E6 showed a clear reduction in tumor progression compared to r3LCMV-GFP or buffer control groups. At early timepoints (˜10 days) post therapy, i.t. and i.v. induced comparable anti tumor effects, whereas the effect of i.t. administration was stronger at later timepoints. Importantly, i.t. but not i.v. treatment with r3LCMV-E7E6 eventually eliminated subcutaneous TC-1 tumors in immunocompetent C57BL/6 mice. Three out of ten tumor bearing mice were cured within approximately 19 days after initiation of r3LCMV-E7E6 therapy, indicating that i.t. administration of r3LCMV-E7E6 eradicates tumors in 30% of mice after a single administration with a dose of 10⁵ RCV FFU in the TC-1 model.

### 8.1.3 Example 2(b)

Tumor-free mice from Example 2(a) are rechallenged with injection of 1×10⁵ TC-1 tumor cells into the contralateral flank to determine whether mice cured of TC-1 tumors acquired tumor-specific immune protection. As a control, untreated mice at similar age are challenged (first-challenge) with TC-1 tumor cells in parallel. Formation and growth of tumor is monitored.

### 8.1.4 Example 3

The antitumoral effect of (i) heterologous prime-boost combinations using replication-competent HPV antigen-expressing vectors derived from different arenaviruses and/or (ii) combinations of different injections routes, i.e., intratumoral and intravenous administration, using replication-competent HPV antigen-expressing vectors derived from the same or different arenaviruses, is analyzed in tumor bearing mice in the TC-1 tumor model.

Study Design:

C57BL/6 mice are inoculated subcutaneously at the right flank with 1×10⁵ TC-1 cells on day 1 (groups 1-15).

When tumors are palpable and reach a size suitable for intratumoral application, mice are either treated intratumorally (groups 1, 2, 4, 5, 7, 8, 10, 11, 13, 14) or intravenously (groups 3, 6, 9, 12, 15) with buffer (groups 1, 2, 3), a replication-competent LCMV vector encoding the artificial fusion protein of HPV16 E6 and E7 (“r3LCMV-E7E6”) (groups 4, 5, 6, 10, 11, 12), or a replication-competent Pichinde virus vector encoding the artificial fusion protein of HPV16 E6 and E7 (“r3PICV-E7E6”) (groups 7, 8, 9, 13, 14, 15). 10 to 15 days after the first injection, mice are either treated intratumorally (groups 1, 3, 4, 6, 7, 9, 10, 12, 13, 15) or intravenously (groups 2, 5, 8, 11, 14) with buffer (groups 1, 2, 3), r3LCMV-E7E6 (groups 4, 5, 6, 7, 8, 9), or r3PICV-E7E6 (groups 10, 11, 12, 13, 14, 15). Tumor growth after tumor challenge as well as animal survival are monitored. The fifteen treatment groups are summarized in Table 5.

TABLE 5
Summary of the fifteen treatment groups mentioned in Example 3.

| | TC-1 | | | | |
|---|---|---|---|---|---|
| group | challenge | 1^{st} injection | | 2^{nd} injection | |
| 1 | yes | IT | buffer | IT | buffer |
| 2 | yes | IT | buffer | IV | buffer |
| 3 | yes | IV | buffer | IT | buffer |
| 4 | yes | IT | r3LCMV-E7E6 | IT | r3LCMV-E7E6 |
| 5 | yes | IT | r3LCMV-E7E6 | IV | r3LCMV-E7E6 |
| 6 | yes | IV | r3LCMV-E7E6 | IT | r3LCMV-E7E6 |
| 7 | yes | IT | r3PICV-E7E6 | IT | r3LCMV-E7E6 |
| 8 | yes | IT | r3PICV-E7E6 | IV | r3LCMV-E7E6 |
| 9 | yes | IV | r3PICV-E7E6 | IT | r3LCMV-E7E6 |
| 10 | yes | IT | r3LCMV-E7E6 | IT | r3PICV-E7E6 |
| 11 | yes | IT | r3LCMV-E7E6 | IV | r3PICV-E7E6 |
| 12 | yes | IV | r3LCMV-E7E6 | IT | r3PICV-E7E6 |
| 13 | yes | IT | r3PICV-E7E6 | IT | r3PICV-E7E6 |
| 14 | yes | IT | r3PICV-E7E6 | IV | r3PICV-E7E6 |
| 15 | yes | IV | r3PICV-E7E6 | IT | r3PICV-E7E6 |

### 8.1.5 Example 4

The antitumoral effect of tri-segmented, replication-competent Pichinde virus (PICV) vector encoding either an artificial fusion protein of HPV16 E6 and E7 proteins, i.e., r3PICV-E7E6, or the reporter gene GFP, i.e., r3PICV-GFP, was analyzed in tumor bearing mice in the TC-1 tumor model after intratumoral administration compared to systemic administration. In addition, the TC-1 tumor model was used to compare the antitumoral effect of different tri-segmented, replication-competent arenavirus vectors encoding an HPV16 E7E6 fusion protein to the antitumoral effect of their respective wild-type virus counterparts. Furthermore, the antitumoral effect of homologous and heterologous prime-boost combinations using replication-competent HPV antigen-expressing vectors derived from different arenaviruses was also analyzed in tumor bearing mice in the TC-1 tumor model.

Study Design:

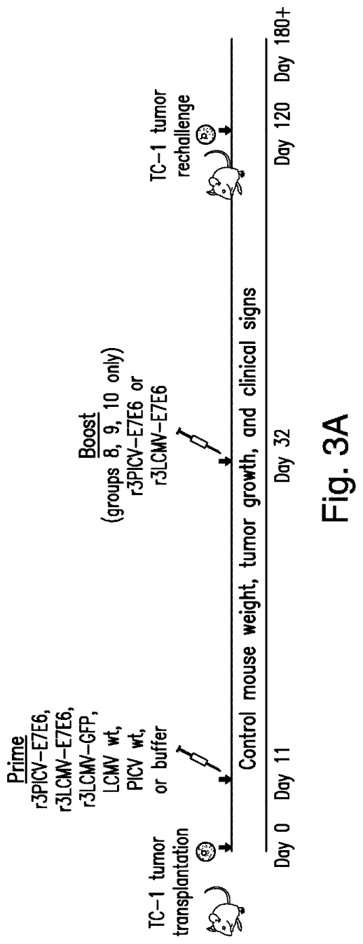

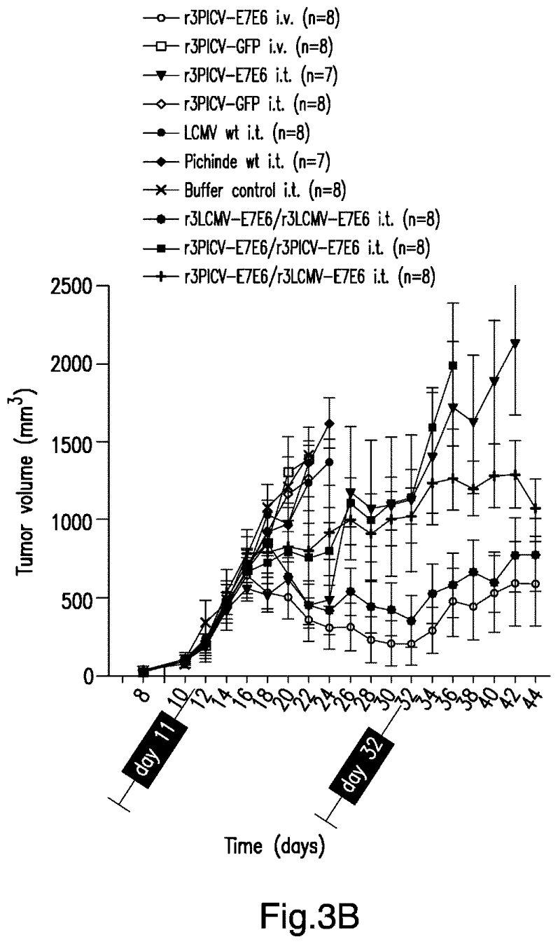

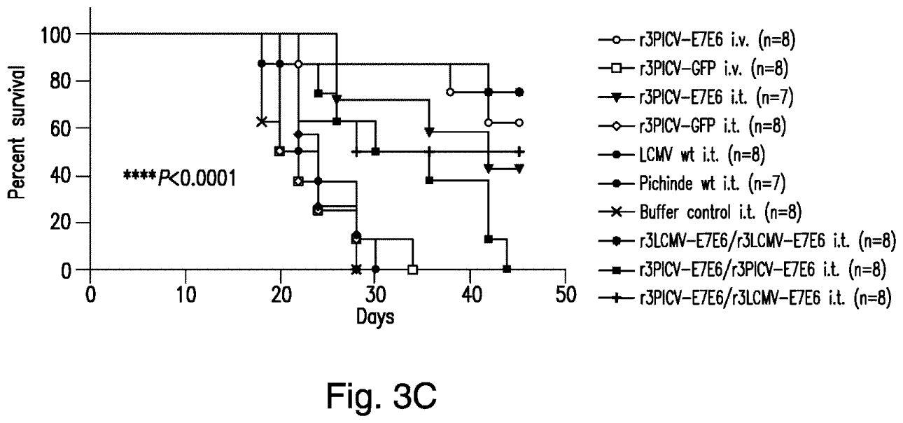

C57BL/6 mice were inoculated subcutaneously at the right flank with 1×10⁵ TC-1 cells on day 0 (groups 1-10). When tumors reached a size of approximately 100 mm³, mice were randomized and injected i.v. (groups 1 and 2) or i.t. (groups 3-10), with either 1×10⁵ RCV FFU of r3PICV-E7E6 (groups 1, 3, 9, 10), with 1×10⁵ RCV FFU of r3PICV-GFP (groups 2 and 4), 1×10⁵ RCV FFU recombinant wild-type LCMV (LCMV Clone 13 expressing the glycoprotein from strain WE) (group 5), 1×10⁵ RCV FFU recombinant wild-type Pichinde virus (group 6), buffer (control group 7), or with 1×10⁵ RCV FFU of r3LCMV-E7E6 (group 8). Mice in groups 8, 9 and 10 were boosted, i.e., immunized a second time, 21 days post prime immunization by intratumoral/subcutaneous administration (i.e., subcutaneous injection was used in animals where no tumor was palpable after the prime immunization) of 1×10⁵ RCV FFU of r3LCMV-E7E6 (groups 8 and 10) or 1×10⁵ RCV FFU of r3PICV-E7E6 (group 9). Eight mice were considered for each group. FIG. 3 provides (A) a schematic representation of the experimental design, (B) tumor growth after tumor challenge, and (C) overall survival of the indicated groups shown by Log-rank Kaplan-Meier plot. Subcutaneous tumor growth was monitored every second day starting on day 4 post tumor inoculation. The animals were sacrificed upon reaching a tumor size of ˜20 mm in diameter. The tumor volume was calculated according to the formula V=0.5 L×W² where L (length) and W (width) are the long and short diameters of the tumor, respectively. Some mice showing defined clinical signs (e.g., ulceration of the tumor or massive body weight loss) had to be sacrificed before reaching the final tumor size in accordance with animal welfare regulations. Measurements for each group are included in the plot until >50% mice per group were sacrificed.

As depicted in FIG. 3, respective results indicate that intratumoral as well as intravenous treatment with r3PICV-GFP (groups 2 and 4) or intratumoral treatment with Pichinde wild-type virus (group 6) did not inhibit tumor growth or increase overall survival in TC-1 tumor bearing mice compared to animals in the buffer control group (group 7). Consistent with a previously published report by Kalkavan et al., Nat. Commun. 2017 Mar. 1; 8:14447 (incorporated herein by reference in its entirety), intratumoral treatment with LCMV wild-type virus (group 5) resulted in (transient) shrinkage of existing TC-1 tumors; however, tumor size increased again and similar tumor growth rates were observed as in the buffer control group, resulting in similar overall survival. In significant contrast, a clear reduction in tumor progression was observed in animals treated intratumorally or intravenously with r3PICV-E7E6 (groups 1, 3, 9, 10) or intratumorally with r3LCMV-E7E6 (group 8). In line with the results depicted in FIG. 2, intratumoral treatment with r3LCMV-E7E6 resulted in elimination of subcutaneous TC-1 tumors in two out of eight tumor bearing, immunocompetent C57BL/6 mice. Surprisingly, in this experiment the strongest antitumoral effect was observed in mice of group 1, treated intravenously with r3PICV-E7E6. In this experimental group, tumors were eliminated in four out of eight mice within approximately 21 days after administration of r3PICV-E7E6.

These results demonstrate that the route of administration is a factor in reduction of tumor progression in mice treated with r3LCMV-E7E6 or r3PICV-E7E6. In particular, intratumoral treatment of mice with r3LCMV-E7E6 provided superior results in comparison to intravenous treatment of mice with r3LCMV-E7E6 (i.e., elimination of subcutaneous TC-1 tumors in two out of eight tumor bearing, immunocompetent C57BL/6 mice treated intratumorally with r3LCMV-E7E6). In contrast, intravenous treatment of mice with r3PICV-E7E6 provided superior results in comparison to intratumoral treatment of mice with r3PICV-E7E6 (i.e., elimination of subcutaneous TC-1 tumors in four out of eight mice within approximately 21 days after intravenous treatment of r3PICV-E7E6). Surprisingly, data from Examples 2 and 4 suggest that the pronounced and sustained anti-tumor control mediated by r3PICV-E7E6 and r3LCMV-E7E6, respectively, is at least partially due to the expression of a tumor-specific antigen by these vectors. Thus, the observed therapeutic efficacy of r3PICV-E7E6 and r3LCMV-E7E6, respectively, cannot be fully (or even largely) accounted for by either i) a direct effect of viral replication on the tumor, or ii) the inflammation resulting from viral replication in and around the tumor, or iii) an immunological attack on the virus, which replicates inside the tumor cells. If either of these mechanisms was chiefly responsible, the irrelevant r3PICV-GFP and r3LCMV-GFP vectors, as well as their wild-type virus counterparts should have had the equivalent effect.

### 8.2 Efficacy of Intratumoral Administration of Replication-Competent Arenavirus Vectors in the B16F10 and/or HCmel3 Mouse Melanoma Model

### 8.2.1 Example 5

The antitumoral effect of intratumoral compared to systemic administration of tri-segmented, replication-competent arenavirus vectors, e.g., r3LCMV, in tumor bearing mice is evaluated in the B16F10 and/or HCmel3 mouse melanoma model.

Study Design:

B16F10/HCmel3 tumor cells are implanted subcutaneously into C57BL/6 mice on day 0. When tumors are palpable and reach a size suitable for intratumoral application, mice are either left untreated (group 1), treated intratumorally with buffer (group 2), a high dose of a tri-segmented, replication-competent arenavirus vector, e.g., r3LCMV, vector mix encoding one or more melanoma antigens (e.g., r3LCMV-GP100, r3LCMV-Trp1 and r3LCMV-Trp2) (group 3), a low dose of a tri-segmented, replication-competent arenavirus vector, e.g., r3LCMV, vector mix (group 4), a high dose of tri-segmented, replication-competent arenavirus vector, e.g., r3LCMV, control, e.g., r3LCMV-GFP vector (group 5), a low dose of tri-segmented, replication-competent arenavirus vector, e.g., r3LCMV, control, e.g., r3LCMV-GFP vector (group 6), or injected intravenously with buffer (group 7), a high dose of the tri-segmented, replication-competent arenavirus vector, e.g., r3LCMV, vector mix (group 8), or a high dose of tri-segmented, replication-competent arenavirus vector, e.g., r3LCMV, control, e.g., r3LCMV-GFP vector (group 9). 5 to 15 days after the first dose, animals are boosted using the same experimental treatment (i.e., vector or buffer) and the same route of administration as for the first dose. Tumor growth after tumor challenge as well as animal survival are monitored.

### 8.2.2 Example 6(a)

The antitumoral effect of intratumoral compared to systemic administration of a tri-segmented, replication-competent arenavirus vector expressing the melanoma antigen Trp2, i.e., r3LCMV-Trp2, in tumor bearing mice was evaluated in the B16F10 mouse melanoma model.

Study Design:

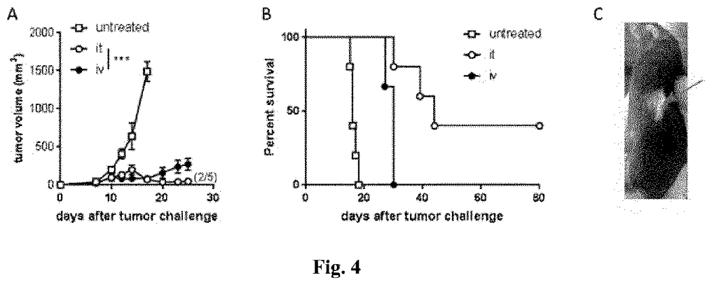

2×10⁵ B16F10 tumor cells were implanted subcutaneously into the flank of C57BL/6 mice on day 0. On day 7, when tumors were palpable and reached a size suitable for intratumoral application, mice were either left untreated (group 1), treated intratumorally with 7×10⁴ Pfu of a tri-segmented, replication-competent arenavirus vector expressing the melanoma antigen Trp2, r3LCMV-Trp2 (group 2), or injected intravenously with 7×10⁴ Pfu of r3LCMV-Trp2 (group 3). (A) Tumor growth after tumor challenge, and (B) animal survival, were monitored over time (FIG. 4).

Both intratumoral as well as intravenous administration of r3LCMV-Trp2 had a strong inhibiting effect on tumor growth and increased survival in test animals. However, best tumor control (A) and highest survival rates (B) (FIG. 4) were achieved after intratumoral injection of r3LCMV-Trp2. Importantly, only intratumoral and not intravenous vector treatment eliminated subcutaneous B16F101 tumors in 40% of the test animals. Surviving mice immunized intratumorally with r3LCMV-Trp2 developed autoimmune-related depigmentation at the site of the injection (FIG. 4(C), red arrow) indicating a strong induction of anti-melanocyte directed CD8+ T cell responses.

8.2.3 Example 6(b):

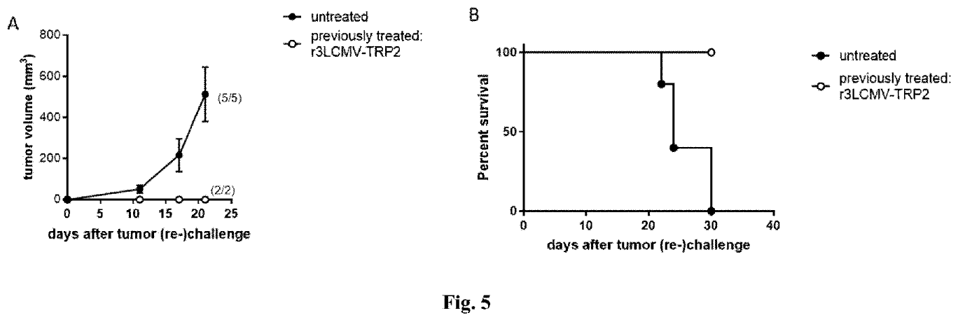

Tumor-free mice from Example 6(a) were re-challenged ˜120 days later by injection of 2×10⁵ B16F10 tumor cells into the contralateral flank to determine whether mice cured of B16F10 tumors acquired tumor-specific immune protection. As a control, untreated mice at similar age were challenged (first-challenge) with 2×10⁵ B16F10 tumor cells. Tumor formation and growth (A) as well as animal survival (B) were monitored (FIG. 5). Control animals showed rapid tumor development, whereas no tumor formation was observed after tumor re-challenge of surviving mice from Example 6(a) (i.e., mice that had completely eliminated subcutaneous B16F101 tumors after intratumoral r3LCMV-Trp2 treatment). Consistently, a 100% survival rate was observed in these pre-treated animals whereas no mouse in the control group survived for longer than 30 days after tumor inoculation.

### 8.2.4 Example 7

The antitumoral effect of intratumorally administered tri-segmented, replication-competent arenavirus vectors expressing either an unrelated control antigen, i.e., the green fluorescent protein (GFP), r3LCMV-GFP, or expressing the melanoma antigen Trp2, i.e., r3LCMV-Trp2, was evaluated and compared in tumor bearing mice in the B16F10 mouse melanoma model.

Study Design:

2×10⁵ B16F10 tumor cells were implanted subcutaneously into the flank of C57BL/6 mice on day 0. On day 7 when tumors were palpable and reached a size suitable for intratumoral application, mice were either left untreated (group 1), treated intratumorally with 7×10⁴ Pfu of a tri-segmented, replication-competent arenavirus vector expressing the green fluorescent protein, r3LCMV-GFP (group 2), or injected intratumorally with 7×10⁴ Pfu of a tri-segmented, replication-competent arenavirus vector expressing the melanoma antigen Trp2, r3LCMV-Trp2 (group 3). Tumor growth after tumor challenge was monitored over time.

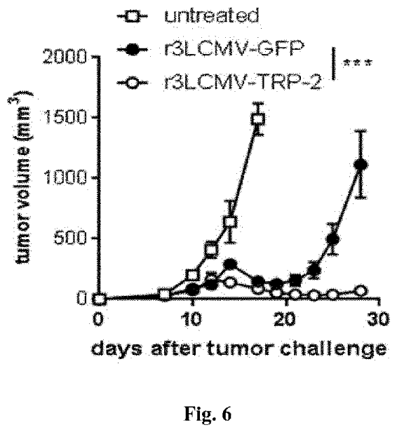

Both intratumoral administration of r3LCMV-GFP and r3LCMV-Trp2 delayed tumor growth compared to the untreated control animals (FIG. 6). However, after initial delayed growth, tumors in mice treated with r3LCMV-GFP increased again and at growth rates comparable to that observed in the control group. In contrast, mice treated with r3LCMV-Trp2 showed a clear and sustained reduction in tumor progression compared to the r3LCMV-GFP or control group.

### 8.2.5 Example 8

The antitumoral effect of (i) heterologous prime-boost combinations using replication-competent melanoma antigen-expressing vectors derived from different arenaviruses and/or (ii) combinations of alternative injections routes, i.e., intratumoral and intravenous administration, using replication-competent melanoma antigen-expressing vectors derived from the same or different arenaviruses, is analyzed in tumor bearing mice in the B16F10 and/or HCmel3 mouse melanoma model.

Study Design:

B16F10/HCmel3 tumor cells are implanted subcutaneously into C57BL/6 mice on day 0 (groups 1-15).

When tumors are palpable and reach a size suitable for intratumoral application, mice are either treated intratumorally (groups 1, 2, 4, 5, 7, 8, 10, 11, 13, 14) or intravenously (groups 3, 6, 9, 12, 15) with buffer (groups 1, 2, 3), a replication-competent LCMV vector mix encoding one or more melanoma antigens (“r3LCMV-MEL”) (groups 4, 5, 6, 10, 11, 12), or a replication-competent Pichinde virus vector mix encoding one or more melanoma antigens (“r3PICV-MEL”) (groups 7, 8, 9, 13, 14, 15). 10 to 15 days after the first injection, mice are either treated intratumorally (groups 1, 3, 4, 6, 7, 9, 10, 12, 13, 15) or intravenously (groups 2, 5, 8, 11, 14) with buffer (groups 1, 2, 3), r3LCMV-MEL (groups 4, 5, 6, 7, 8, 9), or r3PICV-MEL (groups 10, 11, 12, 13, 14, 15). Tumor growth after tumor challenge as well as animal survival are monitored. The fifteen treatment groups are summarized in Table 6.

TABLE 6
Summary of the fifteen treatment groups mentioned in Example 8.

| | TC-1 | | | | |
|---|---|---|---|---|---|
| group | challenge | 1^{st} injection | | 2^{nd} injection | |
| 1 | yes | IT | buffer | IT | buffer |
| 2 | yes | IT | buffer | IV | buffer |
| 3 | yes | IV | buffer | IT | buffer |
| 4 | yes | IT | r3LCMV-MEL | IT | r3LCMV-MEL |
| 5 | yes | IT | r3LCMV-MEL | IV | r3LCMV-MEL |
| 6 | yes | IV | r3LCMV-MEL | IT | r3LCMV-MEL |
| 7 | yes | IT | r3PICV-MEL | IT | r3LCMV-MEL |
| 8 | yes | IT | r3PICV-MEL | IV | r3LCMV-MEL |
| 9 | yes | IV | r3PICV-MEL | IT | r3LCMV-MEL |
| 10 | yes | IT | r3LCMV-MEL | IT | r3PICV-MEL |
| 11 | yes | IT | r3LCMV-MEL | IV | r3PICV-MEL |
| 12 | yes | IV | r3LCMV-MEL | IT | r3PICV-MEL |
| 13 | yes | IT | r3PICV-MEL | IT | r3PICV-MEL |
| 14 | yes | IT | r3PICV-MEL | IV | r3PICV-MEL |
| 15 | yes | IV | r3PICV-MEL | IT | r3PICV-MEL |

### 8.3 Example 9: Efficacy of Combination Treatment in the TC-1 Model

The antitumoral effect of a combination treatment using an intratumorally administered “empty” replication-competent arenavirus vector followed by intratumoral administration of a replication-competent arenavirus vector expressing an HPV antigen is analyzed in tumor bearing mice in the TC-1 tumor model.

Study Design:

C57BL/6 mice are inoculated subcutaneously at the right flank with 1×10⁵ TC-1 cells on day 1 (groups 1-10).

When tumors are palpable and reach a size suitable for intratumoral application (day ˜4), mice are either treated intratumorally with buffer (groups 1, 2 or 3), a high dose of a replication-competent arenavirus vector that does not express a foreign antigen (“r3LCMV-empty”) (groups 4, 5 and 6), a low dose of r3LCMV-empty (groups 7 and 8), a high dose of a replication-competent arenavirus vector encoding an artificial fusion protein of HPV-16 E6 and E7 proteins harboring 5 mutations abrogating the oncogenic potential of E6 and E7 (“r3LCMV-E7E6”) (group 9) or injected intravenously with a high dose of r3LCMV-E7E6 (group 10). 10 to 15 days after the first injection, mice are treated intratumorally with buffer (group 1), a high dose of a r3LCMV-E7E6 (groups 2, 5 and 9), a low dose of r3LCMV-E7E6 (groups 7), a high dose of r3LCMV-empty (group 3 and 6), a low dose of r3LCMV-empty (group 8), or injected intravenously with a high dose of r3LCMV-E7E6 (group 10). Tumor growth after tumor challenge as well as animal survival are monitored. The ten treatment groups are summarized in Table 7.

TABLE 7
Summary of the ten treatment groups mentioned in Example 9.

| | TC-1 | | | | | |
|---|---|---|---|---|---|---|
| group | challenge | route | 1^{st} injection | Dose | 2^{nd} injection | Dose |
| 1 | yes | IT | buffer | — | buffer | — |
| 2 | Yes | IT | buffer | — | r3LCMV- | high |
| | | | | | E7E6 | |
| 3 | Yes | IT | buffer | — | r3LCMV- | high |
| | | | | | empty | |
| 4 | yes | IT | r3LCMV- | high | buffer | — |
| | | | empty | | | |
| 5 | yes | IT | r3LCMV- | high | r3LCMV- | high |
| | | | empty | | E7E6 | |
| 6 | yes | IT | r3LCMV- | high | r3LCMV- | high |
| | | | empty | | empty | |
| 7 | yes | IT | r3LCMV- | low | r3LCMV- | low |
| | | | empty | | E7E6 | |
| 8 | yes | IT | r3LCMV- | low | r3LCMV- | low |
| | | | empty | | empty | |
| 9 | yes | IT | r3LCMV- | high | r3LCMV- | high |
| | | | E7E6 | | E7E6 | |
| 10 | yes | IV | r3LCMV- | high | r3LCMV- | high |
| | | | E7E6 | | E7E6 | |

### 8.4 Example 10: Efficacy of Combination Treatment in the B16F10 and/or HCmel3 Mouse Melanoma Model

The antitumoral effect of a combination treatment using an intratumorally administered “empty” replication-competent arenavirus vector followed by intratumoral administration of a mix of replication-competent arenavirus vectors expressing melanoma antigens is analyzed in tumor bearing mice in the in the B16F10 and/or HCmel3 mouse melanoma model.

Study Design:

B16F10/HCmel3 tumor cells are implanted subcutaneously into C57BL/6 mice on day 0 (groups 1-10).

When tumors are palpable and reach a size suitable for intratumoral application), mice are either treated intratumorally with buffer (groups 1, 2 or 3), a high dose of a replication-competent arenavirus vector that does not express a foreign antigen (groups 4, 5 and 6), a low dose of the replication-competent arenavirus vector that does not express a foreign antigen (groups 7 and 8), a high dose of replication-competent arenavirus vector mix encoding one or more melanoma antigens (“r3LCMV-MEL”) (vector mix of r3LCMV-GP100, r3LCMV-Trp1 and r3LCMV-Trp2) or replication-competent arenavirus vector encoding Trp2 (“r3LCMV-Trp2”) (group 9) or injected intravenously with a high dose of r3LCMV-MEL or r3LCMV-Trp2 (group 10). 10 to 15 days after the first injection, mice are treated intratumorally with buffer (group 1), a high dose of r3LCMV-MEL or r3LCMV-Trp2 (groups 2 or 5), a low dose of r3LCMV-MEL or r3LCMV-Trp2 (groups 7), a high dose of r3LCMV-empty (group 3 and 6), a low dose of r3LCMV-empty (group 8), or injected intravenously with a high dose of r3LCMV-MEL or r3LCMV-Trp2 (group 10). Tumor growth after tumor challenge as well as animal survival are monitored.

What is claimed is:

1. A method for treating a solid tumor in a subject comprising injecting an arenavirus particle directly into the tumor wherein the arenavirus particle expresses a tumor antigen or tumor-associated antigen or antigenic fragment thereof.

2. The method of claim 1, wherein a first arenavirus particle is administered systemically to the subject prior to said injecting.

3. The method of claim 1, wherein a second arenavirus particle is administered systemically to the subject after said injecting.

4. The method of any one of claims 1 to 3, wherein said arenavirus particle that is injected directly into the tumor is engineered to contain an arenavirus genomic segment comprising at least one arenavirus ORF in a position other than the wild-type position of said ORF.

5. The method of any one of claims 1 to 4, wherein said arenavirus particle that is injected directly into the tumor is replication competent.

6. The method of any one of claims 1 to 5, wherein the genome of said arenavirus particle that is injected directly into the tumor is tri-segmented.

7. The method of claim 6, wherein said tri-segmented genome comprises one L segment and two S segments.

8. The method of claim 6 or 7, wherein propagation of said arenavirus particle that is injected directly into the tumor does not result in a replication-competent bi-segmented viral particle.

9. The method of claim 6 or 7, wherein propagation of said arenavirus particle that is injected directly into the tumor does not result in a replication-competent bi-segmented viral particle after 70 days of persistent infection in mice lacking type I interferon receptor, type II interferon receptor and RAG1 and having been infected with 10⁴ PFU of said arenavirus particle.

10. The method of claim 7, wherein one of said two S segments is an S segment, wherein the ORF encoding the GP is under control of an arenavirus 3′ UTR.

11. The method of claim 7, wherein the arenavirus particle that is injected directly into the tumor comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

12. The method of any one of claims 1 to 11, wherein said arenavirus particle that is injected directly into the tumor is derived from lymphocytic choriomeningitis virus (“LCMV”), Junin virus (“JUNV”), or Pichinde virus (“PICV”).

13. The method of claim 12, wherein said arenavirus particle that is injected directly into the tumor is derived from LCMV.

14. The method of claim 13, wherein said LCMV is MP strain, WE strain, Armstrong strain, or Armstrong Clone 13 strain.

15. The method of claim 13, wherein said LCMV is Clone 13 strain with a glycoprotein (GP) from the WE strain.

16. The method of claim 12, wherein said arenavirus particle that is injected directly into the tumor is derived from JUNV.

17. The method of claim 16, wherein said JUNV is JUNV vaccine Candid #1 strain, or JUNV vaccine XJ Clone 3 strain.

18. The method of claim 12, wherein said arenavirus particle that is injected directly into the tumor is derived from PICV.

19. The method of claim 18, wherein said PICV is strain Munchique CoAn4763 isolate P18, or P2 strain.

20. The method of any one of claims 1 to 19, wherein the arenavirus particle that is injected directly into the tumor comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secemin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

21. The method of claim 20, wherein said tumor antigen or tumor associated antigen is selected from the group consisting of artificial fusion protein of HPV16 E7 and E6 proteins, HPV E6, HPV E7, GP100, TRP1, and TRP2.

22. The method of any one of claims 1 to 21, wherein the arenavirus particle that is injected directly into the tumor comprises a nucleotide sequence encoding two, three, four, five, six, seven, eight, nine, ten or more tumor antigens or tumor associated antigens or antigenic fragments thereof.

23. The method of any one of claims 1 to 22, which further comprises administering a chemotherapeutic agent to said subject.

24. The method of claim 23, wherein said chemotherapeutic agent is cyclophosphamide.

25. The method of claim 23 or 24, wherein said arenavirus particle that is injected directly into the tumor and said chemotherapeutic agent are co-administered simultaneously to the subject.

26. The method of claim 23 or 24, wherein said arenavirus particle that is injected directly into the tumor is administered to the subject prior to administration of said chemotherapeutic agent.

27. The method of claim 23 or 24, wherein said arenavirus particle that is injected directly into the tumor is administered to the subject after administration of said chemotherapeutic agent.

28. The method of any one of claims 1 to 27, wherein said subject is suffering from, is susceptible to, or is at risk for melanoma.

29. The method of any one of claims 1 to 28, which further comprises administering an immune checkpoint inhibitor to the subject.

30. The method of claim 29, wherein the immune checkpoint inhibitor is an anti-PD-1 antibody.

31. The method of claim 29, wherein the immune checkpoint inhibitor is an anti-PD-L1 antibody.

32. The method of any one of claims 29 to 31, wherein said arenavirus particle that is injected directly into the tumor and said immune checkpoint inhibitor are co-administered simultaneously.

33. The method of any one of claims 29 to 31, wherein said arenavirus particle that is injected directly into the tumor is administered prior to administration of said immune checkpoint inhibitor.

34. The method of any one of claims 29 to 31, wherein said arenavirus particle that is injected directly into the tumor is administered after administration of said immune checkpoint inhibitor.

35. The method of any one of claims 1 to 34, wherein the arenavirus particle that is injected directly into the tumor comprises a first nucleotide sequence encoding a first human papillomavirus (HPV) antigen.

36. The method of claim 35, wherein the first nucleotide sequence further encodes a second HPV antigen.

37. The method of claim 35 or 36, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

38. The method of claim 35 or 36, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

39. The method of any one of claims 1 to 38, wherein said step of injecting comprises injecting the same arenavirus particle multiple times.

40. The method of any one of claims 1 to 38, wherein said step of injecting comprises injecting arenavirus particles derived from the same arenavirus, but expressing different tumor antigens or tumor-associated antigens or antigenic fragments thereof.

41. The method of any one of claims 1 to 38, wherein said step of injecting comprises injecting arenavirus particles derived from different arenaviruses, but expressing the same tumor antigen or tumor-associated antigen or antigenic fragment thereof.

42. The method of any one of claims 1 to 38, wherein said step of injecting comprises injecting arenavirus particles derived from different arenaviruses and expressing different tumor antigens or tumor-associated antigens or antigenic fragments thereof.

43. The method of any one of claims 2 to 42, wherein said systemically administered first and/or second arenavirus particle is engineered to contain an arenavirus genomic segment comprising at least one arenavirus ORF in a position other than the wild-type position of said ORF.

44. The method of claim 43, wherein said systemically administered first and/or second arenavirus particle is replication deficient.

45. The method of claim 43, wherein said systemically administered first and/or second arenavirus particle is replication competent.

46. The method of claim 43, wherein the genome of said systemically administered first and/or second arenavirus particle is tri-segmented.

47. The method of claim 46, wherein said tri-segmented genome comprises one L segment and two S segments.

48. The method of claim 46 or 47, wherein propagation of said systemically administered first and/or second arenavirus particle does not result in a replication-competent bi-segmented viral particle.

49. The method of claim 46 or 47, wherein propagation of said systemically administered first and/or second arenavirus particle does not result in a replication-competent bi-segmented viral particle after 70 days of persistent infection in mice lacking type I interferon receptor, type II interferon receptor and RAG1 and having been infected with 10⁴ PFU of said arenavirus particle.

50. The method of claim 47, wherein one of said two S segments is an S segment, wherein the ORF encoding the GP is under control of an arenavirus 3′ UTR.

51. The method of claim 47 or 50, wherein the systemically administered first and/or second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

52. The method of any one of claims 43 to 51, wherein said systemically administered first and/or second arenavirus particle is derived from LCMV, JUNV, or PICV.

53. The method of claim 52, wherein said systemically administered first and/or second arenavirus particle is derived from LCMV.

54. The method of claim 53, wherein said LCMV is MP strain, WE strain, Armstrong strain, or Armstrong Clone 13 strain.

55. The method of claim 53, wherein said LCMV is Clone 13 strain with a glycoprotein (GP) from the WE strain.

56. The method of claim 52, wherein said systemically administered first and/or second arenavirus particle is derived from JUNV.

57. The method of claim 56, wherein said JUNV is JUNV vaccine Candid #1 strain, or JUNV vaccine XJ Clone 3 strain.

58. The method of claim 52, wherein said systemically administered first and/or second arenavirus particle is derived from PICV.

59. The method of claim 58, wherein said PICV is strain Munchique CoAn4763 isolate P18, or P2 strain.

60. The method of any one of claims 43 to 59, wherein the systemically administered first and/or second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secemin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

61. The method of claim 60, wherein said tumor antigen or tumor associated antigen is selected from the group consisting of artificial fusion protein of HPV16 E7 and E6 proteins, HPV E6, HPV E7, GP100, TRP1, and TRP2.

62. The method of any one of claims 43 to 61, wherein the systemically administered first and/or second arenavirus particle comprises a nucleotide sequence encoding two, three, four, five, six, seven, eight, nine, ten or more tumor antigens or tumor associated antigens or antigenic fragments thereof.

63. The method of any one of claims 43 to 62, which further comprises administering a chemotherapeutic agent to said subject.

64. The method of claim 63, wherein said chemotherapeutic agent is cyclophosphamide.

65. The method of claim 63 or 64, wherein said systemically administered first and/or second arenavirus particle and said chemotherapeutic agent are co-administered simultaneously to the subject.

66. The method of claim 63 or 64, wherein said systemically administered first and/or second arenavirus particle is administered to the subject prior to administration of said chemotherapeutic agent.

67. The method of claim 63 or 64, wherein said systemically administered first and/or second arenavirus particle is administered to the subject after administration of said chemotherapeutic agent.

68. The method of any one of claims 43 to 67, wherein said subject is suffering from, is susceptible to, or is at risk for melanoma.

69. The method of any one of claims 43 to 68, which further comprises administering an immune checkpoint inhibitor to the subject.

70. The method of claim 69, wherein the immune checkpoint inhibitor is an anti-PD-1 antibody.

71. The method of claim 69, wherein the immune checkpoint inhibitor is an anti-PD-L1 antibody.

72. The method of any one of claims 69 to 71, wherein said systemically administered first and/or second arenavirus particle and said immune checkpoint inhibitor are co-administered simultaneously.

73. The method of any one of claims 69 to 71, wherein said systemically administered first and/or second arenavirus particle is administered prior to administration of said immune checkpoint inhibitor.

74. The method of any one of claims 69 to 71, wherein said systemically administered first and/or second arenavirus particle is administered after administration of said immune checkpoint inhibitor.

75. The method of any one of claims 43 to 74, wherein the systemically administered first and/or second arenavirus particle comprises a first nucleotide sequence encoding a first human papillomavirus (HPV) antigen.

76. The method of claim 75, wherein the first nucleotide sequence further encodes a second HPV antigen.

77. The method of claim 75 or 76, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

78. The method of claim 75 or 76, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

79. A kit comprising a container and instructions for use, wherein said container comprises an arenavirus particle in a pharmaceutical composition suitable for injection directly into a solid tumor, wherein said kit further comprises an injection apparatus suitable for performing an injection directly into a solid tumor, wherein said arenavirus particle expresses a tumor antigen or tumor-associated antigen or antigenic fragment thereof.

80. The kit of claim 79, wherein said arenavirus particle is engineered to contain an arenavirus genomic segment comprising at least one arenavirus open reading frame (“ORF”) in a position other than the wild-type position of said ORF.

81. The kit of claim 79 or 80, wherein said arenavirus particle is replication competent.

82. The kit of any one of claims 79 to 81, wherein the genome of said arenavirus particle is tri-segmented.

83. The kit of claim 82 wherein said tri-segmented genome comprises one L segment and two S segments.

84. The kit of claim 82 or 83, wherein propagation of said arenavirus particle does not result in a replication-competent bi-segmented viral particle.

85. The kit of claim 82 or 83, wherein propagation of said arenavirus particle does not result in a replication-competent bi-segmented viral particle after 70 days of persistent infection in mice lacking type I interferon receptor, type II interferon receptor and RAG1 and having been infected with 10⁴ PFU of said first or second arenavirus particle.

86. The kit of claim 83, wherein one of said two S segments is an S segment, wherein the ORF encoding the GP is under control of an arenavirus 3′ UTR.

87. The kit of claim 83, wherein the arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

88. The kit of any one of claims 79 to 87, wherein said arenavirus particle is derived from LCMV, JUNV, or PICV.

89. The kit of claim 88, wherein said arenavirus particle is derived from LCMV.

90. The kit of claim 89, wherein said LCMV is MP strain, WE strain, Armstrong strain, or Armstrong Clone 13 strain.

91. The kit of claim 89, wherein said LCMV is Clone 13 strain with a GP from the WE strain.

92. The kit of claim 88, wherein said arenavirus particle is derived from JUNV.

93. The kit of claim 92, wherein said JUNV is JUNV vaccine Candid #1 strain, or JUNV vaccine XJ Clone 3 strain.

94. The kit of claim 88, wherein said arenavirus particle is derived from PICV.

95. The kit of claim 94, wherein said PICV is strain Munchique CoAn4763 isolate P18, or P2 strain.

96. The kit of any one of claims 79 to 95, wherein the arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A1, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

97. The kit of claim 96, wherein said tumor antigen or tumor associated antigen is selected from the group consisting of artificial fusion protein of HPV16 E7 and E6 proteins, HPV E6, HPV E7, GP100, TRP1, and TRP2.

98. The kit of any one of claims 79 to 97, wherein the arenavirus particle comprises a nucleotide sequence encoding two, three, four, five, six, seven, eight, nine, ten or more tumor antigens or tumor associated antigens or antigenic fragments thereof.

99. The kit of any one of claims 79 to 98, which further comprises a container comprising a chemotherapeutic agent.

100. The kit of claim 99, wherein said chemotherapeutic agent is cyclophosphamide.

101. The kit of claim 99 or 100, wherein said arenavirus particle and said chemotherapeutic agent are formulated for administration simultaneously to a subject.

102. The kit of claim 99 or 100, wherein said arenavirus particle is formulated for administration to a subject prior to administration of said chemotherapeutic agent.

103. The kit of claim 99 or 100, wherein said arenavirus particle is formulated for administration to a subject after administration of said chemotherapeutic agent.

104. The kit of any one of claims 79 to 103, which further comprises a container comprising an immune checkpoint inhibitor.

105. The kit of claim 104, wherein said immune checkpoint inhibitor is an anti-PD-1 antibody.

106. The kit of claim 104, wherein said immune checkpoint inhibitor is an anti-PD-L 1 antibody.

107. The kit of any one of claims 104 to 106, wherein said arenavirus particle and said immune checkpoint inhibitor are formulated for administration simultaneously to a subject.

108. The kit of claim 104 to 106, wherein said arenavirus particle is formulated for administration to a subject prior to administration of said immune checkpoint inhibitor.

109. The kit of claim 104 to 106, wherein said arenavirus particle is formulated for administration to a subject after administration of said immune checkpoint inhibitor.

110. The kit of any one of claims 79 to 109, wherein the arenavirus particle comprises a first nucleotide sequence encoding a first human papillomavirus (HPV) antigen.

111. The kit of claim 110, wherein the first nucleotide sequence further encodes a second HPV antigen.

112. The kit of claim 110 or 111, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

113. The kit of claim 110 or 111, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

114. The kit of any one of claims 79 to 113, which comprises multiple containers comprising the same arenavirus particle.

115. The kit of any one of claims 79 to 113, which comprises multiple containers, comprising multiple arenavirus particles derived from the same arenavirus, but expressing different tumor antigens or tumor-associated antigens or antigenic fragments thereof.

116. The kit of any one of claims 79 to 113, which comprises multiple containers, comprising multiple arenavirus particles derived from different arenaviruses, but expressing the same tumor antigen or tumor-associated antigen or antigenic fragment thereof.

117. The kit of any one of claims 79 to 113, which comprises multiple containers, comprising multiple arenavirus particles derived from different arenaviruses and expressing different tumor antigens or tumor-associated antigens or antigenic fragments thereof.

118. The kit of any one of claims 79 to 117, which further comprises one or more arenavirus particles in a pharmaceutical composition suitable for intravenous administration.

119. The kit of claim 118, wherein said one or more arenavirus particles in a pharmaceutical composition suitable for intravenous administration are engineered to contain an arenavirus genomic segment comprising at least one arenavirus ORF in a position other than the wild-type position of said ORF.

120. The kit of claim 118 or 119, wherein said one or more arenavirus particles in a pharmaceutical composition suitable for intravenous administration are replication deficient.

121. The kit of claim 118 or 119, wherein said one or more arenavirus particles in a pharmaceutical composition suitable for intravenous administration are replication competent.

122. The kit of claim 118 or 119, wherein the genome of said one or more arenavirus particles in a pharmaceutical composition suitable for intravenous administration are tri-segmented.

123. The kit of claim 122, wherein said tri-segmented genome comprises one L segment and two S segments.

124. The kit of claim 122 or 123, wherein propagation of said one or more arenavirus particles suitable for intravenous administration does not result in a replication-competent bi-segmented viral particle.

125. The kit of claim 122 or 123, wherein propagation of said one or more arenavirus particles in a pharmaceutical composition suitable for intravenous administration does not result in a replication-competent bi-segmented viral particle after 70 days of persistent infection in mice lacking type I interferon receptor, type II interferon receptor and RAG1 and having been infected with 10⁴ PFU of said arenavirus particle.

126. The kit of claim 123, wherein one of said two S segments is an S segment, wherein the ORF encoding the GP is under control of an arenavirus 3′ UTR.

127. The kit of claim 123, wherein said one or more arenavirus particles in a pharmaceutical composition suitable for intravenous administration comprise two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

128. The kit of any one of claims 118 to 127, wherein said one or more arenavirus particles in a pharmaceutical composition suitable for intravenous administration are derived from LCMV, JUNV, or PICV.

129. The kit of claim 128, wherein said one or more arenavirus particles in a pharmaceutical composition suitable for intravenous administration are derived from LCMV.

130. The kit of claim 129, wherein said LCMV is MP strain, WE strain, Armstrong strain, or Armstrong Clone 13 strain.

131. The kit of claim 129, wherein said LCMV is Clone 13 strain with a glycoprotein (GP) from the WE strain.

132. The kit of claim 128, wherein said one or more arenavirus particles in a pharmaceutical composition suitable for intravenous administration are derived from JUNV.

133. The kit of claim 132, wherein said JUNV is JUNV vaccine Candid #1 strain, or JUNV vaccine XJ Clone 3 strain.

134. The kit of claim 128, wherein said one or more arenavirus particles in a pharmaceutical composition suitable for intravenous administration are derived from PICV.

135. The kit of claim 134, wherein said PICV is strain Munchique CoAn4763 isolate P18, or P2 strain.

136. The kit of any one of claims 118 to 135, wherein said one or more arenavirus particles in a pharmaceutical composition suitable for intravenous administration comprise a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

137. The kit of claim 136, wherein said tumor antigen or tumor associated antigen is selected from the group consisting of artificial fusion protein of HPV16 E7 and E6 proteins, HPV E6, HPV E7, GP100, TRP1, and TRP2.

138. The kit of any one of claims 118 to 137, wherein said one or more arenavirus particles in a pharmaceutical composition suitable for intravenous administration comprise a nucleotide sequence encoding two, three, four, five, six, seven, eight, nine, ten or more tumor antigens or tumor associated antigens or antigenic fragments thereof.

139. The kit of any one of claims 118 to 138, wherein said one or more arenavirus particles in a pharmaceutical composition suitable for intravenous administration comprise a first nucleotide sequence encoding a first human papillomavirus (HPV) antigen.

140. The kit of claim 139, wherein the first nucleotide sequence further encodes a second HPV antigen.

141. The kit of claim 139 or 140, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

142. The kit of claim 139 or 140, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

143. The kit of any one of claims 118 to 142, wherein said one or more arenavirus particles in a pharmaceutical composition suitable for intravenous administration are formulated for injection prior to said arenavirus particle in a pharmaceutical composition suitable for injection directly into a solid tumor.

144. The kit of any one of claims 118 to 142, wherein said one or more arenavirus particles in a pharmaceutical composition suitable for intravenous administration are formulated for injection subsequent to said arenavirus particle in a pharmaceutical composition suitable for injection directly into a solid tumor.

145. The kit of any one of claims 118 to 142, wherein said one or more arenavirus particles in a pharmaceutical composition suitable for intravenous administration are formulated for injection concurrently with said arenavirus particle in a pharmaceutical composition suitable for injection directly into a solid tumor.

146. The kit of any one of claims 118 to 145, wherein said kit further comprises an apparatus suitable for performing intravenous administration.

147. The kit of any one of claims 118 to 146, wherein said kit further comprises an injection apparatus suitable for performing an injection directly into a solid tumor.

148. A method for treating a solid tumor in a subject comprising:
(a) administering a first arenavirus particle to the subject, wherein the first arenavirus particle does not express a tumor antigen or tumor-associated antigen or antigenic fragment thereof; and
(b) administering a second arenavirus particle to the subject, wherein the second arenavirus particle expresses a tumor antigen or tumor-associated antigen or antigenic fragment thereof.

149. The method of claim 148, wherein the first and second arenavirus particles are injected directly into the tumor.

150. The method of claim 148, wherein the first arenavirus particle is administered intravenously and the second arenavirus particle is injected directly into the tumor.

151. The method of claim 148, wherein the first arenavirus particle is injected directly into the tumor and the second arenavirus particle is administered intravenously.

152. The method of any one of claims 148 to 151, wherein said first arenavirus particle is engineered to contain an arenavirus genomic segment comprising at least one arenavirus open reading frame (“ORF”) in a position other than the wild-type position of said ORF.

153. The method of any one of claims 148 to 152, wherein said first arenavirus particle is replication competent.

154. The method of any one of claims 148 to 153, wherein the genome of said first arenavirus particle is tri-segmented.

155. The method of any one of claims 148 to 154, wherein said second arenavirus particle is engineered to contain an arenavirus genomic segment comprising:
(i) a nucleotide sequence encoding a tumor antigen, tumor associated antigen or an antigenic fragment thereof; and
(ii) at least one arenavirus ORF in a position other than the wild-type position.

156. The method of any one of claims 148 to 155, wherein said second arenavirus particle is replication competent.

157. The method of any one of claims 148 to 156, wherein the genome of said second arenavirus particle is tri-segmented.

158. The method of claim 154 or 157, wherein said tri-segmented genome comprises one L segment and two S segments.

159. The method of any one of claims 154, 157, and 158, wherein propagation of said first or second arenavirus particle does not result in a replication-competent bi-segmented viral particle.

160. The method of any one of claims 154, 157, and 158, wherein propagation of said first or second arenavirus particle does not result in a replication-competent bi-segmented viral particle after 70 days of persistent infection in mice lacking type I interferon receptor, type II interferon receptor and recombination activating gene 1 (RAG1) and having been infected with 10⁴ PFU of said first or second arenavirus particle.

161. The method of claim 158, wherein one of said two S segments is an S segment, wherein the ORF encoding the GP is under control of an arenavirus 3′ UTR.

162. The method of claim 158, wherein the second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

163. The method of any one of claims 148 to 162, wherein said first arenavirus particle and said second arenavirus particle are derived from different arenavirus species.

164. The method of any one of claims 148 to 163, wherein said first and/or second arenavirus particle is derived from LCMV, JUNV, or PICV.

165. The method of claim 164, wherein said first and/or second arenavirus particle is derived from LCMV.

166. The method of claim 165, wherein said LCMV is MP strain, WE strain, Armstrong strain, or Armstrong Clone 13 strain.

167. The method of claim 165, wherein said LCMV is Clone 13 strain with a glycoprotein (GP) from the WE strain.

168. The method of claim 164, wherein said first and/or second arenavirus particle is derived from JUNV.

169. The method of claim 168, wherein said JUNV is JUNV vaccine Candid #1 strain, or JUNV vaccine XJ Clone 3 strain.

170. The method of claim 164, wherein said first and/or second arenavirus particle is derived from PICV.

171. The method of claim 170, wherein said PICV is strain Munchique CoAn4763 isolate P18, or P2 strain.

172. The method of any one of claims 148 to 171, wherein the second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

173. The method of claim 172, wherein said tumor antigen or tumor associated antigen is selected from the group consisting of artificial fusion protein of HPV16 E7 and E6 proteins, HPV E6, HPV E7, GP100, TRP1, and TRP2.

174. The method of any one of claims 148 to 173, wherein the second arenavirus particle comprises a nucleotide sequence encoding two, three, four, five, six, seven, eight, nine, ten or more tumor antigens or tumor associated antigens or antigenic fragments thereof.

175. The method of any one of claims 148 to 174, which further comprises administering a chemotherapeutic agent to said subject.

176. The method of claim 175, wherein said chemotherapeutic agent is cyclophosphamide.

177. The method of claim 175 or 176, wherein said first or second arenavirus particle and said chemotherapeutic agent are co-administered simultaneously to the subject.

178. The method of claim 175 or 176, wherein said first and/or second arenavirus particles are administered to the subject prior to administration of said chemotherapeutic agent.

179. The method of claim 175 or 176, wherein said first and/or second arenavirus particles are administered to the subject after administration of said chemotherapeutic agent.

180. The method of any one of claims 148 to 179, wherein said subject is suffering from, is susceptible to, or is at risk for melanoma.

181. The method of any one of claims 148 to 180, which further comprises administering an immune checkpoint inhibitor to the subject.

182. The method of claim 181, wherein the immune checkpoint inhibitor is an anti-PD-1 antibody.

183. The method of claim 181, wherein the immune checkpoint inhibitor is an anti-PD-L1 antibody.

184. The method of any one of claims 181 to 183, wherein said first or second arenavirus particle and said immune checkpoint inhibitor are co-administered simultaneously.

185. The method of any one of claims 181 to 183, wherein said first and/or second arenavirus particles are administered prior to administration of said immune checkpoint inhibitor.

186. The method of any one of claims 181 to 183, wherein said first and/or second arenavirus particles are administered after administration of said immune checkpoint inhibitor.

187. The method of any one of claims 148 to 186, wherein the second arenavirus particle comprises a first nucleotide sequence encoding a first human papillomavirus (HPV) antigen.

188. The method of claim 187, wherein the first nucleotide sequence further encodes a second HPV antigen.

189. The method of claim 187 or 188, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

190. The method of claim 187 or 188, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

191. The method of any one of claims 148 to 190, wherein said first and second arenavirus particles are injected concurrently.

192. The method of claim 191, wherein said first and second arenavirus particles are part of the same composition or formulation.

193. The method of any one of claims 148 to 190, wherein said first arenavirus particle is injected prior to said second arenavirus particle.

194. The method of any one of claims 148 to 190, wherein said first arenavirus particle is injected subsequent to said second arenavirus particle.

195. The method of any one of claims 148 to 194, wherein said step of administering said first arenavirus particle comprises administering the same arenavirus particle multiple times.

196. The method of any one of claims 148 to 194, wherein said step of administering said first arenavirus particle comprises administering one or more arenavirus particles derived from different arenaviruses.

197. The method of any one of claims 148 to 196, wherein said step of administering said second arenavirus particle comprises administering the same arenavirus particle multiple times.

198. The method of any one of claims 148 to 196, wherein said step of administering said second arenavirus particle comprises administering one or more arenavirus particles derived from the same arenavirus, but expressing different tumor antigens or tumor-associated antigens or antigenic fragments thereof.

199. The method of any one of claims 148 to 196, wherein said step of administering said second arenavirus particle comprises administering one or more arenavirus particles derived from different arenaviruses, but expressing the same tumor antigen or tumor-associated antigen or antigenic fragment thereof.

200. The method of any one of claims 148 to 196, wherein said step of administering said second arenavirus particle comprises administering one or more arenavirus particles derived from different arenaviruses and expressing different tumor antigens or tumor-associated antigens or antigenic fragments thereof.

201. A kit comprising two or more containers and instructions for use, wherein one of said containers comprises a first arenavirus particle in a pharmaceutical composition suitable for injection directly into a solid tumor or suitable for intravenous administration and another of said containers comprises a second arenavirus particle in a pharmaceutical composition suitable for injection directly into a solid tumor or suitable for intravenous administration, and wherein said first arenavirus particle does not express a tumor antigen or tumor-associated antigen or antigenic fragment thereof and said second arenavirus particle expresses a tumor antigen or tumor-associated antigen or antigenic fragment thereof.

202. The kit of claim 201, wherein the first and second arenavirus particles are in a pharmaceutical composition suitable for injection directly into a solid tumor.

203. The kit of claim 201, wherein the first arenavirus particle is in a pharmaceutical composition suitable for intravenous administration and the second arenavirus particle is in a pharmaceutical composition suitable for injection directly into a solid tumor.

204. The kit of claim 201, wherein the first arenavirus particle is in a pharmaceutical composition suitable for injection directly into a solid tumor and the second arenavirus particle is in a pharmaceutical composition suitable for intravenous administration.

205. The kit of any one of claims 201 to 204, wherein said first arenavirus particle is engineered to contain an arenavirus genomic segment comprising at least one arenavirus open reading frame (“ORF”) in a position other than the wild-type position of said ORF.

206. The kit of any one of claims 201 to 205, wherein said first arenavirus particle is replication competent.

207. The kit of any one of claims 201 to 206, wherein the genome of said first arenavirus particle is tri-segmented.

208. The kit of any one of claims 201 to 207, wherein said second arenavirus particle is engineered to contain an arenavirus genomic segment comprising:
(i) a nucleotide sequence encoding a tumor antigen, tumor associated antigen or an antigenic fragment thereof; and
(ii) at least one arenavirus ORF in a position other than the wild-type position.

209. The kit of any one of claims 201 to 208, wherein said second arenavirus particle is replication competent.

210. The kit of any one of claims 201 to 209, wherein the genome of said second arenavirus particle is tri-segmented.

211. The kit of claim 207 or 210, wherein said tri-segmented genome comprises one L segment and two S segments.

212. The kit of any one of claims 207, 210, and 211, wherein propagation of said first or second arenavirus particle does not result in a replication-competent bi-segmented viral particle.

213. The kit of any one of claims 207, 210, and 211, wherein propagation of said first or second arenavirus particle does not result in a replication-competent bi-segmented viral particle after 70 days of persistent infection in mice lacking type I interferon receptor, type II interferon receptor and RAG1 and having been infected with 10⁴ PFU of said first or second arenavirus particle.

214. The kit of claim 211, wherein one of said two S segments is an S segment, wherein the ORF encoding the GP is under control of an arenavirus 3′ UTR.

215. The kit of claim 210, wherein the second arenavirus particle comprises two S segments, which comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

216. The kit of any one of claims 201 to 215, wherein said first arenavirus particle and said second arenavirus particle are derived from different arenavirus species.

217. The kit of any one of claims 201 to 216, wherein said first and/or second arenavirus particle is derived from LCMV, JUNV, or PICV.

218. The kit of claim 217, wherein said first and/or second arenavirus particle is derived from LCMV.

219. The kit of claim 218, wherein said LCMV is MP strain, WE strain, Armstrong strain, or Armstrong Clone 13 strain.

220. The kit of claim 218, wherein said LCMV is Clone 13 strain with a GP from a WE strain.

221. The kit of claim 217, wherein said first and/or second arenavirus particle is derived from JUNV.

222. The kit of claim 221, wherein said JUNV is JUNV vaccine Candid #1 strain, or JUNV vaccine XJ Clone 3 strain.

223. The kit of claim 217, wherein said first and/or second arenavirus particle is derived from PICV.

224. The kit of claim 223, wherein said PICV is strain Munchique CoAn4763 isolate P18, or P2 strain.

225. The kit of any one of claims 201 to 224, wherein the second arenavirus particle comprises a nucleotide sequence encoding a tumor antigen, tumor associated antigen, or an antigenic fragment thereof, wherein said tumor antigen or tumor associated antigen is selected from the group consisting of artificial fusion protein of HPV16 E7 and E6 proteins, oncogenic viral antigens, cancer-testis antigens, oncofetal antigens, tissue differentiation antigens, mutant protein antigens, Adipophilin, AIM-2, ALDH1A1, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secemin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

226. The kit of claim 225, wherein said tumor antigen or tumor associated antigen is selected from the group consisting of artificial fusion protein of HPV16 E7 and E6 proteins, HPV E6, HPV E7, GP100, TRP1, and TRP2.

227. The kit of any one of claims 201 to 226, wherein the second arenavirus particle comprises a nucleotide sequence encoding two, three, four, five, six, seven, eight, nine, ten or more tumor antigens or tumor associated antigens or antigenic fragments thereof.

228. The kit of any one of claims 201 to 227, which further comprises a container comprising a chemotherapeutic agent.

229. The kit of claim 228, wherein said chemotherapeutic agent is cyclophosphamide.

230. The kit of claim 228 or 229, wherein said first and/or second arenavirus particle and said chemotherapeutic agent are formulated for administration simultaneously to a subject.

231. The kit of claim 228 or 229, wherein said first and/or second arenavirus particles are formulated for administration to a subject prior to administration of said chemotherapeutic agent.

232. The kit of claim 228 or 229, wherein said first and/or second arenavirus particles are formulated for administration to a subject after administration of said chemotherapeutic agent.

233. The kit of any one of claims 201 to 232, which further comprises a container comprising an immune checkpoint inhibitor.

234. The kit of claim 233, wherein said immune checkpoint inhibitor is an anti-PD-1 antibody

235. The kit of claim 233, wherein said immune checkpoint inhibitor is an anti-PD-L1 antibody.

236. The kit of claims 233 to 235, wherein said first and/or second arenavirus particle and said immune checkpoint inhibitor are formulated for administration simultaneously to a subject.

237. The kit of claims 233 to 235, wherein said first and/or second arenavirus particles are formulated for administration to a subject prior to administration of said immune checkpoint inhibitor.

238. The kit of claims 233 to 235, wherein said first and/or second arenavirus particles are formulated for administration to a subject after administration of said immune checkpoint inhibitor.

239. The kit of any one of claims 201 to 238, wherein the second arenavirus particle comprises a first nucleotide sequence encoding a first human papillomavirus (HPV) antigen.

240. The kit of claim 239, wherein the first nucleotide sequence further encodes a second HPV antigen.

241. The kit of claim 239 or 240, wherein the first HPV antigen is selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof.

242. The kit of claim 239 or 240, wherein the first and the second HPV antigens are selected from the group consisting of:
(i) an HPV16 protein E6, or an antigenic fragment thereof;
(ii) an HPV16 protein E7, or an antigenic fragment thereof;
(iii) an HPV18 protein E6, or an antigenic fragment thereof; and
(iv) an HPV18 protein E7, or an antigenic fragment thereof,
and wherein the first and the second antigen are not the same.

243. The kit of any one of claims 201 to 242, wherein said first and second arenavirus particles are formulated for concurrent injection directly into the solid tumor.

244. The kit of any one of claims 201 to 242, wherein said first arenavirus particle is formulated for injection prior to said second arenavirus particle.

245. The kit of any one of claims 201 to 242, wherein said first arenavirus particle is formulated for injection subsequent to said second arenavirus particle.

246. The kit of any one of claims 201 to 245, wherein said kit further comprises an apparatus suitable for performing intravenous administration.

247. The kit of any one of claims 201 to 246, wherein said kit further comprises an injection apparatus suitable for performing an injection directly into a solid tumor.

248. The kit of any one of claims 201 to 247, which comprises multiple containers comprising the same first arenavirus particle.

249. The kit of any one of claims 201 to 247, which comprises multiple containers comprising multiple first arenavirus particles derived from different arenaviruses.

250. The kit of any one of claims 201 to 249, which comprises multiple containers comprising the same second arenavirus particle.

251. The kit of any one of claims 201 to 249, which comprises multiple containers comprising multiple second arenavirus particles derived from the same arenavirus, but expressing different tumor antigens or tumor-associated antigens or antigenic fragments thereof.

252. The kit of any one of claims 201 to 249, which comprises multiple containers comprising multiple second arenavirus particles derived from different arenaviruses, but expressing the same tumor antigen or tumor-associated antigen or antigenic fragment thereof.

253. The kit of any one of claims 201 to 249, which comprises multiple containers comprising multiple second arenavirus particles derived from different arenaviruses and expressing different tumor antigens or tumor-associated antigens or antigenic fragments thereof.

254. The method of any one of claims 1-78 or 148-200, wherein said LCMV is a tri-segmented, replication-competent LCMV vector encoding an artificial fusion protein of HPV16 E6 and E7 proteins.

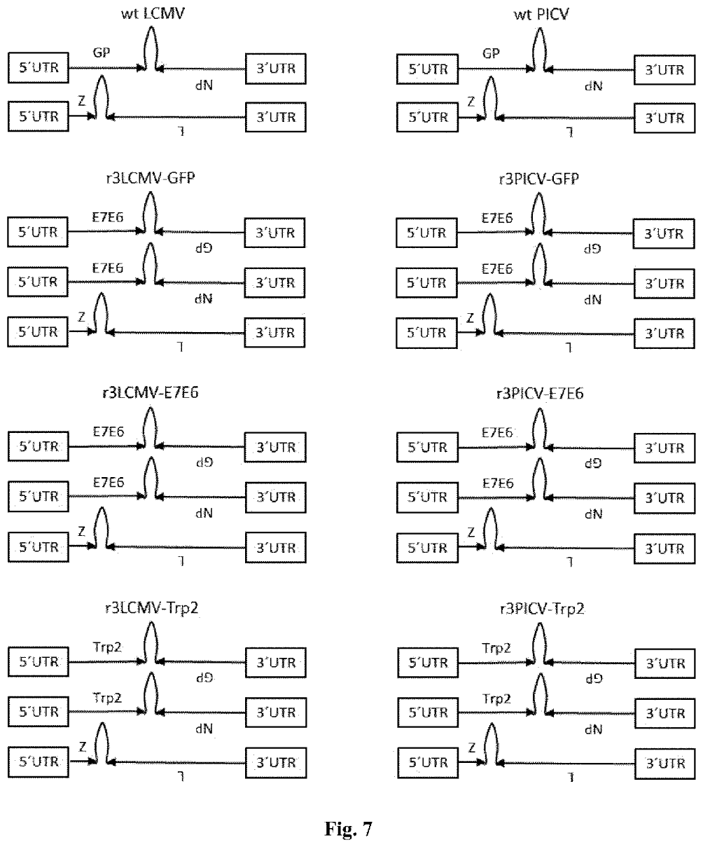

255. The method of any one of claims 1-78, 148-200 or 254, wherein said LCMV has a genomic structure as set forth in FIG. 7.

256. The method of any one of claims 1-78 or 148-200, wherein said PICV is a tri-segmented, replication-competent PICV vector encoding an artificial fusion protein of HPV16 E6 and E7 proteins.

257. The method of any one of claims 1-78, 148-200 or 256, wherein said PICV has a genomic structure as set forth in FIG. 7.

258. The method of any one of claims 1-78 or 148-200, wherein said arenavirus is an r3LCMV^{artificial} (art) construct (as described in WO/2016/075250).

259. The method of any one of claims 1-78 or 148-200, wherein said arenavirus is r3PICV^{artificial} (art) construct (as described in WO/2017/0198726).

260. The kit of any one of claims 79-147 or 201-253, wherein said LCMV is a tri-segmented, replication-competent LCMV vector encoding an artificial fusion protein of HPV16 E6 and E7 proteins.

261. The kit of any one of claims 79-147, 201-253 or 260, wherein said LCMV has a genomic structure as set forth in FIG. 7.

262. The kit of any one of claims 79-147 or 201-253, wherein said PICV is a tri-segmented, replication-competent PICV vector encoding an artificial fusion protein of HPV16 E6 and E7 proteins.

263. The kit of any one of claims 79-147, 201-253 or 262, wherein said PICV has a genomic structure as set forth in FIG. 7.

264. The kit of any one of claims 79-147, 201-253, or 260-261, wherein said arenavirus particle is r3LCMV^{artificial} (art) construct (as described in WO/2016/075250).

265. The kit of any one of claims 79-147, 201-253, or 262-263, wherein said arenavirus particle is r3PICV^{artificial} (art) construct (as described in WO/2017/0198726).


SEQUENCE LISTING (45 sequences)

SEQ ID NO: 1 - DNA, 7229 bp, Artificial Sequence
  Lymphocytic choriomeningitis virus clone 13 segment L (GenBank DQ361066.1)
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 2 - DNA, 3376 bp, Artificial Sequence
  Lymphocytic choriomeningitis virus segment S
cgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 3 - DNA, 3377 bp, Artificial Sequence
  Lymphocytic choriomeningitis virus clone 13 segment S (GenBank DQ361065.2)
gcgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 4 - DNA, 7205 bp, Artificial Sequence
  Lymphocytic choriomeningitis strain MP segment L
gcgcaccggggatcctaggcatttttgttgcgcattttgttgtgttatttgttgcacagcccttcatcgtgggaccttcacaaacaaaccaaaccaccagccatgggccaaggcaagtccaaagagggaagggatgccagcaatacgagcagagctgaaattctgccagacaccacctatctcggacctctgaactgcaagtcatgctggcagagatttgacagtttagtcagatgccatgaccactatctctgcagacactgcctgaacctcctgctgtcagtctccgacaggtgccctctctgcaaacatccattgccaaccaaactgaaaatatccacggccccaagctctccacccccttacgaggagtgacgccccgagccccaacaccgacacaaggaggccaccaacacaacgcccaacacggaacacacacacacacacccacacacacatccacacacacgcgcccccacaacgggggcgcccccccgggggtggccccccgggtgctcgggcggagccccacggagaggccaattagtcgatctcctcgaccaccgacttggtcagccagtcatcacaggacttgcccttaagtctgtacttgcccacaactgtttcatacatcaccgtgttctttgacttactgaaacatagcctacagtctttgaaagtgaaccagtcaggcacaagtgacagcggtaccagtagaatggatctatctatacacaactcttggagaattgtgctaatttccgacccctgtagatgctcaccagttctgaatcgatgtagaagaaggctcccaaggacgtcatcaaaatttccataaccctcgagctctgccaagaaaactctcatatccttggtctccagtttcacaacgatgttctgaacaaggcttcttccctcaaaaagagcacccattctcacagtcaagggcacaggctcccattcaggcccaatcctctcaaaatcaagggatctgatcccgtccagtattttccttgagcctatcagctcaagctcaagagagtcaccgagtatcagggggtcctccatatagtcctcaaactcttcagacctaatgtcaaaaacaccatcgttcaccttgaagatagagtctgatctcaacaggtggaggcattcgtccaagaaccttctgtccacctcacctttaaagaggtgagagcatgataggaactcagctacacctggaccttgtaactggcacttcactaaaaagatcaatgaaaacttcctcaaacaatcagtgttattctggttgtgagtgaaatctactgtaattgagaactctagcactccctctgtattatttatcatgtaatcccacaagtttctcaaagacttgaatgcctttggatttgtcaagccttgtttgattagcatggcagcattgcacacaatatctcccaatcggtaagagaaccatccaaatccaaattgcaagtcattcctaaacatgggcctctccatatttttgttcactacttttaagatgaatgattggaaaggccccaatgcttcagcgccatcttcagatggcatcatgtctttatgagggaaccatgaaaaacttcctagagttctgcttgttgctacaaattctcgtacaaatgactcaaaatacacttgttttaaaaagtttttgcagacatcccttgtactaacgacaaattcatcaacaaggcttgagtcagagcgctgatgggaatttacaagatcagaaaatagaacagtgtagtgttcgtccctcttccacttaactacatgagaaatgagcgataaagattctgaattgatatcgatcaatacgcaaaggtcaaggaatttgattctgggactccatctcatgttttttgagctcatatcagacatgaagggaagcagctgatcttcatagattttagggtacaatcgcctcacagattggattacatggtttaaacttatcttgtcctccagtagccttgaactctcaggcttccttgctacataatcacatgggttcaagtgcttgaggcttgagcttccctcattcttccctttcacaggttcagctaagacccaaacacccaactcaaaggaattactcagtgagatgcaaatatagtcccaaaggaggggcctcaagagactgatgtggtcgcagtgagcttctggatgactttgcctgtcacaaatgtacaacattatgccatcatgtctgtggattgctgtcacatgcgcatccatagctagatcctcaagcacttttctaatgtatagattgtccctatttttatttctcacacatctacttcccaaagttttgcaaagacctataaagcctgatgagatgcaactttgaaaggctgacttattgattgcttctgacagcaacttctgtgcacctcttgtgaacttactgcagagcttgttctggagtgtcttgattaatgatgggattctttcctcttggaaagtcattactgatggataaaccactttctgcctcaagaccattcttaatgggaacaactcattcaaattcagccaatttatgtttgccaattgacttagatcctcttcgaggccaaggatgtttcccaactgaagaatggcttcctttttatccctattgaagaggtctaagaagaattcttcattgaactcaccattcttgagcttatgatgtagtctccttacaagccttctcatgaccttcgtttcactaggacacaattcttcaataagcctttggattctgtaacctctagagccatccaaccaatccttgacatcagtattagtgttaagcaaaaatgggtccaagggaaagttggcatattttaagaggtctaatgttctcttctggatgcagtttaccaatgaaactggaacaccatttgcaacagcttgatcggcaattgtatctattgtttcacagagttggtgtggctctttacacttaacgttgtgtaatgctgctgacacaaattttgttaaaagtgggacctcttccccccacacataaaatctggatttaaattctgcagcaaatcgccccaccacacttttcggactgatgaacttgttaagcaagccactcaaatgagaatgaaattccagcaatacaaggacttcctcagggtcactatcaaccagttcactcaatctcctatcaaataaggtgatctgatcatcacttgatgtgtaagattctggtctctcaccaaaaatgacaccgatacaataattaatgaatctctcactgattaagccgtaaaagtcagaggcattatgtaagattccctgtcccatgtcaatgagactgcttatatgggaaggcactattcctaattcaaaatattctcgaaagattctttcagtcacagttgtctctgaacccctaagaagtttcagctttgatttgatatatgatttcatcattgcattcacaacaggaaaagggacctcaacaagtttgtgcatgtgccaagttaataaggtgctgatatgatcctttccggaacgcacatactggtcatcacccagtttgagattttgaaggagcattaaaaacaaaaatgggcacatcattggcccccatttgctatgatccatactgtagttcaacaacccctctcgcacattgatggtcattgatagaattgcattttcaaattctttgtcattgtttaagcatgaacctgagaagaagctagaaaaagactcaaaataatcctctatcaatcttgtaaacatttttgttctcaaatccccaatataaagttctctgtttcctccaacctgctctttgtatgataacgcaaacttcaaccttccggaatcaggaccaactgaagtgtatgacgttggtgactcctctgagtaaaaacataaattctttaaagcagcactcatgcattttgtcaatgatagagccttacttagagactcagaattactttccctttcactaattctaacatcttcttctagtttgtcccagtcaaacttgaaattcagaccttgtctttgcatgtgcctgtatttccctgagtatgcatttgcattcatttgcagtagaatcattttcatacacgaaaaccaatcaccctctgaaaaaaacttcctgcagaggttttttgccatttcatccagaccacattgttctttgacagctgaagtgaaatacaatggtgacagttctgtagaagtttcaatagcctcacagataaatttcatgtcatcattggtgagacaagatgggtcaaaatcttccacaagatgaaaagaaatttctgataagatgaccttccttaaatatgccattttacctgacaatatagtctgaaggtgatgcaatccttttgtattttcaaaccccacctcattttccccttcattggtcttcttgcttctttcataccgctttattgtggagttgaccttatcttctaaattcttgaagaaacttgtctcttcttccccatcaaagcatatgtctgctgagtcaccttctagtttcccagcttctgtttctttagagccgataaccaatctagagaccaactttgaaaccttgtactcgtaatctgagtggttcaatttgtacttctgctttctcatgaagctctctgtgatctgactcacagcactaacaagcaatttgttaaaatcatactctaggagccgttccccatttaaatgtttgttaacaaccacacttttgttgctggcaaggtctaatgctgttgcacacccagagttagtcatgggatccaagctattgagcctcttctcccctttgaaaatcaaagtgccattgttgaatgaggacaccatcatgctaaaggcctccagattgacacctggggttgtgcgctgacagtcaacttctttcccagtgaacttcttcatttggtcataaaaaacacactcttcctcaggggtgattgactctttagggttaacaaagaagccaaactcacttttaggctcaaagaatttctcaaagcatttaatttgatctgtcagcctatcaggggtttcctttgtgattaaatgacacaggtatgacacattcaacatgaacttgaactttgcgctcaacagtaccttttcaccagtcccaaaaacagttttgatcaaaaatctgagcaatttgtacactactttctcagcaggtgtgatcaaatcctccttcaacttgtccatcaatgatgtggatgagaagtctgagacaatggccatcactaaatacctaatgttttgaacctgtttttgattcctctttgttgggttggtgagcatgagtaataatagggttctcaatgcaatctcaacatcatcaatgctgtccttcaagtcaggacatgatctgatccatgagatcatggtgtcaatcatgttgtgcaacacttcatctgagaagattggtaaaaagaacctttttgggtctgcataaaaagagattagatggccattgggaccttgtatagaataacaccttgaggattctccagtcttttgatacagcaggtgatattcctcagagtccaattttatcacttggcaaaatacctctttacattccaccacttgataccttacagagcccaattggttttgtcttaatctagcaactgaacttgttttcatactgtttgtcaaagctagacagacagatgacaatcttttcaaactatgcatgttccttaattgttccgtattaggctggaaatcataatcttcaaactttgtataatacattataggatgagttccggacctcatgaaattctcaaactcaataaatggtatgtggcactcatgctcaagatgttcagacagaccatagtgcccaaaactaagtcccaccactgacaagcacctttgaacttttaaaatgaactcatttatggatgttctaaacaaatcctcaagagatacctttctatacgcctttgactttctcctgttccttagaagtctgatgaactcttccttggtgctatgaaagctcaccaacctatcattcacactcccatagcaacaaccaacccagtgcttatcattttttgaccctttgagtttagactgtttgatcaacgaagagagacacaagacatccaaattcagtaactgtctccttctggtgttcaataattttaaacttttaactttgttcaacatagagaggagcctctcatactcagtgctagtctcacttcctctctcataaccatgggtatctgctgtgataaatctcatcaaaggacaggattcaactgcctccttgcttagtgctgaaatgtcatcactgtcagcaagagtctcataaagctcagagaattccttaattaaatttccggggttgattttctgaaaactcctcttgagcttcccagtttccaagtctcttctaaacctgctgtaaagggagtttatgccaagaaccacatcatcgcagttcatgtttgggttgacaccatcatggcacattttcataatttcatcattgtgaaatgatcttgcatctttcaagattttcatagagtctataccggaacgcttatcaacagtggtcttgagagattcgcaaagtctgaagtactcagattcctcaaagactttctcatcttggctagaatactctaaaagtttaaacagaaggtctctgaacttgaaattcacccactctggcataaagctgttatcataatcacaccgaccatccactattgggaccaatgtgatacccgcaatggcaaggtcttctttgatacaggctagtttattggtgtcctctataaatttcttctcaaaactagctggtgtgcttctaacgaagcactcaagaagaatgagggaattgtcaatcagtttataaccatcaggaatgatcaaaggcagtcccgggcacacaatcccagactctattagaattgcctcaacagatttatcatcatggttgtgtatgcagccgctcttgtcagcactgtctatctctatacaacgcgacaaaagtttgagtccctctatcaataccattctgggttctctttgccctaaaaagttgagcttctgccttgacaacctctcatcttgttctatgtggtttaagcacaactctctcaactccgaaatagcctcatccattgcgcatcaaaaagcctaggatcctcggtgcg

SEQ ID NO: 5 - DNA, 3359 bp, Artificial Sequence
  Lymphocytic choriomeningitis strain MP segment S
cgcaccggggatcctaggctttttggattgcgctttcctcagctccgtcttgtgggagaatgggtcaaattgtgacgatgtttgaggctctgcctcacatcattgatgaggtcattaacattgtcattatcgtgcttattatcatcacgagcatcaaagctgtgtacaatttcgccacctgcgggatacttgcattgatcagctttctttttctggctggcaggtcctgtggaatgtatggtcttgatgggcctgacatttacaaaggggtttaccgattcaagtcagtggagtttgacatgtcttaccttaacctgacgatgcccaatgcatgttcggcaaacaactcccatcattatataagtatggggacttctggattggagttaaccttcacaaatgactccatcatcacccacaacttttgtaatctgacttccgccctcaacaagaggacttttgaccacacacttatgagtatagtctcaagtctgcacctcagcattagaggggtccccagctacaaagcagtgtcctgtgattttaacaatggcatcactattcaatacaacctgtcattttctaatgcacagagcgctctgagtcaatgtaagaccttcagggggagagtcctggatatgttcagaactgcttttggaggaaagtacatgaggagtggctggggctggacaggttcagatggcaagactacttggtgcagccagacaaactaccaatatctgattatacaaaacaggacttgggaaaaccactgcaggtacgcaggccctttcggaatgtctagaattctcttcgctcaagaaaagacaaggtttctaactagaaggcttgcaggcacattcacttggactttatcagactcatcaggagtggagaatccaggtggttactgcttgaccaagtggatgatcctcgctgcagagctcaagtgttttgggaacacagctgttgcaaagtgcaatgtaaatcatgatgaagagttctgtgatatgctacgactgattgattacaacaaggctgctttgagtaaattcaaagaagatgtagaatccgctctacatctgttcaagacaacagtgaattctttgatttctgatcagcttttgatgagaaatcacctaagagacttgatgggagtgccatactgcaattactcgaaattctggtatctagagcatgcaaagactggtgagactagtgtccccaagtgctggcttgtcagcaatggttcttatttgaatgaaacccatttcagcgaccaaattgagcaggaagcagataatatgatcacagaaatgctgagaaaggactacataaaaaggcaagggagtacccctctagccttgatggatctattgatgttttctacatcagcatatttgatcagcatctttctgcatcttgtgaggataccaacacacagacacataaagggcggctcatgcccaaaaccacatcggttaaccagcaagggaatctgtagttgtggtgcatttaaagtaccaggtgtggaaaccacctggaaaagacgctgaacagcagcgcctccctgactcaccacctcgaaagaggtggtgagtcagggaggcccagagggtcttagagtgttacgacatttggacctctgaagattaggtcatgtggtaggatattgtggacagttttcaggtcggggagccttgccttggaggcgctttcaaagatgatacagtccatgagtgcacagtgtggggtgacctctttctttttcttgtccctcactattccagtgtgcatcttgcatagccagccatatttgtcccagactttgtcctcatattctcttgaagcttctttagtcatctcaacatcgatgagcttaatgtctcttctgttttgtgaatctaggagtttcctgatgtcatcagatccctgacaacttaggaccattccctgtggaagagcacctattactgaagatgtcagcccaggttgtgcattgaagaggtcagcaaggtccatgccatgtgagtatttggagtcctgcttgaattgtttttgatcagtgggttctctatagaaatgtatgtactgcccattctgtggctgaaatattgctatttctaccgggtcattaaatctgccctcaatgtcaatccatgtaggagcgttagggtcaatacctcccatgaggtccttcagcaacattgtttggctgtagcttaagcccacctgaggtgggcccgctgccccaggcgctggtttgggtgagttggccataggcctctcatttgtcagatcaattgttgtgttctcccatgctctccctacaactgatgttctacaagctatgtatggccacccctcccctgaaagacagactttgtagaggatgttctcgtaaggattcctgtctccaacctgatcagaaacaaacatgttgagtttcttcttggccccaagaactgctttcaggagatcctcactgttgcttggcttaattaagatggattccaacatgttacccccatctaacaaggctgcccctgctttcacagcagcaccgagactgaaattgtagccagatatgttgatgctagactgctgctcagtgatgactcccaagactgggtgcttgtctttcagcctttcaaggtcacttaggttcgggtacttgactgtgtaaagcagcccaaggtctgtgagtgcttgcacaacgtcattgagtgaggtttgtgattgtttggccatacaagccattgttaagcttggcattgtgccgaattgattgttcagaagtgatgagtccttcacatcccagaccctcaccacaccatttgcactctgctgaggtctcctcattccaaccatttgcagaatctgagatctttggtcaagctgttgtgctgttaagttccccatgtagactccagaagttagaggcctttcagacctcatgattttagccttcagtttttcaaggtcagctgcaagggacatcagttcttctgcactaagcctccctacttttagaacattcttttttgatgttgactttaggtccacaagggaatacacagtttggttgaggcttctgagtctctgtaaatctttgtcatccctcttctctttcctcatgatcctctgaacattgctcacctcagagaagtctaatccattcagaaggctggtggcatccttgatcacagcagctttcacatctgatgtgaagccttgaagctctctcctcaatgcctgggtccattgaaagcttttaacttctttggacagagacattttgtcactcagtggatttccaagtcaaatgcgcaatcaaaatgcctaggatccactgtgcg

SEQ ID NO: 6 - protein, 558 aa, Artificial Sequence
  NP protein of the MP strain of LCMV
MSLSKEVKSFQWTQALRRELQGFTSDVKAAVIKDATSLLNGLDFSEVSNVQRIMRKEKRDDKDLQRLRSLNQTVYSLVDLKSTSKKNVLKVGRLSAEELMSLAADLEKLKAKIMRSERPLTSGVYMGNLTAQQLDQRSQILQMVGMRRPQQSANGVVRVWDVKDSSLLNNQFGTMPSLTMACMAKQSQTSLNDVVQALTDLGLLYTVKYPNLSDLERLKDKHPVLGVITEQQSSINISGYNFSLGAAVKAGAALLDGGNMLESILIKPSNSEDLLKAVLGAKKKLNMFVSDQVGDRNPYENILYKVCLSGEGWPYIACRTSVVGRAWENTTIDLTNERPMANSPKPAPGAAGPPQVGLSYSQTMLLKDLMGGIDPNAPTWIDIEGRFNDPVEIAIFQPQNGQYIHFYREPTDQKQFKQDSKYSHGMDLADLFNAQPGLTSSVIGALPQGMVLSCQGSDDIRKLLDSQNRRDIKLIDVEMTKEASREYEDKVWDKYGWLCKMHTGIVRDKKKKEVTPHCALMDCIIFESASKARLPDLKTVHNILPHDLIFRGPNVVTL

SEQ ID NO: 7 - protein, 498 aa, Artificial Sequence
  GP protein of the MP strain of LCMV
MGQIVTMFEALPHIIDEVINIVIIVLIIITSIKAVYNFATCGILALISFLFLAGRSCGMYGLDGPDIYKGVYRFKSVEFDMSYLNLTMPNACSANNSHHYISMGTSGLELTFTNDSIITHNFCNLTSALNKRTFDHTLMSIVSSLHLSIRGVPSYKAVSCDFNNGITIQYNLSFSNAQSALSQCKTFRGRVLDMFRTAFGGKYMRSGWGWTGSDGKTTWCSQTNYQYLIIQNRTWENHCRYAGPFGMSRILFAQEKTRFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVSNGSYLNETHFSDQIEQEADNMITEMLRKDYIKRQGSTPLALMDLLMFSTSAYLISIFLHLVRIPTHRHIKGGSCPKPHRLTSKGICSCGAFKVPGVETTWKRR

SEQ ID NO: 8 - protein, 2209 aa, Artificial Sequence
  L protein of the MP strain of LCMV
MDEAISELRELCLNHIEQDERLSRQKLNFLGQREPRMVLIEGLKLLSRCIEIDSADKSGCIHNHDDKSVEAILIESGIVCPGLPLIIPDGYKLIDNSLILLECFVRSTPASFEKKFIEDTNKLACIKEDLAIAGITLVPIVDGRCDYDNSFMPEWVNFKFRDLLFKLLEYSSQDEKVFEESEYFRLCESLKTTVDKRSGIDSMKILKDARSFHNDEIMKMCHDGVNPNMNCDDVVLGINSLYSRFRRDLETGKLKRSFQKINPGNLIKEFSELYETLADSDDISALSKEAVESCPLMRFITADTHGYERGSETSTEYERLLSMLNKVKSLKLLNTRRRQLLNLDVLCLSSLIKQSKLKGSKNDKHWVGCCYGSVNDRLVSFHSTKEEFIRLLRNRRKSKAYRKVSLEDLFRTSINEFILKVQRCLSVVGLSFGHYGLSEHLEHECHIPFIEFENFMRSGTHPIMYYTKFEDYDFQPNTEQLRNMHSLKRLSSVCLALTNSMKTSSVARLRQNQLGSVRYQVVECKEVFCQVIKLDSEEYHLLYQKTGESSRCYSIQGPNGHLISFYADPKRFFLPIFSDEVLHNMIDTMISWIRSCPDLKDSIDDVEIALRTLLLLMLTNPTKRNQKQVQNIRYLVMAIVSDFSSTSLMDKLKEDLITPAEKVVYKLLRFLIKTVFGTGEKVLLSAKFKFMLNVSYLCHLITKETPDRLTDQIKCFEKFFEPKSEFGFFVNPKESITPEEECVFYDQMKKFTGKEVDCQRTTPGVNLEAFSMMVSSFNNGTLIFKGEKRLNSLDPMTNSGCATALDLASNKSVVVNKHLNGERLLEYDFNKLLVSAVSQITESFMRKQKYKLNHSDYEYKVSKLVSRLVIGSKETEAGKLEGDSADICFDGEEETSFFKNLEDKVNSTIKRYERSKKTNEGENEVGFENTKGLHHLQTILSGKMAYLRKVILSEISFHLVEDFDPSCLTNDDMKFICEAIETSTELSPLYFTSAVKEQCGLDEMAKNLCRKFFSEGDWFSCMKMILLQMNANAYSGKYRHMQRQGLNFKFDWDKLEEDVRISERESNSESLSKALSLTKCMSAALKNLCFYSEESPTSYTSVGPDSGRLKFALSYKEQVGGNRELYIGDLRTKMFTRLIEDYFESFSSFFSGSCLNNDKEFENAILSMTINVREGLLNYSMDHSKWGPMMCPFLFLMLLQNLKLGDDQYVRSGKDHISTLLTWHMHKLVEVPFPVVNAMMKSYIKSKLKLLRGSETTVTERIFREYFELGIVPSHISSLIDMGQGILHNASDFYGLISERFINYCIGVIFGERPESYTSSDDQITLFDRRLSELVDSDPEEVLVLLEFHSHLSGLLNKFISPKSVVGRFAAEFKSRFYVWGEEVPLLTKFVSAALHNVKCKEPHQLCETIDTIADQAVANGVPVSLVNCIQKRTLDLLKYANFPLDPFLLNTNTDVKDWLDGSRGYRIQRLIEELCPSETKVMRRLVRRLHHKLKNGEFNEEFFLDLFNRDKKEAILQLGNILGLEEDLSQLANINWLNLNELFPLRMVLRQKVVYPSVMTFQEERIPSLIKTLQNKLCSKFTRGAQKLLSEAINKSAFQSCISSGFIGLCKTLGSRCVRNKNRDNLYIRKVLEDLAMDAHVTAIHRHDGIMLYICDRQSHPEAHCDHISLLRPLLWDYICISLSNSFELGVWVLAEPVKGKNEGSSSLKHLNPCDYVARKPESSRLLEDKISLNHVIQSVRRLYPKIYEDQLLPFMSDMSSKNMRWSPRIKFLDLCVLIDINSESLSLISHVVKWKRDEHYTVLFSDLVNSHQRSDSSLVDEFVVSTRDVCKNFLKQVYFESFVREFVATSRTLGSFSWFPHKDMMPSEDGAEALGPFQSFILKVVNKNMERPMFRNDLQFGFGWFSYRLGDIVCNAAMLIKQGLTNPKAFKSLRNLWDYMINNTEGVLEFSITVDFTHNQNNTDCLRKFSLIFLVKCQLQGPGVAEFLSCSHLFKGEVDRRFLDECLHLLRSDSIFKVNDGVFDIRSEEFEDYMEDPLILGDSLELELIGSRKILDGIRSLDFERIGPEWEPVPLTVRMGALFEGRSLVQNIVVKLETKDMRVFLAELEGYGNFDDVLGSLLLHRFRTGEHLQGSEISTILQELCIDRSILLVPLSLVPDWFTFKDCRLCFSKSKNTVMYETVVGKYRLKGKSCDDWLTKSVVEEID

SEQ ID NO: 9 - protein, 90 aa, Artificial Sequence
  Z protein of the MP strain of LCMV
MGQGKSKEGRDASNTSRAEILPDTTYLGPLNCKSCWQRFDSLVRCHDHYLCRHCLNLLLSVSDRCPLCKHPLPTKLKISTAPSSPPPYEE

SEQ ID NO: 10 - DNA, 7115 bp, Artificial Sequence
  Junin virus Candid No.1 L segment
gcgcaccggggatcctaggcgtaacttcatcattaaaatctcagattctgctctgagtgtgacttactgcgaagaggcagacaaatgggcaactgcaacggggcatccaagtctaaccagccagactcctcaagagccacacagccagccgcagaatttaggagggtagctcacagcagtctatatggtagatataactgtaagtgctgctggtttgctgataccaatttgataacctgtaatgatcactacctttgtttaaggtgccatcagggtatgttaaggaattcagatctctgcaatatctgctggaagcccctgcccaccacaatcacagtaccggtggagccaacagcaccaccaccataggcagactgcacagggtcagacccgaccccccggggggcccccatggggaccccccgtgggggaaccccgggggtgatgcgccattagtcaatgtctttgatctcgactttgtgcttcagtggcctgcatgtcacccctttcaatctgaactgcccttggggatctgatatcagcaggtcatttaaagatctgctgaatgccaccttgaaatttgagaattccaaccagtcaccaaatttatcaagtgaacggatcaactgctctttgtgtagatcataaacgaggacaaagtcctcttgctgaaataatattgtttgtgatgttgtttttagataaggccatagttggcttaataaggtttccacactatcaatgtcctctagtgctccaattgccttgactatgacatccccagacaactcaactctatatgttgacaacctttcattacctctgtaaaagataccctctttcaagacaagaggttctcctgggttatctggcccaatgaggtcatatgcatacttgttacttagttcagaataaaagtcaccaaagttgaacttaacatggctcagaatattgtcatcatttgtcgcagcgtagcctgcatcaataaacaagccagctaggtcaaagctctcatggcctgtgaacaatggtaggctagcgataaccagtgcaccatccaacaatgagtggcttccctcagacccagaaacacattgactcattgcatccacattcagctctaattcaggggtaccgacatcatccactcctagtgaactgacaatggtgtaactgtacaccatctttcttctaagtttaaattttgtcgaaactcgtgtgtgttctacttgaatgatcaattttagtttcacagcttcttggcaagcaacattgcgcaacacagtgtgcaggtccatcatgtcttcctgaggcaacaaggagatgttgtcaacagagacaccctcaaggaaaaccttgatattatcaaagctagaaactacataacccattgcaatgtcttcaacaaacattgctcttgatactttattattcctaactgacaaggtaaaatctgtgagttcagctagatctacttgactgtcatcttctagatctagaacttcattgaaccaaaagaaggatttgagacacgatgttgacatgactagtgggtttatcatcgaagataagacaacttgcaccatgaagttcctgcaaacttgctgtgggctgatgccaacttcccaatttgtatactctgactgtctaacatgggctgaagcgcaatcactctgtttcacaatataaacattattatctcttactttcaataagtgacttataatccctaagttttcattcatcatgtctagagccacacagacatctagaaacttgagtcttccactatccaaagatctgttcacttgaagatcattcataaagggtgccaaatgttcttcaaatagtttggggtaatttcttcgtatagaatgcaatacatggttcatgcctaattggtcttctatctgtcgtactgctttgggtttaacagcccagaagaaattcttattacataagaccagaggggcctgtggactcttaatagcagaaaacacccactcccctaactcacaggcatttgtcagcaccaaagagaagtaatcccacaaaattggtttagaaaattggttaacttctttaagtgatttttgacagtaaataactttaggctttctctcacaaattccacaaagacatggcattattcgagtaaatatgtcctttatatacagaaatccgcctttaccatccctaacacacttactccccatactcttacaaaacccaatgaagcctgaggcaacagaagactgaaatgcagatttgttgattgactctgccaagatcttcttcacgccttttgtgaaatttcttgacagcctggactgtattgtccttatcaatgttggcatctcttctttctctaacactcttcgacttgtcatgagtttggtcctcaagaccaacctcaagtccccaaagctcgctaaattgacccatctgtagtctagagtttgtctgatttcatcttcactacacccggcatattgcaggaatccggataaagcctcatcccctcccctgcttatcaagttgataaggttttcctcaaagattttgcctctcttaatgtcattgaacactttcctcgcgcagttccttataaacattgtctccttatcatcagaaaaaatagcttcaattttcctctgtagacggtaccctctagacccatcaacccagtctttgacatcttgttcttcaatagctccaaacggagtctctctgtatccagagtatctaatcaattggttgactctaatggaaatctttgacactatatgagtgctaaccccattagcaatacattgatcacaaattgtgtctatggtctctgacagttgtgttggagttttacacttaacgttgtgtagagcagcagacacaaacttggtgagtaaaggagtctcttcacccatgacaaaaaatcttgacttaaactcagcaacaaaagttcctatcacactctttgggctgataaacttgtttaatttagaagataagaattcatggaagcacaccatttccagcagttctgtcctgtcttgaaacttttcatcactaaggcaaggaatttttataaggctaacctggtcatcgctggaggtataagtgacaggtatcacatcatacaataagtcaagtgcataacacagaaattgttcagtaattagcccatataaatctgatgtgttgtgcaagattccctggcccatgtccaagacagacattatatggctggggacctggtcccttgactgcagatactggtgaaaaaactcttcaccaacactagtacagtcacaacccattaaacctaaagatctcttcaatttccctacacagtaggcttctgcaacattaattggaacttcaacgaccttatgaagatgccatttgagaatgttcattactggttcaagattcacctttgttctatctctgggattcttcaattctaatgtgtacaaaaaagaaaggaaaagtgctgggctcatagttggtccccatttggagtggtcatatgaacaggacaagtcaccattgttaacagccattttcatatcacagattgcacgttcgaattccttttctgaattcaagcatgtgtatttcattgaactacccacagcttctgagaagtcttcaactaacctggtcatcagcttagtgttgaggtctcccacatacagttctctatttgagccaacctgctccttataacttagtccaaatttcaagttccctgtatttgagctgatgcttgtgaactctgtaggagagtcgtctgaatagaaacataaattccgtagggctgcatttgtaaaataacttttgtctagcttatcagcaatggcttcagaattgctttccctggtactaagccgaacctcatcctttagtctcagaacttcactggaaaagcccaatctagatctacttctatgctcataactacccaatttctgatcataatgtccttgaattaaaagatacttgaagcattcaaagaattcatcttcttggtaggctattgttgtcaaattttttaataacaaacccaaagggcagatgtcctgcggtgcttcaagaaaataagtcaatttaaatggagatagataaacagcatcacataactctttatacacatcagacctgagcacatctggatcaaaatccttcacctcatgcattgacacctctgctttaatctctctcaacactccaaaaggggcccacaatgactcaagagactctcgctcatcaacagatggattttttgatttcaacttggtgatctcaacttttgtcccctcactattagccatcttggctagtgtcatttgtacgtcatttctaataccctcaaaggcccttacttgatcctctgttaaactctcatacatcactgataattcttcttgattggttctggttcttgaaccggtgctcacaagacctgttagattttttaatattaagtagtccatggaatcaggatcaagattatacctgccttttgttttaaacctctcagccatagtagaaacgcatgttgaaacaagtttctccttatcataaacagaaagaatatttccaagttcgtcgagcttggggattaccacacttttattgcttgacagatccagagctgtgctagtgatgttaggcctgtagggattgcttttcagttcacctgtaactttaagtcttcctctattgaagagagaaatgcagaaggacaaaatctctttacacactcctggaatttgagtatctgaggaagtcttagcctctttggaaaagaatctgtccaatcctcttatcatggtgtcctcttgttccagtgttagactcccacttagaggggggtttacaacaacacaatcaaacttgactttgggctcaataaacttctcaaaacactttatttgatctgtcaggcgatcaggtgtctctttggttaccaagtgacacagataactaacatttaatagatatttaaaccttcttgcaaagtaaagatctgcatcttccccttcacccaaaattgtctggaaaagttccacagccatcctctgaatcagcacctctgatccagacatgcagtcgacccttaactttgacatcaaatccacatgatggatttgatttgcatatgccatcaagaaatatcttagaccttgtaaaaatgtctggttccttttggaaggggaacagagtacagctaacactaacaatcttaatattggccttgtcattgtcatgagttcgtggctaaaatccaaccagctggtcatttcctcacacatttcaattaacacatcctccgaaaatataggcaggaaaaatctctttggatcacagtaaaaagagccttgttcttccaataccccattgatggatagatagatagaatagcaccttgacttctcacctgttttttggtaaaacaagagaccaaatgtattctttgtcagatgaaatctttgtacataacactctcttagtctaacattcccaaaatatctagaatactctctttcattgattaacaatcgggaggaaaatgatgtcttcatcgagttgaccaatgcaagggaaatggaggacaaaatcctaaataatttcttctgctcaccttccactaagctgctgaatggctgatgtctacagattttctcaaattccttgttaatagtatatctcatcactggtctgtcagaaacaagtgcctgagctaaaatcatcaagctatccatatcagggtgttttattagtttttccagctgtgaccagagatcttgatgagagttcttcaatgttctggaacacgcttgaacccacttggggctggtcatcaatttcttccttattagtttaatcgcctccagaatatctagaagtctgtcattgactaacattaacatttgtccaacaactattcccgcatttcttaaccttacaattgcatcatcatgcgttttgaaaagatcacaaagtaaattgagtaaaactaagtccagaaacagtaaagtgtttctcctggtgttgaaaacttttagacctttcactttgttacacacggaaagggcttgaagataacacctctctacagcatcaatagatatagaattctcatctgactggctttccatgttgacttcatctattggatgcaatgcgatagagtagactacatccatcaacttgtttgcacaaaaagggcagctgggcacatcactgtctttgtggcttcctaataagatcaagtcatttataagcttagacttttgtgaaaatttgaatttccccaactgcttgtcaaaaatctccttcttaaaccaaaaccttaactttatgagttcttctcttatgacagattctctaatgtctcctctaaccccaacaaagagggattcatttaacctctcatcataacccaaagaattctttttcaagcattcgatgttttctaatcccaagctctggttttttgtgttggacaaactatggatcaatcgctggtattcttgttcttcaatattaatctcttgcataaattttgatttctttaggatgtcgatcagcaaccaccgaactctttcaacaacccaatcagcaaggaatctattgctgtagctagatctgccatcaaccacaggaaccaacgtaatccctgcccttagtaggtcggactttaggtttaagagctttgacatgtcactcttccattttctctcaaactcatcaggattgaccctaacaaaggtttccaataggatgagtgttttccctgtgagtttgaagccatccggaatgacttttggaagggtgggacatagtatgccatagtcagacaggatcacatcaacaaacttctgatctgaattgatctgacaggcgtgtgcctcacaggactcaagctctactaaacttgacagaagtttgaacccttccaacaacagagagctggggtgatgttgagataaaaagatgtccctttggtatgctagctcctgtctttctggaaaatgctttctaataaggctttttatttcatttactgattcctccatgctcaagtgccgcctaggatcctcggtgcg

SEQ ID NO: 11 - DNA, 3411 bp, Artificial Sequence
  Junin virus Candid No.1 S segment
gcgcaccggggatcctaggcgattttggttacgctataattgtaactgttttctgtttggacaacatcaaaaacatccattgcacaatggggcagttcattagcttcatgcaagaaataccaacctttttgcaggaggctctgaacattgctcttgttgcagtcagtctcattgccatcattaagggtatagtgaacttgtacaaaagtggtttattccaattctttgtattcctagcgcttgcaggaagatcctgcacagaagaagctttcaaaatcggactgcacactgagttccagactgtgtccttctcaatggtgggtctcttttccaacaatccacatgacctacctttgttgtgtaccttaaacaagagccatctttacattaaggggggcaatgcttcatttcagatcagctttgatgatattgcagtattgttgccacagtatgatgttataatacaacatccagcagatatgagctggtgttccaaaagtgatgatcaaatttggttgtctcagtggttcatgaatgctgtgggacatgattggcatctagacccaccatttctgtgtaggaaccgtgcaaagacagaaggcttcatctttcaagtcaacacctccaagactggtgtcaatggaaattatgctaagaagtttaagactggcatgcatcatttatatagagaatatcctgacccttgcttgaatggcaaactgtgcttaatgaaggcacaacctaccagttggcctctccaatgtccactcgaccacgttaacacattacacttccttacaagaggtaaaaacattcaacttccaaggaggtccttgaaagcattcttctcctggtctttgacagactcatccggcaaggatacccctggaggctattgtctagaagagtggatgctcgtagcagccaaaatgaagtgttttggcaatactgctgtagcaaaatgcaatttgaatcatgactctgaattctgtgacatgttgaggctctttgattacaacaaaaatgctatcaaaaccctaaatgatgaaactaagaaacaagtaaatctgatggggcagacaatcaatgccctgatatctgacaatttattgatgaaaaacaaaattagggaactgatgagtgtcccttactgcaattacacaaaattttggtatgtcaaccacacactttcaggacaacactcattaccaaggtgctggttaataaaaaacaacagctatttgaacatctctgacttccgtaatgactggatattagaaagtgacttcttaatttctgaaatgctaagcaaagagtattcggacaggcagggtaaaactcctttgactttagttgacatctgtatttggagcacagtattcttcacagcgtcactcttccttcacttggtgggtataccctcccacagacacatcaggggcgaagcatgccctttgccacacaggttgaacagcttgggtggttgcagatgtggtaagtaccccaatctaaagaaaccaacagtttggcgtagaggacactaagacctcctgagggtccccaccagcccgggcactgcccgggctggtgtggccccccagtccgcggcctggccgcggactggggaggcactgcttacagtgcataggctgccttcgggaggaacagcaagctcggtggtaatagaggtgtaggttcctcctcatagagcttcccatctagcactgactgaaacattatgcagtctagcagagcacagtgtggttcactggaggccaacttgaagggagtatccttttccctctttttcttattgacaaccactccattgtgatatttgcataagtgaccatatttctcccagacctgttgatcaaactgcctggcttgttcagatgtgagcttaacatcaaccagtttaagatctcttcttccatggaggtcaaacaacttcctgatgtcatcggatccttgagtagtcacaaccatgtctggaggcagcaagccgatcacgtaactaagaactcctggcattgcatcttctatgtccttcattaagatgccgtgagagtgtctgctaccatttttaaaccctttctcatcatgtggttttctgaagcagtgaatgtactgcttacctgcaggttggaataatgccatctcaacagggtcagtggctggtccttcaatgtcgagccaaagggtgttggtggggtcgagtttccccactgcctctctgatgacagcttcttgtatctctgtcaagttagccaatctcaaattctgaccgtttttttccggctgtctaggaccagcaactggtttccttgtcagatcaatacttgtgttgtcccatgacctgcctgtgatttgtgatctagaaccaatataaggccaaccatcgccagaaagacaaagtttgtacaaaaggttttcataaggatttctattgcctggtttctcatcaataaacatgccttctcttcgtttaacctgaatggttgattttatgagggaagagaagttttctggggtgactctgattgtttccaacatgtttccaccatcaagaatagatgctccagcctttactgcagctgaaagactgaagttgtaaccagaaatattgatggagctttcatctttagtcacaatctgaaggcagtcatgttcctgagtcagtctgtcaaggtcacttaagtttggatacttcacagtgtatagaagcccaagtgaggttaaagcttgtatgacactgttcattgtctcacctccttgaacagtcatgcatgcaattgtcaatgcaggaacagagccaaactgattgtttagctttgaagggtctttaacatcccatatcctcaccacaccatttcccccagtcccttgctgttgaaatcccagtgttctcaatatctctgatcttttagcaagttgtgactgggacaagttacccatgtaaaccccctgagagcctgtctctgctcttcttatcttgttttttaatttctcaaggtcagacgccaactccatcagttcatccctccccagatctcccaccttgaaaactgtgtttcgttgaacactcctcatggacatgagtctgtcaacctctttattcaggtccctcaacttgttgaggtcttcttccccctttttagtctttctgagtgcccgctgcacctgtgccacttggttgaagtcgatgctgtcagcaattagcttggcgtccttcaaaacatctgacttgacagtctgagtgaattggctcaaacctctccttaaggactgagtccatctaaagcttggaacctccttggagtgtgccatgccagaagttctggtgattttgatctagaatagagttgctcagtgaaagtgttagacactatgcctaggatccactgtgcg

SEQ ID NO: 12 - protein, 558 aa, Artificial Sequence
  NP protein of the Clone 13 strain of LCMV (GenBank Accession No. ABC96002.1; GI86440166)
MSLSKEVKSFQWTQALRRELQSFTSDVKAAVIKDATNLLNGLDFSEVSNVQRIMRKEKRDDKDLQRLRSLNQTVHSLVDLKSTSKKNVLKVGRLSAEELMSLAADLEKLKAKIMRSERPQASGVYMGNLTTQQLDQRSQILQIVGMRKPQQGASGVVRVWDVKDSSLLNNQFGTMPSLTMACMAKQSQTPLNDVVQALTDLGLLYTVKYPNLNDLERLKDKHPVLGVITEQQSSINISGYNFSLGAAVKAGAALLDGGNMLESILIKPSNSEDLLKAVLGAKRKLNMFVSDQVGDRNPYENILYKVCLSGEGWPYIACRTSIVGRAWENTTIDLTSEKPAVNSPRPAPGAAGPPQVGLSYSQTMLLKDLMGGIDPNAPTWIDIEGRFNDPVEIAIFQPQNGQFIHFYREPVDQKQFKQDSKYSHGMDLADLFNAQPGLTSSVIGALPQGMVLSCQGSDDIRKLLDSQNRKDIKLIDVEMTREASREYEDKVWDKYGWLCKMHTGIVRDKKKKEITPHCALMDCIIFESASKARLPDLKTVHNILPHDLIFRGPNVVTL

SEQ ID NO: 13 - protein, 498 aa, Artificial Sequence
  GP protein of the Clone 13 strain of LCMV (GenBank Accession No. ABC96001.2; GI116563462)
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 14 - protein, 2210 aa, Artificial Sequence
  L protein of the Clone 13 strain of LCMV (GenBank Accession No. ABC96004.1; GI86440169)
MDEIISELRELCLNYIEQDERLSRQKLNFLGQREPRMVLIEGLKLLSRCIEIDSADKSGCTHNHDDKSVETILVESGIVCPGLPLIIPDGYKLIDNSLILLECFVRSTPASFEKKFIEDTNKLACIREDLAVAGVTLVPIVDGRCDYDNSFMPEWANFKFRDLLFKLLEYSNQNEKVFEESEYFRLCESLKTTIDKRSGMDSMKILKDARSTHNDEIMRMCHEGINPNMSCDDVVFGINSLFSRFRRDLESGKLKRNFQKVNPEGLIKEFSELYENLADSDDILTLSREAVESCPLMRFITAETHGHERGSETSTEYERLLSMLNKVKSLKLLNTRRRQLLNLDVLCLSSLIKQSKFKGLKNDKHWVGCCYSSVNDRLVSFHSTKEEFIRLLRNRKKSKVFRKVSFEELFRASISEFIAKIQKCLLVVGLSFEHYGLSEHLEQECHIPFTEFENFMKIGAHPIMYYTKFEDYNFQPSTEQLKNIQSLRRLSSVCLALTNSMKTSSVARLRQNQIGSVRYQVVECKEVFCQVIKLDSEEYHLLYQKTGESSRCYSIQGPDGHLISFYADPKRFFLPIFSDEVLYNMIDIMISWIRSCPDLKDCLTDIEVALRTLLLLMLTNPTKRNQKQVQSVRYLVMAIVSDFSSTSLMDKLREDLITPAEKVVYKLLRFLIKTIFGTGEKVLLSAKFKFMLNVSYLCHLITKETPDRLTDQIKCFEKFFEPKSQFGFFVNPKEAITPEEECVFYEQMKRFTSKEIDCQHTTPGVNLEAFSLMVSSFNNGTLIFKGEKKLNSLDPMTNSGCATALDLASNKSVVVNKHLNGERLLEYDFNKLLVSAVSQITESFVRKQKYKLSHSDYEYKVSKLVSRLVIGSKGEETGRSEDNLAEICFDGEEETSFFKSLEEKVNTTIARYRRGRRANDKGDGEKLTNTKGLHHLQLILTGKMAHLRKVILSEISFHLVEDFDPSCLTNDDMKFICEAVEGSTELSPLYFTSVIKDQCGLDEMAKNLCRKFFSENDWFSCMKMILLQMNANAYSGKYRHMQRQGLNFKFDWDKLEEDVRISERESNSESLSKALSLTQCMSAALKNLCFYSEESPTSYTSVGPDSGRLKFALSYKEQVGGNRELYIGDLRTKMFTRLIEDYFESFSSFFSGSCLNNDKEFENAILSMTINVREGFLNYSMDHSKWGPMMCPFLFLMFLQNLKLGDDQYVRSGKDHVSTLLTWHMHKLVEVPFPVVNAMMKSYVKSKLKLLRGSETTVTERIFRQYFEMGIVPSHISSLIDMGQGILHNASDFYGLLSERFINYCIGVIFGERPEAYTSSDDQITLFDRRLSDLVVSDPEEVLVLLEFQSHLSGLLNKFISPKSVAGRFAAEFKSRFYVWGEEVPLLTKFVSAALHNVKCKEPHQLCETIDTIADQAIANGVPVSLVNSIQRRTLDLLKYANFPLDPFLLNTNTDVKDWLDGSRGYRIQRLIEELCPNETKVVRKLVRKLHHKLKNGEFNEEFFLDLFNRDKKEAILQLGDLLGLEEDLNQLADVNWLNLNEMFPLRMVLRQKVVYPSVMTFQEERIPSLIKTLQNKLCSKFTRGAQKLLSEAINKSAFQSCISSGFIGLCKTLGSRCVRNKNRENLYIKKLLEDLTTDDHVTRVCNRDGITLYICDKQSHPEAHRDHICLLRPLLWDYICISLSNSFELGVWVLAEPTKGKNNSENLTLKHLNPCDYVARKPESSRLLEDKVNLNQVIQSVRRLYPKIFEDQLLPFMSDMSSKNMRWSPRIKFLDLCVLIDINSESLSLISHVVKWKRDEHYTVLFSDLANSHQRSDSSLVDEFVVSTRDVCKNFLKQVYFESFVREFVATTRTLGNFSWFPHKEMMPSEDGAEALGPFQSFVSKVVNKNVERPMFRNDLQFGFGWFSYRMGDVVCNAAMLIRQGLTNPKAFKSLKDLWDYMLNYTKGVLEFSISVDFTHNQNNTDCLRKFSLIFLVRCQLQNPGVAELLSCSHLFKGEIDRRMLDECLHLLRTDSVFKVNDGVFDIRSEEFEDYMEDPLILGDSLELELLGSKRILDGIRSIDFERVGPEWEPVPLTVKMGALFEGRNLVQNIIVKLETKDMKVFLAGLEGYEKISDVLGNLFLHRFRTGEHLLGSEISVILQELCIDRSILLIPLSLLPDWFAFKDCRLCFSKSRSTLMYETVGGRFRLKGRSCDDWLGGSVAEDID

SEQ ID NO: 15 - protein, 90 aa, Artificial Sequence
  Z protein of the Clone 13 strain of LCMV (GenBank Accession No. ABC96003.1; GI86440168)
MGQGKSREEKGTNSTNRAEILPDTTYLGPLSCKSCWQKFDSLVRCHDHYLCRHCLNLLLSVSDRCPLCKYPLPTRLKISTAPSSPPPYEE

SEQ ID NO: 16 - protein, 498 aa, Artificial Sequence
  GP protein of the WE strain of LCMV
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 17 - DNA, 35 bp, Artificial Sequence
  WE specific primer
aatcgtctctaaggatgggtcagattgtgacaatg

SEQ ID NO: 18 - DNA, 35 bp, Artificial Sequence; identical to SEQ ID NO: 17
  WE specific fusion-primer carrying an overhang complementary to the WET-specific primer

SEQ ID NO: 19 - DNA, 37 bp, Artificial Sequence
  WE specific primer
ctcggtgatcatgttatctgcttcttgttcgatttga

SEQ ID NO: 20 - DNA, 34 bp, Artificial Sequence
  WE specific fusion-primer complementary to the WE-sequence
aatcgtctctttctttatctcctcttccagatgg

SEQ ID NO: 21 - DNA, 23 bp, Artificial Sequence
  Primer specific for LCMV NP
ggctcccagatctgaaaactgtt

SEQ ID NO: 22 - DNA, 22 bp, Artificial Sequence
  NP- and GP-specific primers; NP-specific same as in RT reaction, GP- specific 5′
gctggcttgtcactaatggctc

SEQ ID NO: 23 - DNA, 3377 bp, Artificial Sequence
  Lymphocytic choriomeningitis virus clone 13 wildtype - Segment S with WE - GP
gcgcaccggggatcctaggctttttggattgcgctttcctctagatcaactgggtgtcaggccctatcctacagaaggatgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 24 - DNA, 3422 bp, Artificial Sequence
  Pichinde virus  wildtype - Segment S, (Reference Sequence GenBank EF529746.1)
gcgcaccggggatcctaggcataccttggacgcgcatattacttgatcaaagatgggacaagttgtgactttgatccagtctatacccgaagtcctgcaggaggtgttcaatgtcgccttaatcattgtctcaaccctatgcatcatcaaaggatttgtcaatctgatgagatgtggcctattccaactcatcaccttcctcattttggctggcagaagttgtgatggcatgatgattgataggaggcacaatctcacccacgttgagttcaacctcacaagaatgtttgacaacttgccacaatcatgtagcaagaacaacacacatcattactacaaaggaccatctaacacaacatggggaattgaactcactttgacaaacacatccattgcaaatgaaactactggaaacttttccaacatcagaagccttgcatatggtaacattagtaattgtgataagacagaagaagcaggtcacacattaaaatggttgcttaatgagttacacttcaatgtgctccatgtcactcgtcatgtaggtgccagatgcaaaacagttgagggtgctggggtgttgatccagtacaacttgacagttggggatagaggaggtgaggttggcagacatcttattgcgtcgcttgctcaaatcattggggacccaaaaattgcgtgggttggaaaatgtttcaataactgtagtggagggtcttgcagactaacaaactgtgaaggtgggacacattacaatttcctgatcatacagaacaccacatgggaaaatcactgtacatatactccaatggcaacaataaggatggctctccaaaaaactgcttatagttctgtgagcaggaaactccttggctttttcacttgggacttgagtgactctactgggcaacatgtcccaggtggttactgtttggagcaatgggctattgtttgggctggaataaaatgttttgataacactgtgatggcaaaatgcaacaaagatcacaatgaagaattttgcgatacgatgaggttatttgatttcaatcagaatgctatcaaaaccttacaacttaatgttgagaattcgttgaatctctttaaaaagactatcaacggacttatttctgactcacttgtgattagaaacagtctcaaacagcttgccaaaatcccttattgcaactatacaaaattttggtacatcaatgataccatcacagggagacattctttaccgcagtgttggttagttcacaatggctcgtacctcaatgaaacgcattttaagaatgattggttgtgggagagccagaatctgtacaatgaaatgctgataaaagaatatgaagaaagacaaggtaagactccactagcattgacagacatttgcttctggtctttggtgttttacaccatcacagtgtttctccacttagttggaatacccactcataggcacatcattggtgatggctgtccgaagccacataggattactaggaactctctttgcagctgtgggtattataaaatcccaaagaaaccctacaaatgggtgagactgggtaaataagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 25 - DNA, 7058 bp, Artificial Sequence
  Pichinde virus  wildtype - Segment L, (Reference Sequence GenBank EF529747.1)
gcgcaccggggatcctaggcatctttgggtcacgcttcaaatttgtccaatttgaacccagctcaagtcctggtcaaaacttgggatgggactcagatatagcaaagaggtcaggaagagacatggcgacgaagatgtggtgggaagggtccccatgaccctcaatctaccacagggcctgtatggcaggttcaactgcaaatcttgctggttcgtcaacaaaggtctcatcaggtgcaaagaccactatctgtgtcttgggtgcttaaccaaaatgcactccagaggcaatctctgcgagatatgcggccactcactgccaaccaagatggagttcctagaaagcccctctgcaccaccctacgagccataaaccagggcccctgggcgcacccccctccgggggtgcgcccgggggcccccggccccatggggccggttgtttactcgatctccactgactcattgtcctcaaacaactttcgacacctgattcccttgatcttgaagggtcctgtctcgtctgcaatcataacagatcctagagtcttacttcttattatactaaagtgaccacaattcaaccaatctttggcatcatgcaacatgtgttcaaacacttcggggaaattttcaatcatgagtcttaaatcctgctcgttcatacttattcccttgttgtgagactgtgcacttgaaaggtactgaaaaaggttggcaataaatcttggccttttctcaggttctaatgcttccagtgcaatgatgaccacctttgagtctaagttcacttccaatctagaaaccactctgttgccctctttgatcaacccaccctctaaaatgaggggttgcatcccaacatcaggaccaatcaacttataggaaaatttgtttttcaaatccttgaaacgatttttcaaatctattctcaccttctggaacacagttgaccttgacttgaagtgaatgtcttgaccttccaatagatcattgaagtctagaacatcttttccgttgatgagaggattcagaaccaaaagtgacacaccatccagacttatgtgattcccggaagattgagaaacataatactcaacagaatgggggttcaacaataggtaaccatcagagtccaatgagtccagcaatgactccctttcaataagaaatcttaattttaatatgtaattggtagacctctcatatctaaatttgtggctcactctcttatgagaaaatgttaggttgagctcaatgggaatgacctcagaaggtgatgctaaaatgagttgttcaatgttctcatagttatctctattcacccagtcaagttcattaataaatacactaatgttcaaattaacacaggacaaaatcagtttgctgcttacaaagccaacatccaagtcatccagattcattgtcctagaagtgttattctttttgcagtcacaaatgaactgggttaattgtttcagatcatgttgtgcattgtttggcaacaattcaagctcaccaaaccaaaaatatttcttgaactgagatgttgacataatcacaggcaccaacattgactcaaacaaaatctgtatcaagaaatttgtgcacacttcttctggttcaaggttgaatcctctctccagtggatgagactctctgctatgggacattgcaagctcattttgctttacaatatacaattcttctctgcgatgttttataatatgactaacaataccaagacattctgatgttatatcaattgccacacaaaggtctaagaactttatcctctgaacccatgatagcctcagcatattcaaatcagacaggaaaggggatatgtgttcatcaaatagtgtagggaagttcctcctgattgagtaaagtatgtggttgatgcccaccttgtcctcaagctcagaatgtgtgcttggttttattggccagaagtgattgggattgtttaggtgagtgactatcttgggtacttcagctttttgaaacacccagttacccaactcgcaagcattggttaacacaagagcaaaataatcccaaattaagggtctggagtactcacttacttcaccaagtgctgctttacaataaacacctttgcgctgattacaaaagtgacaatcacggtgtaagataatcttgcttgtaatatccctgatatacttaaatcctcctttcccatctcttacacattttgagcccatacttttgcaaactcctatgaatcctgatgctatgctgctctgaaaagctgatttgttgatagcatcagccaaaatcttcttagcccctctgacatagttctttgataatttggactgtacggatttgacaagactgggtatttcttctcgctgcacagttcttgttgtgctcattaacttagtacgaagcaccaatctgagatcaccatgaacccttaaatttaaccacctaatattaagagcatcctcaatagcctcagtctcgacatcacaagtctctaataactgttttaagcagtcatccggtgattgctgaagagttgttacaatataactttcttccagggctccagactgtattttgtaaaatattttcctgcatgcctttctgattattgaaagtagcagatcatcaggaaatagtgtctcaattgatcgctgaagtctgtaccctctcgacccattaacccaatcgagtacatccatttcttccaggcacaaaaatggatcatttggaaacccactatagattatcatgctatttgttcgttttgcaatggcccctacaacctctattgacaccccgttagcaacacattggtccagtattgtgtcaattgtatctgcttgctgattgggtgctttagcctttatgttgtgtagagctgcagcaacaaactttgtaaggagggggacttcttgtgaccaaatgaagaatctcgatttgaactcacttgcaaaggtccccacaactgttttagggctcacaaacttgttgagtttgtctgatagaaagtagtgaaactccatacagtccaataccaattcaacattcaactcatctctgtccttaaatttgaaaccctcattcaaggataacatgatctcatcatcactcgaagtatatgagatgaaccgtgctccataacaaagctccaatgcgtaattgatgaactgctcagtgattagaccatataagtcagaggtgttgtgtaggatgccctgacccatatctaagactgaagagatgtgtgatggtaccttgcccttctcaaagtacccaaacataaattcctctgcaattgtgcacccccctttatccatcatacccaacccccttttcaagaaacctttcatgtatgcctcaacgacattgaagggcacttccaccatcttgtgaatgtgccatagcaatatgttgatgactgcagcattgggaacttctgacccatctttgagtttgaactcaagaccttttaataatgcggcaaagataaccggcgacatgtgtggcccccattttgaatggtccattgacaccgcaagaccactttgcctaacaactgacttcatgtctaataatgctctctcaaactctttctcgttgttcagacaagtatacctcatgttttgcataagggattcagagtaatcctcaatgagtctggttgtgagtttagtatttaaatcaccgacataaagctccctgttgccacccacctgttctttataagaaagaccaaatttcaatctccctacattggtggatacaccagacctctctgtgggagactcatctgaatagaaacagagatttcgtaaggatgagttggtaaaaaagctttgatccaatcttttagctatcgattcagaattgctctctcttgagcttatacgtgatgtctctctaatttgtagtgctgcatctgtgaacccaagtctgcttctacttttgtgatcatatcttccgactcgattatcataatcgcttgcaatgagaatgtatttaaagcactcaaaataatcagcttctttgtacgccttcaatgtgaggttctttattaaaaactccagaggacacggattcattagtctgtctgcaaagtacactgatctagcagtgacatcctcatagatcaagtttacaagatcctcatacacttctgctgaaaacaggctgtaatcaaaatcctttacatcatgaagtgaagtctctcttttgatgacaaccattgtcgatttgggccataatctctctagtggacatgaagtcttaaggttggttttgacattggtgtcaaccttagacaatacttttgcaactctggtctcaatttctttaagacagtcaccctgatcttctgatagtaactcttcaactccatcaggctctattgactccttttttatttggatcaatgatgacaacctcttcagaatcttgaaatttacctcctttggatctaacttgtatttacccttagttttgaaatgttcaatcatttccacaacaacagcagacacaatggaagagtaatcatattcagtgatgacctcaccaacttcattgagttttggaaccaccacacttttgttgctggacatatccaaggctgtacttgtgaaggagggagtcatagggtcacaaggaagcaggggtttcacttccaatgagctactgttaaatagtgatagacaaacactaagtacatccttattcaaccccggccttccctcacatttggattccagctttttaccaagtagtctctctatatcatgcaccatcttctcttcttcctcagtaggaagttccatactattagaagggttgaccaagactgaatcaaactttaactttggttccaagaacttctcaaaacatttgatttgatcagttaatctatcaggggtttctttggttataaaatggcataaataggagacattcaaaacaaacttaaagatcttagccatatcttcctctctggagttgctgagtaccagaagtatcaaatcatcaataagcattgctgtctgccattctgaaggtgttagcataacgactttcaatttctcaaacaattctttaaaatgaacttcatttacaaaggccataatgtaatatctaaagccttgcaagtaaacttgaatacgcttggaaggggtgcacagtatgcagagaataagtcgtctgagtaaatcagaaacagaatccaagaggggttgggacataaagtccaaccaggataacatctccacacaagtcctttgaatcacatctgcactaaagatcggtaagaaaaatctcttgggatcacagtaaaaagacgcttttgtttcatacaaacccccacttttggatctataagcaacagcataacacctggacctctcccctgtcttctggtacagtagtgtgagagaacctccttctccaaatcgctggaagaaaacttcgtcacagtaaaccttcccataaaactcatcagcattgttcaccttcatcttaggaactgctgctgtcttcatgctattaatgagtgacaaactcaaacttgacaatgttttcagcaattcctcaaactcactttcgcccatgatggtataatcaggctgccctcttcctggcctacccccacacatacactgtgactttgtcttgtattgaagacagggtttagcaccccattcatctaacactgatgttttcagattgaagtaatattcaacatcaggttcccgtagaagagggagaatgtcatcaaggggaagttcaccacagaccgagctcagtctcttcttagccttctctaaccagttggggtttttaatgaattttttagtgatttgttccatcaggaagtcgacattaatcaacctgtcatttacagacggtaacccttgcattaggagcacctctctgaacacagcacctggagaagacttgtccaagtcacacaaaatgttgtacatgataaggtccagaaccaacatggtgttcctccttgtgttaaaaaccttttgagacttaattttgttgcatattgaaagtactctaaaatattctctgctttcagttgatgaatgcttgacctcagattgcctgagttggcctattatgcccaaaatgtgtactgagcaaaactcacataatctgatttctgatttaggtacatctttgacagaacattggataaattcatggttctgaagtctagaaatcatatcttccctatctgtagcctgcagtttcctatcgagttgaccagcaagttgcaacattttaaattgctgaaagatttccatgatttttgttctacattgatctgttgtcagtttattattaatgccagacattaatgccttttccaacctcactttgtaaggaagtcccctttcctttacagcaagtagtgactccagaccgagactctgattttctaaggatgagagggaacttataaggcgttcgtactccaactcctcaacttcttcaccagatgtccttaatccatccatgagttttaaaagcaaccaccgaagtctctctaccacccaatcaggaacaaattctacataataactggatctaccgtcaataacaggtactaaggttatgttctgtctcttgagatcagaactaagctgcaacagcttcaaaaagtcctggttgtatttcttctcaaatgcttcttgactggtcctcacaaacacttccaaaagaatgaggacatctccaaccatacagtaaccatctggtgtaacatccggcaatgtaggacatgttactctcaactccctaaggatagcattgacagtcatctttgtgttgtgtttgcaggagtgtttcttgcatgaatccacttccactagcatggacaaaagcttcaggccctctatcgtgatggccctatctttgacttgtgcaagaacgttgtttttctgttcagatagctcttcccattcgggaacccattttctgactatgtctttaagttcgaaaacgtattcctccatgatcaagaaatgcctaggatcctcggtgcg

SEQ ID NO: 26 - DNA, 2648 bp, Artificial Sequence
  Genomic sequence of LCMV vector (r3LCMV)encoding HPV16 E7E6 fusion S Segment 1 (containing NP)
gcgcaccggggatcctaggctttttggattgcgctttcctctagatcaactgggtgtcaggccctatcctacagaaggatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 27 - DNA, 2468 bp, Artificial Sequence
  Genomic sequence of LCMV vector (r3LCMV)encoding HPV16 E7E6 fusion S Segment 2 (containing GP)
gcgcaccggggatcctaggctttttggattgcgctttcctctagatcaactgggtgtcaggccctatcctacagaaggatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 28 - DNA, 2663 bp, Artificial Sequence
  Genomic sequence of Pichinde vector (r3PICV)encoding HPV16 E7E6 fusion S Segment 1 (containing NP)
gcgcaccggggatcctaggcataccttggacgcgcatattacttgatcaaagatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 29 - DNA, 2504 bp, Artificial Sequence
  Genomic sequence of Pichinde vector (r3PICV)encoding HPV16 E7E6 fusion S Segment 2 (containing GP)
gcgcaccggggatcctaggcataccttggacgcgcatattacttgatcaaagatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 30 - DNA, 3434 bp, Artificial Sequence
  Genomic sequence of LCMV vector (r3LCMV)encoding TRP2 fusion S Segment 1 (containing NP)
gcgcaccggggatcctaggctttttggattgcgctttcctctagatcaactgggtgtcaggccctatcctacagaaggatgggccttgtgggatgggggcttctgctgggttgtctgggctgtggaattctgctcagagccagggctcagtttcccagagtctgcatgaccttggatggggtgctgaacaaggaatgctgcccccctctgggtccagaggcaaccaacatctgtggatttctggagggcagggggcagtgtgcagaggtgcaaacagacaccagaccctggagtggcccttacatcctcagaaaccaggatgacagggagcaatggccaagaaaattcttcaacaggacatgcaaatgcacaggaaactttgctggttacaattgtggaggctgcaagtttggctggactggcccagactgcaacaggaagaagccagccatcctcagaaggaacatccattccctgactgcccaggagagggagcagttcttgggagccttggacctggccaagaagagcatccatccagactatgtgatcaccacacaacactggctggggctgctgggacccaatgggacccagccccagattgccaactgcagtgtgtatgacttttttgtgtggctccattattattctgtgagagacacattgttgggtccaggaagaccctacaaggccattgatttctctcaccaagggcctgcctttgtcacctggcacaggtaccatctgttgtggctggaaagagaactccagagactcactggcaatgagtcctttgccttgccctactggaactttgcaactgggaagaatgagtgtgatgtgtgcacagatgagctgcttggagcagcaagacaagatgacccaacactgatcagcaggaactcaagattctcaacctgggagattgtgtgtgacagcttggatgactacaacaggagggtcacactgtgcaatggaacctatgaaggtttgctgagaagaaacaaagtgggcagaaacaatgagaaactgccaaccttgaaaaatgtgcaagattgcctgtctctccagaagtttgacagccctcccttcttccagaactccaccttcagcttcaggaatgcactggaagggtttgacaaagcagatggaacactggactctcaagtcatgaaccttcacaacttggctcactccttcctgaatgggaccaatgccttgccacactcagcagccaatgaccctgtgtttgtggtcctccactctttcacagatgccatctttgatgagtggctgaagagaaacaacccttccacagatgcctggcctcaggaactggcacccattggtcacaacagaatgtacaacatggtccccttcttcccacctgtgaccaatgaggagctcttcctcactgcagagcaacttggctacaattatgcagttgatctgtcagaggaagaagctccagtttggtccacaactctctcagtggtcattggaatcctgggagcttttgtcttgctcttggggttgctggcttttcttcaatacagaaggctgaggaaaggctatgctcccttgatggagacaggtctcagcagcaagagatacacagaggaagcctagagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 31 - DNA, 3254 bp, Artificial Sequence
  Genomic sequence of LCMV vector (r3LCMV)encoding TRP2 fusion S Segment 2 (containing GP)
gcgcacagtggatcctaggcatttgattgcgcatttgtcttgagaaaccattgagcaacaagatgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctgagaccctctgggcctccctgactctccacctctttcgaggtggagagtcagggaggcgctgttctctaggcttcctctgtgtatctcttgctgctgagacctgtctccatcaagggagcatagcctttcctcagccttctgtattgaagaaaagccagcaaccccaagagcaagacaaaagctcccaggattccaatgaccactgagagagttgtggaccaaactggagcttcttcctctgacagatcaactgcataattgtagccaagttgctctgcagtgaggaagagctcctcattggtcacaggtgggaagaaggggaccatgttgtacattctgttgtgaccaatgggtgccagttcctgaggccaggcatctgtggaagggttgtttctcttcagccactcatcaaagatggcatctgtgaaagagtggaggaccacaaacacagggtcattggctgctgagtgtggcaaggcattggtcccattcaggaaggagtgagccaagttgtgaaggttcatgacttgagagtccagtgttccatctgctttgtcaaacccttccagtgcattcctgaagctgaaggtggagttctggaagaagggagggctgtcaaacttctggagagacaggcaatcttgcacatttttcaaggttggcagtttctcattgtttctgcccactttgtttcttctcagcaaaccttcataggttccattgcacagtgtgaccctcctgttgtagtcatccaagctgtcacacacaatctcccaggttgagaatcttgagttcctgctgatcagtgttgggtcatcttgtcttgctgctccaagcagctcatctgtgcacacatcacactcattcttcccagttgcaaagttccagtagggcaaggcaaaggactcattgccagtgagtctctggagttctctttccagccacaacagatggtacctgtgccaggtgacaaaggcaggcccttggtgagagaaatcaatggccttgtagggtcttcctggacccaacaatgtgtctctcacagaataataatggagccacacaaaaaagtcatacacactgcagttggcaatctggggctgggtcccattgggtcccagcagccccagccagtgttgtgtggtgatcacatagtctggatggatgctcttcttggccaggtccaaggctcccaagaactgctccctctcctgggcagtcagggaatggatgttccttctgaggatggctggcttcttcctgttgcagtctgggccagtccagccaaacttgcagcctccacaattgtaaccagcaaagtttcctgtgcatttgcatgtcctgttgaagaattttcttggccattgctccctgtcatcctggtttctgaggatgtaagggccactccagggtctggtgtctgtttgcacctctgcacactgccccctgccctccagaaatccacagatgttggttgcctctggacccagaggggggcagcattccttgttcagcaccccatccaaggtcatgcagactctgggaaactgagccctggctctgagcagaattccacagcccagacaacccagcagaagcccccatcccacaaggcccatccttctgtaggatagggcctgacacccagttgatctagaggaaagcgcaatccaaaaagcctaggatccccggtgcg

SEQ ID NO: 32 - DNA, 3449 bp, Artificial Sequence
  Genomic sequence of Pichinde vector (r3PICV)encoding TRP2 fusion S Segment 1 (containing NP)
gcgcaccggggatcctaggcataccttggacgcgcatattacttgatcaaagatgggccttgtgggatgggggcttctgctgggttgtctgggctgtggaattctgctcagagccagggctcagtttcccagagtctgcatgaccttggatggggtgctgaacaaggaatgctgcccccctctgggtccagaggcaaccaacatctgtggatttctggagggcagggggcagtgtgcagaggtgcaaacagacaccagaccctggagtggcccttacatcctcagaaaccaggatgacagggagcaatggccaagaaaattcttcaacaggacatgcaaatgcacaggaaactttgctggttacaattgtggaggctgcaagtttggctggactggcccagactgcaacaggaagaagccagccatcctcagaaggaacatccattccctgactgcccaggagagggagcagttcttgggagccttggacctggccaagaagagcatccatccagactatgtgatcaccacacaacactggctggggctgctgggacccaatgggacccagccccagattgccaactgcagtgtgtatgacttttttgtgtggctccattattattctgtgagagacacattgttgggtccaggaagaccctacaaggccattgatttctctcaccaagggcctgcctttgtcacctggcacaggtaccatctgttgtggctggaaagagaactccagagactcactggcaatgagtcctttgccttgccctactggaactttgcaactgggaagaatgagtgtgatgtgtgcacagatgagctgcttggagcagcaagacaagatgacccaacactgatcagcaggaactcaagattctcaacctgggagattgtgtgtgacagcttggatgactacaacaggagggtcacactgtgcaatggaacctatgaaggtttgctgagaagaaacaaagtgggcagaaacaatgagaaactgccaaccttgaaaaatgtgcaagattgcctgtctctccagaagtttgacagccctcccttcttccagaactccaccttcagcttcaggaatgcactggaagggtttgacaaagcagatggaacactggactctcaagtcatgaaccttcacaacttggctcactccttcctgaatgggaccaatgccttgccacactcagcagccaatgaccctgtgtttgtggtcctccactctttcacagatgccatctttgatgagtggctgaagagaaacaacccttccacagatgcctggcctcaggaactggcacccattggtcacaacagaatgtacaacatggtccccttcttcccacctgtgaccaatgaggagctcttcctcactgcagagcaacttggctacaattatgcagttgatctgtcagaggaagaagctccagtttggtccacaactctctcagtggtcattggaatcctgggagcttttgtcttgctcttggggttgctggcttttcttcaatacagaaggctgaggaaaggctatgctcccttgatggagacaggtctcagcagcaagagatacacagaggaagcctaggccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 33 - DNA, 3290 bp, Artificial Sequence
  Genomic sequence of Pichinde vector (r3PICV)encoding TRP2 fusion S Segment 2 (containing GP)
gcgcaccggggatcctaggcataccttggacgcgcatattacttgatcaaagatgggccttgtgggatgggggcttctgctgggttgtctgggctgtggaattctgctcagagccagggctcagtttcccagagtctgcatgaccttggatggggtgctgaacaaggaatgctgcccccctctgggtccagaggcaaccaacatctgtggatttctggagggcagggggcagtgtgcagaggtgcaaacagacaccagaccctggagtggcccttacatcctcagaaaccaggatgacagggagcaatggccaagaaaattcttcaacaggacatgcaaatgcacaggaaactttgctggttacaattgtggaggctgcaagtttggctggactggcccagactgcaacaggaagaagccagccatcctcagaaggaacatccattccctgactgcccaggagagggagcagttcttgggagccttggacctggccaagaagagcatccatccagactatgtgatcaccacacaacactggctggggctgctgggacccaatgggacccagccccagattgccaactgcagtgtgtatgacttttttgtgtggctccattattattctgtgagagacacattgttgggtccaggaagaccctacaaggccattgatttctctcaccaagggcctgcctttgtcacctggcacaggtaccatctgttgtggctggaaagagaactccagagactcactggcaatgagtcctttgccttgccctactggaactttgcaactgggaagaatgagtgtgatgtgtgcacagatgagctgcttggagcagcaagacaagatgacccaacactgatcagcaggaactcaagattctcaacctgggagattgtgtgtgacagcttggatgactacaacaggagggtcacactgtgcaatggaacctatgaaggtttgctgagaagaaacaaagtgggcagaaacaatgagaaactgccaaccttgaaaaatgtgcaagattgcctgtctctccagaagtttgacagccctcccttcttccagaactccaccttcagcttcaggaatgcactggaagggtttgacaaagcagatggaacactggactctcaagtcatgaaccttcacaacttggctcactccttcctgaatgggaccaatgccttgccacactcagcagccaatgaccctgtgtttgtggtcctccactctttcacagatgccatctttgatgagtggctgaagagaaacaacccttccacagatgcctggcctcaggaactggcacccattggtcacaacagaatgtacaacatggtccccttcttcccacctgtgaccaatgaggagctcttcctcactgcagagcaacttggctacaattatgcagttgatctgtcagaggaagaagctccagtttggtccacaactctctcagtggtcattggaatcctgggagcttttgtcttgctcttggggttgctggcttttcttcaatacagaaggctgaggaaaggctatgctcccttgatggagacaggtctcagcagcaagagatacacagaggaagcctaggccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 34 - protein, 255 aa, Artificial Sequence
  E7E6 Fusion protein
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVGPICSQKPHQKRTAMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINGQKPLCPEEKQRHLDKKQRFHNIRGRWTGRCMSCCRSSRTRRETQL

SEQ ID NO: 35 - protein, 517 aa, Artificial Sequence
  murine TRP2 protein (Reference Sequence NM_010024)
MGLVGWGLLLGCLGCGILLRARAQFPRVCMTLDGVLNKECCPPLGPEATNICGFLEGRGQCAEVQTDTRPWSGPYILRNQDDREQWPRKFFNRTCKCTGNFAGYNCGGCKFGWTGPDCNRKKPAILRRNIHSLTAQEREQFLGALDLAKKSIHPDYVITTQHWLGLLGPNGTQPQIANCSVYDFFVWLHYYSVRDTLLGPGRPYKAIDFSHQGPAFVTWHRYHLLWLERELQRLTGNESFALPYWNFATGKNECDVCTDELLGAARQDDPTLISRNSRFSTWEIVCDSLDDYNRRVTLCNGTYEGLLRRNKVGRNNEKLPTLKNVQDCLSLQKFDSPPFFQNSTFSFRNALEGFDKADGTLDSQVMNLHNLAHSFLNGTNALPHSAANDPVFVVLHSFTDAIFDEWLKRNNPSTDAWPQELAPIGHNRMYNMVPFFPPVTNEELFLTAEQLGYNYAVDLSEEEAPVWSTTLSVVIGILGAFVLLLGLLAFLQYRRLRKGYAPLMETGLSSKRYTEEA

SEQ ID NO: 36 - protein, 239 aa, Artificial Sequence
  GFP (reporter antigen)
MVSKGEELFTGVVPILVELDGDVNGHKFSVSGEGEGDATYGKLTLKFICTTGKLPVPWPTLVTTFTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 37 - protein, 558 aa, Artificial Sequence; identical to SEQ ID NO: 12
  LCMV cl13 Nucleoprotein Sequence

SEQ ID NO: 38 - protein, 498 aa, Artificial Sequence; identical to SEQ ID NO: 13
  LCMV cl13 Glycoprotein Sequence

SEQ ID NO: 39 - protein, 498 aa, Artificial Sequence; identical to SEQ ID NO: 16
  LCMV WE Glycoprotein Sequence

SEQ ID NO: 40 - protein, 2210 aa, Artificial Sequence; identical to SEQ ID NO: 14
  LCMV cl13 Polymerase Sequence

SEQ ID NO: 41 - protein, 90 aa, Artificial Sequence; identical to SEQ ID NO: 15
  LCMV cl13 Z protein Sequence

SEQ ID NO: 42 - protein, 561 aa, Artificial Sequence
  Pichinde Nucleoprotein Sequence
MSDNIPSFRWVQSLRRGLSNWTHPVKADVLSDTRALLSALDFHKVAQVQRMMRKDKRTDSDLTKLRDMNKEVDALMNMRSIQRDNVLKVGGLAKEELMELASDLDKLRKKVTRTESLSQPGVYGGNLTNTQLEQRAEILRSMGFANARPTGNRDGVVKIWDIKDNTLLINQFGSMPALTIACMTEQGGEQLNDVVQALSALGLLYTVKFPNMTDLEKLTQQHSALKIISNEPSAINISGYNLSLSAAVKAAACMIDGGNMLETIQVKPSMFSTLIKSLLQIKNREGMFVSTTPGQRNPYENLLYKICLSGDGWPYIGSRSQVQGRAWDNTTVDLDSKPSAIQPPVRNGGSPDLKQIPKEKEDTVVSSIQMLDSKATTWIDIEGTPNDPVEMAIYQPDTGNYIHCYRFPHDEKSFKEQSKYSHGLLLKDLADAQPGLISSIIRHLPQNMVFTAQGSDDIISLFEMHGRRDLKVLDVKLSAEQARTFEDEIWERYNLLCTKHKGLVIKKKKKGAAQTTANPHCALLDTIMFDATVTGWVRDQKPMRCLPIDTLYRNNTDLINL

SEQ ID NO: 43 - protein, 508 aa, Artificial Sequence
  Pichinde Glycoprotein Sequence
MGQVVTLIQSIPEVLQEVFNVALIIVSTLCIIKGFVNLMRCGLFQLITFLILAGRSCDGMMIDRRHNLTHVEFNLTRMFDNLPQSCSKNNTHHYYKGPSNTTWGIELTLTNTSIANETTGNFSNIRSLAYGNISNCDKTEEAGHTLKWLLNELHFNVLHVTRHVGARCKTVEGAGVLIQYNLTVGDRGGEVGRHLIASLAQIIGDPKIAWVGKCFNNCSGGSCRLTNCEGGTHYNFLIIQNTTWENHCTYTPMATIRMALQKTAYSSVSRKLLGFFTWDLSDSTGQHVPGGYCLEQWAIVWAGIKCFDNTVMAKCNKDHNEEFCDTMRLFDFNQNAIKTLQLNVENSLNLFKKTINGLISDSLVIRNSLKQLAKIPYCNYTKFWYINDTITGRHSLPQCWLVHNGSYLNETHFKNDWLWESQNLYNEMLIKEYEERQGKTPLALTDICFWSLVFYTITVFLHLVGIPTHRHIIGDGCPKPHRITRNSLCSCGYYKIPKKPYKWVRLGK

SEQ ID NO: 44 - protein, 2194 aa, Artificial Sequence
  Pichinde Polymerase Sequence
MEEYVFELKDIVRKWVPEWEELSEQKNNVLAQVKDRAITIEGLKLLSMLVEVDSCKKHSCKHNTKMTVNAILRELRVTCPTLPDVTPDGYCMVGDVLILLEVFVRTSQEAFEKKYNQDFLKLLQLSSDLKRQNITLVPVIDGRSSYYVEFVPDWVVERLRWLLLKLMDGLRTSGEEVEELEYERLISSLSSLENQSLGLESLLAVKERGLPYKVRLEKALMSGINNKLTTDQCRTKIMEIFQQFKMLQLAGQLDRKLQATDREDMISRLQNHEFIQCSVKDVPKSEIRLCEFCSVHILGIIGQLRQSEVKHSSTESREYFRVLSICNKIKSQKVFNTRRNTMLVLDLIMYNILCDLDKSSPGAVFREVLLMQGLPSVNDRLINVDFLMEQITKKFIKNPNWLEKAKKRLSSVCGELPLDDILPLLREPDVEYYFNLKTSVLDEWGAKPCLQYKTKSQCMCGGRPGRGQPDYTIMGESEFEELLKTLSSLSLSLINSMKTAAVPKMKVNNADEFYGKVYCDEVFFQRFGEGGSLTLLYQKTGERSRCYAVAYRSKSGGLYETKASFYCDPKRFFLPIFSADVIQRTCVEMLSWLDFMSQPLLDSVSDLLRRLILCILCTPSKRIQVYLQGFRYYIMAFVNEVHFKELFEKLKVVMLTPSEWQTAMLIDDLILLVLSNSREEDMAKIFKFVLNVSYLCHFITKETPDRLTDQIKCFEKFLEPKLKFDSVLVNPSNSMELPTEEEEKMVHDIERLLGKKLESKCEGRPGLNKDVLSVCLSLFNSSSLEVKPLLPCDPMTPSFTSTALDMSSNKSVVVPKLNEVGEVITEYDYSSIVSAVVVEMIEHFKTKGKYKLDPKEVNFKILKRLSSLIQIKKESIEPDGVEELLSEDQGDCLKEIETRVAKVLSKVDTNVKTNLKTSCPLERLWPKSTMVVIKRETSLHDVKDFDYSLFSAEVYEDLVNLIYEDVTARSVYFADRLMNPCPLEFLIKNLTLKAYKEADYFECFKYILIASDYDNRVGRYDHKSRSRLGFTDAALQIRETSRISSRESNSESIAKRLDQSFFTNSSLRNLCFYSDESPTERSGVSTNVGRLKFGLSYKEQVGGNRELYVGDLNTKLTTRLIEDYSESLMQNMRYTCLNNEKEFERALLDMKSVVRQSGLAVSMDHSKWGPHMSPVIFAALLKGLEFKLKDGSEVPNAAVINILLWHIHKMVEVPFNVVEAYMKGFLKRGLGMMDKGGCTIAEEFMFGYFEKGKVPSHISSVLDMGQGILHNTSDLYGLITEQFINYALELCYGARFISYTSSDDEIMLSLNEGFKFKDRDELNVELVLDCMEFHYFLSDKLNKFVSPKTVVGTFASEFKSRFFIWSQEVPLLTKFVAAALHNIKAKAPNQQADTIDTILDQCVANGVSIEVVGAIAKRTNSMIIYSGFPNDPFLCLEEMDVLDWVNGSRGYRLQRSIETLFPDDLLLSIIRKACRKIFYKIQSGALEESYIVTTLQQSPDDCLKQLLETCDVETEAIEDALNIRWLNLRVHGDLRLVLRTKLMSTTRTVQREEIPSLVKSVQSKLSKNYVRGAKKILADAINKSAFQSSIASGFIGVCKSMGSKCVRDGKGGFKYIRDITSKIILHRDCHFCNQRKGVYCKAALGEVSEYSRPLIWDYFALVLTNACELGNWVFQKAEVPKIVTHLNNPNHFWPIKPSTHSELEDKVGINHILYSIRRNFPTLFDEHISPFLSDLNMLRLSWVQRIKFLDLCVAIDITSECLGIVSHIIKHRREELYIVKQNELAMSHSRESHPLERGFNLEPEEVCTNFLIQILFESMLVPVIMSTSQFKKYFWFGELELLPNNAQHDLKQLTQFICDCKKNNTSRTMNLDDLDVGFVSSKLILSCVNLNISVFINELDWVNRDNYENIEQLILASPSEVIPIELNLTFSHKRVSHKFRYERSTNYILKLRFLIERESLLDSLDSDGYLLLNPHSVEYYVSQSSGNHISLDGVSLLVLNPLINGKDVLDFNDLLEGQDIHFKSRSTVFQKVRIDLKNRFKDLKNKFSYKLIGPDVGMQPLILEGGLIKEGNRVVSRLEVNLDSKVVIIALEALEPEKRPRFIANLFQYLSSAQSHNKGISMNEQDLRLMIENFPEVFEHMLHDAKDWLNCGHFSIIRSKTLGSVMIADETGPFKIKGIRCRKLFEDNESVEIE

SEQ ID NO: 45 - protein, 95 aa, Artificial Sequence
  Pichinde Z protein Sequence
MGLRYSKEVRKRHGDEDVVGRVPMTLNLPQGLYGRFNCKSCWFVNKGLIRCKDHYLCLGCLTKMHSRGNLCEICGHSLPTKMEFLESPSAPPYEP